SEQUENCE LISTING (475 sequences)

SEQ ID NO: 1 - protein, 5 aa, Artificial Sequence
  source: /note=“Description of Artificial Sequence: Synthetic peptide”
SYAIS

SEQ ID NO: 2 - protein, 5 aa, Artificial Sequence
  source: /note=“Description of Artificial Sequence: Synthetic peptide”
SYYMH

SEQ ID NO: 3 - protein, 5 aa, Artificial Sequence
  source: /note=“Description of Artificial Sequence: Synthetic peptide”
RHLLH

SEQ ID NO: 4 - protein, 5 aa, Artificial Sequence
  source: /note=“Description of Artificial Sequence: Synthetic peptide”
SHHMH

SEQ ID NO: 5 - protein, 4 aa, Artificial Sequence
  source: /note=“Description of Artificial Sequence: Synthetic peptide”
RFMH

SEQ ID NO: 6 - protein, 5 aa, Artificial Sequence
  source: /note=“Description of Artificial Sequence: Synthetic peptide”
SYYIH

SEQ ID NO: 7 - protein, 5 aa, Artificial Sequence
  source: /note=“Description of Artificial Sequence: Synthetic peptide”
NYYIH

SEQ ID NO: 8 - protein, 5 aa, Artificial Sequence
  source: /note=“Description of Artificial Sequence: Synthetic peptide”
HYYMH

SEQ ID NO: 9 - protein, 5 aa, Artificial Sequence
  source: /note=“Description of Artificial Sequence: Synthetic peptide”
GYYMH

SEQ ID NO: 10 - protein, 5 aa, Artificial Sequence
  source: /note=“Description of Artificial Sequence: Synthetic peptide”
NYMYH

SEQ ID NO: 11 - protein, 5 aa, Artificial Sequence
  source: /note=“Description of Artificial Sequence: Synthetic peptide”
TYYVH

SEQ ID NO: 12 - protein, 5 aa, Artificial Sequence
  source: /note=“Description of Artificial Sequence: Synthetic peptide”
SYALS

SEQ ID NO: 13 - protein, 5 aa, Artificial Sequence
  source: /note=“Description of Artificial Sequence: Synthetic peptide”
RHYVH

SEQ ID NO: 14 - protein, 5 aa, Artificial Sequence
  source: /note=“Description of Artificial Sequence: Synthetic peptide”
SHYMH

SEQ ID NO: 15 - protein, 5 aa, Artificial Sequence
  source: /note=“Description of Artificial Sequence: Synthetic peptide”
DYYMH

SEQ ID NO: 16 - protein, 5 aa, Artificial Sequence
  source: /note=“Description of Artificial Sequence: Synthetic peptide”
GYTLH

SEQ ID NO: 17 - protein, 5 aa, Artificial Sequence
  source: /note=“Description of Artificial Sequence: Synthetic peptide”
NHYMH

SEQ ID NO: 18 - protein, 5 aa, Artificial Sequence
  source: /note=“Description of Artificial Sequence: Synthetic peptide”
NSYMH

SEQ ID NO: 19 - protein, 5 aa, Artificial Sequence
  source: /note=“Description of Artificial Sequence: Synthetic peptide”
TYYMH

SEQ ID NO: 20 - protein, 5 aa, Artificial Sequence
  source: /note=“Description of Artificial Sequence: Synthetic peptide”
RHFIH

SEQ ID NO: 21 - protein, 5 aa, Artificial Sequence
  source: /note=“Description of Artificial Sequence: Synthetic peptide”
TFGIS

SEQ ID NO: 22 - protein, 5 aa, Artificial Sequence
  source: /note=“Description of Artificial Sequence: Synthetic peptide”
SYGIN

SEQ ID NO: 23 - protein, 5 aa, Artificial Sequence
  source: /note=“Description of Artificial Sequence: Synthetic peptide”
NHYVH

SEQ ID NO: 24 - protein, 17 aa, Artificial Sequence
  source: /note=“Description of Artificial Sequence: Synthetic peptide”
VINPSAGSTDYAQKFQG

SEQ ID NO: 25 - protein, 17 aa, Artificial Sequence
  source: /note=“Description of Artificial Sequence: Synthetic peptide”
WMNPNSDIAGYAQKFQG

SEQ ID NO: 26 - protein, 17 aa, Artificial Sequence
  source: /note=“Description of Artificial Sequence: Synthetic peptide”
WISPQHGVRNYAQKFQG

SEQ ID NO: 27 - protein, 17 aa, Artificial Sequence
  source: /note=“Description of Artificial Sequence: Synthetic peptide”
WVSPSHGLTGYAQKFQG

SEQ ID NO: 28 - protein, 17 aa, Artificial Sequence
  source: /note=“Description of Artificial Sequence: Synthetic peptide”
WMSLNSGLTGYAQKFQG

SEQ ID NO: 29 - protein, 17 aa, Artificial Sequence
  source: /note=“Description of Artificial Sequence: Synthetic peptide”
WMKPSSGTTGYAQKFQG

SEQ ID NO: 30 - protein, 17 aa, Artificial Sequence
  source: /note=“Description of Artificial Sequence: Synthetic peptide”
WMNPNGDVAGYAQKFQG

SEQ ID NO: 31 - protein, 17 aa, Artificial Sequence
  source: /note=“Description of Artificial Sequence: Synthetic peptide”
GIDPNSGGTNYAQKFQG

SEQ ID NO: 32 - protein, 17 aa, Artificial Sequence
  source: /note=“Description of Artificial Sequence: Synthetic peptide”
WMNPDSGSTGYAQKFQG

SEQ ID NO: 33 - protein, 17 aa, Artificial Sequence
  source: /note=“Description of Artificial Sequence: Synthetic peptide”
WMSLNSGLTGYAQKFQG

SEQ ID NO: 34 - protein, 17 aa, Artificial Sequence
  source: /note=“Description of Artificial Sequence: Synthetic peptide”
WMNPNGDVAGYADSFQG

SEQ ID NO: 35 - protein, 17 aa, Artificial Sequence
  source: /note=“Description of Artificial Sequence: Synthetic peptide”
WISTYHGSTNYAQKFQG

SEQ ID NO: 36 - protein, 17 aa, Artificial Sequence
  source: /note=“Description of Artificial Sequence: Synthetic peptide”
WMNPNTVYTGSAQKFQG

SEQ ID NO: 37 - protein, 17 aa, Artificial Sequence
  source: /note=“Description of Artificial Sequence: Synthetic peptide”
RIIPAVGSVTYAQKFQG

SEQ ID NO: 38 - protein, 17 aa, Artificial Sequence
  source: /note=“Description of Artificial Sequence: Synthetic peptide”
GIIPIFGTANYAQKFQG

SEQ ID NO: 39 - protein, 17 aa, Artificial Sequence
  source: /note=“Description of Artificial Sequence: Synthetic peptide”
WMSPSSGITGYAQKFQG

SEQ ID NO: 40 - protein, 17 aa, Artificial Sequence
  source: /note=“Description of Artificial Sequence: Synthetic peptide”
WMTPSTGNAGYAQKFQG

SEQ ID NO: 41 - protein, 17 aa, Artificial Sequence
  source: /note=“Description of Artificial Sequence: Synthetic peptide”
WMNPNSGNTGYAQKFQG

SEQ ID NO: 42 - protein, 17 aa, Artificial Sequence
  source: /note=“Description of Artificial Sequence: Synthetic peptide”
WMHPNSGHTGYAQKFQG

SEQ ID NO: 43 - protein, 17 aa, Artificial Sequence
  source: /note=“Description of Artificial Sequence: Synthetic peptide”
WMNPNSGHTGNAQKFQG

SEQ ID NO: 44 - protein, 17 aa, Artificial Sequence
  source: /note=“Description of Artificial Sequence: Synthetic peptide”
WIDPNSGVTSSAQKFQG

SEQ ID NO: 45 - protein, 17 aa, Artificial Sequence
  source: /note=“Description of Artificial Sequence: Synthetic peptide”
WISPNSGVTDFTQKFQG

SEQ ID NO: 46 - protein, 17 aa, Artificial Sequence
  source: /note=“Description of Artificial Sequence: Synthetic peptide”
WMNPNSGHTGYAQRFQG

SEQ ID NO: 47 - protein, 17 aa, Artificial Sequence
  source: /note=“Description of Artificial Sequence: Synthetic peptide”
WMSPNGGNTGYAQKFQG

SEQ ID NO: 48 - protein, 17 aa, Artificial Sequence
  source: /note=“Description of Artificial Sequence: Synthetic peptide”
WMDPSSGYTGSAHKFQG

SEQ ID NO: 49 - protein, 17 aa, Artificial Sequence
  source: /note=“Description of Artificial Sequence: Synthetic peptide”
WMNPHSADTGYAEKFQG

SEQ ID NO: 50 - protein, 17 aa, Artificial Sequence
  source: /note=“Description of Artificial Sequence: Synthetic peptide”
WLTPSTGHAGYAQKFQG

SEQ ID NO: 51 - protein, 17 aa, Artificial Sequence
  source: /note=“Description of Artificial Sequence: Synthetic peptide”
WMNPNSGHTGYAQKFQG

SEQ ID NO: 52 - protein, 17 aa, Artificial Sequence
  source: /note=“Description of Artificial Sequence: Synthetic peptide”
WISPQHGVRNYAHKFQG

SEQ ID NO: 53 - protein, 17 aa, Artificial Sequence
  source: /note=“Description of Artificial Sequence: Synthetic peptide”
MINPSGGSTSYAQKFQG

SEQ ID NO: 54 - protein, 17 aa, Artificial Sequence
  source: /note=“Description of Artificial Sequence: Synthetic peptide”
WISPRSGVTSYAQKFQG

SEQ ID NO: 55 - protein, 17 aa, Artificial Sequence
  source: /note=“Description of Artificial Sequence: Synthetic peptide”
WMDPNSGNTGYAQKFQG

SEQ ID NO: 56 - protein, 17 aa, Artificial Sequence
  source: /note=“Description of Artificial Sequence: Synthetic peptide”
WMNPTGGITGYAQKFQG

SEQ ID NO: 57 - protein, 17 aa, Artificial Sequence
  source: /note=“Description of Artificial Sequence: Synthetic peptide”
WVSPIHGLTGYAPRFQG

SEQ ID NO: 58 - protein, 18 aa, Artificial Sequence
  source: /note=“Description of Artificial Sequence: Synthetic peptide”
DLYPYVVVVAAGSYGMDV

SEQ ID NO: 59 - protein, 12 aa, Artificial Sequence
  source: /note=“Description of Artificial Sequence: Synthetic peptide”
PSIVGAYDAFDI

SEQ ID NO: 60 - protein, 9 aa, Artificial Sequence
  source: /note=“Description of Artificial Sequence: Synthetic peptide”
ESVEGYFDL

SEQ ID NO: 61 - protein, 11 aa, Artificial Sequence
  source: /note=“Description of Artificial Sequence: Synthetic peptide”
DNWNVHDAFDI

SEQ ID NO: 62 - protein, 9 aa, Artificial Sequence
  source: /note=“Description of Artificial Sequence: Synthetic peptide”
GTYNDAFDI

SEQ ID NO: 63 - protein, 11 aa, Artificial Sequence
  source: /note=“Description of Artificial Sequence: Synthetic peptide”
EQWLVNDAFDI

SEQ ID NO: 64 - protein, 13 aa, Artificial Sequence
  source: /note=“Description of Artificial Sequence: Synthetic peptide”
DSSGWMRNDAFDI

SEQ ID NO: 65 - protein, 14 aa, Artificial Sequence
  source: /note=“Description of Artificial Sequence: Synthetic peptide”
SMFPTIFGDNAFDI

SEQ ID NO: 66 - protein, 14 aa, Artificial Sequence
  source: /note=“Description of Artificial Sequence: Synthetic peptide”
ALFPYPFYYYYMDV

SEQ ID NO: 67 - protein, 7 aa, Artificial Sequence
  source: /note=“Description of Artificial Sequence: Synthetic peptide”
DRGWFDP

SEQ ID NO: 68 - protein, 10 aa, Artificial Sequence
  source: /note=“Description of Artificial Sequence: Synthetic peptide”
DARGYSGYDL

SEQ ID NO: 69 - protein, 9 aa, Artificial Sequence
  source: /note=“Description of Artificial Sequence: Synthetic peptide”
EGRHGEYLY

SEQ ID NO: 70 - protein, 11 aa, Artificial Sequence
  source: /note=“Description of Artificial Sequence: Synthetic peptide”
EGWGSSGYFDY

SEQ ID NO: 71 - protein, 15 aa, Artificial Sequence
  source: /note=“Description of Artificial Sequence: Synthetic peptide”
HLFPTVFDDYYGMDV

SEQ ID NO: 72 - protein, 10 aa, Artificial Sequence
  source: /note=“Description of Artificial Sequence: Synthetic peptide”
GGYSYGSFQH

SEQ ID NO: 73 - protein, 10 aa, Artificial Sequence
  source: /note=“Description of Artificial Sequence: Synthetic peptide”
VRWSSDAFDI

SEQ ID NO: 74 - protein, 9 aa, Artificial Sequence
  source: /note=“Description of Artificial Sequence: Synthetic peptide”
EEWLGHFQH

SEQ ID NO: 75 - protein, 9 aa, Artificial Sequence
  source: /note=“Description of Artificial Sequence: Synthetic peptide”
ERFLGGMDV

SEQ ID NO: 76 - protein, 9 aa, Artificial Sequence
  source: /note=“Description of Artificial Sequence: Synthetic peptide”
GNWVDAFDI

SEQ ID NO: 77 - protein, 11 aa, Artificial Sequence
  source: /note=“Description of Artificial Sequence: Synthetic peptide”
ESEVMMAYFQH

SEQ ID NO: 78 - protein, 9 aa, Artificial Sequence
  source: /note=“Description of Artificial Sequence: Synthetic peptide”
ESWSGEFDY

SEQ ID NO: 79 - protein, 9 aa, Artificial Sequence
  source: /note=“Description of Artificial Sequence: Synthetic peptide”
EAVAGPMDV

SEQ ID NO: 80 - protein, 10 aa, Artificial Sequence
  source: /note=“Description of Artificial Sequence: Synthetic peptide”
DAWELLAFDI

SEQ ID NO: 81 - protein, 10 aa, Artificial Sequence
  source: /note=“Description of Artificial Sequence: Synthetic peptide”
DRWDGDYYSA

SEQ ID NO: 82 - protein, 10 aa, Artificial Sequence
  source: /note=“Description of Artificial Sequence: Synthetic peptide”
ESWELTGFDY

SEQ ID NO: 83 - protein, 9 aa, Artificial Sequence
  source: /note=“Description of Artificial Sequence: Synthetic peptide”
ERFAGGMDA

SEQ ID NO: 84 - protein, 8 aa, Artificial Sequence
  source: /note=“Description of Artificial Sequence: Synthetic peptide”
DSGGAFDI

SEQ ID NO: 85 - protein, 9 aa, Artificial Sequence
  source: /note=“Description of Artificial Sequence: Synthetic peptide”
EVFEGGMDV

SEQ ID NO: 86 - protein, 9 aa, Artificial Sequence
  source: /note=“Description of Artificial Sequence: Synthetic peptide”
EGYGGNYGN

SEQ ID NO: 87 - protein, 9 aa, Artificial Sequence
  source: /note=“Description of Artificial Sequence: Synthetic peptide”
EDFYGDFDY

SEQ ID NO: 88 - protein, 9 aa, Artificial Sequence
  source: /note=“Description of Artificial Sequence: Synthetic peptide”
ELSRWGFDY

SEQ ID NO: 89 - protein, 14 aa, Artificial Sequence
  source: /note=“Description of Artificial Sequence: Synthetic peptide”
DIFPTMIAGGGFDL

SEQ ID NO: 90 - protein, 10 aa, Artificial Sequence
  source: /note=“Description of Artificial Sequence: Synthetic peptide”
GGYSYGSFDY

SEQ ID NO: 91 - protein, 15 aa, Artificial Sequence
  source: /note=“Description of Artificial Sequence: Synthetic peptide”
GSFPLVFTIFGVGDV

SEQ ID NO: 92 - protein, 10 aa, Artificial Sequence
  source: /note=“Description of Artificial Sequence: Synthetic peptide”
DLDYVRAFDI

SEQ ID NO: 93 - protein, 9 aa, Artificial Sequence
  source: /note=“Description of Artificial Sequence: Synthetic peptide”
ESWGGYFDL

SEQ ID NO: 94 - protein, 9 aa, Artificial Sequence
  source: /note=“Description of Artificial Sequence: Synthetic peptide”
DRTTYAFDI

SEQ ID NO: 95 - protein, 11 aa, Artificial Sequence
  source: /note=“Description of Artificial Sequence: Synthetic peptide”
VHGSGSDGMDV

SEQ ID NO: 96 - protein, 11 aa, Artificial Sequence
  source: /note=“Description of Artificial Sequence: Synthetic peptide”
RASQGIDSYLA

SEQ ID NO: 97 - protein, 11 aa, Artificial Sequence
  source: /note=“Description of Artificial Sequence: Synthetic peptide”
RASQSISSWLA

SEQ ID NO: 98 - protein, 11 aa, Artificial Sequence
  source: /note=“Description of Artificial Sequence: Synthetic peptide”
RASQSISSYLN

SEQ ID NO: 99 - protein, 11 aa, Artificial Sequence
  source: /note=“Description of Artificial Sequence: Synthetic peptide”
RASQGISSYLA

SEQ ID NO: 100 - protein, 11 aa, Artificial Sequence
  source: /note=“Description of Artificial Sequence: Synthetic peptide”
RASQTISNYLN

SEQ ID NO: 101 - protein, 12 aa, Artificial Sequence
  source: /note=“Description of Artificial Sequence: Synthetic peptide”
RASQSVDRNYVT

SEQ ID NO: 102 - protein, 11 aa, Artificial Sequence
  source: /note=“Description of Artificial Sequence: Synthetic peptide”
RASQGISQYLA

SEQ ID NO: 103 - protein, 11 aa, Artificial Sequence
  source: /note=“Description of Artificial Sequence: Synthetic peptide”
QASQDIGNYLN

SEQ ID NO: 104 - protein, 11 aa, Artificial Sequence
  source: /note=“Description of Artificial Sequence: Synthetic peptide”
RASQGIRNDLG

SEQ ID NO: 105 - protein, 11 aa, Artificial Sequence
  source: /note=“Description of Artificial Sequence: Synthetic peptide”
RASQIIGNYLA

SEQ ID NO: 106 - protein, 11 aa, Artificial Sequence
  source: /note=“Description of Artificial Sequence: Synthetic peptide”
RASQIISSYLN

SEQ ID NO: 107 - protein, 11 aa, Artificial Sequence
  source: /note=“Description of Artificial Sequence: Synthetic peptide”
QASQDISNYLN

SEQ ID NO: 108 - protein, 11 aa, Artificial Sequence
  source: /note=“Description of Artificial Sequence: Synthetic peptide”
RASQGISNYLA

SEQ ID NO: 109 - protein, 11 aa, Artificial Sequence
  source: /note=“Description of Artificial Sequence: Synthetic peptide”
RASQGISNNLN

SEQ ID NO: 110 - protein, 11 aa, Artificial Sequence
  source: /note=“Description of Artificial Sequence: Synthetic peptide”
RASQGISNGLS

SEQ ID NO: 111 - protein, 11 aa, Artificial Sequence
  source: /note=“Description of Artificial Sequence: Synthetic peptide”
RASQSITGWLA

SEQ ID NO: 112 - protein, 11 aa, Artificial Sequence
  source: /note=“Description of Artificial Sequence: Synthetic peptide”
RASQGIRNDLA

SEQ ID NO: 113 - protein, 11 aa, Artificial Sequence
  source: /note=“Description of Artificial Sequence: Synthetic peptide”
QASQDISSYLN

SEQ ID NO: 114 - protein, 11 aa, Artificial Sequence
  source: /note=“Description of Artificial Sequence: Synthetic peptide”
RASQSITTYLN

SEQ ID NO: 115 - protein, 11 aa, Artificial Sequence
  source: /note=“Description of Artificial Sequence: Synthetic peptide”
RASQSVSTWLA

SEQ ID NO: 116 - protein, 11 aa, Artificial Sequence
  source: /note=“Description of Artificial Sequence: Synthetic peptide”
RASQSISNWLA

SEQ ID NO: 117 - protein, 11 aa, Artificial Sequence
  source: /note=“Description of Artificial Sequence: Synthetic peptide”
QASQGISNYLA

SEQ ID NO: 118 - protein, 12 aa, Artificial Sequence
  source: /note=“Description of Artificial Sequence: Synthetic peptide”
RASQSLSSSSLA

SEQ ID NO: 119 - protein, 11 aa, Artificial Sequence
  source: /note=“Description of Artificial Sequence: Synthetic peptide”
RASEHIANWLA

SEQ ID NO: 120 - protein, 11 aa, Artificial Sequence
  source: /note=“Description of Artificial Sequence: Synthetic peptide”
RASQSVGSWVA

SEQ ID NO: 121 - protein, 11 aa, Artificial Sequence
  source: /note=“Description of Artificial Sequence: Synthetic peptide”
RASQSISPWLA

SEQ ID NO: 122 - protein, 11 aa, Artificial Sequence
  source: /note=“Description of Artificial Sequence: Synthetic peptide”
RASQGISRYLA

SEQ ID NO: 123 - protein, 12 aa, Artificial Sequence
  source: /note=“Description of Artificial Sequence: Synthetic peptide”
RASQTVSSNYLA

SEQ ID NO: 124 - protein, 11 aa, Artificial Sequence
  source: /note=“Description of Artificial Sequence: Synthetic peptide”
RSSQGIRNDLS

SEQ ID NO: 125 - protein, 11 aa, Artificial Sequence
  source: /note=“Description of Artificial Sequence: Synthetic peptide”
RDSHSITTWLA

SEQ ID NO: 126 - protein, 11 aa, Artificial Sequence
  source: /note=“Description of Artificial Sequence: Synthetic peptide”
RASQSISRWLA

SEQ ID NO: 127 - protein, 11 aa, Artificial Sequence
  source: /note=“Description of Artificial Sequence: Synthetic peptide”
RASQVIRNDLA

SEQ ID NO: 128 - skipped

SEQ ID NO: 129 - protein, 7 aa, Artificial Sequence
  source: /note=“Description of Artificial Sequence: Synthetic peptide”
AASSLQS

SEQ ID NO: 130 - protein, 7 aa, Artificial Sequence
  source: /note=“Description of Artificial Sequence: Synthetic peptide”
GASNLQS

SEQ ID NO: 131 - protein, 7 aa, Artificial Sequence
  source: /note=“Description of Artificial Sequence: Synthetic peptide”
GASTRAT

SEQ ID NO: 132 - protein, 7 aa, Artificial Sequence
  source: /note=“Description of Artificial Sequence: Synthetic peptide”
GASNLHS

SEQ ID NO: 133 - protein, 7 aa, Artificial Sequence
  source: /note=“Description of Artificial Sequence: Synthetic peptide”
AASSLES

SEQ ID NO: 134 - protein, 7 aa, Artificial Sequence
  source: /note=“Description of Artificial Sequence: Synthetic peptide”
SASNLQS

SEQ ID NO: 135 - protein, 7 aa, Artificial Sequence
  source: /note=“Description of Artificial Sequence: Synthetic peptide”
AASTLES

SEQ ID NO: 136 - protein, 7 aa, Artificial Sequence
  source: /note=“Description of Artificial Sequence: Synthetic peptide”
HASILET

SEQ ID NO: 137 - protein, 7 aa, Artificial Sequence
  source: /note=“Description of Artificial Sequence: Synthetic peptide”
AASTLQS

SEQ ID NO: 138 - protein, 7 aa, Artificial Sequence
  source: /note=“Description of Artificial Sequence: Synthetic peptide”
AATTLQS

SEQ ID NO: 139 - protein, 7 aa, Artificial Sequence
  source: /note=“Description of Artificial Sequence: Synthetic peptide”
DATHLET

SEQ ID NO: 140 - protein, 7 aa, Artificial Sequence
  source: /note=“Description of Artificial Sequence: Synthetic peptide”
AASSLQT

SEQ ID NO: 141 - protein, 7 aa, Artificial Sequence
  source: /note=“Description of Artificial Sequence: Synthetic peptide”
AASSLQG

SEQ ID NO: 142 - protein, 7 aa, Artificial Sequence
  source: /note=“Description of Artificial Sequence: Synthetic peptide”
AASNLES

SEQ ID NO: 143 - protein, 7 aa, Artificial Sequence
  source: /note=“Description of Artificial Sequence: Synthetic peptide”
DVSHLES

SEQ ID NO: 144 - protein, 7 aa, Artificial Sequence
  source: /note=“Description of Artificial Sequence: Synthetic peptide”
DASSLQS

SEQ ID NO: 145 - protein, 7 aa, Artificial Sequence
  source: /note=“Description of Artificial Sequence: Synthetic peptide”
GVSSLES

SEQ ID NO: 146 - protein, 7 aa, Artificial Sequence
  source: /note=“Description of Artificial Sequence: Synthetic peptide”
PASTLQS

SEQ ID NO: 147 - protein, 7 aa, Artificial Sequence
  source: /note=“Description of Artificial Sequence: Synthetic peptide”
DASNLET

SEQ ID NO: 148 - protein, 7 aa, Artificial Sequence
  source: /note=“Description of Artificial Sequence: Synthetic peptide”
GASTRAS

SEQ ID NO: 149 - protein, 7 aa, Artificial Sequence
  source: /note=“Description of Artificial Sequence: Synthetic peptide”
DSSSLQT

SEQ ID NO: 150 - protein, 7 aa, Artificial Sequence
  source: /note=“Description of Artificial Sequence: Synthetic peptide”
ATSTLQS

SEQ ID NO: 151 - protein, 7 aa, Artificial Sequence
  source: /note=“Description of Artificial Sequence: Synthetic peptide”
LASNSHS

SEQ ID NO: 152 - protein, 9 aa, Artificial Sequence
  source: /note=“Description of Artificial Sequence: Synthetic peptide”
QQSYSTPIT

SEQ ID NO: 153 - protein, 9 aa, Artificial Sequence
  source: /note=“Description of Artificial Sequence: Synthetic peptide”
QQSYTTPIT

SEQ ID NO: 154 - protein, 9 aa, Artificial Sequence
  source: /note=“Description of Artificial Sequence: Synthetic peptide”
QQIFSTPLT

SEQ ID NO: 155 - protein, 9 aa, Artificial Sequence
  source: /note=“Description of Artificial Sequence: Synthetic peptide”
QQSYSTPLT

SEQ ID NO: 156 - protein, 9 aa, Artificial Sequence
  source: /note=“Description of Artificial Sequence: Synthetic peptide”
QQSYTTPYT

SEQ ID NO: 157 - protein, 9 aa, Artificial Sequence
  source: /note=“Description of Artificial Sequence: Synthetic peptide”
QQTFTTPLT

SEQ ID NO: 158 - protein, 9 aa, Artificial Sequence
  source: /note=“Description of Artificial Sequence: Synthetic peptide”
QQANSFPFT

SEQ ID NO: 159 - protein, 9 aa, Artificial Sequence
  source: /note=“Description of Artificial Sequence: Synthetic peptide”
QQSYTTPYS

SEQ ID NO: 160 - protein, 9 aa, Artificial Sequence
  source: /note=“Description of Artificial Sequence: Synthetic peptide”
QQTFITPLT

SEQ ID NO: 161 - protein, 8 aa, Artificial Sequence
  source: /note=“Description of Artificial Sequence: Synthetic peptide”
QQSYSTPT

SEQ ID NO: 162 - protein, 9 aa, Artificial Sequence
  source: /note=“Description of Artificial Sequence: Synthetic peptide”
QQGFSTPFT

SEQ ID NO: 163 - protein, 9 aa, Artificial Sequence
  source: /note=“Description of Artificial Sequence: Synthetic peptide”
QQSFTNPVT

SEQ ID NO: 164 - protein, 9 aa, Artificial Sequence
  source: /note=“Description of Artificial Sequence: Synthetic peptide”
QQSYSAPYT

SEQ ID NO: 165 - protein, 9 aa, Artificial Sequence
  source: /note=“Description of Artificial Sequence: Synthetic peptide”
QQSYSTPYT

SEQ ID NO: 166 - protein, 9 aa, Artificial Sequence
  source: /note=“Description of Artificial Sequence: Synthetic peptide”
QQSHSTPLT

SEQ ID NO: 167 - protein, 9 aa, Artificial Sequence
  source: /note=“Description of Artificial Sequence: Synthetic peptide”
QQSYSTPFT

SEQ ID NO: 168 - protein, 9 aa, Artificial Sequence
  source: /note=“Description of Artificial Sequence: Synthetic peptide”
QQYGSSPFT

SEQ ID NO: 169 - protein, 9 aa, Artificial Sequence
  source: /note=“Description of Artificial Sequence: Synthetic peptide”
QQTYSTPIT

SEQ ID NO: 170 - protein, 9 aa, Artificial Sequence
  source: /note=“Description of Artificial Sequence: Synthetic peptide”
QQYYTTPLT

SEQ ID NO: 171 - protein, 9 aa, Artificial Sequence
  source: /note=“Description of Artificial Sequence: Synthetic peptide”
QQSFSTPLT

SEQ ID NO: 172 - protein, 9 aa, Artificial Sequence
  source: /note=“Description of Artificial Sequence: Synthetic peptide”
QQSYSTPPT

SEQ ID NO: 173 - protein, 9 aa, Artificial Sequence
  source: /note=“Description of Artificial Sequence: Synthetic peptide”
LQHNSYPLT

SEQ ID NO: 174 - protein, 9 aa, Artificial Sequence
  source: /note=“Description of Artificial Sequence: Synthetic peptide”
QQSYSTPVT

SEQ ID NO: 175 - protein, 9 aa, Artificial Sequence
  source: /note=“Description of Artificial Sequence: Synthetic peptide”
QHFYNTQYT

SEQ ID NO: 176 - protein, 10 aa, Artificial Sequence
  source: /note=“Description of Artificial Sequence: Synthetic peptide”
QQSLQYPSHF

SEQ ID NO: 177 - protein, 30 aa, Artificial Sequence
  source: /note=“Description of Artificial Sequence: Synthetic polypeptide”
QVQLVQSGAEVKKPGASVKVSCKASGGTFS

SEQ ID NO: 178 - protein, 30 aa, Artificial Sequence
  source: /note=“Description of Artificial Sequence: Synthetic polypeptide”
QVQLVQSGAEVKKPGASVKVSCKASGYTFT

SEQ ID NO: 179 - protein, 30 aa, Artificial Sequence
  source: /note=“Description of Artificial Sequence: Synthetic polypeptide”
QVQLVQSGAEVKKPGASVKVSCKASGYTFN

SEQ ID NO: 180 - protein, 30 aa, Artificial Sequence
  source: /note=“Description of Artificial Sequence: Synthetic polypeptide”
QVQLVQSGAEVKKPGASVKVSCKASGNTFT

SEQ ID NO: 181 - protein, 30 aa, Artificial Sequence
  source: /note=“Description of Artificial Sequence: Synthetic polypeptide”
QVQLVQSGAEVKKPGASVKVSCKASGHSFT

SEQ ID NO: 182 - protein, 30 aa, Artificial Sequence
  source: /note=“Description of Artificial Sequence: Synthetic polypeptide”
QVQLVQSGAEVKKPGASVKVSCKASGYPFT

SEQ ID NO: 183 - protein, 30 aa, Artificial Sequence
  source: /note=“Description of Artificial Sequence: Synthetic polypeptide”
QVQLVQSGAEVKKPGASVKVSCKASGYRFT

SEQ ID NO: 184 - protein, 30 aa, Artificial Sequence
  source: /note=“Description of Artificial Sequence: Synthetic polypeptide”
QVQLVQSGAEVKKPGSSVKVSCKASGGTFS

SEQ ID NO: 185 - protein, 30 aa, Artificial Sequence
  source: /note=“Description of Artificial Sequence: Synthetic polypeptide”
QVQLVQSGAEVKKPGASVKVSCKASGDTFT

SEQ ID NO: 186 - protein, 30 aa, Artificial Sequence
  source: /note=“Description of Artificial Sequence: Synthetic polypeptide”
QVQLAQSGAEVKKPGASVKVSCKASGYTFT

SEQ ID NO: 187 - protein, 30 aa, Artificial Sequence
  source: /note=“Description of Artificial Sequence: Synthetic polypeptide”
QVQLVQSGAEVKKPGASVKVSCKASGYTFS

SEQ ID NO: 188 - protein, 30 aa, Artificial Sequence
  source: /note=“Description of Artificial Sequence: Synthetic polypeptide”
QVQLVQSGAEVKKPGASVKVSCKASGYPFS

SEQ ID NO: 189 - protein, 30 aa, Artificial Sequence
  source: /note=“Description of Artificial Sequence: Synthetic polypeptide”
QVQLVQSGAEVKKPGASVKVSCKASGYSFT

SEQ ID NO: 190 - protein, 30 aa, Artificial Sequence
  source: /note=“Description of Artificial Sequence: Synthetic polypeptide”
QVQLVQSGAEVKKPGASVKVSCKASGYTFI

SEQ ID NO: 191 - protein, 30 aa, Artificial Sequence
  source: /note=“Description of Artificial Sequence: Synthetic polypeptide”
QVQLVQSGAEVKKPGSSVKVSCKASGYTFT

SEQ ID NO: 192 - protein, 14 aa, Artificial Sequence
  source: /note=“Description of Artificial Sequence: Synthetic peptide”
WVRQAPGQGLEWMG

SEQ ID NO: 193 - protein, 14 aa, Artificial Sequence
  source: /note=“Description of Artificial Sequence: Synthetic peptide”
WVRQAPGQGLEWLG

SEQ ID NO: 194 - protein, 14 aa, Artificial Sequence
  source: /note=“Description of Artificial Sequence: Synthetic peptide”
WVRQAPGQGLEWIG

SEQ ID NO: 195 - protein, 14 aa, Artificial Sequence
  source: /note=“Description of Artificial Sequence: Synthetic peptide”
WVRQAPGQGLEWVG

SEQ ID NO: 196 - protein, 32 aa, Artificial Sequence
  source: /note=“Description of Artificial Sequence: Synthetic polypeptide”
RVTMTRDTSTSTVYMELSSLRSEDTAVYYCAR

SEQ ID NO: 197 - protein, 32 aa, Artificial Sequence
  source: /note=“Description of Artificial Sequence: Synthetic polypeptide”
RVTMTRDTSTSTVYMELSSLRSEDTAVYYCAI

SEQ ID NO: 198 - protein, 32 aa, Artificial Sequence
  source: /note=“Description of Artificial Sequence: Synthetic polypeptide”
RVTMTRDTSTSTVYMELSSLRSEDTAVYYCTR

SEQ ID NO: 199 - protein, 32 aa, Artificial Sequence
  source: /note=“Description of Artificial Sequence: Synthetic polypeptide”
RVTITADESTSTAYMELSSLRSEDTAVYYCAR

SEQ ID NO: 200 - protein, 32 aa, Artificial Sequence
  source: /note=“Description of Artificial Sequence: Synthetic polypeptide”
RVTMTRDTSTSTVNMELSSLRSEDTAVYYCAR

SEQ ID NO: 201 - protein, 32 aa, Artificial Sequence
  source: /note=“Description of Artificial Sequence: Synthetic polypeptide”
RVTMTRDTSTSTVYMELSSLRSEDTAVYYCAE

SEQ ID NO: 202 - protein, 11 aa, Artificial Sequence
  source: /note=“Description of Artificial Sequence: Synthetic peptide”
WGQGTLVTVSS

SEQ ID NO: 203 - protein, 11 aa, Artificial Sequence
  source: /note=“Description of Artificial Sequence: Synthetic peptide”
WGQGTTVTVSS

SEQ ID NO: 204 - protein, 11 aa, Artificial Sequence
  source: /note=“Description of Artificial Sequence: Synthetic peptide”
WGQGTMVTVSS

SEQ ID NO: 205 - protein, 11 aa, Artificial Sequence
  source: /note=“Description of Artificial Sequence: Synthetic peptide”
WGPGTMVTVSS

SEQ ID NO: 206 - protein, 11 aa, Artificial Sequence
  source: /note=“Description of Artificial Sequence: Synthetic peptide”
WGRGTLVTVSS

SEQ ID NO: 207 - protein, 23 aa, Artificial Sequence
  source: /note=“Description of Artificial Sequence: Synthetic peptide”
DIQMTQSPSSLSASVGDRVTITC

SEQ ID NO: 208 - protein, 23 aa, Artificial Sequence
  source: /note=“Description of Artificial Sequence: Synthetic peptide”
EIVMTQSPATLSVSPGERATLSC

SEQ ID NO: 209 - protein, 23 aa, Artificial Sequence
  source: /note=“Description of Artificial Sequence: Synthetic peptide”
DIQITHSPSSLSASVGYRLTITC

SEQ ID NO: 210 - protein, 15 aa, Artificial Sequence
  source: /note=“Description of Artificial Sequence: Synthetic peptide”
WYQQKPGKAPKLLIY

SEQ ID NO: 211 - protein, 15 aa, Artificial Sequence
  source: /note=“Description of Artificial Sequence: Synthetic peptide”
WYQQKPGQAPRLLIY

SEQ ID NO: 212 - protein, 15 aa, Artificial Sequence
  source: /note=“Description of Artificial Sequence: Synthetic peptide”
WYHQKPWNAPKLMIY

SEQ ID NO: 213 - protein, 32 aa, Artificial Sequence
  source: /note=“Description of Artificial Sequence: Synthetic polypeptide”
GVPSRFSGSGSGTDFTLTISSLQPEDFATYYC

SEQ ID NO: 214 - protein, 32 aa, Artificial Sequence
  source: /note=“Description of Artificial Sequence: Synthetic polypeptide”
GIPARFSGSGSGTEFTLTISSLQSEDFAVYYC

SEQ ID NO: 215 - protein, 32 aa, Artificial Sequence
  source: /note=“Description of Artificial Sequence: Synthetic polypeptide”
GVPSRFSGSGSGTYFTLTISSLQPEDFATYYC

SEQ ID NO: 216 - protein, 32 aa, Artificial Sequence
  source: /note=“Description of Artificial Sequence: Synthetic polypeptide”
GVPSRFSGSGSGTDFTLTISSLQPEDFAPYYC

SEQ ID NO: 217 - protein, 10 aa, Artificial Sequence
  source: /note=“Description of Artificial Sequence: Synthetic peptide”
FGGGTKVEIK

SEQ ID NO: 218 - protein, 10 aa, Artificial Sequence
  source: /note=“Description of Artificial Sequence: Synthetic peptide”
FGQGTKVEIK

SEQ ID NO: 219 - protein, 10 aa, Artificial Sequence
  source: /note=“Description of Artificial Sequence: Synthetic peptide”
FGQGTRLEIK

SEQ ID NO: 220 - protein, 10 aa, Artificial Sequence
  source: /note=“Description of Artificial Sequence: Synthetic peptide”
FGGGTKLEIK

SEQ ID NO: 221 - protein, 10 aa, Artificial Sequence
  source: /note=“Description of Artificial Sequence: Synthetic peptide”
FGQGTKLEIK

SEQ ID NO: 222 - protein, 10 aa, Artificial Sequence
  source: /note=“Description of Artificial Sequence: Synthetic peptide”
FGPGTKVDIK

SEQ ID NO: 223 - protein, 127 aa, Artificial Sequence
  source: /note=“Description of Artificial Sequence: Synthetic polypeptide”
QVQLVQSGAEVKKPGASVKVSCKASGGTFSSYAISWVRQAPGQGLEWMGVINPSAGSTDYAQKFQGRVTMTRDTSTSTVYMELSSLRSEDTAVYYCARDLYPYVVVVAAGSYGMDVWGQGTLVTVSS

SEQ ID NO: 224 - protein, 121 aa, Artificial Sequence
  source: /note=“Description of Artificial Sequence: Synthetic polypeptide”
QVQLVQSGAEVKKPGASVKVSCKASGYTFTSYYMHWVRQAPGQGLEWMGWMNPNSDIAGYAQKFQGRVTMTRDTSTSTVYMELSSLRSEDTAVYYCAIPSIVGAYDAFDIWGQGTLVTVSS

SEQ ID NO: 225 - protein, 118 aa, Artificial Sequence
  source: /note=“Description of Artificial Sequence: Synthetic polypeptide”
QVQLVQSGAEVKKPGASVKVSCKASGYTFTRHLLHWVRQAPGQGLEWMGWISPQHGVRNYAQKFQGRVTMTRDTSTSTVYMELSSLRSEDTAVYYCARESVEGYFDLWGQGTLVTVSS

SEQ ID NO: 226 - protein, 120 aa, Artificial Sequence
  source: /note=“Description of Artificial Sequence: Synthetic polypeptide”
QVQLVQSGAEVKKPGASVKVSCKASGYTFTSHHMHWVRQAPGQGLEWMGWVSPSHGLTGYAQKFQGRVTMTRDTSTSTVYMELSSLRSEDTAVYYCARDNWNVHDAFDIWGQGTLVTVSS

SEQ ID NO: 227 - protein, 117 aa, Artificial Sequence
  source: /note=“Description of Artificial Sequence: Synthetic polypeptide”
QVQLVQSGAEVKKPGASVKVSCKASGYTFNRFMHWVRQAPGQGLEWMGWMSLNSGLTGYAQKFQGRVTMTRDTSTSTVYMELSSLRSEDTAVYYCTRGTYNDAFDIWGQGTLVTVSS

SEQ ID NO: 228 - protein, 120 aa, Artificial Sequence
  source: /note=“Description of Artificial Sequence: Synthetic polypeptide”
QVQLVQSGAEVKKPGASVKVSCKASGYTFTSYYIHWVRQAPGQGLEWMGWMKPSSGTTGYAQKFQGRVTMTRDTSTSTVYMELSSLRSEDTAVYYCAREQWLVNDAFDIWGQGTLVTVSS

SEQ ID NO: 229 - protein, 120 aa, Artificial Sequence; identical to SEQ ID NO: 228
  source: /note=“Description of Artificial Sequence: Synthetic polypeptide”

SEQ ID NO: 230 - protein, 123 aa, Artificial Sequence
  source: /note=“Description of Artificial Sequence: Synthetic polypeptide”
QVQLVQSGAEVKKPGASVKVSCKASGNTFTSYYMHWVRQAPGQGLEWMGGIDPNSGGTNYAQKFQGRVTMTRDTSTSTVYMELSSLRSEDTAVYYCARSMFPTIFGDNAFDIWGQGTLVTVSS

SEQ ID NO: 231 - protein, 123 aa, Artificial Sequence
  source: /note=“Description of Artificial Sequence: Synthetic polypeptide”
QVQLVQSGAEVKKPGASVKVSCKASGHSFTHYYMHWVRQAPGQGLEWMGWMNPDSGSTGYAQKFQGRVTMTRDTSTSTVYMELSSLRSEDTAVYYCARALFPYPFYYYYMDVWGQGTLVTVSS

SEQ ID NO: 232 - protein, 116 aa, Artificial Sequence
  source: /note=“Description of Artificial Sequence: Synthetic polypeptide”
QVQLVQSGAEVKKPGASVKVSCKASGYTFTGYYMHWVRQAPGQGLEWIGWMSLNSGLTGYAQKFQGRVTMTRDTSTSTVYMELSSLRSEDTAVYYCARDRGWFDPWGQGTLVTVSS

SEQ ID NO: 233 - protein, 122 aa, Artificial Sequence
  source: /note=“Description of Artificial Sequence: Synthetic polypeptide”
QVQLVQSGAEVKKPGASVKVSCKASGYTFTNYYIHWVRQAPGQGLEWLGWMNPNGDVAGYADSFQGRVTMTRDTSTSTVYMELSSLRSEDTAVYYCAIDSSGWMRNDAFDIWGQGTLVTVSS

SEQ ID NO: 234 - protein, 119 aa, Artificial Sequence
  source: /note=“Description of Artificial Sequence: Synthetic polypeptide”
QVQLVQSGAEVKKPGASVKVSCKASGGTFSNYMYHWVRQAPGQGLEWMGWISTYHGSTNYAQKFQGRVTMTRDTSTSTVYMELSSLRSEDTAVYYCARDARGYSGYDLWGQGTLVTVSS

SEQ ID NO: 235 - protein, 118 aa, Artificial Sequence
  source: /note=“Description of Artificial Sequence: Synthetic polypeptide”
QVQLVQSGAEVKKPGASVKVSCKASGYPFTSYYMHWVRQAPGQGLEWMGWMNPNSDIAGYAQKFQGRVTMTRDTSTSTVYMELSSLRSEDTAVYYCAREGRHGEYLYWGQGTLVTVSS

SEQ ID NO: 236 - protein, 120 aa, Artificial Sequence
  source: /note=“Description of Artificial Sequence: Synthetic polypeptide”
QVQLVQSGAEVKKPGASVKVSCKASGYRFTTYYVHWVRQAPGQGLEWMGWMNPNTVYTGSAQKFQGRVTMTRDTSTSTVYMELSSLRSEDTAVYYCAREGWGSSGYFDYWGQGTLVTVSS

SEQ ID NO: 237 - protein, 124 aa, Artificial Sequence
  source: /note=“Description of Artificial Sequence: Synthetic polypeptide”
QVQLVQSGAEVKKPGSSVKVSCKASGGTFSSYALSWVRQAPGQGLEWMGRIIPAVGSVTYAQKFQGRVTITADESTSTAYMELSSLRSEDTAVYYCARHLFPTVFDDYYGMDVWGQGTLVTVSS

SEQ ID NO: 238 - protein, 119 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGYSYGSFQHWGQGTLVTVSS

SEQ ID NO: 239 - protein, 119 aa, Artificial Sequence
  source: /note=“Description of Artificial Sequence: Synthetic polypeptide”
QVQLVQSGAEVKKPGASVKVSCKASGDTFTRHYVHWVRQAPGQGLEWMGWMSPSSGITGYAQKFQGRVTMTRDTSTSTVYMELSSLRSEDTAVYYCARVRWSSDAFDIWGQGTLVTVSS

SEQ ID NO: 240 - protein, 118 aa, Artificial Sequence
  source: /note=“Description of Artificial Sequence: Synthetic polypeptide”
QVQLVQSGAEVKKPGASVKVSCKASGYTFTSYYMHWVRQAPGQGLEWVGWMTPSTGNAGYAQKFQGRVTMTRDTSTSTVYMELSSLRSEDTAVYYCAREEWLGHFQHWGQGTLVTVSS

SEQ ID NO: 241 - protein, 118 aa, Artificial Sequence
  source: /note=“Description of Artificial Sequence: Synthetic polypeptide”
QVQLVQSGAEVKKPGASVKVSCKASGYTFTSHYMHWVRQAPGQGLEWMGWMNPNSGNTGYAQKFQGRVTMTRDTSTSTVYMELSSLRSEDTAVYYCARERFLGGMDVWGQGTTVTVSS

SEQ ID NO: 242 - protein, 118 aa, Artificial Sequence
  source: /note=“Description of Artificial Sequence: Synthetic polypeptide”
QVQLVQSGAEVKKPGASVKVSCKASGYTFTDYYMHWVRQAPGQGLEWMGWMHPNSGHTGYAQKFQGRVTMTRDTSTSTVNMELSSLRSEDTAVYYCAREEWLGHFQHWGQGTLVTVSS

SEQ ID NO: 243 - protein, 118 aa, Artificial Sequence
  source: /note=“Description of Artificial Sequence: Synthetic polypeptide”
QVQLVQSGAEVKKPGASVKVSCKASGYTFTSHYMHWVRQAPGQGLEWMGWMNPNSGHTGNAQKFQGRVTMTRDTSTSTVYMELSSLRSEDTAVYYCARGNWVDAFDIWGQGTMVTVSS

SEQ ID NO: 244 - protein, 120 aa, Artificial Sequence
  source: /note=“Description of Artificial Sequence: Synthetic polypeptide”
QVQLVQSGAEVKKPGASVKVSCKASGYTFTGYTLHWVRQAPGQGLEWMGWIDPNSGVTSSAQKFQGRVTMTRDTSTSTVYMELSSLRSEDTAVYYCARESEVMMAYFQHWGQGTLVTVSS

SEQ ID NO: 245 - protein, 118 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWISPNSGVTDFTQKFQGRVTMTRDTSTSTVYMELSSLRSEDTAVYYCARESWSGEFDYWGQGTLVTVSS

SEQ ID NO: 246 - protein, 118 aa, Artificial Sequence
  source: /note=“Description of Artificial Sequence: Synthetic polypeptide”
QVQLVQSGAEVKKPGASVKVSCKASGDTFTNHYMHWVRQAPGQGLEWMGWMNPNSGHTGYAQRFQGRVTMTRDTSTSTVYMELSSLRSEDTAVYYCAREAVAGPMDVWGQGTTVTVSS

SEQ ID NO: 247 - protein, 119 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWMNPNSDIAGYAQKFQGRVTMTRDTSTSTVYMELSSLRSEDTAVYYCARDAWELLAFDIWGQGTLVTVSS

SEQ ID NO: 248 - protein, 119 aa, Artificial Sequence
  source: /note=“Description of Artificial Sequence: Synthetic polypeptide”
QVQLAQSGAEVKKPGASVKVSCKASGYTFTNHYMHWVRQAPGQGLEWMGWMNPNSGNTGYAQKFQGRVTMTRDTSTSTVYMELSSLRSEDTAVYYCARDRWDGDYYSAWGQGTLVTVSS

SEQ ID NO: 249 - protein, 119 aa, Artificial Sequence
  source: /note=“Description of Artificial Sequence: Synthetic polypeptide”
QVQLVQSGAEVKKPGASVKVSCKASGYTFTNYYIHWVRQAPGQGLEWMGWMSPNGGNTGYAQKFQGRVTMTRDTSTSTVYMELSSLRSEDTAVYYCARESWELTGFDYWGQGTLVTVSS

SEQ ID NO: 250 - protein, 118 aa, Artificial Sequence
  source: /note=“Description of Artificial Sequence: Synthetic polypeptide”
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARERFAGGMDAWGQGTTVTVSS

SEQ ID NO: 251 - protein, 117 aa, Artificial Sequence
  source: /note=“Description of Artificial Sequence: Synthetic polypeptide”
QVQLVQSGAEVKKPGASVKVSCKASGYTFSNSYMHWVRQAPGQGLEWMGWMDPSSGYTGSAHKFQGRVTMTRDTSTSTVYMELSSLRSEDTAVYYCAEDSGGAFDIWGQGTMVTVSS

SEQ ID NO: 252 - protein, 118 aa, Artificial Sequence
  source: /note=“Description of Artificial Sequence: Synthetic polypeptide”
QVQLVQSGAEVKKPGASVKVSCKASGYPFSTYYMHWVRQAPGQGLEWMGWMNPHSADTGYAEKFQGRVTMTRDTSTSTVYMELSSLRSEDTAVYYCAREVFEGGMDVWGQGTTVTVSS

SEQ ID NO: 253 - protein, 118 aa, Artificial Sequence
  source: /note=“Description of Artificial Sequence: Synthetic polypeptide”
QVQLVQSGAEVKKPGASVKVSCKASGYTFTSYYMHWVRQAPGQGLEWMGWLTPSTGHAGYAQKFQGRVTMTRDTSTSTVYMELSSLRSEDTAVYYCAREGYGGNYGNWGQGTLVTVSS

SEQ ID NO: 254 - protein, 118 aa, Artificial Sequence
  source: /note=“Description of Artificial Sequence: Synthetic polypeptide”
QVQLVQSGAEVKKPGASVKVSCKASGYTFTSYYMHWVRQAPGQGLEWMGWMNPNSGHTGYAQKFQGRVTMTRDTSTSTVYMELSSLRSEDTAVYYCAREDFYGDFDYWGQGTLVTVSS

SEQ ID NO: 255 - protein, 118 aa, Artificial Sequence
  source: /note=“Description of Artificial Sequence: Synthetic polypeptide”
QVQLVQSGAEVKKPGASVKVSCKASGYTFTRHFIHWVRQAPGQGLEWMGWIDPNSGVTSSAQKFQGRVTMTRDTSTSTVYMELSSLRSEDTAVYYCARELSRWGFDYWGPGTMVTVSS

SEQ ID NO: 256 - protein, 118 aa, Artificial Sequence
  source: /note=“Description of Artificial Sequence: Synthetic polypeptide”
QVQLVQSGAEVKKPGASVKVSCKASGYTFTRHLLHWVRQAPGQGLEWMGWISPQHGVRNYAHKFQGRVTMTRDTSTSTVYMELSSLRSEDTAVYYCARESVEGYFDLWGRGTLVTVSS

SEQ ID NO: 257 - protein, 123 aa, Artificial Sequence
  source: /note=“Description of Artificial Sequence: Synthetic polypeptide”
QVQLVQSGAEVKKPGASVKVSCKASGYPFSSYYMHWVRQAPGQGLEWMGMINPSGGSTSYAQKFQGRVTMTRDTSTSTVYMELSSLRSEDTAVYYCARDIFPTMIAGGGFDLWGRGTLVTVSS

SEQ ID NO: 258 - protein, 119 aa, Artificial Sequence
  source: /note=“Description of Artificial Sequence: Synthetic polypeptide”
QVQLVQSGAEVKKPGSSVKVSCKASGGTFSTFGISWVRQAPGQGLEWMGGIIPIFGTANYAQKFQGRVTITADESTSTAYMELSSLRSEDTAVYYCARGGYSYGSFDYWGQGTLVTVSS

SEQ ID NO: 259 - protein, 124 aa, Artificial Sequence
  source: /note=“Description of Artificial Sequence: Synthetic polypeptide”
QVQLVQSGAEVKKPGASVKVSCKASGYTFTSYGINWVRQAPGQGLEWMGWMNPNSGNTGYAQKFQGRVTMTRDTSTSTVYMELSSLRSEDTAVYYCARGSFPLVFTIFGVGDVWGQGTMVTVSS

SEQ ID NO: 260 - protein, 119 aa, Artificial Sequence
  source: /note=“Description of Artificial Sequence: Synthetic polypeptide”
QVQLVQSGAEVKKPGASVKVSCKASGYTFTSYYMHWVRQAPGQGLEWMGWISPRSGVTSYAQKFQGRVTMTRDTSTSTVYMELSSLRSEDTAVYYCARDLDYVRAFDIWGQGTTVTVSS

SEQ ID NO: 261 - protein, 118 aa, Artificial Sequence
  source: /note=“Description of Artificial Sequence: Synthetic polypeptide”
QVQLVQSGAEVKKPGASVKVSCKASGYSFTSYYMHWVRQAPGQGLEWMGWMDPNSGNTGYAQKFQGRVTMTRDTSTSTVYMELSSLRSEDTAVYYCARESWGGYFDLWGRGTLVTVSS

SEQ ID NO: 262 - protein, 118 aa, Artificial Sequence
  source: /note=“Description of Artificial Sequence: Synthetic polypeptide”
QVQLVQSGAEVKKPGASVKVSCKASGYTFINHYVHWVRQAPGQGLEWMGWMNPTGGITGYAQKFQGRVTMTRDTSTSTVYMELSSLRSEDTAVYYCARDRTTYAFDIWGQGTMVTVSS

SEQ ID NO: 263 - protein, 118 aa, Artificial Sequence; identical to SEQ ID NO: 243
  source: /note=“Description of Artificial Sequence: Synthetic polypeptide”

SEQ ID NO: 264 - protein, 120 aa, Artificial Sequence
  source: /note=“Description of Artificial Sequence: Synthetic polypeptide”
QVQLVQSGAEVKKPGSSVKVSCKASGYTFTRHLLHWVRQAPGQGLEWMGWVSPIHGLTGYAPRFQGRVTITADESTSTAYMELSSLRSEDTAVYYCARVHGSGSDGMDVWGQGTTVTVSS

SEQ ID NO: 265 - protein, 107 aa, Artificial Sequence
  source: /note=“Description of Artificial Sequence: Synthetic polypeptide”
DIQMTQSPSSLSASVGDRVTITCRASQGIDSYLAWYQQKPGKAPKLLIYAASSLQSGVPSRFSGSGSGTDFTLTISSLQPEDFATYYCQQSYSTPITFGGGTKVEIK

SEQ ID NO: 266 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYAASSLQSGVPSRFSGSGSGTDFTLTISSLQPEDFATYYCQQSYTTPITFGGGTKVEIK

SEQ ID NO: 267 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQIFSTPLTFGGGTKVEIK

SEQ ID NO: 268 - protein, 107 aa, Artificial Sequence
  source: /note=“Description of Artificial Sequence: Synthetic polypeptide”
DIQMTQSPSSLSASVGDRVTITCRASQGISSYLAWYQQKPGKAPKLLIYGASNLQSGVPSRFSGSGSGTDFTLTISSLQPEDFATYYCQQSYSTPLTFGGGTKVEIK

SEQ ID NO: 269 - protein, 107 aa, Artificial Sequence
  source: /note=“Description of Artificial Sequence: Synthetic polypeptide”
DIQMTQSPSSLSASVGDRVTITCRASQTISNYLNWYQQKPGKAPKLLIYAASSLQSGVPSRFSGSGSGTDFTLTISSLQPEDFATYYCQQSYTTPITFGGGTKVEIK

SEQ ID NO: 270 - protein, 108 aa, Artificial Sequence
  source: /note=“Description of Artificial Sequence: Synthetic polypeptide”
EIVMTQSPATLSVSPGERATLSCRASQSVDRNYVTWYQQKPGQAPRLLIYGASTRATGIPARFSGSGSGTEFTLTISSLQSEDFAVYYCQQSYTTPYTFGQGTKVEIK

SEQ ID NO: 271 - protein, 107 aa, Artificial Sequence
  source: /note=“Description of Artificial Sequence: Synthetic polypeptide”
DIQMTQSPSSLSASVGDRVTITCRASQGISQYLAWYQQKPGKAPKLLIYGASNLHSGVPSRFSGSGSGTDFTLTISSLQPEDFATYYCQQTFTTPLTFGGGTKVEIK

SEQ ID NO: 272 - protein, 107 aa, Artificial Sequence
  source: /note=“Description of Artificial Sequence: Synthetic polypeptide”
DIQMTQSPSSLSASVGDRVTITCQASQDIGNYLNWYQQKPGKAPKLLIYAASSLESGVPSRFSGSGSGTDFTLTISSLQPEDFATYYCQQSYSTPLTFGGGTKVEIK

SEQ ID NO: 273 - protein, 107 aa, Artificial Sequence
  source: /note=“Description of Artificial Sequence: Synthetic polypeptide”
DIQMTQSPSSLSASVGDRVTITCRASQGIRNDLGWYQQKPGKAPKLLIYSASNLQSGVPSRFSGSGSGTDFTLTISSLQPEDFATYYCQQANSFPFTFGGGTKVEIK

SEQ ID NO: 274 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYAASTLESGVPSRFSGSGSGTDFTLTISSLQPEDFATYYCQQSYTTPYSFGGGTKVEIK

SEQ ID NO: 275 - protein, 107 aa, Artificial Sequence
  source: /note=“Description of Artificial Sequence: Synthetic polypeptide”
DIQMTQSPSSLSASVGDRVTITCRASQGISQYLAWYQQKPGKAPKLLIYGASNLHSGVPSRFSGSGSGTDFTLTISSLQPEDFATYYCQQTFITPLTFGGGTKVEIK

SEQ ID NO: 276 - protein, 106 aa, Artificial Sequence
  source: /note=“Description of Artificial Sequence: Synthetic polypeptide”
DIQMTQSPSSLSASVGDRVTITCRASQIIGNYLAWYQQKPGKAPKLLIYHASILETGVPSRFSGSGSGTDFTLTISSLQPEDFATYYCQQSYSTPTFGGGTKVEIK

SEQ ID NO: 277 - protein, 107 aa, Artificial Sequence
  source: /note=“Description of Artificial Sequence: Synthetic polypeptide”
DIQMTQSPSSLSASVGDRVTITCRASQIISSYLNWYQQKPGKAPKLLIYAASSLQSGVPSRFSGSGSGTDFTLTISSLQPEDFATYYCQQGFSTPFTFGGGTKVEIK

SEQ ID NO: 278 - protein, 107 aa, Artificial Sequence
  source: /note=“Description of Artificial Sequence: Synthetic polypeptide”
DIQMTQSPSSLSASVGDRVTITCQASQDISNYLNWYQQKPGKAPKLLIYAASSLQSGVPSRFSGSGSGTDFTLTISSLQPEDFATYYCQQSFTNPVTFGGGTKVEIK

SEQ ID NO: 279 - protein, 107 aa, Artificial Sequence
  source: /note=“Description of Artificial Sequence: Synthetic polypeptide”
DIQMTQSPSSLSASVGDRVTITCRASQGISNYLAWYQQKPGKAPKLLIYAASTLQSGVPSRFSGSGSGTDFTLTISSLQPEDFATYYCQQSYSAPYTFGGGTKVEIK

SEQ ID NO: 280 - protein, 107 aa, Artificial Sequence
  source: /note=“Description of Artificial Sequence: Synthetic polypeptide”
DIQMTQSPSSLSASVGDRVTITCRASQGISNNLNWYQQKPGKAPKLLIYAATTLQSGVPSRFSGSGSGTDFTLTISSLQPEDFATYYCQQSYSTPYTFGGGTKVEIK

SEQ ID NO: 281 - protein, 107 aa, Artificial Sequence [sequence omitted]

SEQ ID NO: 282 - protein, 107 aa, Artificial Sequence
  source: /note=“Description of Artificial Sequence: Synthetic polypeptide”
DIQMTQSPSSLSASVGDRVTITCRASQGISNGLSWYQQKPGKAPKLLIYAASTLQSGVPSRFSGSGSGTDFTLTISSLQPEDFATYYCQQSHSTPLTFGGGTKVEIK

SEQ ID NO: 283 - protein, 107 aa, Artificial Sequence
  source: /note=“Description of Artificial Sequence: Synthetic polypeptide”
DIQMTQSPSSLSASVGDRVTITCRASQSITGWLAWYQQKPGKAPKLLIYAASSLQSGVPSRFSGSGSGTDFTLTISSLQPEDFATYYCQQSYSTPYTFGQGTKVEIK

SEQ ID NO: 284 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYDATHLETGVPSRFSGSGSGTDFTLTISSLQPEDFATYYCQQSYSTPITFGQGTRLEIK

SEQ ID NO: 285 - protein, 107 aa, Artificial Sequence
  source: /note=“Description of Artificial Sequence: Synthetic polypeptide”
DIQMTQSPSSLSASVGDRVTITCRASQGIRNDLAWYQQKPGKAPKLLIYAASTLQSGVPSRFSGSGSGTDFTLTISSLQPEDFATYYCQQSYSTPLTFGGGTKVEIK

SEQ ID NO: 286 - protein, 107 aa, Artificial Sequence
  source: /note=“Description of Artificial Sequence: Synthetic polypeptide”
DIQMTQSPSSLSASVGDRVTITCQASQDISSYLNWYQQKPGKAPKLLIYAASSLQTGVPSRFSGSGSGTDFTLTISSLQPEDFATYYCQQSYSTPYTFGQGTKLEIK

SEQ ID NO: 287 - protein, 107 aa, Artificial Sequence
  source: /note=“Description of Artificial Sequence: Synthetic polypeptide”
DIQMTQSPSSLSASVGDRVTITCRASQSITTYLNWYQQKPGKAPKLLIYAASSLQGGVPSRFSGSGSGTDFTLTISSLQPEDFATYYCQQSYSTPYTFGQGTKVEIK

SEQ ID NO: 288 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 289 - protein, 107 aa, Artificial Sequence
  source: /note=“Description of Artificial Sequence: Synthetic polypeptide”
DIQMTQSPSSLSASVGDRVTITCRASQSVSTWLAWYQQKPGKAPKLLIYAASNLESGVPSRFSGSGSGTDFTLTISSLQPEDFATYYCQQSYSTPYTFGQGTKLEIK

SEQ ID NO: 290 - protein, 107 aa, Artificial Sequence
  source: /note=“Description of Artificial Sequence: Synthetic polypeptide”
DIQMTQSPSSLSASVGDRVTITCRASQSISNWLAWYQQKPGKAPKLLIYDVSHLESGVPSRFSGSGSGTDFTLTISSLQPEDFATYYCQQSYSTPFTFGPGTKVDIK

SEQ ID NO: 291 - protein, 107 aa, Artificial Sequence
  source: /note=“Description of Artificial Sequence: Synthetic polypeptide”
DIQMTQSPSSLSASVGDRVTITCQASQGISNYLAWYQQKPGKAPKLLIYDASSLQSGVPSRFSGSGSGTDFTLTISSLQPEDFATYYCQQSYSTPLTFGGGTKVEIK

SEQ ID NO: 292 - protein, 108 aa, Artificial Sequence
  source: /note=“Description of Artificial Sequence: Synthetic polypeptide”
EIVMTQSPATLSVSPGERATLSCRASQSLSSSSLAWYQQKPGQAPRLLIYGASTRATGIPARFSGSGSGTEFTLTISSLQSEDFAVYYCQQYGSSPFTFGPGTKVDIK

SEQ ID NO: 293 - protein, 107 aa, Artificial Sequence
  source: /note=“Description of Artificial Sequence: Synthetic polypeptide”
DIQMTQSPSSLSASVGDRVTITCRASEHIANWLAWYQQKPGKAPKLLIYGVSSLESGVPSRFSGSGSGTDFTLTISSLQPEDFATYYCQQSYSTPYTFGQGTKLEIK

SEQ ID NO: 294 - protein, 107 aa, Artificial Sequence
  source: /note=“Description of Artificial Sequence: Synthetic polypeptide”
DIQMTQSPSSLSASVGDRVTITCRASQSVGSWVAWYQQKPGKAPKLLIYPASTLQSGVPSRFSGSGSGTDFTLTISSLQPEDFATYYCQQSYSTPLTFGGGTKVEIK

SEQ ID NO: 295 - protein, 107 aa, Artificial Sequence
  source: /note=“Description of Artificial Sequence: Synthetic polypeptide”
DIQMTQSPSSLSASVGDRVTITCRASQSISPWLAWYQQKPGKAPKLLIYDASNLETGVPSRFSGSGSGTDFTLTISSLQPEDFATYYCQQTYSTPITFGQGTRLEIK

SEQ ID NO: 296 - protein, 107 aa, Artificial Sequence
  source: /note=“Description of Artificial Sequence: Synthetic polypeptide”
DIQMTQSPSSLSASVGDRVTITCRASQGISRYLAWYQQKPGKAPKLLIYAASTLQSGVPSRFSGSGSGTDFTLTISSLQPEDFATYYCQQSYSTPLTFGQGTKVEIK

SEQ ID NO: 297 - protein, 108 aa, Artificial Sequence
  source: /note=“Description of Artificial Sequence: Synthetic polypeptide”
EIVMTQSPATLSVSPGERATLSCRASQTVSSNYLAWYQQKPGQAPRLLIYGASTRASGIPARFSGSGSGTEFTLTISSLQSEDFAVYYCQQYYTTPLTFGGGTKVEIK

SEQ ID NO: 298 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSFSTPLTFGQGTRLEIK

SEQ ID NO: 299 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYDASNLETGVPSRFSGSGSGTDFTLTISSLQPEDFATYYCQQSYSTPPTFGQGTKVEIK

SEQ ID NO: 300 - protein, 107 aa, Artificial Sequence
  source: /note=“Description of Artificial Sequence: Synthetic polypeptide”
DIQMTQSPSSLSASVGDRVTITCRASQGISNNLNWYQQKPGKAPKLLIYATSTLQSGVPSRFSGSGSGTDFTLTISSLQPEDFATYYCQQSYSTPYTFGQGTKVEIK

SEQ ID NO: 301 - protein, 107 aa, Artificial Sequence
  source: /note=“Description of Artificial Sequence: Synthetic polypeptide”
DIQMTQSPSSLSASVGDRVTITCRSSQGIRNDLSWYQQKPGKAPKLLIYLASNSHSGVPSRFSGSGSGTDFTLTISSLQPEDFATYYCLQHNSYPLTFGGGTKVEIK

SEQ ID NO: 302 - protein, 107 aa, Artificial Sequence
  source: /note=“Description of Artificial Sequence: Synthetic polypeptide”
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKLEIK

SEQ ID NO: 303 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYDSSSLQTGVPSRFSGSGSGTDFTLTISSLQPEDFATYYCQQSYSTPVTFGQGTKVEIK

SEQ ID NO: 304 - protein, 107 aa, Artificial Sequence
  source: /note=“Description of Artificial Sequence: Synthetic polypeptide”
DIQITHSPSSLSASVGYRLTITCRDSHSITTWLAWYHQKPWNAPKLMIYAASNLESGVPSRFSGSGSGTYFTLTISSLQPEDFATYYCQHFYNTQYTFGQGTKVEIK

SEQ ID NO: 305 - protein, 108 aa, Artificial Sequence
  source: /note=“Description of Artificial Sequence: Synthetic polypeptide”
DIQMTQSPSSLSASVGDRVTITCRASQVIRNDLAWYQQKPGKAPKLLIYAASTLQSGVPSRFSGSGSGTDFTLTISSLQPEDFATYYCQQSLQYPSHFFGQGTKVEIK

SEQ ID NO: 306 - protein, 107 aa, Artificial Sequence
  source: /note=“Description of Artificial Sequence: Synthetic polypeptide”
DIQMTQSPSSLSASVGDRVTITCRASQSISRYLNWYQQKPGKAPKLLIYAASTLQSGVPSRFSGSGSGTDFTLTISSLQPEDFAPYYCQQSYSTPLTFGPGTKVDIK

SEQ ID NO: 307 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 308 - protein, 5 aa, Artificial Sequence
  source: /note=“Description of Artificial Sequence: Synthetic peptide”
SDYMH

SEQ ID NO: 309 - protein, 5 aa, Artificial Sequence
  source: /note=“Description of Artificial Sequence: Synthetic peptide”
GYYMH

SEQ ID NO: 310 - protein, 5 aa, Artificial Sequence
  source: /note=“Description of Artificial Sequence: Synthetic peptide”
SYYMH

SEQ ID NO: 311 - protein, 5 aa, Artificial Sequence
  source: /note=“Description of Artificial Sequence: Synthetic peptide”
GYYIH

SEQ ID NO: 312 - protein, 5 aa, Artificial Sequence
  source: /note=“Description of Artificial Sequence: Synthetic peptide”
THYMH

SEQ ID NO: 313 - protein, 5 aa, Artificial Sequence
  source: /note=“Description of Artificial Sequence: Synthetic peptide”
SHDIN

SEQ ID NO: 314 - protein, 5 aa, Artificial Sequence
  source: /note=“Description of Artificial Sequence: Synthetic peptide”
DHYLH

SEQ ID NO: 315 - protein, 5 aa, Artificial Sequence
  source: /note=“Description of Artificial Sequence: Synthetic peptide”
NYYMH

SEQ ID NO: 316 - protein, 5 aa, Artificial Sequence
  source: /note=“Description of Artificial Sequence: Synthetic peptide”
AYYVH

SEQ ID NO: 317 - protein, 5 aa, Artificial Sequence
  source: /note=“Description of Artificial Sequence: Synthetic peptide”
RHYVH

SEQ ID NO: 318 - protein, 4 aa, Artificial Sequence
  source: /note=“Description of Artificial Sequence: Synthetic peptide”
NYIH

SEQ ID NO: 319 - protein, 5 aa, Artificial Sequence
  source: /note=“Description of Artificial Sequence: Synthetic peptide”
NHYVH

SEQ ID NO: 320 - protein, 5 aa, Artificial Sequence
  source: /note=“Description of Artificial Sequence: Synthetic peptide”
SHYMH

SEQ ID NO: 321 - protein, 5 aa, Artificial Sequence
  source: /note=“Description of Artificial Sequence: Synthetic peptide”
RHLLH

SEQ ID NO: 322 - protein, 17 aa, Artificial Sequence
  source: /note=“Description of Artificial Sequence: Synthetic peptide”
WMSPYNGITGYAQKFQG

SEQ ID NO: 323 - protein, 17 aa, Artificial Sequence
  source: /note=“Description of Artificial Sequence: Synthetic peptide”
WMSPSSGITGYAQKFQG

SEQ ID NO: 324 - protein, 17 aa, Artificial Sequence
  source: /note=“Description of Artificial Sequence: Synthetic peptide”
WMTTNSGITGYAQKFQG

SEQ ID NO: 325 - protein, 17 aa, Artificial Sequence
  source: /note=“Description of Artificial Sequence: Synthetic peptide”
GIIPIFGTASYAQKFQG

SEQ ID NO: 326 - protein, 17 aa, Artificial Sequence
  source: /note=“Description of Artificial Sequence: Synthetic peptide”
WMNPNSGHAGSAQKFQG

SEQ ID NO: 327 - protein, 17 aa, Artificial Sequence
  source: /note=“Description of Artificial Sequence: Synthetic peptide”
WMNPNSGNTGYAQKFQG

SEQ ID NO: 328 - protein, 17 aa, Artificial Sequence
  source: /note=“Description of Artificial Sequence: Synthetic peptide”
WMNPNSGNTGYSQKFQG

SEQ ID NO: 329 - protein, 17 aa, Artificial Sequence
  source: /note=“Description of Artificial Sequence: Synthetic peptide”
WMNPNIGNTGYAQKFQG

SEQ ID NO: 330 - protein, 17 aa, Artificial Sequence
  source: /note=“Description of Artificial Sequence: Synthetic peptide”
WMNPNGGTTGYAQNFQG

SEQ ID NO: 331 - protein, 17 aa, Artificial Sequence
  source: /note=“Description of Artificial Sequence: Synthetic peptide”
WMNPNRGITDSAQKFQG

SEQ ID NO: 332 - protein, 17 aa, Artificial Sequence
  source: /note=“Description of Artificial Sequence: Synthetic peptide”
WMNPNSGSAGYAQKFQG

SEQ ID NO: 333 - protein, 17 aa, Artificial Sequence
  source: /note=“Description of Artificial Sequence: Synthetic peptide”
WIHPRSGATGYAPKFQG

SEQ ID NO: 334 - protein, 17 aa, Artificial Sequence
  source: /note=“Description of Artificial Sequence: Synthetic peptide”
WISPRSGVTSYAQKFQG

SEQ ID NO: 335 - protein, 17 aa, Artificial Sequence
  source: /note=“Description of Artificial Sequence: Synthetic peptide”
WMDPNSGNTGYAQKFQG

SEQ ID NO: 336 - protein, 17 aa, Artificial Sequence
  source: /note=“Description of Artificial Sequence: Synthetic peptide”
WMNPTGGITGYAQKFQG

SEQ ID NO: 337 - protein, 17 aa, Artificial Sequence
  source: /note=“Description of Artificial Sequence: Synthetic peptide”
WMNPNSGHTGNAQKFQG

SEQ ID NO: 338 - protein, 17 aa, Artificial Sequence
  source: /note=“Description of Artificial Sequence: Synthetic peptide”
WVSPIHGLTGYAPRFQG

SEQ ID NO: 339 - protein, 9 aa, Artificial Sequence
  source: /note=“Description of Artificial Sequence: Synthetic peptide”
DRFSGSYDY

SEQ ID NO: 340 - protein, 7 aa, Artificial Sequence
  source: /note=“Description of Artificial Sequence: Synthetic peptide”
DRGWFDP

SEQ ID NO: 341 - protein, 9 aa, Artificial Sequence
  source: /note=“Description of Artificial Sequence: Synthetic peptide”
EGYSSGLDY

SEQ ID NO: 342 - protein, 11 aa, Artificial Sequence
  source: /note=“Description of Artificial Sequence: Synthetic peptide”
DGRFWSGYPDY

SEQ ID NO: 343 - protein, 10 aa, Artificial Sequence
  source: /note=“Description of Artificial Sequence: Synthetic peptide”
ESIAVAGYDY

SEQ ID NO: 344 - protein, 10 aa, Artificial Sequence
  source: /note=“Description of Artificial Sequence: Synthetic peptide”
DRWYMGSADY

SEQ ID NO: 345 - protein, 10 aa, Artificial Sequence
  source: /note=“Description of Artificial Sequence: Synthetic peptide”
DDWGGDWFDP

SEQ ID NO: 346 - protein, 10 aa, Artificial Sequence
  source: /note=“Description of Artificial Sequence: Synthetic peptide”
ERLSVAGFDY

SEQ ID NO: 347 - protein, 10 aa, Artificial Sequence
  source: /note=“Description of Artificial Sequence: Synthetic peptide”
EPLQLGGFDY

SEQ ID NO: 348 - protein, 10 aa, Artificial Sequence
  source: /note=“Description of Artificial Sequence: Synthetic peptide”
EGFGPNAFDI

SEQ ID NO: 349 - protein, 10 aa, Artificial Sequence
  source: /note=“Description of Artificial Sequence: Synthetic peptide”
DSWYGDWFDP

SEQ ID NO: 350 - protein, 9 aa, Artificial Sequence
  source: /note=“Description of Artificial Sequence: Synthetic peptide”
EVIEVGMDV

SEQ ID NO: 351 - protein, 9 aa, Artificial Sequence
  source: /note=“Description of Artificial Sequence: Synthetic peptide”
EAWFGELST

SEQ ID NO: 352 - protein, 9 aa, Artificial Sequence
  source: /note=“Description of Artificial Sequence: Synthetic peptide”
EAYVAAFDI

SEQ ID NO: 353 - protein, 9 aa, Artificial Sequence
  source: /note=“Description of Artificial Sequence: Synthetic peptide”
ERGYNAFDY

SEQ ID NO: 354 - protein, 8 aa, Artificial Sequence
  source: /note=“Description of Artificial Sequence: Synthetic peptide”
DSVFGLDY

SEQ ID NO: 355 - protein, 10 aa, Artificial Sequence
  source: /note=“Description of Artificial Sequence: Synthetic peptide”
DLDYVRAFDI

SEQ ID NO: 356 - protein, 9 aa, Artificial Sequence
  source: /note=“Description of Artificial Sequence: Synthetic peptide”
ESWGGYFDL

SEQ ID NO: 357 - protein, 9 aa, Artificial Sequence
  source: /note=“Description of Artificial Sequence: Synthetic peptide”
DRTTYAFDI

SEQ ID NO: 358 - protein, 9 aa, Artificial Sequence
  source: /note=“Description of Artificial Sequence: Synthetic peptide”
GNWVDAFDI

SEQ ID NO: 359 - protein, 11 aa, Artificial Sequence
  source: /note=“Description of Artificial Sequence: Synthetic peptide”
VHGSGSDGMDV

SEQ ID NO: 360 - protein, 11 aa, Artificial Sequence
  source: /note=“Description of Artificial Sequence: Synthetic peptide”
RASQSISSWLA

SEQ ID NO: 361 - protein, 11 aa, Artificial Sequence
  source: /note=“Description of Artificial Sequence: Synthetic peptide”
RASQSVGTWLA

SEQ ID NO: 362 - protein, 11 aa, Artificial Sequence
  source: /note=“Description of Artificial Sequence: Synthetic peptide”
RASQGISNYLA

SEQ ID NO: 363 - protein, 11 aa, Artificial Sequence
  source: /note=“Description of Artificial Sequence: Synthetic peptide”
RASQSISTWLA

SEQ ID NO: 364 - protein, 11 aa, Artificial Sequence
  source: /note=“Description of Artificial Sequence: Synthetic peptide”
QASQDISNHLN

SEQ ID NO: 365 - protein, 11 aa, Artificial Sequence
  source: /note=“Description of Artificial Sequence: Synthetic peptide”
RASQGISSWLA

SEQ ID NO: 366 - protein, 11 aa, Artificial Sequence
  source: /note=“Description of Artificial Sequence: Synthetic peptide”
RASESISSWLA

SEQ ID NO: 367 - protein, 11 aa, Artificial Sequence
  source: /note=“Description of Artificial Sequence: Synthetic peptide”
RASQSVGSWLA

SEQ ID NO: 368 - protein, 11 aa, Artificial Sequence
  source: /note=“Description of Artificial Sequence: Synthetic peptide”
RASQNISNFLN

SEQ ID NO: 369 - protein, 12 aa, Artificial Sequence
  source: /note=“Description of Artificial Sequence: Synthetic peptide”
RASQSLSSSYLA

SEQ ID NO: 370 - protein, 11 aa, Artificial Sequence
  source: /note=“Description of Artificial Sequence: Synthetic peptide”
RASQSISSYLN

SEQ ID NO: 371 - protein, 11 aa, Artificial Sequence
  source: /note=“Description of Artificial Sequence: Synthetic peptide”
RASQSISRWLA

SEQ ID NO: 372 - protein, 11 aa, Artificial Sequence
  source: /note=“Description of Artificial Sequence: Synthetic peptide”
RDSHSITTWLA

SEQ ID NO: 373 - protein, 11 aa, Artificial Sequence
  source: /note=“Description of Artificial Sequence: Synthetic peptide”
RASQVIRNDLA

SEQ ID NO: 374 - protein, 11 aa, Artificial Sequence
  source: /note=“Description of Artificial Sequence: Synthetic peptide”
RASQSISRYLN

SEQ ID NO: 375 - protein, 7 aa, Artificial Sequence
  source: /note=“Description of Artificial Sequence: Synthetic peptide”
AASSLQS

SEQ ID NO: 376 - protein, 7 aa, Artificial Sequence
  source: /note=“Description of Artificial Sequence: Synthetic peptide”
AASTLEN

SEQ ID NO: 377 - protein, 7 aa, Artificial Sequence
  source: /note=“Description of Artificial Sequence: Synthetic peptide”
RASNLES

SEQ ID NO: 378 - protein, 7 aa, Artificial Sequence
  source: /note=“Description of Artificial Sequence: Synthetic peptide”
AASTLQR

SEQ ID NO: 379 - protein, 7 aa, Artificial Sequence
  source: /note=“Description of Artificial Sequence: Synthetic peptide”
AASTLQS

SEQ ID NO: 380 - protein, 7 aa, Artificial Sequence
  source: /note=“Description of Artificial Sequence: Synthetic peptide”
GASNLQR

SEQ ID NO: 381 - protein, 7 aa, Artificial Sequence
  source: /note=“Description of Artificial Sequence: Synthetic peptide”
AASNLQS

SEQ ID NO: 382 - protein, 7 aa, Artificial Sequence
  source: /note=“Description of Artificial Sequence: Synthetic peptide”
GASSLQS

SEQ ID NO: 383 - protein, 7 aa, Artificial Sequence
  source: /note=“Description of Artificial Sequence: Synthetic peptide”
AASHLQS

SEQ ID NO: 384 - skipped

SEQ ID NO: 385 - protein, 7 aa, Artificial Sequence
  source: /note=“Description of Artificial Sequence: Synthetic peptide”
DSSSLQT

SEQ ID NO: 386 - protein, 7 aa, Artificial Sequence
  source: /note=“Description of Artificial Sequence: Synthetic peptide”
AASNLES

SEQ ID NO: 387 - protein, 9 aa, Artificial Sequence
  source: /note=“Description of Artificial Sequence: Synthetic peptide”
QQSYSTPYT

SEQ ID NO: 388 - protein, 9 aa, Artificial Sequence
  source: /note=“Description of Artificial Sequence: Synthetic peptide”
QQSFSTPYT

SEQ ID NO: 389 - protein, 9 aa, Artificial Sequence
  source: /note=“Description of Artificial Sequence: Synthetic peptide”
QQSYSTPLT

SEQ ID NO: 390 - protein, 9 aa, Artificial Sequence
  source: /note=“Description of Artificial Sequence: Synthetic peptide”
QQSYSTPFT

SEQ ID NO: 391 - protein, 9 aa, Artificial Sequence
  source: /note=“Description of Artificial Sequence: Synthetic peptide”
QQSYSTPIT

SEQ ID NO: 392 - protein, 9 aa, Artificial Sequence
  source: /note=“Description of Artificial Sequence: Synthetic peptide”
QQYYSTPYT

SEQ ID NO: 393 - protein, 9 aa, Artificial Sequence
  source: /note=“Description of Artificial Sequence: Synthetic peptide”
QQSYSLPYT

SEQ ID NO: 394 - protein, 9 aa, Artificial Sequence
  source: /note=“Description of Artificial Sequence: Synthetic peptide”
HQYFTTPLT

SEQ ID NO: 395 - protein, 9 aa, Artificial Sequence
  source: /note=“Description of Artificial Sequence: Synthetic peptide”
QQSYSMPYT

SEQ ID NO: 396 - protein, 9 aa, Artificial Sequence
  source: /note=“Description of Artificial Sequence: Synthetic peptide”
QQSYSTPVT

SEQ ID NO: 397 - protein, 9 aa, Artificial Sequence
  source: /note=“Description of Artificial Sequence: Synthetic peptide”
QHFYNTQYT

SEQ ID NO: 398 - protein, 10 aa, Artificial Sequence
  source: /note=“Description of Artificial Sequence: Synthetic peptide”
QQSLQYPSHF

SEQ ID NO: 399 - protein, 30 aa, Artificial Sequence; identical to SEQ ID NO: 187
  source: /note=“Description of Artificial Sequence: Synthetic polypeptide”

SEQ ID NO: 400 - protein, 30 aa, Artificial Sequence; identical to SEQ ID NO: 178
  source: /note=“Description of Artificial Sequence: Synthetic polypeptide”

SEQ ID NO: 401 - protein, 30 aa, Artificial Sequence; identical to SEQ ID NO: 191
  source: /note=“Description of Artificial Sequence: Synthetic polypeptide”

SEQ ID NO: 402 - protein, 30 aa, Artificial Sequence
  source: /note=“Description of Artificial Sequence: Synthetic polypeptide”
QVQLVQSGAEVKEPGASVKVSCKASGYTFT

SEQ ID NO: 403 - protein, 30 aa, Artificial Sequence
  source: /note=“Description of Artificial Sequence: Synthetic polypeptide”
QVQLVQSGAEVKKPGASVKVSCKASGYNFS

SEQ ID NO: 404 - skipped

SEQ ID NO: 405 - protein, 30 aa, Artificial Sequence; identical to SEQ ID NO: 189
  source: /note=“Description of Artificial Sequence: Synthetic polypeptide”

SEQ ID NO: 406 - protein, 30 aa, Artificial Sequence; identical to SEQ ID NO: 190
  source: /note=“Description of Artificial Sequence: Synthetic polypeptide”

SEQ ID NO: 407 - protein, 14 aa, Artificial Sequence
  source: /note=“Description of Artificial Sequence: Synthetic peptide”
WVRQAPGQGLEWMG

SEQ ID NO: 408 - protein, 14 aa, Artificial Sequence
  source: /note=“Description of Artificial Sequence: Synthetic peptide”
WMRQAPGQGLEWIG

SEQ ID NO: 409 - protein, 32 aa, Artificial Sequence; identical to SEQ ID NO: 196
  source: /note=“Description of Artificial Sequence: Synthetic polypeptide”

SEQ ID NO: 410 - protein, 32 aa, Artificial Sequence; identical to SEQ ID NO: 199
  source: /note=“Description of Artificial Sequence: Synthetic polypeptide”

SEQ ID NO: 411 - protein, 11 aa, Artificial Sequence
  source: /note=“Description of Artificial Sequence: Synthetic peptide”
WGQGTLVTVSS

SEQ ID NO: 412 - protein, 11 aa, Artificial Sequence
  source: /note=“Description of Artificial Sequence: Synthetic peptide”
WGQGTTVTVSS

SEQ ID NO: 413 - protein, 11 aa, Artificial Sequence
  source: /note=“Description of Artificial Sequence: Synthetic peptide”
WGRGTLVTVSS

SEQ ID NO: 414 - protein, 11 aa, Artificial Sequence
  source: /note=“Description of Artificial Sequence: Synthetic peptide”
WGQGTMVTVSS

SEQ ID NO: 415 - protein, 23 aa, Artificial Sequence
  source: /note=“Description of Artificial Sequence: Synthetic peptide”
DIQMTQSPSSLSASVGDRVTITC

SEQ ID NO: 416 - protein, 23 aa, Artificial Sequence
  source: /note=“Description of Artificial Sequence: Synthetic peptide”
EIVMTQSPATLSVSPGERATLSC

SEQ ID NO: 417 - protein, 23 aa, Artificial Sequence
  source: /note=“Description of Artificial Sequence: Synthetic peptide”
DIQITHSPSSLSASVGYRLTITC

SEQ ID NO: 418 - protein, 15 aa, Artificial Sequence
  source: /note=“Description of Artificial Sequence: Synthetic peptide”
WYQQKPGKAPKLLIY

SEQ ID NO: 419 - protein, 15 aa, Artificial Sequence
  source: /note=“Description of Artificial Sequence: Synthetic peptide”
WYQQKPGQAPRLLIY

SEQ ID NO: 420 - protein, 15 aa, Artificial Sequence
  source: /note=“Description of Artificial Sequence: Synthetic peptide”
WYHQKPWNAPKLMIY

SEQ ID NO: 421 - protein, 32 aa, Artificial Sequence; identical to SEQ ID NO: 213
  source: /note=“Description of Artificial Sequence: Synthetic polypeptide”

SEQ ID NO: 422 - protein, 32 aa, Artificial Sequence; identical to SEQ ID NO: 214
  source: /note=“Description of Artificial Sequence: Synthetic polypeptide”

SEQ ID NO: 423 - protein, 32 aa, Artificial Sequence; identical to SEQ ID NO: 215
  source: /note=“Description of Artificial Sequence: Synthetic polypeptide”

SEQ ID NO: 424 - protein, 32 aa, Artificial Sequence; identical to SEQ ID NO: 216
  source: /note=“Description of Artificial Sequence: Synthetic polypeptide”

SEQ ID NO: 425 - protein, 10 aa, Artificial Sequence
  source: /note=“Description of Artificial Sequence: Synthetic peptide”
FGGGTKVEIK

SEQ ID NO: 426 - protein, 10 aa, Artificial Sequence
  source: /note=“Description of Artificial Sequence: Synthetic peptide”
FGQGTKLEIK

SEQ ID NO: 427 - protein, 10 aa, Artificial Sequence
  source: /note=“Description of Artificial Sequence: Synthetic peptide”
FGQGTKVEIK

SEQ ID NO: 428 - protein, 10 aa, Artificial Sequence
  source: /note=“Description of Artificial Sequence: Synthetic peptide”
FGQGTRLEIK

SEQ ID NO: 429 - protein, 10 aa, Artificial Sequence
  source: /note=“Description of Artificial Sequence: Synthetic peptide”
FGPGTKVDIK

SEQ ID NO: 430 - protein, 118 aa, Artificial Sequence
  source: /note=“Description of Artificial Sequence: Synthetic polypeptide”
QVQLVQSGAEVKKPGASVKVSCKASGYTFSSDYMHWVRQAPGQGLEWMGWMSPYNGITGYAQKFQGRVTMTRDTSTSTVYMELSSLRSEDTAVYYCARDRFSGSYDYWGQGTLVTVSS

SEQ ID NO: 431 - protein, 116 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWMSPSSGITGYAQKFQGRVTMTRDTSTSTVYMELSSLRSEDTAVYYCARDRGWFDPWGQGTLVTVSS

SEQ ID NO: 432 - protein, 118 aa, Artificial Sequence
  source: /note=“Description of Artificial Sequence: Synthetic polypeptide”
QVQLVQSGAEVKKPGASVKVSCKASGYTFSSYYMHWVRQAPGQGLEWMGWMTTNSGITGYAQKFQGRVTMTRDTSTSTVYMELSSLRSEDTAVYYCAREGYSSGLDYWGQGTLVTVSS

SEQ ID NO: 433 - protein, 120 aa, Artificial Sequence
  source: /note=“Description of Artificial Sequence: Synthetic polypeptide”
QVQLVQSGAEVKKPGSSVKVSCKASGYTFTGYYIHWVRQAPGQGLEWMGGIIPIFGTASYAQKFQGRVTITADESTSTAYMELSSLRSEDTAVYYCARDGRFWSGYPDYWGQGTLVTVSS

SEQ ID NO: 434 - protein, 119 aa, Artificial Sequence
  source: /note=“Description of Artificial Sequence: Synthetic polypeptide”
QVQLVQSGAEVKKPGASVKVSCKASGYTFSTHYMHWVRQAPGQGLEWMGWMNPNSGHAGSAQKFQGRVTMTRDTSTSTVYMELSSLRSEDTAVYYCARESIAVAGYDYWGQGTLVTVSS

SEQ ID NO: 435 - protein, 119 aa, Artificial Sequence
  source: /note=“Description of Artificial Sequence: Synthetic polypeptide”
QVQLVQSGAEVKKPGASVKVSCKASGYTFTSHDINWVRQAPGQGLEWMGWMNPNSGNTGYAQKFQGRVTMTRDTSTSTVYMELSSLRSEDTAVYYCARDRWYMGSADYWGQGTLVTVSS

SEQ ID NO: 436 - protein, 119 aa, Artificial Sequence
  source: /note=“Description of Artificial Sequence: Synthetic polypeptide”
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARDDWGGDWFDPWGQGTLVTVSS

SEQ ID NO: 437 - protein, 119 aa, Artificial Sequence
  source: /note=“Description of Artificial Sequence: Synthetic polypeptide”
QVQLVQSGAEVKKPGASVKVSCKASGYTFSTHYMHWVRQAPGQGLEWMGWMNPNSGNTGYSQKFQGRVTMTRDTSTSTVYMELSSLRSEDTAVYYCARERLSVAGFDYWGQGTLVTVSS

SEQ ID NO: 438 - protein, 119 aa, Artificial Sequence
  source: /note=“Description of Artificial Sequence: Synthetic polypeptide”
QVQLVQSGAEVKEPGASVKVSCKASGYTFTDHYLHWVRQAPGQGLEWMGWMNPNIGNTGYAQKFQGRVTMTRDTSTSTVYMELSSLRSEDTAVYYCAREPLQLGGFDYWGQGTLVTVSS

SEQ ID NO: 439 - protein, 119 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWMNPNGGTTGYAQNFQGRVTMTRDTSTSTVYMELSSLRSEDTAVYYCAREGFGPNAFDIWGQGTTVTVSS

SEQ ID NO: 440 - protein, 119 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCARDSWYGDWFDPWGQGTLVTVSS

SEQ ID NO: 441 - protein, 118 aa, Artificial Sequence
  source: /note=“Description of Artificial Sequence: Synthetic polypeptide”
QVQLVQSGAEVKKPGASVKVSCKASGYTFTGYYMHWMRQAPGQGLEWIGWMSPYNGITGYAQKFQGRVTMTRDTSTSTVYMELSSLRSEDTAVYYCAREVIEVGMDVWGQGTTVTVSS

SEQ ID NO: 442 - protein, 118 aa, Artificial Sequence
  source: /note=“Description of Artificial Sequence: Synthetic polypeptide”
QVQLVQSGAEVKKPGASVKVSCKASGYTFTNYYMHWVRQAPGQGLEWMGWMSPSSGITGYAQKFQGRVTMTRDTSTSTVYMELSSLRSEDTAVYYCAREAWFGELSTWGQGTLVTVSS

SEQ ID NO: 443 - protein, 118 aa, Artificial Sequence
  source: /note=“Description of Artificial Sequence: Synthetic polypeptide”
QVQLVQSGAEVKKPGASVKVSCKASGYNFSAYYVHWVRQAPGQGLEWMGWMNPNRGITDSAQKFQGRVTMTRDTSTSTVYMELSSLRSEDTAVYYCAREAYVAAFDIWGQGTTVTVSS

SEQ ID NO: 444 - protein, 118 aa, Artificial Sequence
  source: /note=“Description of Artificial Sequence: Synthetic polypeptide”
QVQLVQSGAEVKKPGASVKVSCKASGYTFTRHYVHWVRQAPGQGLEWMGWMNPNSGSAGYAQKFQGRVTMTRDTSTSTVYMELSSLRSEDTAVYYCARERGYNAFDYWGQGTLVTVSS

SEQ ID NO: 445 - protein, 116 aa, Artificial Sequence
  source: /note=“Description of Artificial Sequence: Synthetic polypeptide”
QVQLVQSGAEVKKPGASVKVSCKASGYTLPNYIHWVRQAPGQGLEWMGWIHPRSGATGYAPKFQGRVTMTRDTSTSTVYMELSSLRSEDTAVYYCARDSVFGLDYWGQGTLVTVSS

SEQ ID NO: 446 - protein, 119 aa, Artificial Sequence; identical to SEQ ID NO: 260
  source: /note=“Description of Artificial Sequence: Synthetic polypeptide”

SEQ ID NO: 447 - protein, 118 aa, Artificial Sequence; identical to SEQ ID NO: 261
  source: /note=“Description of Artificial Sequence: Synthetic polypeptide”

SEQ ID NO: 448 - protein, 118 aa, Artificial Sequence; identical to SEQ ID NO: 262
  source: /note=“Description of Artificial Sequence: Synthetic polypeptide”

SEQ ID NO: 449 - protein, 118 aa, Artificial Sequence; identical to SEQ ID NO: 243
  source: /note=“Description of Artificial Sequence: Synthetic polypeptide”

SEQ ID NO: 450 - protein, 120 aa, Artificial Sequence; identical to SEQ ID NO: 264
  source: /note=“Description of Artificial Sequence: Synthetic polypeptide”

SEQ ID NO: 451 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYAASSLQSGVPSRFSGSGSGTDFTLTISSLQPEDFATYYCQQSYSTPYTFGGGTKVEIK

SEQ ID NO: 452 - protein, 107 aa, Artificial Sequence
  source: /note=“Description of Artificial Sequence: Synthetic polypeptide”
DIQMTQSPSSLSASVGDRVTITCRASQSVGTWLAWYQQKPGKAPKLLIYAASTLENGVPSRFSGSGSGTDFTLTISSLQPEDFATYYCQQSFSTPYTFGGGTKVEIK

SEQ ID NO: 453 - protein, 107 aa, Artificial Sequence; identical to SEQ ID NO: 451
  source: /note=“Description of Artificial Sequence: Synthetic polypeptide”

SEQ ID NO: 454 - protein, 107 aa, Artificial Sequence
  source: /note=“Description of Artificial Sequence: Synthetic polypeptide”
DIQMTQSPSSLSASVGDRVTITCRASQGISNYLAWYQQKPGKAPKLLIYRASNLESGVPSRFSGSGSGTDFTLTISSLQPEDFATYYCQQSYSTPLTFGGGTKVEIK

SEQ ID NO: 455 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYAASTLQRGVPSRFSGSGSGTDFTLTISSLQPEDFATYYCQQSYSTPYTFGQGTKLEIK

SEQ ID NO: 456 - protein, 107 aa, Artificial Sequence
  source: /note=“Description of Artificial Sequence: Synthetic polypeptide”
DIQMTQSPSSLSASVGDRVTITCRASQSISTWLAWYQQKPGKAPKLLIYAASTLQSGVPSRFSGSGSGTDFTLTISSLQPEDFATYYCQQSYSTPFTFGQGTKVEIK

SEQ ID NO: 457 - protein, 107 aa, Artificial Sequence
  source: /note=“Description of Artificial Sequence: Synthetic polypeptide”
DIQMTQSPSSLSASVGDRVTITCQASQDISNHLNWYQQKPGKAPKLLIYGASNLQRGVPSRFSGSGSGTDFTLTISSLQPEDFATYYCQQSYSTPITFGQGTRLEIK

SEQ ID NO: 458 - protein, 107 aa, Artificial Sequence
  source: /note=“Description of Artificial Sequence: Synthetic polypeptide”
DIQMTQSPSSLSASVGDRVTITCRASQGISSWLAWYQQKPGKAPKLLIYAASSLQSGVPSRFSGSGSGTDFTLTISSLQPEDFATYYCQQSYSTPYTFGQGTKVEIK

SEQ ID NO: 459 - protein, 107 aa, Artificial Sequence
  source: /note=“Description of Artificial Sequence: Synthetic polypeptide”
DIQMTQSPSSLSASVGDRVTITCRASESISSWLAWYQQKPGKAPKLLIYAASSLQSGVPSRFSGSGSGTDFTLTISSLQPEDFATYYCQQSYSTPLTFGQGTKLEIK

SEQ ID NO: 460 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYAASNLQSGVPSRFSGSGSGTDFTLTISSLQPEDFATYYCQQYYSTPYTFGQGTKVEIK

SEQ ID NO: 461 - protein, 107 aa, Artificial Sequence
  source: /note=“Description of Artificial Sequence: Synthetic polypeptide”
DIQMTQSPSSLSASVGDRVTITCRASQSVGSWLAWYQQKPGKAPKLLIYGASSLQSGVPSRFSGSGSGTDFTLTISSLQPEDFATYYCQQSYSTPLTFGGGTKVEIK

SEQ ID NO: 462 - protein, 107 aa, Artificial Sequence
  source: /note=“Description of Artificial Sequence: Synthetic polypeptide”
DIQMTQSPSSLSASVGDRVTITCRASQSISSWLAWYQQKPGKAPKLLIYAASHLQSGVPSRFSGSGSGTDFTLTISSLQPEDFATYYCQQSYSTPYTFGQGTRLEIK

SEQ ID NO: 463 - protein, 107 aa, Artificial Sequence
  source: /note=“Description of Artificial Sequence: Synthetic polypeptide”
DIQMTQSPSSLSASVGDRVTITCRASQNISNFLNWYQQKPGKAPKLLIYAASSLQSGVPSRFSGSGSGTDFTLTISSLQPEDFATYYCQQSYSLPYTFGQGTKVEIK

SEQ ID NO: 464 - protein, 107 aa, Artificial Sequence
  source: /note=“Description of Artificial Sequence: Synthetic polypeptide”
DIQMTQSPSSLSASVGDRVTITCRASQGISSWLAWYQQKPGKAPKLLIYAASSLQSGVPSRFSGSGSGTDFTLTISSLQPEDFATYYCQQSYSTPLTFGGGTKVEIK

SEQ ID NO: 465 - protein, 108 aa, Artificial Sequence
  source: /note=“Description of Artificial Sequence: Synthetic polypeptide”
EIVMTQSPATLSVSPGERATLSCRASQSLSSSYLAWYQQKPGQAPRLLIYGASTRATGIPARFSGSGSGTEFTLTISSLQSEDFAVYYCHQYFTTPLTFGQGTRLEIK

SEQ ID NO: 466 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYAASTLQSGVPSRFSGSGSGTDFTLTISSLQPEDFATYYCQQSYSMPYTFGQGTKLEIK

SEQ ID NO: 467 - protein, 107 aa, Artificial Sequence; identical to SEQ ID NO: 302
  source: /note=“Description of Artificial Sequence: Synthetic polypeptide”

SEQ ID NO: 468 - protein, 107 aa, Artificial Sequence; identical to SEQ ID NO: 303
  source: /note=“Description of Artificial Sequence: Synthetic polypeptide”

SEQ ID NO: 469 - protein, 107 aa, Artificial Sequence; identical to SEQ ID NO: 304
  source: /note=“Description of Artificial Sequence: Synthetic polypeptide”

SEQ ID NO: 470 - protein, 108 aa, Artificial Sequence; identical to SEQ ID NO: 305
  source: /note=“Description of Artificial Sequence: Synthetic polypeptide”

SEQ ID NO: 471 - protein, 107 aa, Artificial Sequence; identical to SEQ ID NO: 306
  source: /note=“Description of Artificial Sequence: Synthetic polypeptide”

SEQ ID NO: 472 - protein, 30 aa, Artificial Sequence
  source: /note=“Description of Artificial Sequence: Synthetic polypeptide”
QVQLVQSGAEVKKPGASVKVSCKASGYTLP

SEQ ID NO: 473 - protein, 4 aa, Artificial Sequence
  source: /note=“Description of Artificial Sequence: Synthetic peptide”
AAPA

SEQ ID NO: 474 - protein, 4 aa, Artificial Sequence
  source: /note=“Description of Artificial Sequence: Synthetic peptide”
AAPV

SEQ ID NO: 475 - protein, 4 aa, Artificial Sequence
  source: /note=“Description of Artificial Sequence: Synthetic peptide”
  MOD_RES (4)..(4): Nva
AAPX

(19) United States
(12) Patent Application Publication
(10) Pub. No.: US 2024/0033370 A1
(43) Pub. Date: Feb. 1, 2024

(54) ANTI-PD-L1 IMMUNOCONJUGATES, AND USES THEREOF

(71) Applicant: BOLT BIOTHERAPEUTICS, INC., Redwood City, CA (US)

(72) Inventors: Shelley Erin ACKERMAN, Redwood City, CA (US); Michael N. ALONSO, Redwood City, CA (US); David DORNAN, Redwood City, CA (US); Marcin KOWANETZ, Redwood City, CA (US); Romas KUDIRKA, Redwood City, CA (US); Arthur LEE, Redwood City, CA (US); William MALLET, Redwood City, CA (US); Brian SAFINA, Redwood City, CA (US); Matthew ZHOU, Redwood City, CA (US)

(73) Assignee: BOLT BIOTHERAPEUTICS, INC., Redwood City, CA (US)

(21) Appl. No.: 18/038,910

(22) PCT Filed: Dec. 10, 2021
(86) PCT No.: PCT/US2021/062824

§ 371 Date: May 25, 2023

Related U.S. Application Data
(60) Provisional application No. 63/124,396, filed on Dec. 11, 2020.

(51) Int. Cl. A61K 47/68; A61P 35/00

(52) U.S. Cl. CPC A61K 47/6849; A61K 47/6803; A61K 47/6889; A61P 35/00

(57) ABSTRACT

The invention provides immunoconjugates of Formula I comprising an anti-PD-L1 antibody linked by conjugation to one or more 8-Cyc-2-aminobenzazepine derivatives. The invention also provides 8-Cyc-2-aminobenzazepine derivative intermediate compositions comprising a reactive functional group. Such intermediate compositions are suitable substrates for formation of the immunoconjugates through a linker or linking moiety. The invention further provides methods of treating cancer with the immunoconjugates.

### CROSS REFERENCE TO RELATED APPLICATIONS

This non-provisional application claims the benefit of priority to U.S. Provisional Application No. 63/124,396, filed 11 Dec. 2020, which is incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 6, 2021, is named 17019 013WO1_SL.txt and is 244,809 bytes in size.

### FIELD OF THE INVENTION

The invention relates generally to an immunoconjugate comprising an anti-PD-L1 antibody conjugated to one or more 8-cyclyl-2-aminobenzazepine molecules.

### BACKGROUND OF THE INVENTION

New compositions and methods for the delivery of antibodies and immune adjuvants are needed in order to reach inaccessible tumors and/or to expand treatment options for cancer patients and other subjects. The invention provides such compositions and methods.

### SUMMARY OF THE INVENTION

The invention is generally directed to immunoconjugates comprising an anti-PD-L1 antibody linked by conjugation to one or more 8-cycyl-2-aminobenzazepine derivatives. The invention is further directed to 8-cyclyl-2-aminobenzazepine derivative intermediate compositions comprising a reactive functional group. Such intermediate compositions are suitable substrates for formation of immunoconjugates wherein an antibody may be covalently bound by a linker L to a 8-Cyc-2-aminobenzazepine (CycBz) moiety having the formula:

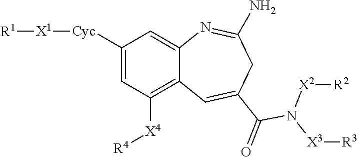

where Cyc is selected from phenyldiyl, heterocyclyldiyl and heteroaryldiyl; and one of R¹, R², R³ and R⁴ is attached to L. The R¹⁻⁴ and X¹⁻⁴ substituents are defined herein.

The invention is further directed to use of such an immunoconjugates in the treatment of an illness, in particular cancer.

An aspect of the invention is an immunoconjugate comprising an anti PD-L1 antibody covalently attached to a linker which is covalently attached to one or more 8-Cyc-2-aminobenzazepine moieties.

Another aspect of the invention is a 8-cyclyl-2-aminobenzazepine-linker compound.

Another aspect of the invention is a method for treating cancer comprising administering a therapeutically effective amount of an immunoconjugate comprising an anti PD-L1 antibody linked by conjugation to one or more 8-Cyc-2-aminobenzazepine moieties.

Another aspect of the invention is a use of an immunoconjugate comprising an anti PD-L1 ntibody linked by conjugation to one or more 8-Cyc-2-aminobenzazepine moieties for treating cancer.

Another aspect of the invention is a method of preparing an immunoconjugate by conjugation of one or more 8-Cyc-2-aminobenzazepine moieties with an anti PD-L1 antibody.

### BRIEF DESCRIPTION OF THE DRAWINGS

FIGS. 1A-D show heavy chain and light chain CDRs of PD-L1 Type A binding agents 1-42.

FIGS. 2A-D show first (HFW1), second (HFW2), third (HFW3), and fourth (HFW4) heavy chain framework region polypeptides of PD-L1 Type A binding agents 1-42.

FIGS. 3A-D show first (LFW1), second (LFW2), third (LFW3), and fourth (LFW4) light chain framework region polypeptides of PD-L1 Type A binding agents 1-42.

FIGS. 4 A-D show heavy chain variable region (VH) of PD-L1 Type A binding agents 1-42.

FIGS. 4 E-G show light chain variable region (VL) of PD-L1 Type A binding agents 1-42.

FIGS. 5A-B show heavy chain and light chain CDRs of PD-L1 Type B binding agents 1-21.

FIGS. 6A-B show first (HFW1), second (HFW2), third (HFW3), and fourth (HFW4) heavy chain framework region polypeptides of PD-L1 Type B binding agents 1-21.

FIGS. 7A-B show first (LFW1), second (LFW2), third (LFW3), and fourth (LFW4) light chain framework region polypeptides of PD-L1 Type B binding agents 1-21.

FIGS. 8A-B show heavy chain variable region (VH) of PD-L1 Type B binding agents 1-21.

FIGS. 8C-D show light chain variable region (VL) of PD-L1 Type B binding agents 1-21.

FIG. 9a shows a graph of an in vivo study of tumor volume over time after treatment comparing the efficacy of immunoconjugate PD-L1 IC-51 versus unconjugated anti-PD-L1 antibody in a syngeneic MB49 tumor model. Mice bearing large (˜200 mm³) syngeneic MB49 tumors were treated with immunoconjugate IC-51 from Table 3b, comprising CycBzL-24 (Table 2b), rat IgG2b isotype control immunoconjugate comprising CycBzL-24, unconjugated anti-PD-L1, and unconjugated IgG2b isotype antibody at 10 mg/kg IP, Q3Dx4 (n=6 per group).

FIG. 9b shows a graph of an in vivo study of tumor volume over time after implantation of MB49 tumor cells, assessing the immunological memory in a re-challenge study. All 6 mice that experienced complete regression of MB49 tumors following immunoconjugate PD-L1 IC-51 from Table 3b treatment in the study from FIG. 9a were re-challenged with MB49 tumor cells about 1 month after tumor clearance (about 7 weeks post final dose).

FIG. 10a shows a graph of tumor volume over time after treatment of parental syngeneic MB49 tumors with immunoconjugate IC-51 from Table 3b, comprising CycBzL-24 (Table 2b), rat IgG2b isotype control immunoconjugate comprising CycBzL-24, unconjugated anti-PD-L1, and unconjugated IgG2b isotype antibody at 5 mg/kg IP, Q3Dx4.

FIG. 10b shows a graph of tumor volume over time after treatment of PD-L1 knockout (PD-L1 KO) syngeneic MB49 tumors lacking PD-L1 expression on tumor cells with immunoconjugate IC-51 from Table 3b, comprising CycBzL-24 (Table 2b), rat IgG2b isotype control immunoconjugate comprising CycBzL-24, unconjugated anti-PD-L1, and unconjugated IgG2b isotype antibody at 5 mg/kg IP, Q3Dx4.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The term “immunoconjugate” or “immune-stimulating antibody conjugate” refers to an antibody construct that is covalently bonded to an adjuvant moiety via a linker. the term “adjuvant” refers to a substance capable of eliciting an immune response in a subject exposed to the adjuvant.

“Adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

“Adjuvant” refers to a substance capable of eliciting an immune response in a subject exposed to the adjuvant.

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively.

IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Antibody construct” refers to an antibody or a fusion protein comprising (i) an antigen binding domain and (ii) an Fc domain.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100× [(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The binding agent comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The binding agent can be any of a variety of types of binding agents known in the art that comprise Ig heavy and light chains. For instance, the binding agent can be an antibody, an antigen-binding antibody “fragment,” or a T-cell receptor.

“Biosimilar” refers to an approved antibody construct that has active properties similar to, for example, a PD-L1-targeting antibody such as atezolizumab, durvalumab, and avelumab

“Biobetter” refers to an approved antibody construct that is an improvement of a previously approved antibody construct, such as atezolizumab, durvalumab, and avelumab. The biobetter can have one or more modifications (e.g., an altered glycan profile, or a unique epitope) over the previously approved antibody construct. A biobetter is a recombinant protein drug from the same class as an existing biopharmaceutical but is not identical; and is superior to the original. A biobetter is not exclusively a new drug, neither a generic version of a drug. Biosimilars and biobetters are both variants of a biologic; with the former being close copies of the originator, while the latter ones have been improved in terms of efficacy, safety, and tolerability or dosing regimen.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line (“ [symbol] ”) represents a point of attachment of the specified chemical moiety. If the specified chemical moiety has two wavy lines present, it will be understood that the chemical moiety can be used bilaterally, i.e., as read from left to right or from right to left.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)3,1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C≡CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer.

To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR) “Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to L1X. A reference to “about X” or “around X” specifically indicates at least the values X, 0.95X, 0.96X, 0.97X, 0.98X, 0.99X, L1X, 1.02X, 1.03X, 1.04X, and 1.05X. Accordingly, “about X” and “around X” are intended to teach and provide written description support for a claim limitation of, e.g., “0.98X.”

### PD-L1 Antibodies

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80), and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers or infections. The PD-L1/PD-1 pathway also contributes to preventing autoimmunity; therefore, agonistic agents against PD-L1 or agents that deliver immune inhibitory payloads may help in the treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1-binding agents, including agents that bind PD-L1 with high affinity and effectively prevent PD-L1/PD-1 signaling, and agents that can deliver therapeutic payloads to PD-L1 expressing cells. In addition, there is a need for new PD-L1-binding agents to treat autoimmune disorders and infections.

A method is provided of delivering a 8-Cyc-2-aminobenzazepine derivative payload to a cell expressing PD-L1 comprising administering to the cell, or mammal comprising the cell, an immunoconjugate comprising an anti-PD-L1 antibody covalently attached to a linker which is covalently attached to one or more 8-Cyc-2-aminobenzazepine moieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 binding agent comprising an immunoglobulin heavy chain variable region polypeptide and an immunoglobulin light chain variable region polypeptide.

The PD-L1 binding agent specifically binds PD-L1. The binding specificity of the agent allows for targeting PD-L1 expressing cells, for instance, to deliver therapeutic payloads to such cells.

In some embodiments, the PD-L1 binding agent (Type A or Type B) binds to human PD-L1, for example, a protein comprising SEQ ID NO: 307. However, binding agents that bind to any PD-L1 homolog or paralog also are encompassed. In some embodiments, the PD-L1 protein comprises at least about 70%, about 75%, about 80%, about 85%, about 90%, about 91%, about 92%, about 93%, about 94%, about 95%, about 96%, about 97%, about 98%, about 99%, or more sequence identity to SEQ ID NO: 307. In some embodiments, the binding agent binds human PD-L1 and cynomolgus PD-L1; or human, cynomolgus and mouse PD-L1.

SEQ ID NO: 307
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLENVTST
LRINTTTNEIFYCIFRRLDPEENHTAELVIPELPLAHPPNERTHLVILG
AILLCLGVALTFIFRLRKGRMMDVKKCGIQDTNSKKQSDTHLEET

In some embodiments, the PD-L1 binding agent binds PD-L1 without substantially inhibiting or preventing PD-L1 from binding to its receptor, PD-1. However, in other embodiments, the PD-L1 binding agent can completely or partially block (inhibit or prevent) binding of PD-L1 to its receptor, PD-1, such that the antibody can be used to inhibit PD-L1/PD-signaling (e.g., for therapeutic purposes).

The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1-binding agent also can be an antibody conjugate. In this respect, the PD-L1-binding agent can be a conjugate of (1) an antibody, an alternative scaffold, or fragments thereof, and (2) a protein or non-protein moiety. For example, the PD-L1 binding agent can be conjugated to a peptide, a fluorescent molecule, chemotherapeutic or other cytotoxic payload, immune-activating or immune-suppressive agent.

The PD-L1-binding agent can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

### PD-L1-Binding Agents—Type A

Provided herein are PD-L1 binding agents comprising an immunoglobulin heavy chain variable region polypeptide and an immunoglobulin light chain variable region polypeptide. In some embodiments, the PD-L1 binding agents (Type A) comprise an immunoglobulin heavy chain variable region of any one of SEQ ID NOs: 223-264, or at least the CDRs thereof; and an immunoglobulin light chain variable region of any one of SEQ ID NOs: 265-306 or at least the CDRs thereof. In other embodiments, the PD-L1 binding agents (Type A) comprise an immunoglobulin heavy chain variable region polypeptide with an amino acid sequence that is at least 90% identical to any one of SEQ ID NOs: 223-264, and an immunoglobulin light chain variable region polypeptide with an amino acid sequence that is at least 90% identical to any one of SEQ ID NOs: 265-306. In yet other embodiments, the PD-L1 binding agent (Type A), the immunoglobulin heavy chain variable region polypeptide comprises a complementarity determining region 1 (HCDR1) comprising any one of SEQ ID NOs: 1-23, a complementarity determining region 2 (HCDR2) comprising any one of SEQ ID NOs: 24-57, and a complementarity determining region 3 (HCDR3) comprising any one of SEQ ID NOs: 58-95; and/or the immunoglobulin light chain variable region polypeptide comprises a complementarity determining region 1 (LCDR1) comprising any one of SEQ ID NOs: 96-128, a complementarity determining region 2 (LCDR2) comprising any one of SEQ ID NOs: 129-151, and a complementarity determining region 3 (LCDR3) comprising any one of SEQ ID NOs: 152-155. Also provided are nucleic acids encoding the PD-L1 binding agents, or the individual heavy and light chains thereof; vectors and cells comprising the nucleic acids; and compositions comprising the binding agents or nucleic acids.

Furthermore, in some embodiments, the PD-L1 binding agents (Type A) provided herein cause cellular internalization of PD-L1 or the PD-L1/PD-L1 binding agent complex upon binding to PD-L1 on the cell surface. Without wishing to be bound by any particular theory or mechanism of action, it is believed that the PD-L1 binding agents according to this embodiment cause PD-L1 internalization upon binding, and remain bound to PD-L1 during internalization, resulting in internalization of the binding agent along with PD-L1. Cellular internalization of PD-L1 and bound PD-L1 binding agent can be determined by any suitable method, such as assaying for persistence on the cell surface and/or detection of internalized antibodies. In some embodiments, the PD-L1 binding agent internalizes strongly enough that at least about 25% (e.g., at least about 35%, at least about 50%, at least about 75%, or at least about 90%) of the PD-L1 binding agent that binds PD-L1 on the cell surface is internalized (e.g., using a surface persistence assay, about 75% or less, about 65% or less, about 50% or less, about 25% or less or about 10% or less of PD-L1 binding agent molecules bound to PD-L1 on the cell surface at the beginning of the assay remain bound at the end of the assay).

In an embodiment, the PD-L1 binding agent (Type A) comprises an immunoglobulin heavy chain variable region of any one of SEQ ID NOs: 223-264, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 223-264, or at least the CDRs thereof, and/or an immunoglobulin light chain variable region of any one of SEQ ID NOs: 265-306, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 265-306, or at least the CDRs thereof.

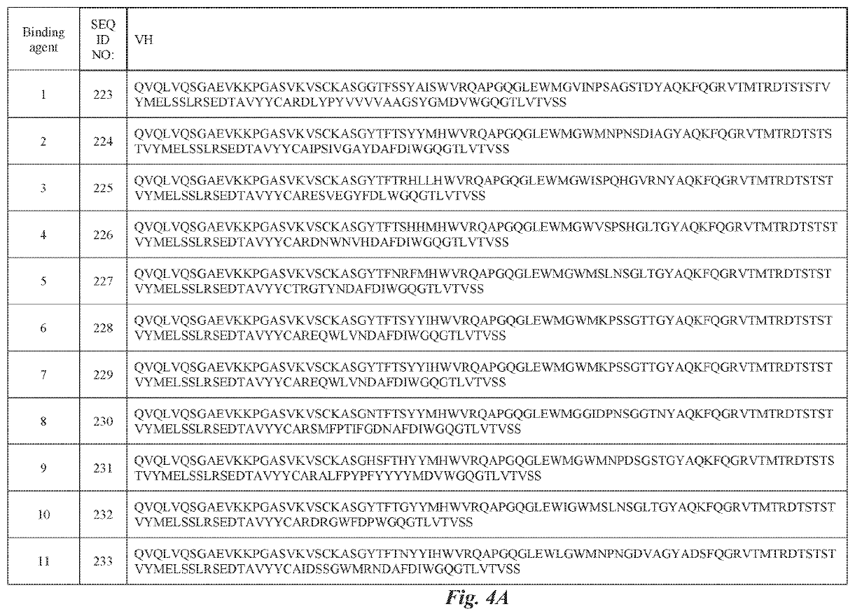

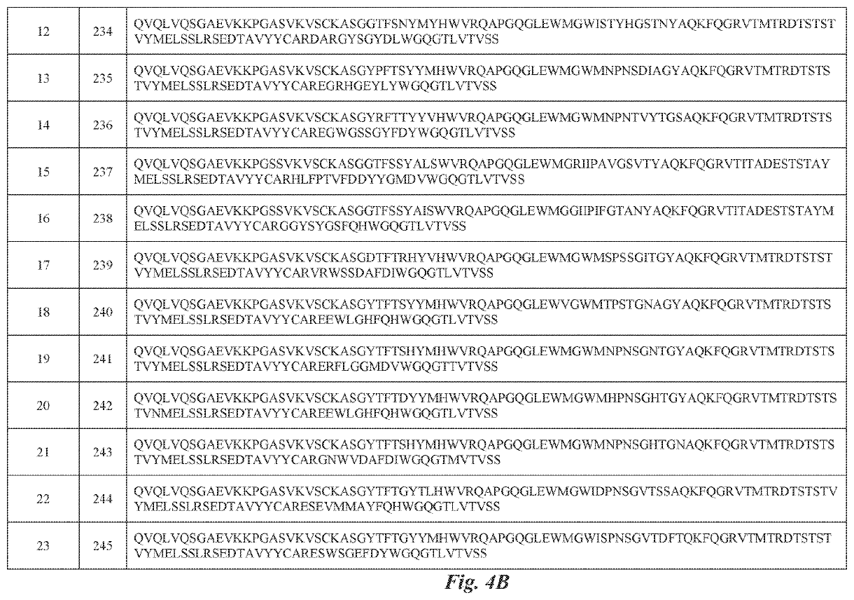

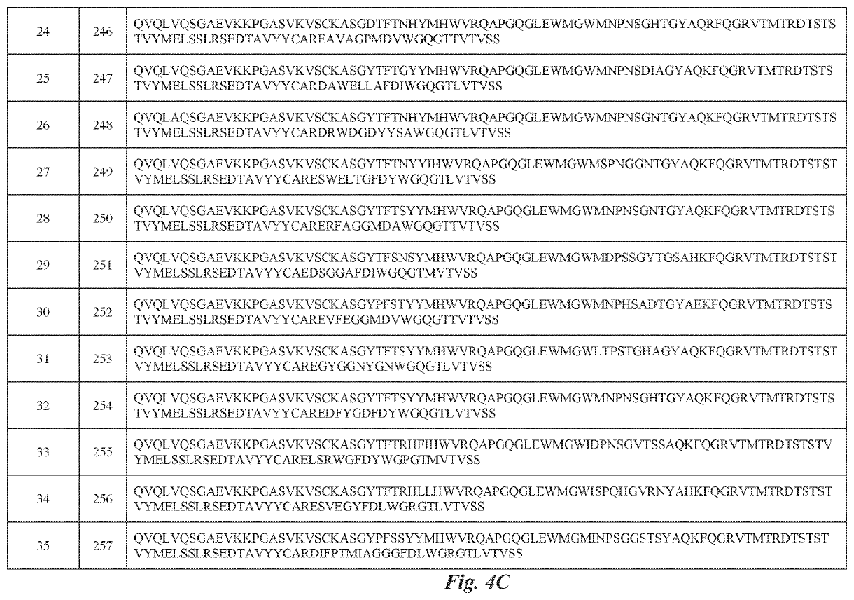

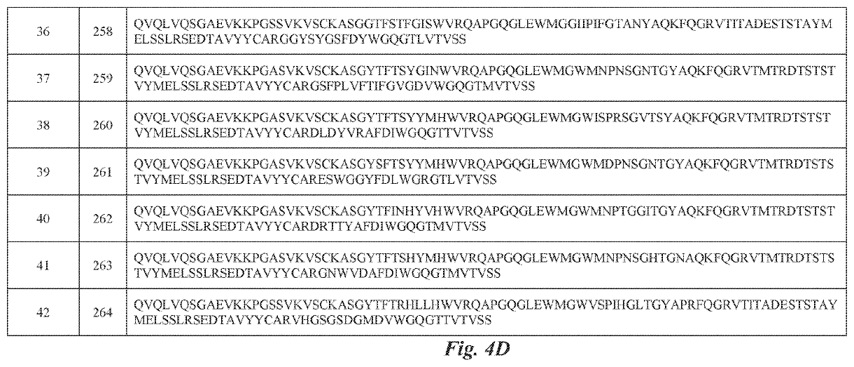

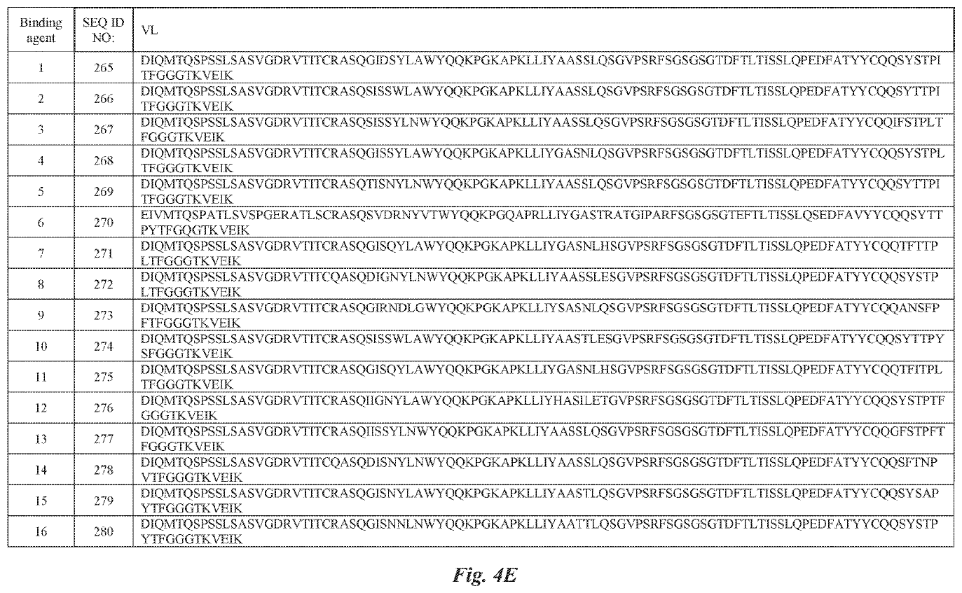

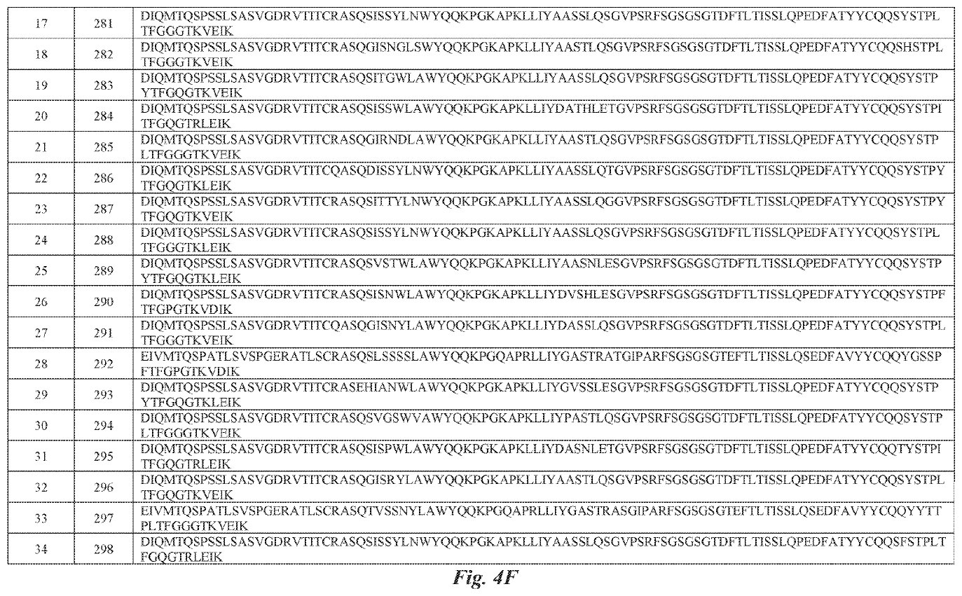

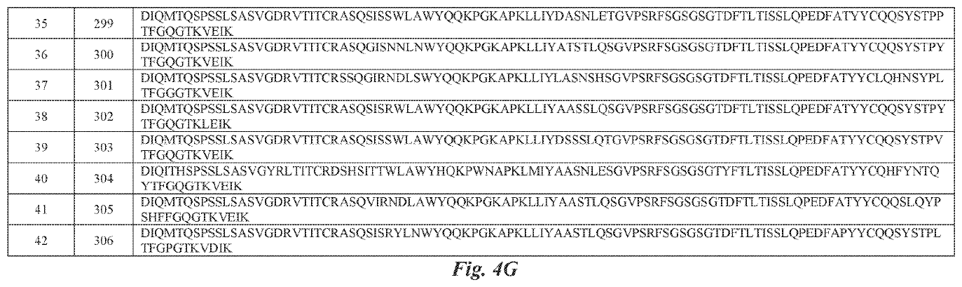

By way of further illustration, the PD-L1 binding agent (Type A) can comprise:
(1) an immunoglobulin heavy chain variable region of SEQ ID NO: 223, or at least the CDRs thereof, and/or an immunoglobulin light chain variable region of SEQ ID NO: 265, or at least the CDRs thereof;
(2) an immunoglobulin heavy chain variable region of SEQ ID NO: 224, or at least the CDRs thereof, and/or an immunoglobulin light chain variable region of SEQ ID NO: 266, or at least the CDRs thereof;
(3) an immunoglobulin heavy chain variable region of SEQ ID NO: 225, or at least the CDRs thereof, and/or an immunoglobulin light chain variable region of SEQ ID NO: 267, or at least the CDRs thereof;
(4) an immunoglobulin heavy chain variable region of SEQ ID NO: 226, or at least the CDRs thereof, and/or an immunoglobulin light chain variable region of SEQ ID NO: 268, or at least the CDRs thereof;
(5) an immunoglobulin heavy chain variable region of SEQ ID NO: 227, or at least the CDRs thereof, and/or an immunoglobulin light chain variable region of SEQ ID NO: 269, or at least the CDRs thereof;
(6) an immunoglobulin heavy chain variable region of SEQ ID NO: 228, or at least the CDRs thereof, and/or an immunoglobulin light chain variable region of SEQ ID NO: 270, or at least the CDRs thereof;
(7) an immunoglobulin heavy chain variable region of SEQ ID NO: 229, or at least the CDRs thereof, and/or an immunoglobulin light chain variable region of SEQ ID NO: 271, or at least the CDRs thereof;
(8) an immunoglobulin heavy chain variable region of SEQ ID NO: 230, or at least the CDRs thereof, and/or an immunoglobulin light chain variable region of SEQ ID NO: 272, or at least the CDRs thereof;
(9) an immunoglobulin heavy chain variable region of SEQ ID NO: 231, or at least the CDRs thereof, and/or an immunoglobulin light chain variable region of SEQ ID NO: 273, or at least the CDRs thereof;
(10) an immunoglobulin heavy chain variable region of SEQ ID NO: 232, or at least the CDRs thereof, and/or an immunoglobulin light chain variable region of SEQ ID NO: 274, or at least the CDRs thereof;
(11) an immunoglobulin heavy chain variable region of SEQ ID NO: 233, or at least the CDRs thereof, and/or an immunoglobulin light chain variable region of SEQ ID NO: 275, or at least the CDRs thereof;
(12) an immunoglobulin heavy chain variable region of SEQ ID NO: 234, or at least the CDRs thereof, and/or an immunoglobulin light chain variable region of SEQ ID NO: 276, or at least the CDRs thereof;
(13) an immunoglobulin heavy chain variable region of SEQ ID NO: 235, or at least the CDRs thereof, and/or an immunoglobulin light chain variable region of SEQ ID NO: 277, or at least the CDRs thereof;
(14) an immunoglobulin heavy chain variable region of SEQ ID NO: 236, or at least the CDRs thereof, and/or an immunoglobulin light chain variable region of SEQ ID NO: 278, or at least the CDRs thereof;
(15) an immunoglobulin heavy chain variable region of SEQ ID NO: 237, or at least the CDRs thereof, and/or an immunoglobulin light chain variable region of SEQ ID NO: 279, or at least the CDRs thereof;
(16) an immunoglobulin heavy chain variable region of SEQ ID NO: 238, or at least the CDRs thereof, and/or an immunoglobulin light chain variable region of SEQ ID NO: 280, or at least the CDRs thereof;
(17) an immunoglobulin heavy chain variable region of SEQ ID NO: 239, or at least the CDRs thereof, and/or an immunoglobulin light chain variable region of SEQ ID NO: 281, or at least the CDRs thereof;
(18) an immunoglobulin heavy chain variable region of SEQ ID NO: 240, or at least the CDRs thereof, and/or an immunoglobulin light chain variable region of SEQ ID NO: 282, or at least the CDRs thereof;
(19) an immunoglobulin heavy chain variable region of SEQ ID NO: 241, or at least the CDRs thereof, and/or an immunoglobulin light chain variable region of SEQ ID NO: 283, or at least the CDRs thereof;
(20) an immunoglobulin heavy chain variable region of SEQ ID NO: 242, or at least the CDRs thereof, and/or an immunoglobulin light chain variable region of SEQ ID NO: 284, or at least the CDRs thereof;
(21) an immunoglobulin heavy chain variable region of SEQ ID NO: 243, or at least the CDRs thereof, and/or an immunoglobulin light chain variable region of SEQ ID NO: 285, or at least the CDRs thereof;
(22) an immunoglobulin heavy chain variable region of SEQ ID NO: 244, or at least the CDRs thereof, and/or an immunoglobulin light chain variable region of SEQ ID NO: 286, or at least the CDRs thereof;
(23) an immunoglobulin heavy chain variable region of SEQ ID NO: 245, or at least the CDRs thereof, and/or an immunoglobulin light chain variable region of SEQ ID NO: 287, or at least the CDRs thereof;
(24) an immunoglobulin heavy chain variable region of SEQ ID NO: 246, or at least the CDRs thereof, and/or an immunoglobulin light chain variable region of SEQ ID NO: 288, or at least the CDRs thereof;
(25) an immunoglobulin heavy chain variable region of SEQ ID NO: 247, or at least the CDRs thereof, and/or an immunoglobulin light chain variable region of SEQ ID NO: 289, or at least the CDRs thereof;
(26) an immunoglobulin heavy chain variable region of SEQ ID NO: 248, or at least the CDRs thereof, and/or an immunoglobulin light chain variable region of SEQ ID NO: 290, or at least the CDRs thereof;
(27) an immunoglobulin heavy chain variable region of SEQ ID NO: 249, or at least the CDRs thereof, and/or an immunoglobulin light chain variable region of SEQ ID NO: 291, or at least the CDRs thereof;
(28) an immunoglobulin heavy chain variable region of SEQ ID NO: 250, or at least the CDRs thereof, and/or an immunoglobulin light chain variable region of SEQ ID NO: 292, or at least the CDRs thereof;
(29) an immunoglobulin heavy chain variable region of SEQ ID NO: 251, or at least the CDRs thereof, and/or an immunoglobulin light chain variable region of SEQ ID NO: 293, or at least the CDRs thereof;
(30) an immunoglobulin heavy chain variable region of SEQ ID NO: 252, or at least the CDRs thereof, and/or an immunoglobulin light chain variable region of SEQ ID NO: 294, or at least the CDRs thereof;
(31) an immunoglobulin heavy chain variable region of SEQ ID NO: 253, or at least the CDRs thereof, and/or an immunoglobulin light chain variable region of SEQ ID NO: 295, or at least the CDRs thereof;
(32) an immunoglobulin heavy chain variable region of SEQ ID NO: 254, or at least the CDRs thereof, and/or an immunoglobulin light chain variable region of SEQ ID NO: 296, or at least the CDRs thereof;
(33) an immunoglobulin heavy chain variable region of SEQ ID NO: 255, or at least the CDRs thereof, and/or an immunoglobulin light chain variable region of SEQ ID NO: 297, or at least the CDRs thereof;
(34) an immunoglobulin heavy chain variable region of SEQ ID NO: 256, or at least the CDRs thereof, and/or an immunoglobulin light chain variable region of SEQ ID NO: 298, or at least the CDRs thereof;
(35) an immunoglobulin heavy chain variable region of SEQ ID NO: 257, or at least the CDRs thereof, and/or an immunoglobulin light chain variable region of SEQ ID NO: 299, or at least the CDRs thereof;
(36) an immunoglobulin heavy chain variable region of SEQ ID NO: 258, or at least the CDRs thereof, and/or an immunoglobulin light chain variable region of SEQ ID NO: 300, or at least the CDRs thereof;
(37) an immunoglobulin heavy chain variable region of SEQ ID NO: 259, or at least the CDRs thereof, and/or an immunoglobulin light chain variable region of SEQ ID NO: 301, or at least the CDRs thereof;
(38) an immunoglobulin heavy chain variable region of SEQ ID NO: 260, or at least the CDRs thereof, and/or an immunoglobulin light chain variable region of SEQ ID NO: 302, or at least the CDRs thereof;
(39) an immunoglobulin heavy chain variable region of SEQ ID NO: 261, or at least the CDRs thereof, and/or an immunoglobulin light chain variable region of SEQ ID NO: 303, or at least the CDRs thereof;
(40) an immunoglobulin heavy chain variable region of SEQ ID NO: 262, or at least the CDRs thereof, and/or an immunoglobulin light chain variable region of SEQ ID NO: 304, or at least the CDRs thereof;
(41) an immunoglobulin heavy chain variable region of SEQ ID NO: 263, or at least the CDRs thereof, and/or an immunoglobulin light chain variable region of SEQ ID NO: 305, or at least the CDRs thereof;
(42) an immunoglobulin heavy chain variable region of SEQ ID NO: 264, or at least the CDRs thereof, and/or an immunoglobulin light chain variable region of SEQ ID NO: 306, or at least the CDRs thereof; and/or
(43) an immunoglobulin heavy chain variable region of FIGS. 4A-D and/or an immunoglobulin light chain variable region of FIGS. 4E-G, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent (Type A) comprises one or more of the following CDRs:
a HCDR1 comprising or consisting of any one of SEQ ID NOs: 1-23 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 1-23;
a HCDR2 comprising or consisting of any one of SEQ ID NOs: 24-57 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 24-57; and
a HCDR3 comprising or consisting of any one of SEQ ID NOs: 58-95 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 58-95; and/or the immunoglobulin light chain polypeptide comprises
a LCDR1 comprising or consisting of any one of SEQ ID NOs: 96-128 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 96-128;
a LCDR2 comprising or consisting of any one of SEQ ID NOs: 129-151 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 129-151; and
a LCDR3 comprising or consisting of any one of SEQ ID NOs: 152-155 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 152-155.

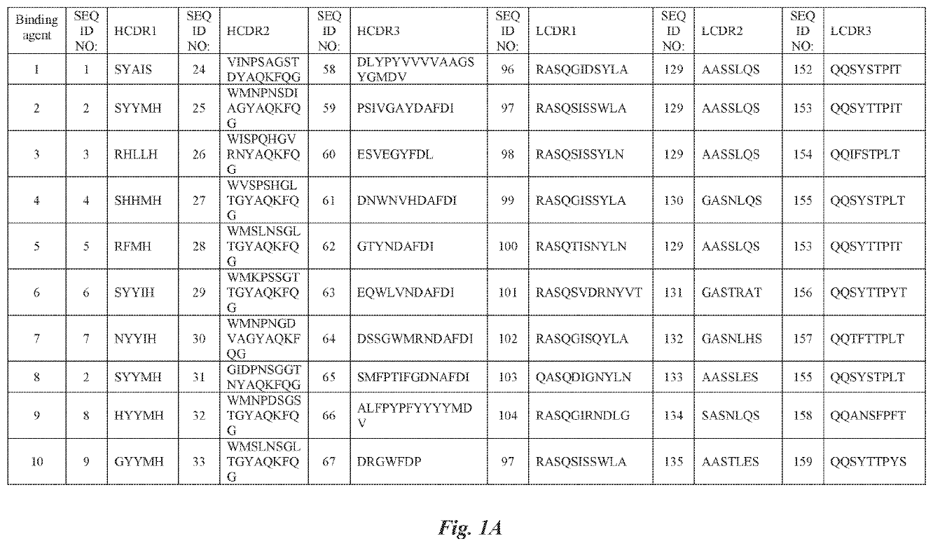

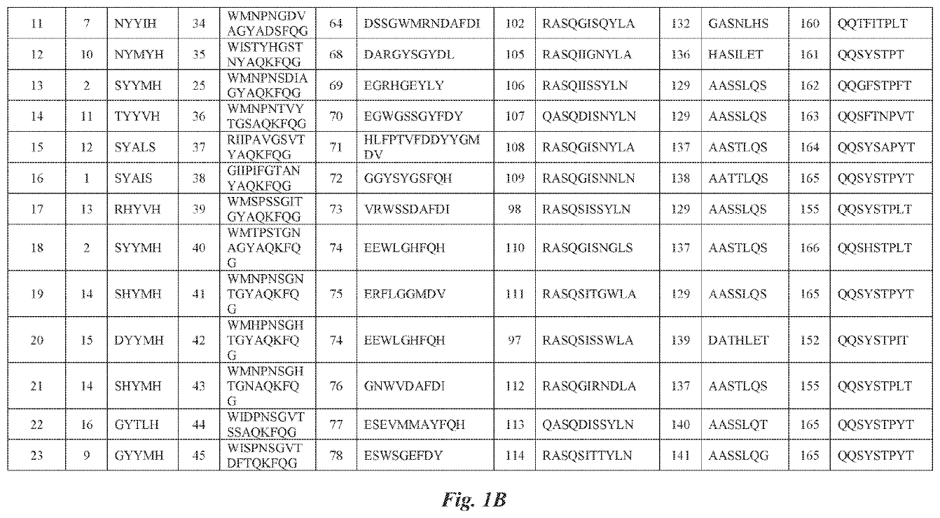

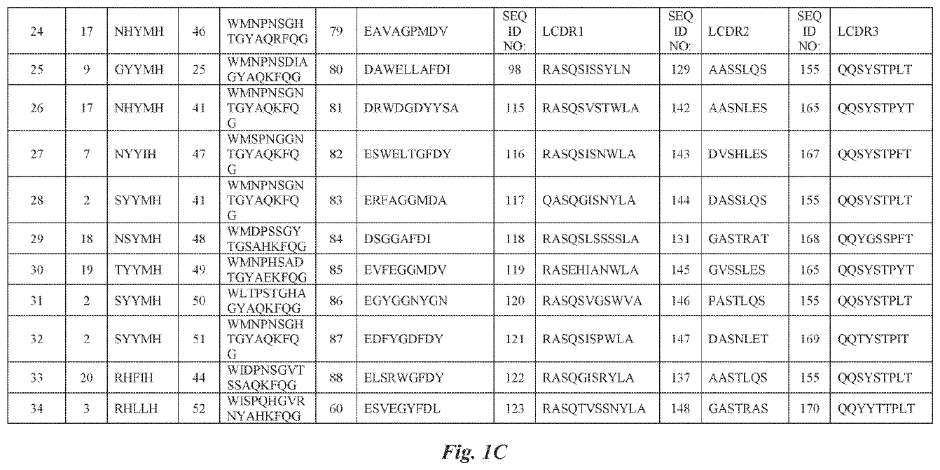

In particular embodiments, the binding agent (Type A) comprises an immunoglobulin heavy chain polypeptide and an immunoglobulin light chain polypeptide, wherein:
(1) the immunoglobulin heavy chain polypeptide comprises a HCDR1 comprising or consisting of SEQ ID NO: 1, a HCDR2 comprising or consisting of SEQ ID NO: 24, and a HCDR3 comprising or consisting of SEQ ID NO: 58; and/or the immunoglobulin light chain polypeptide comprises a LCDR1 comprising or consisting of SEQ ID NO: 96, a LCDR2 comprising or consisting of SEQ ID NO: 129, and a LCDR3 comprising or consisting of SEQ ID NO: 152;
(2) the immunoglobulin heavy chain polypeptide comprises a HCDR1 comprising or consisting of SEQ ID NO: 2, a HCDR2 comprising or consisting of SEQ ID NO: 25, and a HCDR3 comprising or consisting of SEQ ID NO: 59; and/or the immunoglobulin light chain polypeptide comprises a LCDR1 comprising or consisting of SEQ ID NO: 97, a LCDR2 comprising or consisting of SEQ ID NO: 129, and a LCDR3 comprising or consisting of SEQ ID NO: 153;
(3) the immunoglobulin heavy chain polypeptide comprises a HCDR1 comprising or consisting of SEQ ID NO: 3, a HCDR2 comprising or consisting of SEQ ID NO: 26, and a HCDR3 comprising or consisting of SEQ ID NO: 60; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 154;
(4) the immunoglobulin heavy chain polypeptide comprises a HCDR1 comprising or consisting of SEQ ID NO: 4, a HCDR2 comprising or consisting of SEQ ID NO: 27, and a HCDR3 comprising or consisting of SEQ ID NO: 61; and/or the immunoglobulin light chain polypeptide comprises a LCDR1 comprising or consisting of SEQ ID NO: 99, a LCDR2 comprising or consisting of SEQ ID NO: 130, and a LCDR3 comprising or consisting of SEQ ID NO: 155;
(5) the immunoglobulin heavy chain polypeptide comprises a HCDR1 comprising or consisting of SEQ ID NO: 5, a HCDR2 comprising or consisting of SEQ ID NO: 28, and a HCDR3 comprising or consisting of SEQ ID NO: 62; and/or the immunoglobulin light chain polypeptide comprises a LCDR1 comprising or consisting of SEQ ID NO: 100, a LCDR2 comprising or consisting of SEQ ID NO: 129, and a LCDR3 comprising or consisting of SEQ ID NO: 153;
(6) the immunoglobulin heavy chain polypeptide comprises a HCDR1 comprising or consisting of SEQ ID NO: 6, a HCDR2 comprising or consisting of SEQ ID NO: 29, and a HCDR3 comprising or consisting of SEQ ID NO: 63; and/or the immunoglobulin light chain polypeptide comprises a LCDR1 comprising or consisting of SEQ ID NO: 101, a LCDR2 comprising or consisting of SEQ ID NO: 131, and a LCDR3 comprising or consisting of SEQ ID NO: 156;
(7) the immunoglobulin heavy chain polypeptide comprises a HCDR1 comprising or consisting of SEQ ID NO: 7, a HCDR2 comprising or consisting of SEQ ID NO: 30, and a HCDR3 comprising or consisting of SEQ ID NO: 64; and/or the immunoglobulin light chain polypeptide comprises a LCDR1 comprising or consisting of SEQ ID NO: 102, a LCDR2 comprising or consisting of SEQ ID NO: 132, and a LCDR3 comprising or consisting of SEQ ID NO: 157;
(8) the immunoglobulin heavy chain polypeptide comprises a HCDR1 comprising or consisting of SEQ ID NO: 2, a HCDR2 comprising or consisting of SEQ ID NO: 31, and a HCDR3 comprising or consisting of SEQ ID NO: 65; and/or the immunoglobulin light chain polypeptide comprises a LCDR1 comprising or consisting of SEQ ID NO: 103, a LCDR2 comprising or consisting of SEQ ID NO: 133, and a LCDR3 comprising or consisting of SEQ ID NO: 155;
(9) the immunoglobulin heavy chain polypeptide comprises a HCDR1 comprising or consisting of SEQ ID NO: 8, a HCDR2 comprising or consisting of SEQ ID NO: 32, and a HCDR3 comprising or consisting of SEQ ID NO: 66; and/or the immunoglobulin light chain polypeptide comprises a LCDR1 comprising or consisting of SEQ ID NO: 104, a LCDR2 comprising or consisting of SEQ ID NO: 134, and a LCDR3 comprising or consisting of SEQ ID NO: 158;
(10) the immunoglobulin heavy chain polypeptide comprises a HCDR1 comprising or consisting of SEQ ID NO: 9, a HCDR2 comprising or consisting of SEQ ID NO: 33, and a HCDR3 comprising or consisting of SEQ ID NO: 67; and/or the immunoglobulin light chain polypeptide comprises a LCDR1 comprising or consisting of SEQ ID NO: 97, a LCDR2 comprising or consisting of SEQ ID NO: 135, and a LCDR3 comprising or consisting of SEQ ID NO: 159;
(11) the immunoglobulin heavy chain polypeptide comprises a HCDR1 comprising or consisting of SEQ ID NO: 7, a HCDR2 comprising or consisting of SEQ ID NO: 34, and a HCDR3 comprising or consisting of SEQ ID NO: 64; and/or the immunoglobulin light chain polypeptide comprises a LCDR1 comprising or consisting of SEQ ID NO: 102, a LCDR2 comprising or consisting of SEQ ID NO: 132, and a LCDR3 comprising or consisting of SEQ ID NO: 160;
(12) the immunoglobulin heavy chain polypeptide comprises a HCDR1 comprising or consisting of SEQ ID NO: 10, a HCDR2 comprising or consisting of SEQ ID NO: 35, and a HCDR3 comprising or consisting of SEQ ID NO: 68; and/or the immunoglobulin light chain polypeptide comprises a LCDR1 comprising or consisting of SEQ ID NO: 105, a LCDR2 comprising or consisting of SEQ ID NO: 136, and a LCDR3 comprising or consisting of SEQ ID NO: 161;
(13) the immunoglobulin heavy chain polypeptide comprises a HCDR1 comprising or consisting of SEQ ID NO: 2, a HCDR2 comprising or consisting of SEQ ID NO: 25, and a HCDR3 comprising or consisting of SEQ ID NO: 69; and/or the immunoglobulin light chain polypeptide comprises a LCDR1 comprising or consisting of SEQ ID NO: 106, a LCDR2 comprising or consisting of SEQ ID NO: 129, and a LCDR3 comprising or consisting of SEQ ID NO: 162;
(14) the immunoglobulin heavy chain polypeptide comprises a HCDR1 comprising or consisting of SEQ ID NO: 11, a HCDR2 comprising or consisting of SEQ ID NO: 36, and a HCDR3 comprising or consisting of SEQ ID NO: 70; and/or the immunoglobulin light chain polypeptide comprises a LCDR1 comprising or consisting of SEQ ID NO: 107, a LCDR2 comprising or consisting of SEQ ID NO: 129, and a LCDR3 comprising or consisting of SEQ ID NO: 163;
(15) the immunoglobulin heavy chain polypeptide comprises a HCDR1 comprising or consisting of SEQ ID NO: 12, a HCDR2 comprising or consisting of SEQ ID NO: 37, and a HCDR3 comprising or consisting of SEQ ID NO: 71; and/or the immunoglobulin light chain polypeptide comprises a LCDR1 comprising or consisting of SEQ ID NO: 108, a LCDR2 comprising or consisting of SEQ ID NO: 137, and a LCDR3 comprising or consisting of SEQ ID NO: 164;
(16) the immunoglobulin heavy chain polypeptide comprises a HCDR1 comprising or consisting of SEQ ID NO: 1, a HCDR2 comprising or consisting of SEQ ID NO: 38, and a HCDR3 comprising or consisting of SEQ ID NO: 72; and/or the immunoglobulin light chain polypeptide comprises a LCDR1 comprising or consisting of SEQ ID NO: 109, a LCDR2 comprising or consisting of SEQ ID NO: 138, and a LCDR3 comprising or consisting of SEQ ID NO: 165;
(17) the immunoglobulin heavy chain polypeptide comprises a HCDR1 comprising or consisting of SEQ ID NO: 13, a HCDR2 comprising or consisting of SEQ ID NO: 39, and a HCDR3 comprising or consisting of SEQ ID NO: 73; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 155;
(18) the immunoglobulin heavy chain polypeptide comprises a HCDR1 comprising or consisting of SEQ ID NO: 2, a HCDR2 comprising or consisting of SEQ ID NO: 40, and a HCDR3 comprising or consisting of SEQ ID NO: 74; and/or the immunoglobulin light chain polypeptide comprises a LCDR1 comprising or consisting of SEQ ID NO: 110, a LCDR2 comprising or consisting of SEQ ID NO: 137, and a LCDR3 comprising or consisting of SEQ ID NO: 166;
(19) the immunoglobulin heavy chain polypeptide comprises a HCDR1 comprising or consisting of SEQ ID NO: 14, a HCDR2 comprising or consisting of SEQ ID NO: 41, and a HCDR3 comprising or consisting of SEQ ID NO: 75; and/or the immunoglobulin light chain polypeptide comprises a LCDR1 comprising or consisting of SEQ ID NO: 111, a LCDR2 comprising or consisting of SEQ ID NO: 129, and a LCDR3 comprising or consisting of SEQ ID NO: 165;
(20) the immunoglobulin heavy chain polypeptide comprises a HCDR1 comprising or consisting of SEQ ID NO: 15, a HCDR2 comprising or consisting of SEQ ID NO: 42, and a HCDR3 comprising or consisting of SEQ ID NO: 74; and/or the immunoglobulin light chain polypeptide comprises a LCDR1 comprising or consisting of SEQ ID NO: 97, a LCDR2 comprising or consisting of SEQ ID NO: 139, and a LCDR3 comprising or consisting of SEQ ID NO: 152;
(21) the immunoglobulin heavy chain polypeptide comprises a HCDR1 comprising or consisting of SEQ ID NO: 14, a HCDR2 comprising or consisting of SEQ ID NO: 43, and a HCDR3 comprising or consisting of SEQ ID NO: 76; and/or the immunoglobulin light chain polypeptide comprises a LCDR1 comprising or consisting of SEQ ID NO: 112, a LCDR2 comprising or consisting of SEQ ID NO: 137, and a LCDR3 comprising or consisting of SEQ ID NO: 155;
(22) the immunoglobulin heavy chain polypeptide comprises a HCDR1 comprising or consisting of SEQ ID NO: 16, a HCDR2 comprising or consisting of SEQ ID NO: 44, and a HCDR3 comprising or consisting of SEQ ID NO: 77; and/or the immunoglobulin light chain polypeptide comprises a LCDR1 comprising or consisting of SEQ ID NO: 113, a LCDR2 comprising or consisting of SEQ ID NO: 140, and a LCDR3 comprising or consisting of SEQ ID NO: 165;
(23) the immunoglobulin heavy chain polypeptide comprises a HCDR1 comprising or consisting of SEQ ID NO: 9, a HCDR2 comprising or consisting of SEQ ID NO: 45, and a HCDR3 comprising or consisting of SEQ ID NO: 78; and/or the immunoglobulin light chain polypeptide comprises a LCDR1 comprising or consisting of SEQ ID NO: 114, a LCDR2 comprising or consisting of SEQ ID NO: 141, and a LCDR3 comprising or consisting of SEQ ID NO: 165;
(24) the immunoglobulin heavy chain polypeptide comprises a HCDR1 comprising or consisting of SEQ ID NO: 17, a HCDR2 comprising or consisting of SEQ ID NO: 46, and a HCDR3 comprising or consisting of SEQ ID NO: 79; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 155;
(25) the immunoglobulin heavy chain polypeptide comprises a HCDR1 comprising or consisting of SEQ ID NO: 9, a HCDR2 comprising or consisting of SEQ ID NO: 25, and a HCDR3 comprising or consisting of SEQ ID NO: 80; and/or the immunoglobulin light chain polypeptide comprises a LCDR1 comprising or consisting of SEQ ID NO: 115, a LCDR2 comprising or consisting of SEQ ID NO: 142, and a LCDR3 comprising or consisting of SEQ ID NO: 165;
(26) the immunoglobulin heavy chain polypeptide comprises a HCDR1 comprising or consisting of SEQ ID NO: 17, a HCDR2 comprising or consisting of SEQ ID NO: 41, and a HCDR3 comprising or consisting of SEQ ID NO: 81; and/or the immunoglobulin light chain polypeptide comprises a LCDR1 comprising or consisting of SEQ ID NO: 116, a LCDR2 comprising or consisting of SEQ ID NO: 143, and a LCDR3 comprising or consisting of SEQ ID NO: 167;
(27) the immunoglobulin heavy chain polypeptide comprises a HCDR1 comprising or consisting of SEQ ID NO: 7, a HCDR2 comprising or consisting of SEQ ID NO: 47, and a HCDR3 comprising or consisting of SEQ ID NO: 82; and/or the immunoglobulin light chain polypeptide comprises a LCDR1 comprising or consisting of SEQ ID NO: 117, a LCDR2 comprising or consisting of SEQ ID NO: 144, and a LCDR3 comprising or consisting of SEQ ID NO: 155;
(28) the immunoglobulin heavy chain polypeptide comprises a HCDR1 comprising or consisting of SEQ ID NO: 2, a HCDR2 comprising or consisting of SEQ ID NO: 41, and a HCDR3 comprising or consisting of SEQ ID NO: 83; and/or the immunoglobulin light chain polypeptide comprises a LCDR1 comprising or consisting of SEQ ID NO: 118, a LCDR2 comprising or consisting of SEQ ID NO: 131, and a LCDR3 comprising or consisting of SEQ ID NO: 168;
(29) the immunoglobulin heavy chain polypeptide comprises a HCDR1 comprising or consisting of SEQ ID NO: 18, a HCDR2 comprising or consisting of SEQ ID NO: 48, and a HCDR3 comprising or consisting of SEQ ID NO: 84; and/or the immunoglobulin light chain polypeptide comprises a LCDR1 comprising or consisting of SEQ ID NO: 119, a LCDR2 comprising or consisting of SEQ ID NO: 145, and a LCDR3 comprising or consisting of SEQ ID NO: 165;
(30) the immunoglobulin heavy chain polypeptide comprises a HCDR1 comprising or consisting of SEQ ID NO: 19, a HCDR2 comprising or consisting of SEQ ID NO: 49, and a HCDR3 comprising or consisting of SEQ ID NO: 85; and/or the immunoglobulin light chain polypeptide comprises a LCDR1 comprising or consisting of SEQ ID NO: 120, a LCDR2 comprising or consisting of SEQ ID NO: 146, and a LCDR3 comprising or consisting of SEQ ID NO: 155;
(31) the immunoglobulin heavy chain polypeptide comprises a HCDR1 comprising or consisting of SEQ ID NO: 2, a HCDR2 comprising or consisting of SEQ ID NO: 50, and a HCDR3 comprising or consisting of SEQ ID NO: 86; and/or the immunoglobulin light chain polypeptide comprises a LCDR1 comprising or consisting of SEQ ID NO: 121, a LCDR2 comprising or consisting of SEQ ID NO: 147, and a LCDR3 comprising or consisting of SEQ ID NO: 169;
(32) the immunoglobulin heavy chain polypeptide comprises a HCDR1 comprising or consisting of SEQ ID NO: 2, a HCDR2 comprising or consisting of SEQ ID NO: 51, and a HCDR3 comprising or consisting of SEQ ID NO: 87; and/or the immunoglobulin light chain polypeptide comprises a LCDR1 comprising or consisting of SEQ ID NO: 122, a LCDR2 comprising or consisting of SEQ ID NO: 137, and a LCDR3 comprising or consisting of SEQ ID NO: 155;
(33) the immunoglobulin heavy chain polypeptide comprises a HCDR1 comprising or consisting of SEQ ID NO: 20, a HCDR2 comprising or consisting of SEQ ID NO: 44, and a HCDR3 comprising or consisting of SEQ ID NO: 88; and/or the immunoglobulin light chain polypeptide comprises a LCDR1 comprising or consisting of SEQ ID NO: 123, a LCDR2 comprising or consisting of SEQ ID NO: 148, and a LCDR3 comprising or consisting of SEQ ID NO: 170;
(34) the immunoglobulin heavy chain polypeptide comprises a HCDR1 comprising or consisting of SEQ ID NO: 3, a HCDR2 comprising or consisting of SEQ ID NO: 52, and a HCDR3 comprising or consisting of SEQ ID NO: 60; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 171;
(35) the immunoglobulin heavy chain polypeptide comprises a HCDR1 comprising or consisting of SEQ ID NO: 2, a HCDR2 comprising or consisting of SEQ ID NO: 53, and a HCDR3 comprising or consisting of SEQ ID NO: 89; and/or the immunoglobulin light chain polypeptide comprises a LCDR1 comprising or consisting of SEQ ID NO: 97, a LCDR2 comprising or consisting of SEQ ID NO: 147, and a LCDR3 comprising or consisting of SEQ ID NO: 172;
(36) the immunoglobulin heavy chain polypeptide comprises a HCDR1 comprising or consisting of SEQ ID NO: 21, a HCDR2 comprising or consisting of SEQ ID NO: 38, and a HCDR3 comprising or consisting of SEQ ID NO: 90; and/or the immunoglobulin light chain polypeptide comprises a LCDR1 comprising or consisting of SEQ ID NO: 109, a LCDR2 comprising or consisting of SEQ ID NO: 150, and a LCDR3 comprising or consisting of SEQ ID NO: 165;
(37) the immunoglobulin heavy chain polypeptide comprises a HCDR1 comprising or consisting of SEQ ID NO: 22, a HCDR2 comprising or consisting of SEQ ID NO: 41, and a HCDR3 comprising or consisting of SEQ ID NO: 91; and/or the immunoglobulin light chain polypeptide comprises a LCDR1 comprising or consisting of SEQ ID NO: 124, a LCDR2 comprising or consisting of SEQ ID NO: 151, and a LCDR3 comprising or consisting of SEQ ID NO: 173;
(38) the immunoglobulin heavy chain polypeptide comprises a HCDR1 comprising or consisting of SEQ ID NO: 2, a HCDR2 comprising or consisting of SEQ ID NO: 54, and a HCDR3 comprising or consisting of SEQ ID NO: 92; and/or the immunoglobulin light chain polypeptide comprises a LCDR1 comprising or consisting of SEQ ID NO: 126, a LCDR2 comprising or consisting of SEQ ID NO: 129, and a LCDR3 comprising or consisting of SEQ ID NO: 165;
(39) the immunoglobulin heavy chain polypeptide comprises a HCDR1 comprising or consisting of SEQ ID NO: 2, a HCDR2 comprising or consisting of SEQ ID NO: 55, and a HCDR3 comprising or consisting of SEQ ID NO: 93; and/or the immunoglobulin light chain polypeptide comprises a LCDR1 comprising or consisting of SEQ ID NO: 97, a LCDR2 comprising or consisting of SEQ ID NO: 149, and a LCDR3 comprising or consisting of SEQ ID NO: 174;
(40) the immunoglobulin heavy chain polypeptide comprises a HCDR1 comprising or consisting of SEQ ID NO: 23, a HCDR2 comprising or consisting of SEQ ID NO: 56, and a HCDR3 comprising or consisting of SEQ ID NO: 94; and/or the immunoglobulin light chain polypeptide comprises a LCDR1 comprising or consisting of SEQ ID NO: 125, a LCDR2 comprising or consisting of SEQ ID NO: 142, and a LCDR3 comprising or consisting of SEQ ID NO: 175;
(41) the immunoglobulin heavy chain polypeptide comprises a HCDR1 comprising or consisting of SEQ ID NO: 14, a HCDR2 comprising or consisting of SEQ ID NO: 43, and a HCDR3 comprising or consisting of SEQ ID NO: 76; and/or the immunoglobulin light chain polypeptide comprises a LCDR1 comprising or consisting of SEQ ID NO: 127, a LCDR2 comprising or consisting of SEQ ID NO: 137, and a LCDR3 comprising or consisting of SEQ ID NO: 176;
(42) the immunoglobulin heavy chain polypeptide comprises a HCDR1 comprising or consisting of SEQ ID NO: 3, a HCDR2 comprising or consisting of SEQ ID NO: 57, and a HCDR3 comprising or consisting of SEQ ID NO: 95; and/or the immunoglobulin light chain polypeptide comprises a LCDR1 comprising or consisting of SEQ ID NO: 128, a LCDR2 comprising or consisting of SEQ ID NO: 137, and a LCDR3 comprising or consisting of SEQ ID NO: 155; and/or
(43) the immunoglobulin heavy chain polypeptide and light chain polypeptide comprises any combination of the CDRs listed in FIGS. 1A-D of PD-L1 Type A binding agents 1-42.

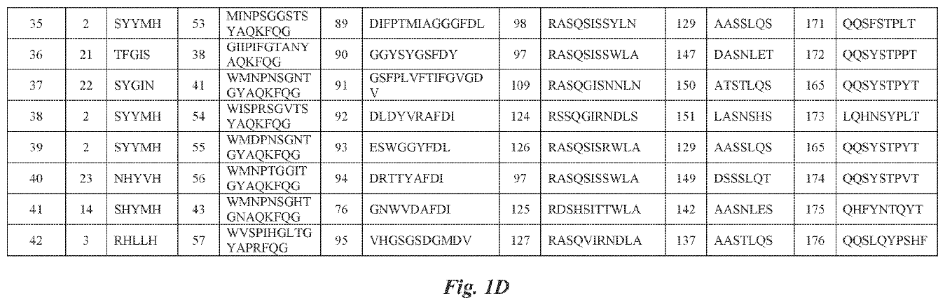

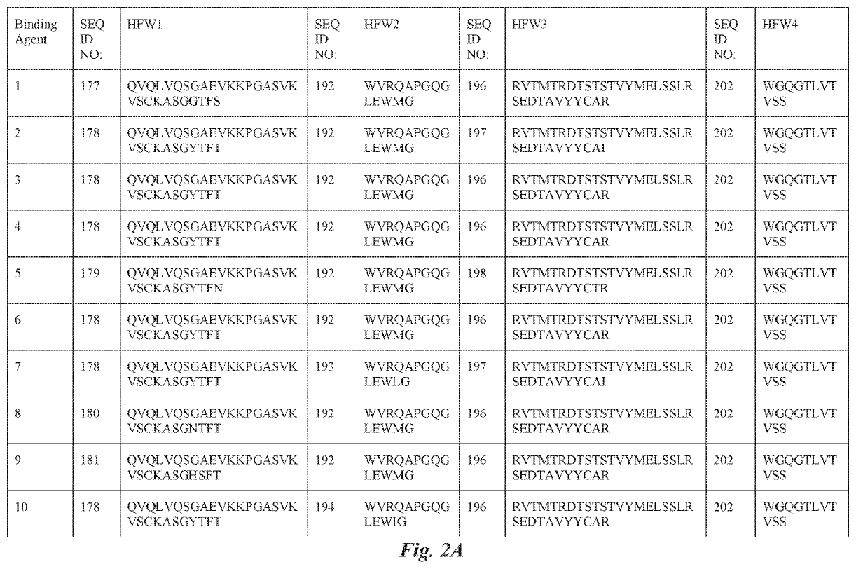

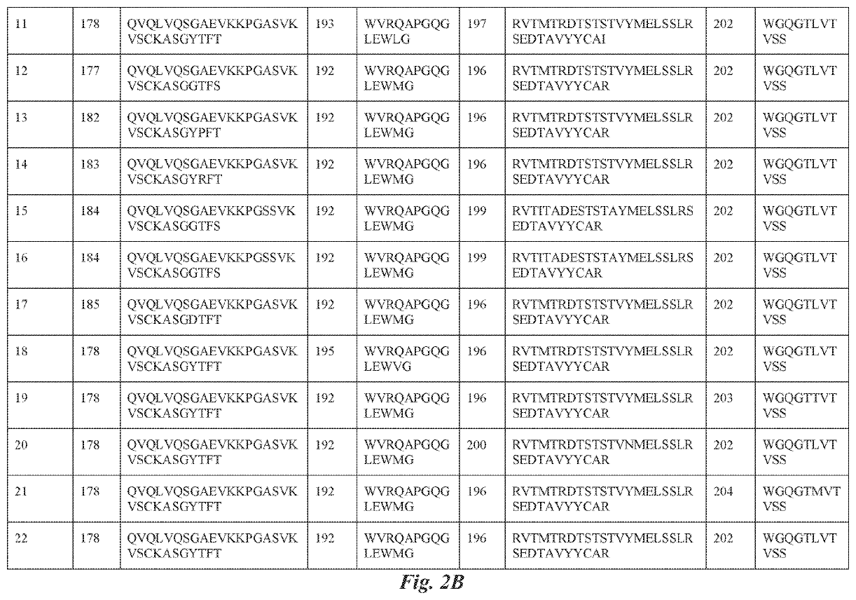

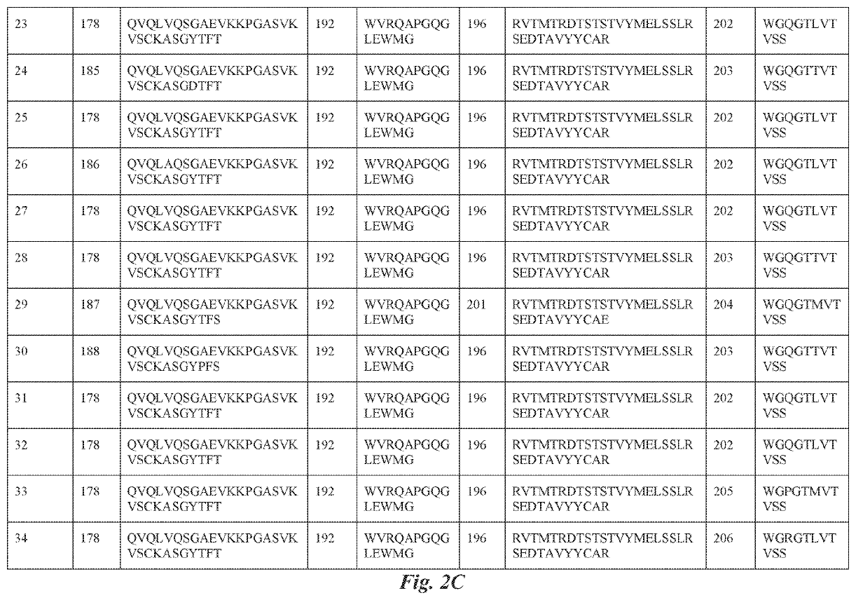

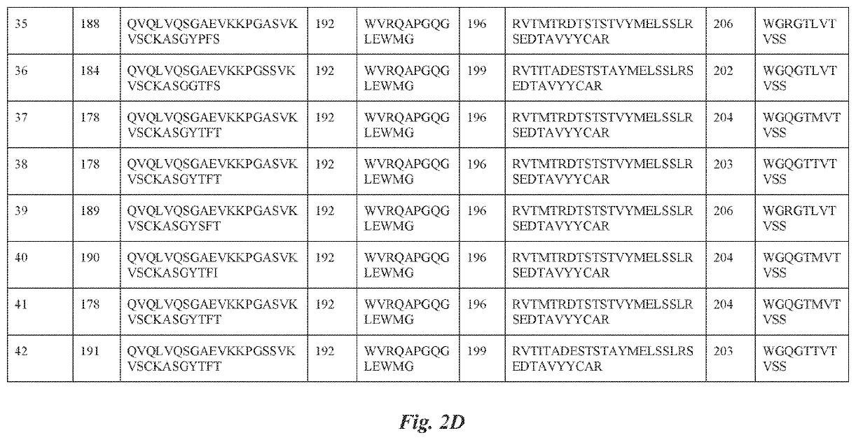

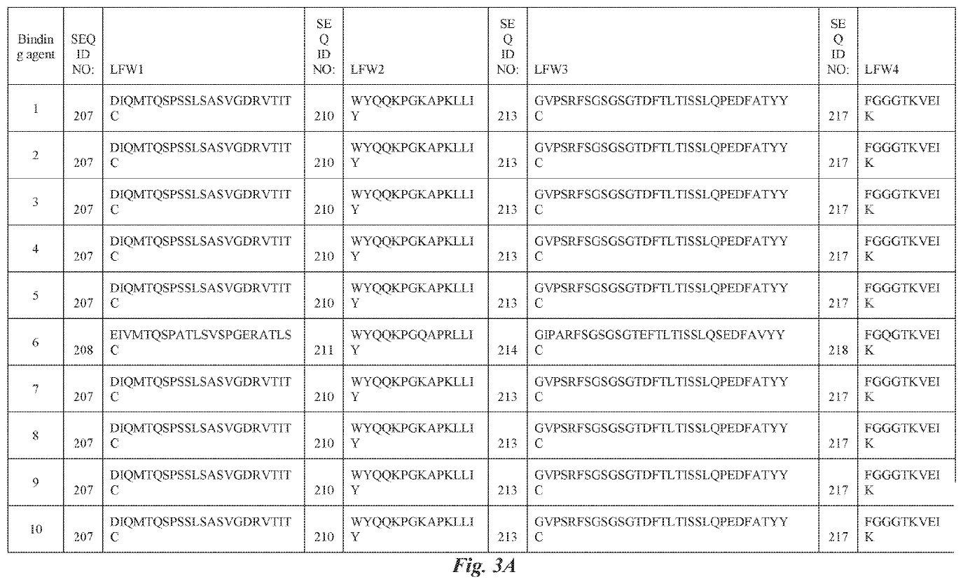

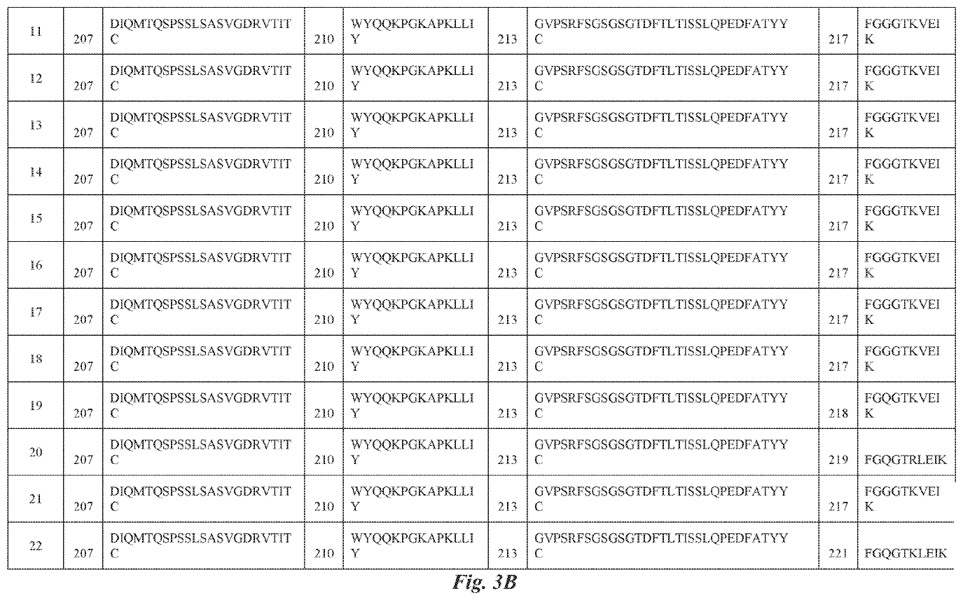

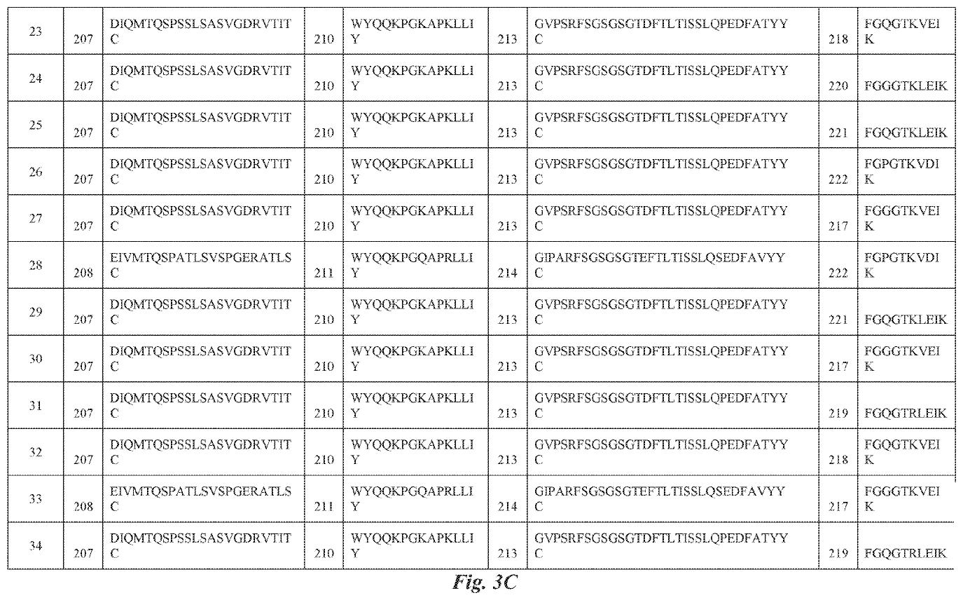

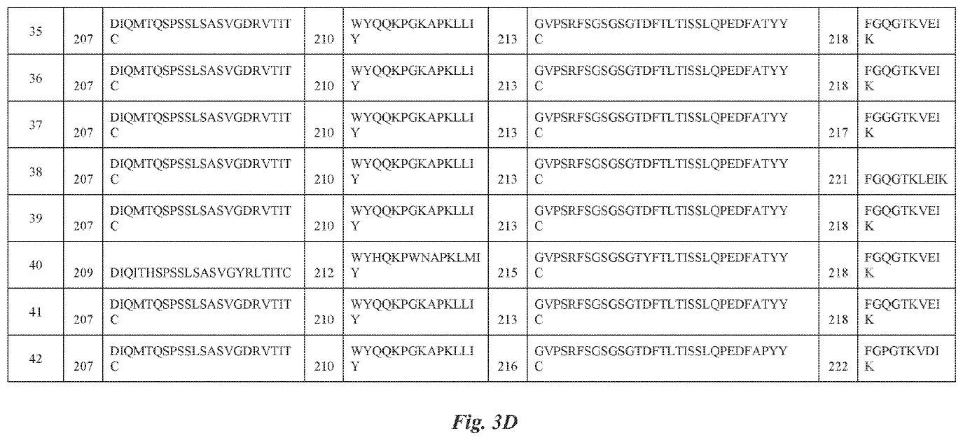

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in FIGS. 2A-D and FIGS. 3A-D, respectively.

### PD-L1-Binding Agents—Type B

Provided herein are PD-L1 binding agents (Type B) comprising an immunoglobulin heavy chain variable region polypeptide and an immunoglobulin light chain variable region polypeptide. In some embodiments, the PD-L1 binding agents (Type B) comprise an immunoglobulin heavy chain variable region of any one of SEQ ID NOs: 430-450, or at least the CDRs thereof, and an immunoglobulin light chain variable region of any one of SEQ ID NOs: 451-471, or at least the CDRs thereof. In other embodiments, the PD-L1 binding agents comprise an immunoglobulin heavy chain variable region polypeptide with an amino acid sequence that is at least 90% identical to any one of SEQ ID NOs: 430-450, and an immunoglobulin light chain variable region polypeptide with an amino acid sequence that is at least 90% identical to any one of SEQ ID NOs: 451-471. In yet other embodiments, the PD-L1 binding agent, the immunoglobulin heavy chain variable region polypeptide comprises a complementarity determining region 1 (HCDR1) comprising any one of SEQ ID NOs: 308-321, a complementarity determining region 2 (HCDR2) comprising any one of SEQ ID NOs: 322-338, and a complementarity determining region 3 (HCDR3) comprising any one of SEQ ID NOs: 339-359; and/or the immunoglobulin light chain variable region polypeptide comprises a complementarity determining region 1 (LCDR1) comprising any one of SEQ ID NOs: 360-374, a complementarity determining region 2 (LCDR2) comprising any one of SEQ ID NOs: 131 and 375-386, and a complementarity determining region 3 (LCDR3) comprising any one of SEQ ID NOs: 387-398. Also provided are nucleic acids encoding the PD-L1 binding agents, or the individual heavy and light chains thereof; vectors and cells comprising the nucleic acids; and compositions comprising the binding agents or nucleic acids.

In an embodiment, the PD-L1 binding agent (Type B) comprises an immunoglobulin heavy chain variable region of any one of SEQ ID NOs: 430-450, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 430-450, or at least the CDRs thereof; and/or an immunoglobulin light chain variable region of any one of SEQ ID NOs: 451-471, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 451-471, or at least the CDRs thereof.

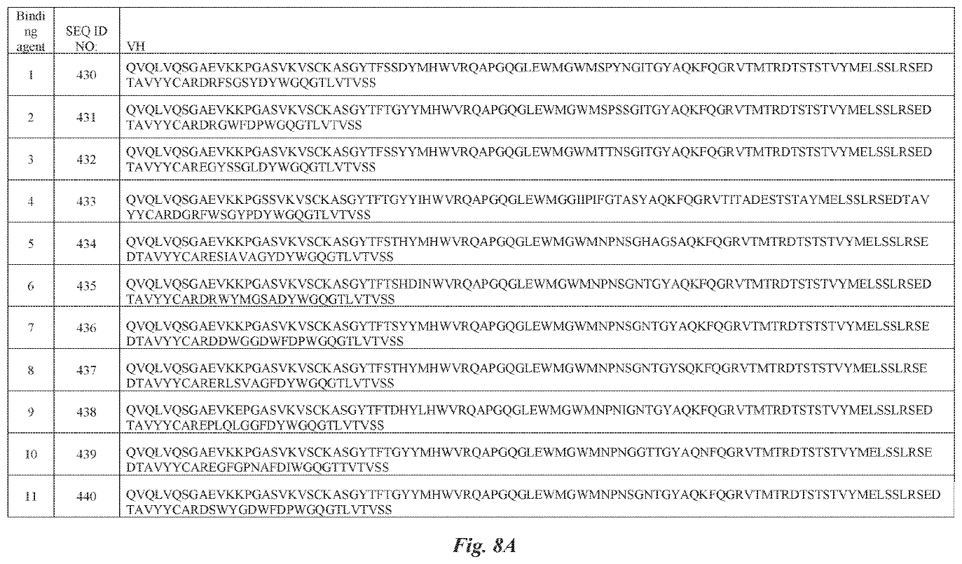

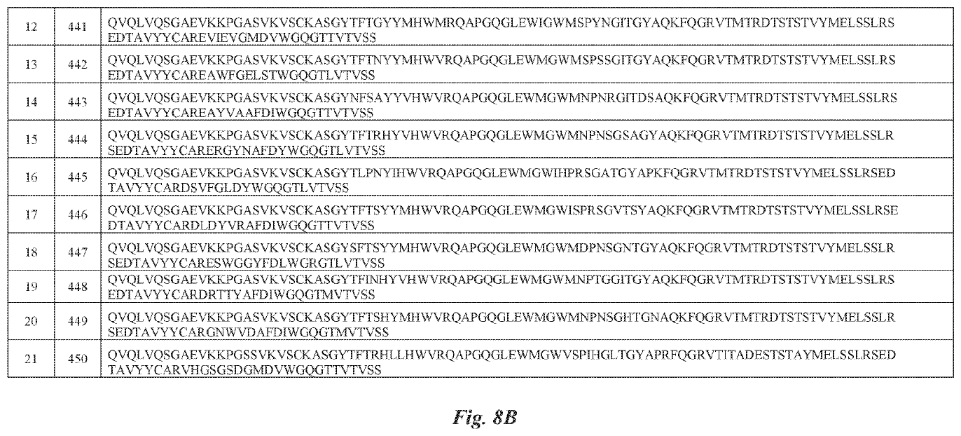

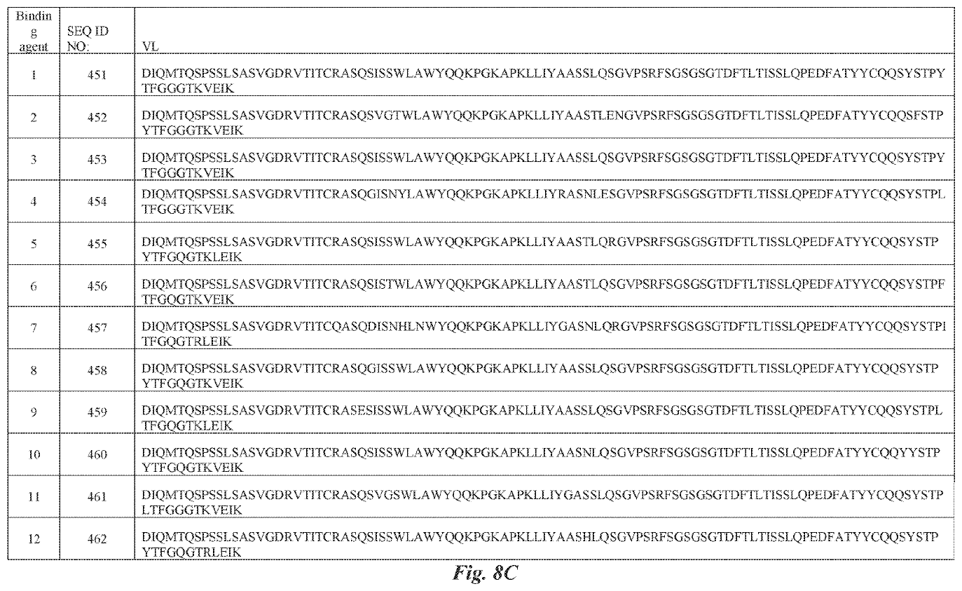

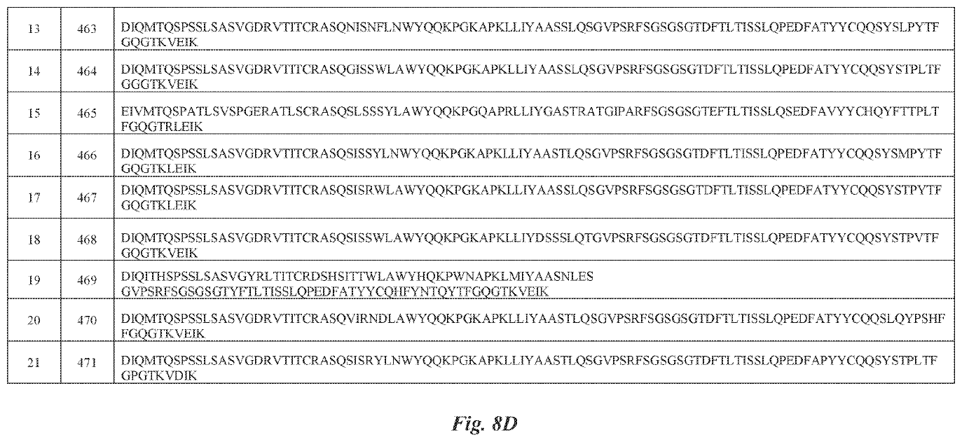

By way of further illustration, the PD-L1 binding agent (Type B) can comprise:
(1) an immunoglobulin heavy chain variable region of SEQ ID NO: 430, or at least the CDRs thereof, and/or an immunoglobulin light chain variable region of SEQ ID NO: 451, or at least the CDRs thereof;
(2) an immunoglobulin heavy chain variable region of SEQ ID NO: 430, or at least the CDRs thereof, and/or an immunoglobulin light chain variable region of SEQ ID NO: 451, or at least the CDRs thereof;
(3) an immunoglobulin heavy chain variable region of SEQ ID NO: 431, or at least the CDRs thereof, and/or an immunoglobulin light chain variable region of SEQ ID NO: 452, or at least the CDRs thereof;
(4) an immunoglobulin heavy chain variable region of SEQ ID NO: 432, or at least the CDRs thereof, and/or an immunoglobulin light chain variable region of SEQ ID NO: 453, or at least the CDRs thereof;
(5) an immunoglobulin heavy chain variable region of SEQ ID NO: 433, or at least the CDRs thereof, and/or an immunoglobulin light chain variable region of SEQ ID NO: 454, or at least the CDRs thereof;
(6) an immunoglobulin heavy chain variable region of SEQ ID NO: 434, or at least the CDRs thereof, and/or an immunoglobulin light chain variable region of SEQ ID NO: 455, or at least the CDRs thereof;
(7) an immunoglobulin heavy chain variable region of SEQ ID NO: 435, or at least the CDRs thereof, and/or an immunoglobulin light chain variable region of SEQ ID NO: 456, or at least the CDRs thereof;
(8) an immunoglobulin heavy chain variable region of SEQ ID NO: 436, or at least the CDRs thereof, and/or an immunoglobulin light chain variable region of SEQ ID NO: 457, or at least the CDRs thereof;
(9) an immunoglobulin heavy chain variable region of SEQ ID NO: 437, or at least the CDRs thereof, and/or an immunoglobulin light chain variable region of SEQ ID NO: 458, or at least the CDRs thereof;
(10) an immunoglobulin heavy chain variable region of SEQ ID NO: 438, or at least the CDRs thereof, and/or an immunoglobulin light chain variable region of SEQ ID NO: 459, or at least the CDRs thereof;
(11) an immunoglobulin heavy chain variable region of SEQ ID NO: 439, or at least the CDRs thereof, and/or an immunoglobulin light chain variable region of SEQ ID NO: 460, or at least the CDRs thereof;
(12) an immunoglobulin heavy chain variable region of SEQ ID NO: 440, or at least the CDRs thereof, and/or an immunoglobulin light chain variable region of SEQ ID NO: 461, or at least the CDRs thereof;
(13) an immunoglobulin heavy chain variable region of SEQ ID NO: 441, or at least the CDRs thereof, and/or an immunoglobulin light chain variable region of SEQ ID NO: 462, or at least the CDRs thereof;
(14) an immunoglobulin heavy chain variable region of SEQ ID NO: 442, or at least the CDRs thereof, and/or an immunoglobulin light chain variable region of SEQ ID NO: 463, or at least the CDRs thereof;
(15) an immunoglobulin heavy chain variable region of SEQ ID NO: 443, or at least the CDRs thereof, and/or an immunoglobulin light chain variable region of SEQ ID NO: 464, or at least the CDRs thereof;
(16) an immunoglobulin heavy chain variable region of SEQ ID NO: 444, or at least the CDRs thereof, and/or an immunoglobulin light chain variable region of SEQ ID NO: 465, or at least the CDRs thereof;
(17) an immunoglobulin heavy chain variable region of SEQ ID NO: 445, or at least the CDRs thereof, and/or an immunoglobulin light chain variable region of SEQ ID NO: 466, or at least the CDRs thereof;
(18) an immunoglobulin heavy chain variable region of SEQ ID NO: 446, or at least the CDRs thereof, and/or an immunoglobulin light chain variable region of SEQ ID NO: 467, or at least the CDRs thereof;
(19) an immunoglobulin heavy chain variable region of SEQ ID NO: 447, or at least the CDRs thereof, and/or an immunoglobulin light chain variable region of SEQ ID NO: 468, or at least the CDRs thereof;
(20) an immunoglobulin heavy chain variable region of SEQ ID NO: 448, or at least the CDRs thereof, and/or an immunoglobulin light chain variable region of SEQ ID NO: 469, or at least the CDRs thereof; and/or
(21) an immunoglobulin heavy chain variable region of SEQ ID NO: 449, or at least the CDRs thereof, and/or an immunoglobulin light chain variable region of SEQ ID NO: 470, or at least the CDRs thereof;
(22) an immunoglobulin heavy chain variable region of SEQ ID NO: 450, or at least the CDRs thereof, and/or an immunoglobulin light chain variable region of SEQ ID NO: 471, or at least the CDRs thereof; and/or
(23) an immunoglobulin heavy chain variable region of FIGS. 8A-B and/or an immunoglobulin light chain variable region of FIGS. 8C-D, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent comprises one or more of the following CDRs:
a HCDR1 comprising or consisting of any one of SEQ ID NOs: 308-321 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 308-321;
a HCDR2 comprising or consisting of any one of SEQ ID NOs: 322-338 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 322-338; and
a HCDR3 comprising or consisting of any one of SEQ ID NOs: 339-359 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 339-359; and/or the immunoglobulin light chain polypeptide comprises
a LCDR1 comprising or consisting of any one of SEQ ID NOs: 360-374 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 360-374;
a LCDR2 comprising or consisting of any one of SEQ ID NOs: 375-386 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 375-386; and
a LCDR3 comprising or consisting of any one of SEQ ID NOs: 387-398 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 387-398.

In particular embodiments, the binding agent comprises an immunoglobulin heavy chain polypeptide and an immunoglobulin light chain polypeptide, wherein:
(1) the immunoglobulin heavy chain polypeptide comprises a HCDR1 comprising or consisting of SEQ ID NO: 308, a HCDR2 comprising or consisting of SEQ ID NO: 322, and a HCDR3 comprising or consisting of SEQ ID NO: 339; and/or the immunoglobulin light chain polypeptide comprises a LCDR1 comprising or consisting of SEQ ID NO: 360, a LCDR2 comprising or consisting of SEQ ID NO: 375, and a LCDR3 comprising or consisting of SEQ ID NO: 387;
(2) the immunoglobulin heavy chain polypeptide comprises a HCDR1 comprising or consisting of SEQ ID NO: 309, a HCDR2 comprising or consisting of SEQ ID NO: 323, and a HCDR3 comprising or consisting of SEQ ID NO: 340; and/or the immunoglobulin light chain polypeptide comprises a LCDR1 comprising or consisting of SEQ ID NO: 361, a LCDR2 comprising or consisting of SEQ ID NO: 376, and a LCDR3 comprising or consisting of SEQ ID NO: 388;
(3) the immunoglobulin heavy chain polypeptide comprises a HCDR1 comprising or consisting of SEQ ID NO: 310, a HCDR2 comprising or consisting of SEQ ID NO: 324, and a HCDR3 comprising or consisting of SEQ ID NO: 341; and/or the immunoglobulin light chain polypeptide comprises a LCDR1 comprising or consisting of SEQ ID NO: 360, a LCDR2 comprising or consisting of SEQ ID NO: 375, and a LCDR3 comprising or consisting of SEQ ID NO: 387;
(4) the immunoglobulin heavy chain polypeptide comprises a HCDR1 comprising or consisting of SEQ ID NO: 311, a HCDR2 comprising or consisting of SEQ ID NO: 325, and a HCDR3 comprising or consisting of SEQ ID NO: 342; and/or the immunoglobulin light chain polypeptide comprises a LCDR1 comprising or consisting of SEQ ID NO: 362, a LCDR2 comprising or consisting of SEQ ID NO: 377, and a LCDR3 comprising or consisting of SEQ ID NO: 389;
(5) the immunoglobulin heavy chain polypeptide comprises a HCDR1 comprising or consisting of SEQ ID NO: 312, a HCDR2 comprising or consisting of SEQ ID NO: 326, and a HCDR3 comprising or consisting of SEQ ID NO: 343; and/or the immunoglobulin light chain polypeptide comprises a LCDR1 comprising or consisting of SEQ ID NO: 360, a LCDR2 comprising or consisting of SEQ ID NO: 378, and a LCDR3 comprising or consisting of SEQ ID NO: 387;
(6) the immunoglobulin heavy chain polypeptide comprises a HCDR1 comprising or consisting of SEQ ID NO: 313, a HCDR2 comprising or consisting of SEQ ID NO: 327, and a HCDR3 comprising or consisting of SEQ ID NO: 344; and/or the immunoglobulin light chain polypeptide comprises a LCDR1 comprising or consisting of SEQ ID NO: 363, a LCDR2 comprising or consisting of SEQ ID NO: 379, and a LCDR3 comprising or consisting of SEQ ID NO: 390;
(7) the immunoglobulin heavy chain polypeptide comprises a HCDR1 comprising or consisting of SEQ ID NO: 314, a HCDR2 comprising or consisting of SEQ ID NO: 327, and a HCDR3 comprising or consisting of SEQ ID NO: 345; and/or the immunoglobulin light chain polypeptide comprises a LCDR1 comprising or consisting of SEQ ID NO: 364, a LCDR2 comprising or consisting of SEQ ID NO: 380, and a LCDR3 comprising or consisting of SEQ ID NO: 391;
(8) the immunoglobulin heavy chain polypeptide comprises a HCDR1 comprising or consisting of SEQ ID NO: 312, a HCDR2 comprising or consisting of SEQ ID NO: 328, and a HCDR3 comprising or consisting of SEQ ID NO: 346; and/or the immunoglobulin light chain polypeptide comprises a LCDR1 comprising or consisting of SEQ ID NO: 365, a LCDR2 comprising or consisting of SEQ ID NO: 375, and a LCDR3 comprising or consisting of SEQ ID NO: 387;
(9) the immunoglobulin heavy chain polypeptide comprises a HCDR1 comprising or consisting of SEQ ID NO: 314, a HCDR2 comprising or consisting of SEQ ID NO: 329, and a HCDR3 comprising or consisting of SEQ ID NO: 347; and/or the immunoglobulin light chain polypeptide comprises a LCDR1 comprising or consisting of SEQ ID NO: 366, a LCDR2 comprising or consisting of SEQ ID NO: 375, and a LCDR3 comprising or consisting of SEQ ID NO: 389;
(10) the immunoglobulin heavy chain polypeptide comprises a HCDR1 comprising or consisting of SEQ ID NO: 309, a HCDR2 comprising or consisting of SEQ ID NO: 330, and a HCDR3 comprising or consisting of SEQ ID NO: 348; and/or the immunoglobulin light chain polypeptide comprises a LCDR1 comprising or consisting of SEQ ID NO: 360, a LCDR2 comprising or consisting of SEQ ID NO: 381, and a LCDR3 comprising or consisting of SEQ ID NO: 392;
(11) the immunoglobulin heavy chain polypeptide comprises a HCDR1 comprising or consisting of SEQ ID NO: 309, a HCDR2 comprising or consisting of SEQ ID NO: 327, and a HCDR3 comprising or consisting of SEQ ID NO: 349; and/or the immunoglobulin light chain polypeptide comprises a LCDR1 comprising or consisting of SEQ ID NO: 367, a LCDR2 comprising or consisting of SEQ ID NO: 382, and a LCDR3 comprising or consisting of SEQ ID NO: 389;
(12) the immunoglobulin heavy chain polypeptide comprises a HCDR1 comprising or consisting of SEQ ID NO: 309, a HCDR2 comprising or consisting of SEQ ID NO: 322, and a HCDR3 comprising or consisting of SEQ ID NO: 350; and/or the immunoglobulin light chain polypeptide comprises a LCDR1 comprising or consisting of SEQ ID NO: 360, a LCDR2 comprising or consisting of SEQ ID NO: 383, and a LCDR3 comprising or consisting of SEQ ID NO: 387;
(13) the immunoglobulin heavy chain polypeptide comprises a HCDR1 comprising or consisting of SEQ ID NO: 315, a HCDR2 comprising or consisting of SEQ ID NO: 323, and a HCDR3 comprising or consisting of SEQ ID NO: 351; and/or the immunoglobulin light chain polypeptide comprises a LCDR1 comprising or consisting of SEQ ID NO: 368, a LCDR2 comprising or consisting of SEQ ID NO: 375, and a LCDR3 comprising or consisting of SEQ ID NO: 393;
(14) the immunoglobulin heavy chain polypeptide comprises a HCDR1 comprising or consisting of SEQ ID NO:316, a HCDR2 comprising or consisting of SEQ ID NO: 331, and a HCDR3 comprising or consisting of SEQ ID NO: 352; and/or the immunoglobulin light chain polypeptide comprises a LCDR1 comprising or consisting of SEQ ID NO: 365, a LCDR2 comprising or consisting of SEQ ID NO: 375, and a LCDR3 comprising or consisting of SEQ ID NO: 389;
(15) the immunoglobulin heavy chain polypeptide comprises a HCDR1 comprising or consisting of SEQ ID NO: 317, a HCDR2 comprising or consisting of SEQ ID NO: 332, and a HCDR3 comprising or consisting of SEQ ID NO: 353; and/or the immunoglobulin light chain polypeptide comprises a LCDR1 comprising or consisting of SEQ ID NO: 369, a LCDR2 comprising or consisting of SEQ ID NO: 384, and a LCDR3 comprising or consisting of SEQ ID NO: 394;
(16) the immunoglobulin heavy chain polypeptide comprises a HCDR1 comprising or consisting of SEQ ID NO: 318, a HCDR2 comprising or consisting of SEQ ID NO: 333, and a HCDR3 comprising or consisting of SEQ ID NO: 354; and/or the immunoglobulin light chain polypeptide comprises a LCDR1 comprising or consisting of SEQ ID NO: 370, a LCDR2 comprising or consisting of SEQ ID NO: 379, and a LCDR3 comprising or consisting of SEQ ID NO: 395;
(17) the immunoglobulin heavy chain polypeptide comprises a HCDR1 comprising or consisting of SEQ ID NO:310, a HCDR2 comprising or consisting of SEQ ID NO: 334, and a HCDR3 comprising or consisting of SEQ ID NO: 355; and/or the immunoglobulin light chain polypeptide comprises a LCDR1 comprising or consisting of SEQ ID NO: 371, a LCDR2 comprising or consisting of SEQ ID NO: 375, and a LCDR3 comprising or consisting of SEQ ID NO: 387;
(18) the immunoglobulin heavy chain polypeptide comprises a HCDR1 comprising or consisting of SEQ ID NO:310, a HCDR2 comprising or consisting of SEQ ID NO: 335, and a HCDR3 comprising or consisting of SEQ ID NO: 356; and/or the immunoglobulin light chain polypeptide comprises a LCDR1 comprising or consisting of SEQ ID NO: 360, a LCDR2 comprising or consisting of SEQ ID NO: 385, and a LCDR3 comprising or consisting of SEQ ID NO: 396;
(19) the immunoglobulin heavy chain polypeptide comprises a HCDR1 comprising or consisting of SEQ ID NO: 319, a HCDR2 comprising or consisting of SEQ ID NO: 336, and a HCDR3 comprising or consisting of SEQ ID NO: 357; and/or the immunoglobulin light chain polypeptide comprises a LCDR1 comprising or consisting of SEQ ID NO: 372, a LCDR2 comprising or consisting of SEQ ID NO: 386, and a LCDR3 comprising or consisting of SEQ ID NO: 397;
(20) the immunoglobulin heavy chain polypeptide comprises a HCDR1 comprising or consisting of SEQ ID NO: 320, a HCDR2 comprising or consisting of SEQ ID NO: 337, and a HCDR3 comprising or consisting of SEQ ID NO: 358; and/or the immunoglobulin light chain polypeptide comprises a LCDR1 comprising or consisting of SEQ ID NO: 373, a LCDR2 comprising or consisting of SEQ ID NO: 379, and a LCDR3 comprising or consisting of SEQ ID NO: 398;
(21) the immunoglobulin heavy chain polypeptide comprises a HCDR1 comprising or consisting of SEQ ID NO: 321, a HCDR2 comprising or consisting of SEQ ID NO: 338, and a HCDR3 comprising or consisting of SEQ ID NO: 359; and/or the immunoglobulin light chain polypeptide comprises a LCDR1 comprising or consisting of SEQ ID NO: 374, a LCDR2 comprising or consisting of SEQ ID NO: 379, and a LCDR3 comprising or consisting of SEQ ID NO: 389; and/or
(22) the immunoglobulin heavy chain polypeptide and light chain polypeptide comprises any combination of the CDRs listed in FIGS. 5A-B (Type B).

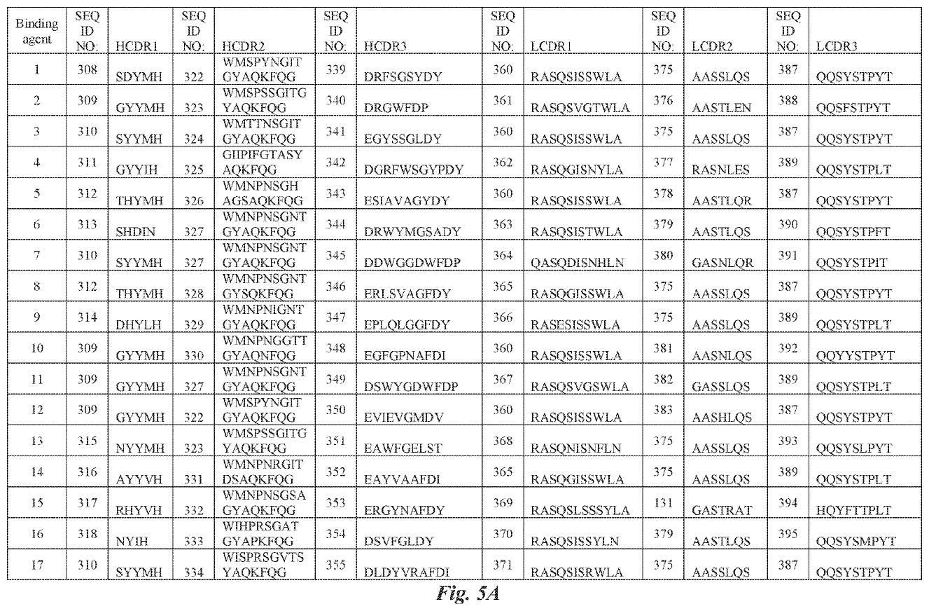

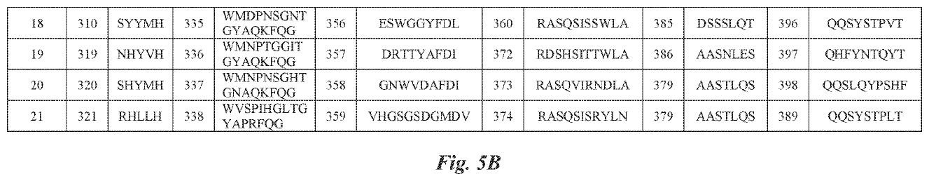

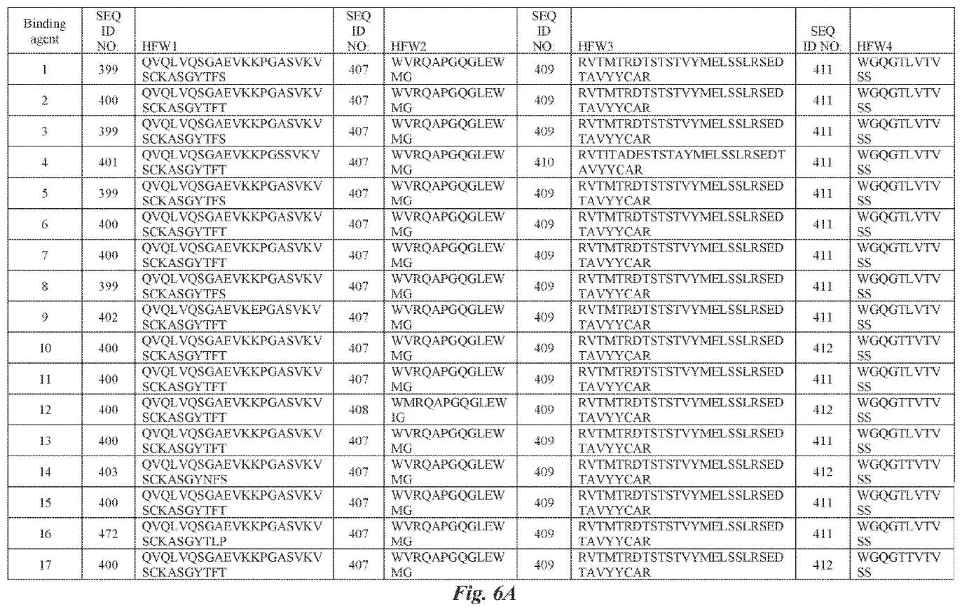

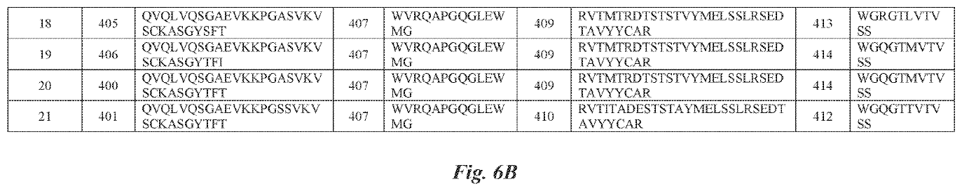

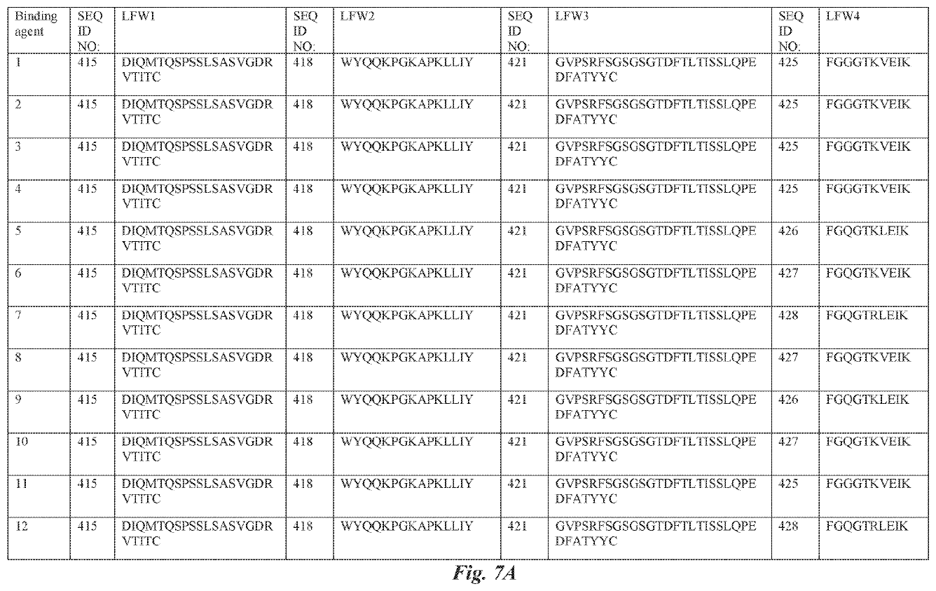

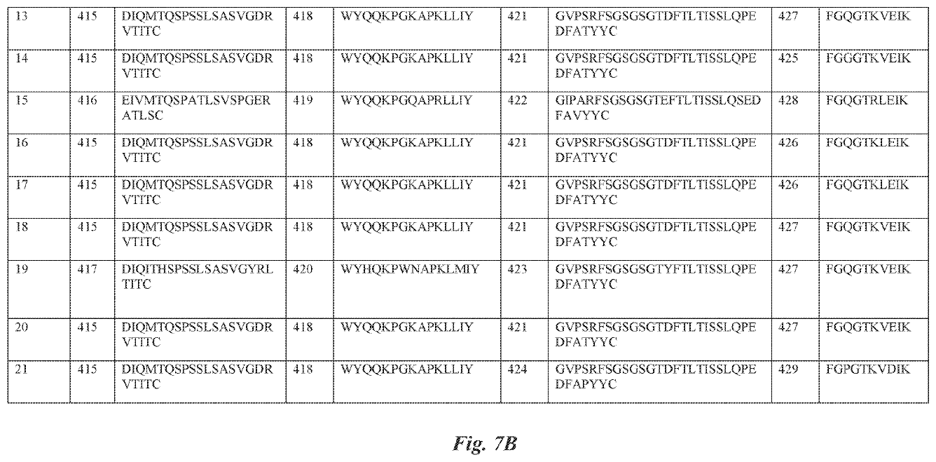

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in FIGS. 6A-B and/or FIGS. 7A-B (Type B), respectively.

The immunoconjugate of the invention comprises an antibody which targets, binds, or recognizes PD-L1. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing PD-L1 to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

The antibodies comprising the immunoconjugates of the invention include Fe engineered variants. In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/1332E), SDIEAL (S239D/I332E/A330L), GA (G236A), ALIE (A330L/1332E), GASDALIE (G236A/S239D/A330L/1332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, U.S. Patent Application Publication 2016/0145350 and U.S. Pat. Nos. 7,416,726 and 5,624,821, which are hereby incorporated by reference in their entireties herein.

The antibodies comprising the immunoconjugates of the invention include glycan variants, such as afucosylation. In some embodiments, the Fc region of the binding agents are modified to have an altered glycosylation pattern of the Fc region compared to the native non-modified Fc region.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), or an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/1332E), GASDALIE (G236A/S239D/A330L/1332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, US 2016/0145350, U.S. Pat. Nos. 7,416,726 and 5,624,821, which are hereby incorporated by reference in their entireties.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIα binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, the mutation can be a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726, US 2007/0014795 and US 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA; or the Fab region of nivolumab, which normally comprises an IgG4 Fe region, can be conjugated to IgG1, IgG2, IgG3, IgA1, or IgG2. In some embodiments, the Fe modified antibody with a non-native Fc domain also comprises one or more amino acid modifications, such as the S228P mutation within the IgG4 Fc, that modulate the stability of the Fc domain described. In some embodiments, the Fe modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR increase the binding of the Fab region of the antibody to its antigen when compared to the native non-modified antibody.

In some embodiments, the antibody construct further comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-PD-L1antibody, each variable region comprising a CDR1, a CDR2, and a CDR3.

In some embodiments, the antibodies in the immunoconjugates contain a modified Fc region, wherein the modification modulates the binding of the Fc region to one or more Fc receptors.

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, the TGFβ receptor is a human TGFβ receptor. In some embodiments, the IgG has a C-terminal fusion to a TGFβRII extracellular domain (ECD) as described in U.S. Pat. No. 9,676,863, incorporated herein in its entirety. An “Fc linker” may be used to attach the IgG to the TGFβRII extracellular domain. The Fe linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibody in the immunoconjugates is a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., 2008b Nature Biotech., 26(8):925-932; Dornan et al. (2009) Blood 114(13):2721-2729; and the following patents, each of which is hereby incorporated by reference in its entirety: U.S. Pat. Nos. 7,521,541, 7,723,485, US 2012/0121615, WO 2009/052249). A “cysteine engineered antibody” or “cysteine engineered antibody variant” is an antibody in which one or more residues of an antibody are substituted with cysteine residues. Cysteine-engineered antibodies can be conjugated to the 8-Cyc-2-aminobenzazepine adjuvant moiety as an 8-cyclyl-2-aminobenzazepine-linker compound with uniform stoichiometry (e.g., up to two 8-Cyc-2-aminobenzazepine moieties per antibody in an antibody that has a single engineered cysteine site).

In some embodiments, cysteine-engineered antibodies used to prepare the immunoconjugates of Table 3 have a cysteine residue introduced at the 149-lysine site of the light chain (LC K149C). In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in the light chain at G64C or R142C according to Kabat numbering, or in the heavy chain at D101C, V184C or T205C according to Kabat numbering.

### 8-Cyclyl-2-Aminobenzazepine Adjuvant Compounds

The immunoconjugate of the invention comprises an 8-Cyc-2-aminobenzazepine adjuvant moiety. The adjuvant moiety described herein is a compound that elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several TRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

Exemplary 8-cyclyl-2-aminobenzazepine compounds (CycBz) of the invention are shown in Tables 1a and 1b. Each compound was synthesized, purified, and characterized by mass spectrometry and shown to have the mass indicated. Additional experimental procedures are found in the Examples. Activity against HEK293 NFKB reporter cells expressing human TLR7 or human TLR8 was measured according to Example 202. The 8-cyclyl-2-aminobenzazepine compounds of Tables 1a and 1b demonstrate the surprising and unexpected property of TLR8 agonist selectivity which may predict useful therapeutic activity to treat cancer and other disorders.

TABLE 1a
8-Cyc-2-aminobenzazepine compounds (CycBz), where Cyc is heterocyclyldiyl or heteroaryldiyl

| | | | HEK293 | HEK293 |
|---|---|---|---|---|
| | | | hTLR7 | hTLR8 |
| Hx | | | EC50 | EC50 |
| No. | Structure | MW | (nM) | (nM) |
| CycBz-1 | 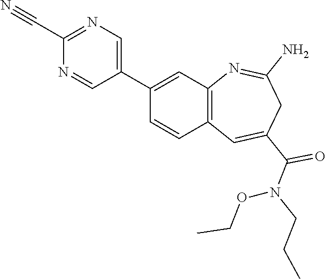 [SMILES: CCCN(OCC)C(=O)C1=Cc2ccc(-c3cnc(C#N)nc3)cc2N=C(N)C1] | 390.44 | 2536 | 163 |
| CycBz-2 | 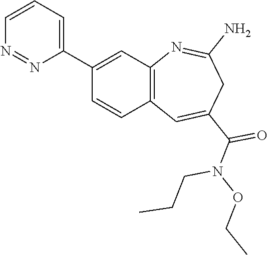 | 365.4 | 2238 | 276 |
| CycBz-3 | 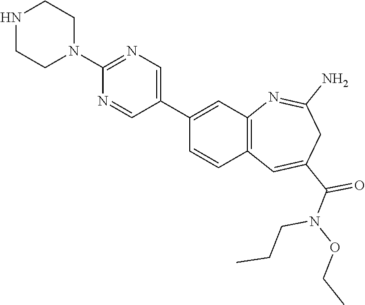 [SMILES: CCCN(OCC)C(=O)C1=Cc2ccc(-c3cnc(N4CCNCC4)nc3)cc2N=C(N)C1] | 449.6 | 562 | 43 |
| CycBz-4 | 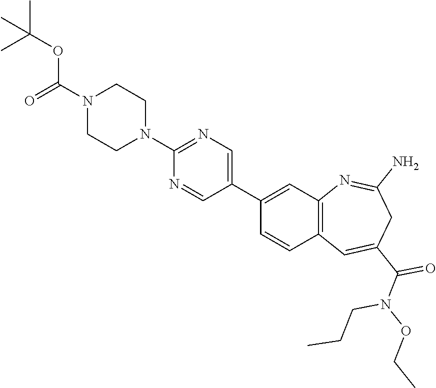 [SMILES: CCCN(OCC)C(=O)C1=Cc2ccc(-c3cnc(N4CCN(C(=O)OC(C)(C)C)CC4)nc3)cc2N=C(N)C1] | 549.7 | 3259 | 350 |
| CycBz-5 | 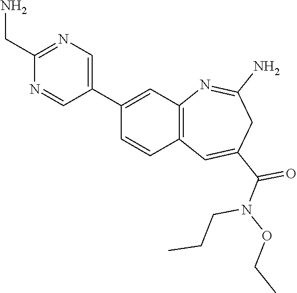 | 394.5 | 525 | 17 |
| CycBz-6 | 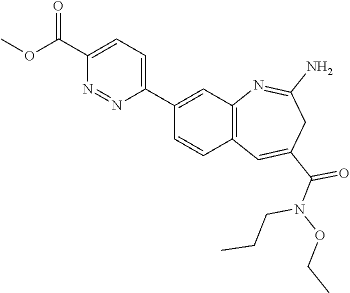 [SMILES: CCCN(OCC)C(=O)C1=Cc2ccc(-c3ccc(C(=O)OC)nn3)cc2N=C(N)C1] | 423.5 | 2659 | 339 |
| CycBz-7 | 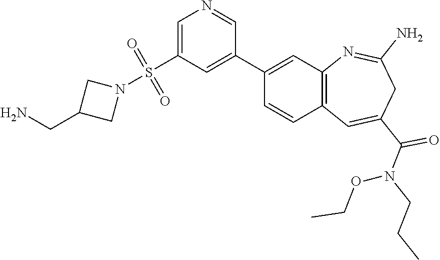 [SMILES: CCCN(OCC)C(=O)C1=Cc2ccc(-c3cncc(S(=O)(=O)N4CC(CN)C4)c3)cc2N=C(N)C1] | 512.6 | 3633 | 335 |
| CycBz-8 | 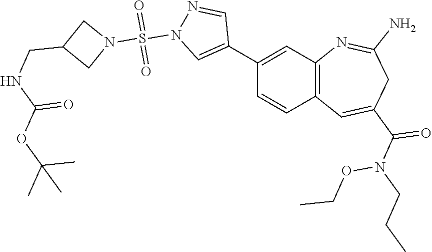 [SMILES: CCCN(OCC)C(=O)C1=Cc2ccc(-c3cnn(S(=O)(=O)N4CC(CNC(=O)OC(C)(C)C)C4)c3)cc2N=C(N)C1] | 601.7 | | |
| CycBz-9 | 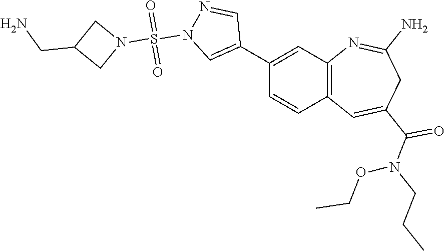 [SMILES: CCCN(OCC)C(=O)C1=Cc2ccc(-c3cnn(S(=O)(=O)N4CC(CN)C4)c3)cc2N=C(N)C1] | 501.6 | 8630 | 397 |
| CycBz-10 | 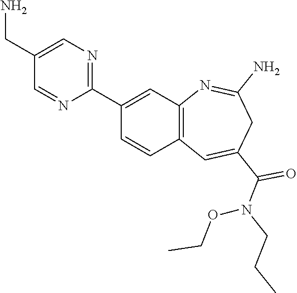 [SMILES: CCCN(OCC)C(=O)C1=Cc2ccc(-c3ncc(CN)cn3)cc2N=C(N)C1] | 394.5 | 9000 | 814 |
| CycBz-11 | 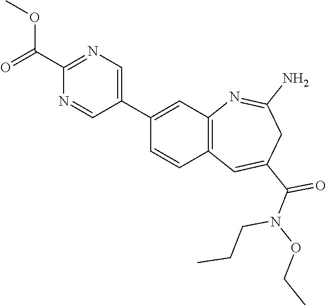 [SMILES: CCCN(OCC)C(=O)C1=Cc2ccc(-c3cnc(C(=O)OC)nc3)cc2N=C(N)C1] | 423.5 | 4070 | 161 |
| CycBz-12 | 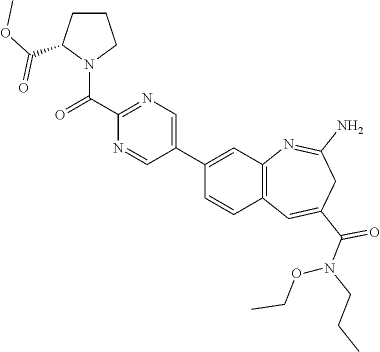 [SMILES: CCCN(OCC)C(=O)C1=Cc2ccc(-c3cnc(C(=O)N4CCC[C@H]4C(=O)OC)nc3)cc2N=C(N)C1] | 520.6 | 159 | 6 |
| CycBz-13 | 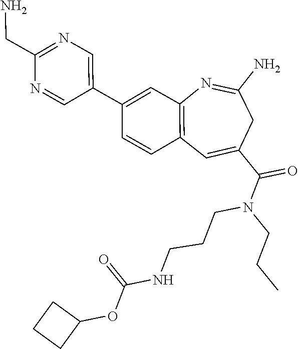 [SMILES: CCCN(CCCNC(=O)OC1CCC1)C(=O)C1=Cc2ccc(-c3cnc(CN)nc3)cc2N=C(N)C1] | 505.6 | 242 | 274 |
| CycBz-14 | 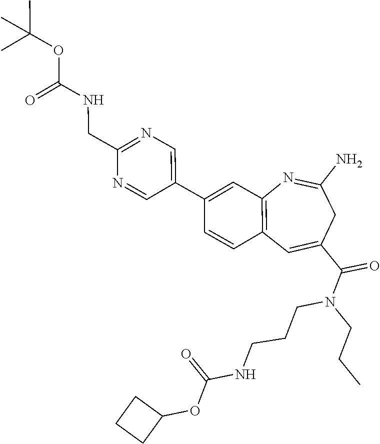 [SMILES: CCCN(CCCNC(=O)OC1CCC1)C(=O)C1=Cc2ccc(-c3cnc(CNC(=O)OC(C)(C)C)nc3)cc2N=C(N)C1] | 605.7 | | |
| CycBz-15 | 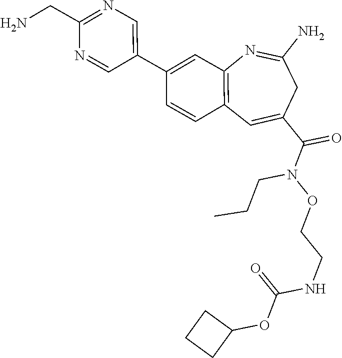 | 507.6 | 35 | 10 |
| CycBz-16 | 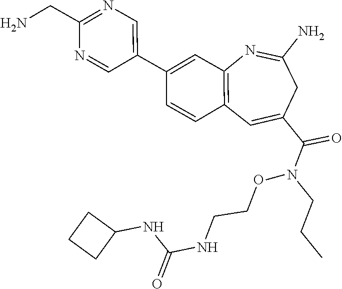 [SMILES: CCCN(OCCNC(=O)NC1CCC1)C(=O)C1=Cc2ccc(-c3cnc(CN)nc3)cc2N=C(N)C1] | 506.6 | 4602 | 399 |
| CycBz-17 | 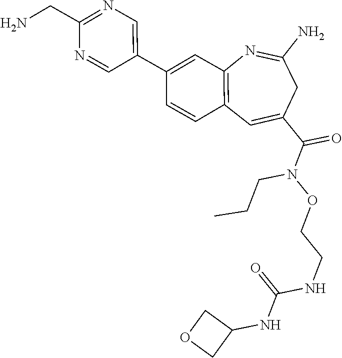 [SMILES: CCCN(OCCNC(=O)NC1COC1)C(=O)C1=Cc2ccc(-c3cnc(CN)nc3)cc2N=C(N)C1] | 508.6 | 9000 | 9000 |
| CycBz-18 | 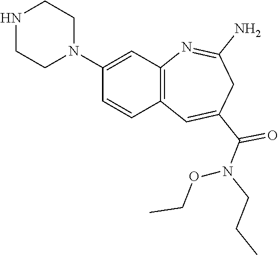 [SMILES: CCCN(OCC)C(=O)C1=Cc2ccc(N3CCNCC3)cc2N=C(N)C1] | 371.5 | 6310 | 281 |
| CycBz-19 | 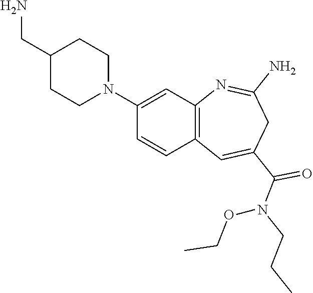 [SMILES: CCCN(OCC)C(=O)C1=Cc2ccc(N3CCC(CN)CC3)cc2N=C(N)C1] | 399.5 | | |
| CycBz-20 | 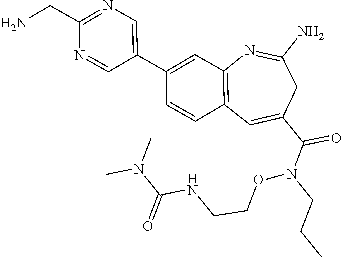 [SMILES: CCCN(OCCNC(=O)N(C)C)C(=O)C1=Cc2ccc(-c3cnc(CN)nc3)cc2N=C(N)C1] | 480.6 | 2943 | 3691 |
| CycBz-21 | 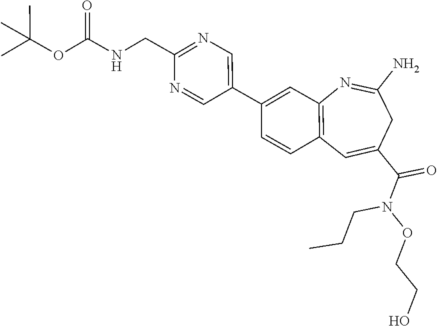 | 510.6 | | |
| CycBz-22 | 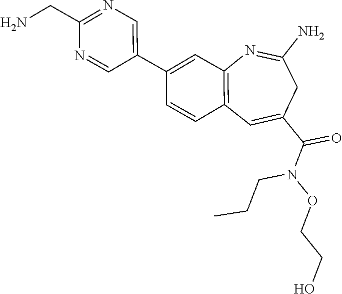 | 410.5 | 3916 | 1147 |
| CycBz-23 | 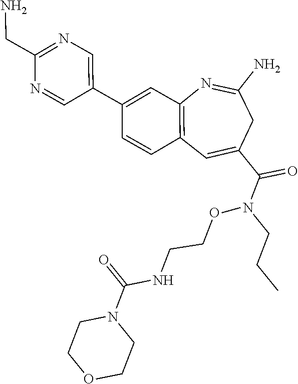 [SMILES: CCCN(OCCNC(=O)N1CCOCC1)C(=O)C1=Cc2ccc(-c3cnc(CN)nc3)cc2N=C(N)C1] | 522.6 | 6875 | 6176 |
| CycBz-24 | 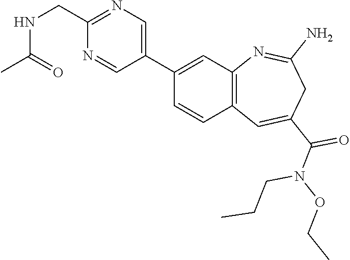 [SMILES: CCCN(OCC)C(=O)C1=Cc2ccc(-c3cnc(CNC(C)=O)nc3)cc2N=C(N)C1] | 436.5 | | |
| CycBz-25 | 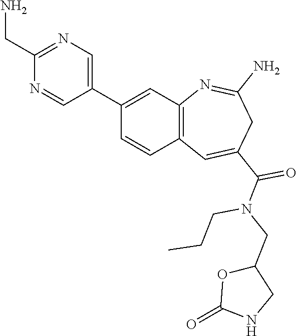 [SMILES: CCCN(CC1CNC(=O)O1)C(=O)C1=Cc2ccc(-c3cnc(CN)nc3)cc2N=C(N)C1] | 449.5 | 9000 | 3161 |
| CycBz-26 | 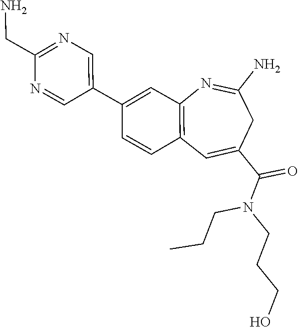 [SMILES: CCCN(CCCO)C(=O)C1=Cc2ccc(-c3cnc(CN)nc3)cc2N=C(N)C1] | 408.5 | 9000 | 9000 |
| CycBz-27 | 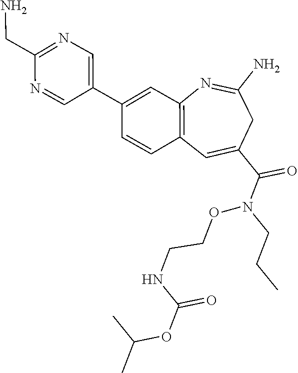 [SMILES: CCCN(OCCNC(=O)OC(C)C)C(=O)C1=Cc2ccc(-c3cnc(CN)nc3)cc2N=C(N)C1] | 495.6 | 26 | 9 |
| CycBz-28 | 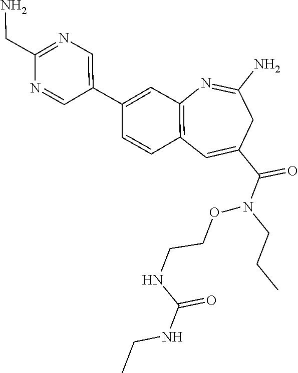 [SMILES: CCCN(OCCNC(=O)NCC)C(=O)C1=Cc2ccc(-c3cnc(CN)nc3)cc2N=C(N)C1] | 480.6 | 3771 | 2929 |
| CycBz-29 | 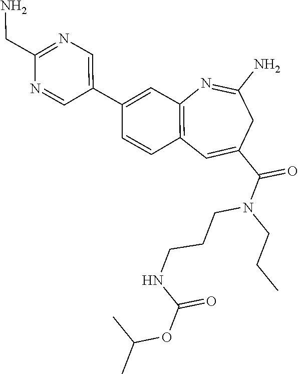 [SMILES: CCCN(CCCNC(=O)OC(C)C)C(=O)C1=Cc2ccc(-c3cnc(CN)nc3)cc2N=C(N)C1] | 493.6 | 134 | 296 |
| CycBz-30 | 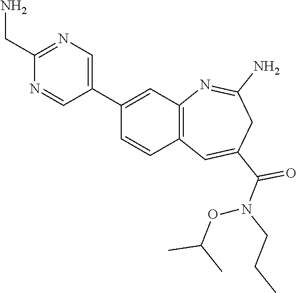 [SMILES: CCCN(OC(C)C)C(=O)C1=Cc2ccc(-c3cnc(CN)nc3)cc2N=C(N)C1] | 408.5 | 393 | 40 |
| CycBz-31 | 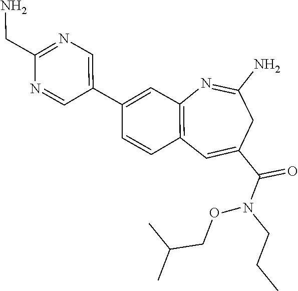 [SMILES: CCCN(OCC(C)C)C(=O)C1=Cc2ccc(-c3cnc(CN)nc3)cc2N=C(N)C1] | 422.5 | 763 | 358 |
| CycBz-32 | 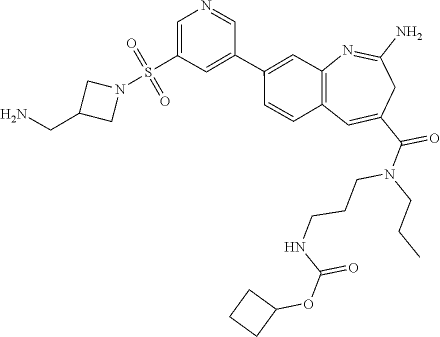 [SMILES: CCCN(CCCNC(=O)OC1CCC1)C(=O)C1=Cc2ccc(-c3cncc(S(=O)(=O)N4CC(CN)C4)c3)cc2N=C(N)C1] | 623.8 | 1280 | 1519 |
| CycBz-33 | 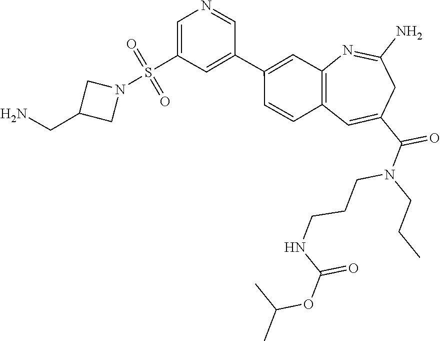 [SMILES: CCCN(CCCNC(=O)OC(C)C)C(=O)C1=Cc2ccc(-c3cncc(S(=O)(=O)N4CC(CN)C4)c3)cc2N=C(N)C1] | 611.8 | 7633 | 2876 |
| CycBz-34 | 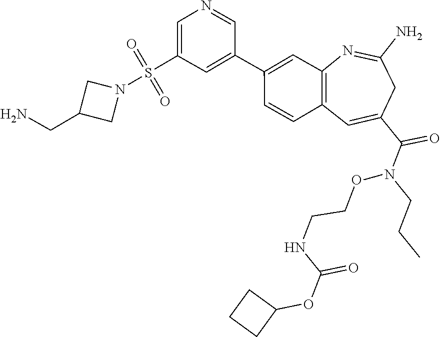 [SMILES: CCCN(OCCNC(=O)OC1CCC1)C(=O)C1=Cc2ccc(-c3cncc(S(=O)(=O)N4CC(CN)C4)c3)cc2N=C(N)C1] | 625.7 | 322 | 79 |
| CycBz-35 | 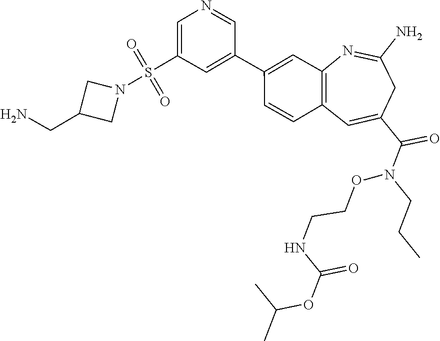 [SMILES: CCCN(OCCNC(=O)OC(C)C)C(=O)C1=Cc2ccc(-c3cncc(S(=O)(=O)N4CC(CN)C4)c3)cc2N=C(N)C1] | 613.7 | 684 | 174 |
| CycBz-36 | 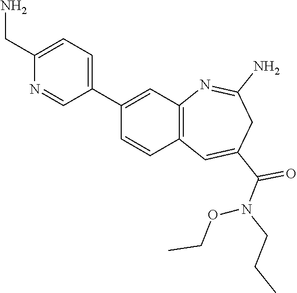 [SMILES: CCCN(OCC)C(=O)C1=Cc2ccc(-c3ccc(CN)nc3)cc2N=C(N)C1] | 393.5 | 439 | 54 |
| CycBz-37 | 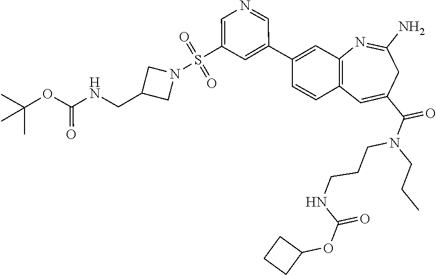 [SMILES: CCCN(CCCNC(=O)OC1CCC1)C(=O)C1=Cc2ccc(-c3cncc(S(=O)(=O)N4CC(CNC(=O)OC(C)(C)C)C4)c3)cc2N=C(N)C1] | 723.9 | | |
| CycBz-38 | 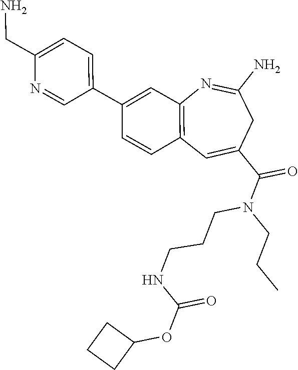 [SMILES: CCCN(CCCNC(=O)OC1CCC1)C(=O)C1=Cc2ccc(-c3ccc(CN)nc3)cc2N=C(N)C1] | 504.6 | 56 | 153 |
| CycBz-39 | 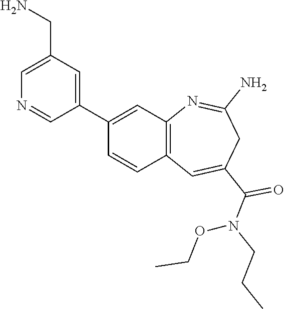 [SMILES: CCCN(OCC)C(=O)C1=Cc2ccc(-c3cncc(CN)c3)cc2N=C(N)C1] | 393.5 | 1780 | 65 |
| CycBz-40 | 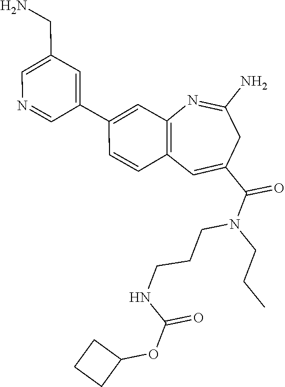 [SMILES: CCCN(CCCNC(=O)OC1CCC1)C(=O)C1=Cc2ccc(-c3cncc(CN)c3)cc2N=C(N)C1] | 504.6 | 357 | 755 |
| CycBz-41 | 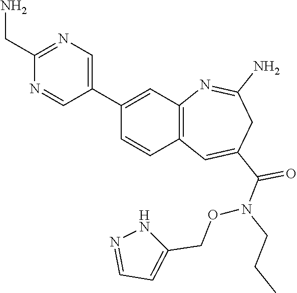 [SMILES: CCCN(OCc1ccn[nH]1)C(=O)C1=Cc2ccc(-c3cnc(CN)nc3)cc2N=C(N)C1] | 446.5 | 3926 | 128 |
| CycBz-42 | 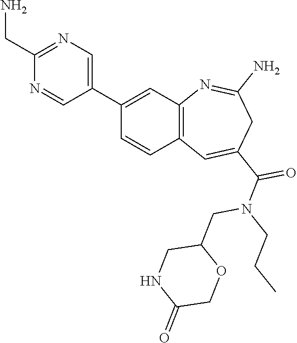 [SMILES: CCCN(CC1CNC(=O)CO1)C(=O)C1=Cc2ccc(-c3cnc(CN)nc3)cc2N=C(N)C1] | 463.5 | 9000 | 9000 |
| CycBz-43 | 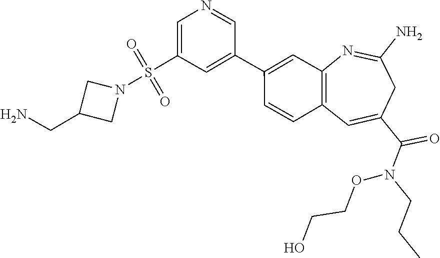 [SMILES: CCCN(OCCO)C(=O)C1=Cc2ccc(-c3cncc(S(=O)(=O)N4CC(CN)C4)c3)cc2N=C(N)C1] | 528.6 | 9000 | 6164 |
| CycBz-44 | 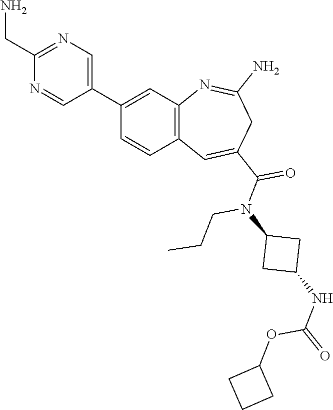 [SMILES: CCCN(C(=O)C1=Cc2ccc(-c3cnc(CN)nc3)cc2N=C(N)C1)[C@H]1C[C@H](NC(=O)OC2CCC2)C1] | 517.6 | 9000 | 6346 |
| CycBz-45 | 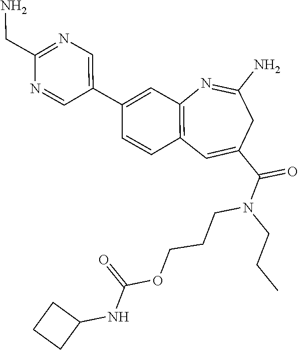 [SMILES: CCCN(CCCOC(=O)NC1CCC1)C(=O)C1=Cc2ccc(-c3cnc(CN)nc3)cc2N=C(N)C1] | 505.6 | 825 | 325 |
| CycBz-46 | 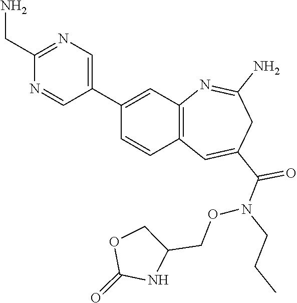 [SMILES: CCCN(OCC1COC(=O)N1)C(=O)C1=Cc2ccc(-c3cnc(CN)nc3)cc2N=C(N)C1] | 465.5 | 9000 | 3578 |
| CycBz-47 | 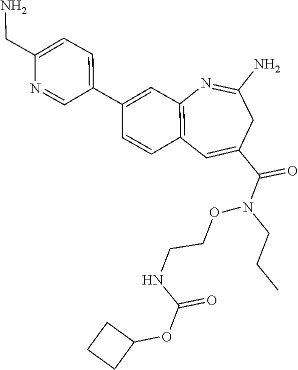 [SMILES: CCCN(OCCNC(=O)OC1CCC1)C(=O)C1=Cc2ccc(-c3ccc(CN)nc3)cc2N=C(N)C1] | 506.6 | 35 | 12 |
| CycBz-48 | 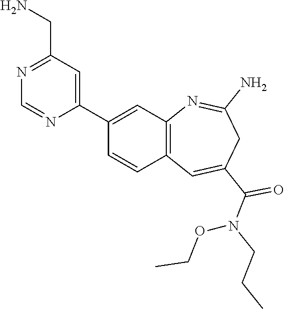 [SMILES: CCCN(OCC)C(=O)C1=Cc2ccc(-c3cc(CN)ncn3)cc2N=C(N)C1] | 394.5 | 9000 | 2164 |

TABLE 1b
8-Cyc-2-aminobenzazepine compounds (CycBz), where Cyc is phenyldiyl

| | | | HEK293 | |
|---|---|---|---|---|
| | | | hTLR7 | HEK293 |
| CycBz | | | EC50 | hTLR8 |
| No. | Structure | MW | (nM) | EC50 (nM) |
| CycBz-49 | 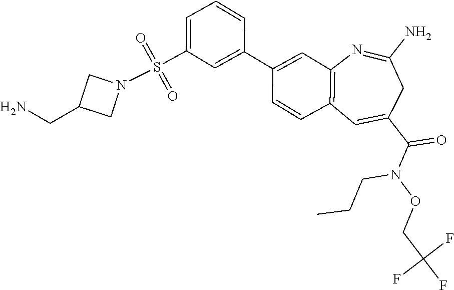 [SMILES: CCCN(OCC(F)(F)F)C(=O)C1=Cc2ccc(-c3cccc(S(=O)(=O)N4CC(CN)C4)c3)cc2N=C(N)C1] | 565.6 | 3932 | 461 |
| CycBz-50 | 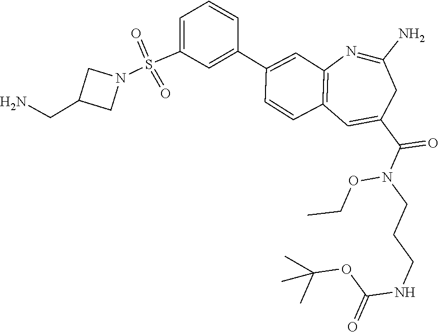 [SMILES: CCON(CCCNC(=O)OC(C)(C)C)C(=O)C1=Cc2ccc(-c3cccc(S(=O)(=O)N4CC(CN)C4)c3)cc2N=C(N)C1] | 626.8 | | |
| CycBz-51 | 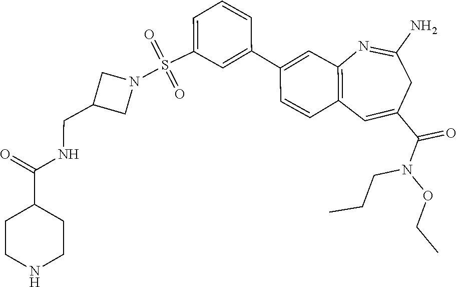 [SMILES: CCCN(OCC)C(=O)C1=Cc2ccc(-c3cccc(S(=O)(=O)N4CC(CNC(=O)C5CCNCC5)C4)c3)cc2N=C(N)C1] | 622.8 | 9000 | 4189 |
| CycBz-52 | 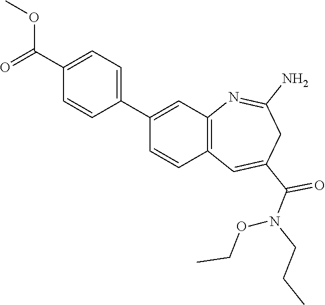 [SMILES: CCCN(OCC)C(=O)C1=Cc2ccc(-c3ccc(C(=O)OC)cc3)cc2N=C(N)C1] | 421.5 | 5764 | 629 |
| CycBz-53 | 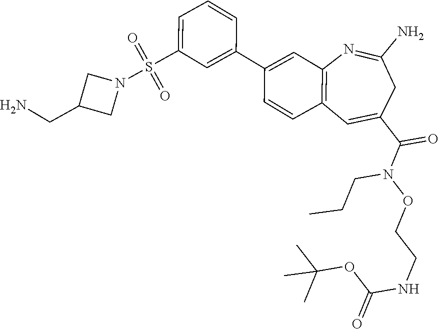 [SMILES: CCCN(OCCNC(=O)OC(C)(C)C)C(=O)C1=Cc2ccc(-c3cccc(S(=O)(=O)N4CC(CN)C4)c3)cc2N=C(N)C1] | 626.8 | | |
| CycBz-54 | 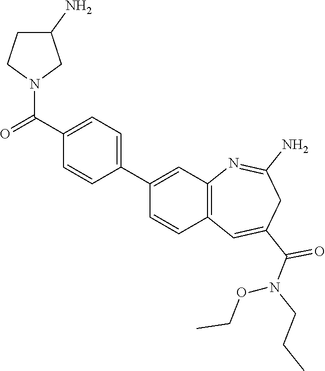 [SMILES: CCCN(OCC)C(=O)C1=Cc2ccc(-c3ccc(C(=O)N4CCC(N)C4)cc3)cc2N=C(N)C1] | 475.6 | 3089 | 860 |
| CycBz-55 | 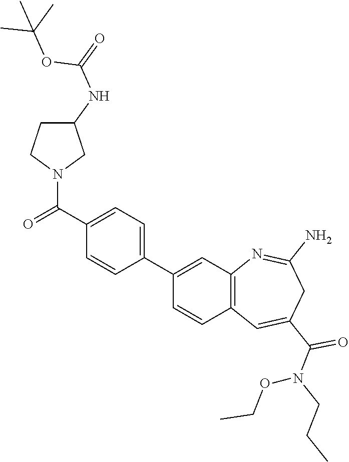 [SMILES: CCCN(OCC)C(=O)C1=Cc2ccc(-c3ccc(C(=O)N4CCC(NC(=O)OC(C)(C)C)C4)cc3)cc2N=C(N)C1] | 575.7 | 3682 | 377 |
| CycBz-56 | 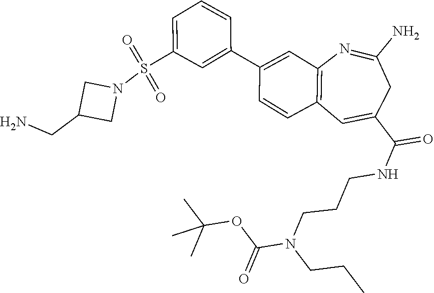 [SMILES: CCCN(CCCNC(=O)C1=Cc2ccc(-c3cccc(S(=O)(=O)N4CC(CN)C4)c3)cc2N=C(N)C1)C(=O)OC(C)(C)C] | 624.8 | | |
| CycBz-57 | 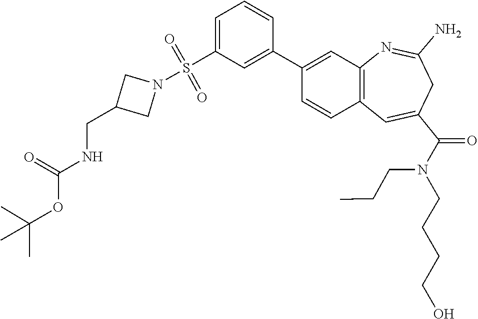 [SMILES: CCCN(CCCCO)C(=O)C1=Cc2ccc(-c3cccc(S(=O)(=O)N4CC(CNC(=O)OC(C)(C)C)C4)c3)cc2N=C(N)C1] | 639.8 | 5140 | 1080 |
| CycBz-58 | 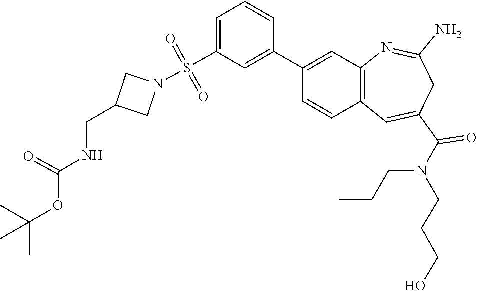 [SMILES: CCCN(CCCO)C(=O)C1=Cc2ccc(-c3cccc(S(=O)(=O)N4CC(CNC(=O)OC(C)(C)C)C4)c3)cc2N=C(N)C1] | 625.8 | 7152 | 374 |
| CycBz-59 | 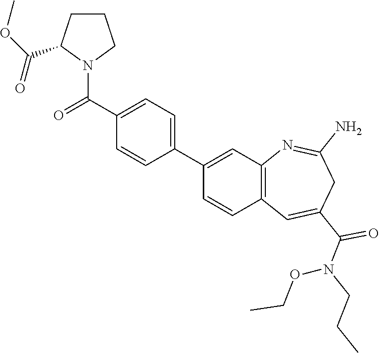 [SMILES: CCCN(OCC)C(=O)C1=Cc2ccc(-c3ccc(C(=O)N4CCC[C@H]4C(=O)OC)cc3)cc2N=C(N)C1] | 518.6 | 47 | 6 |
| CycBz-60 | 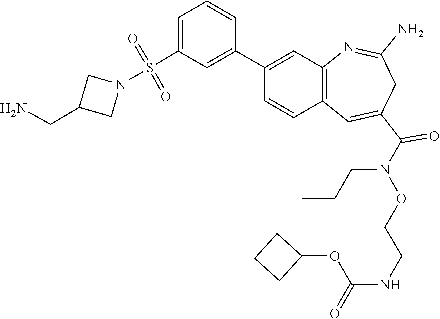 [SMILES: CCCN(OCCNC(=O)OC1CCC1)C(=O)C1=Cc2ccc(-c3cccc(S(=O)(=O)N4CC(CN)C4)c3)cc2N=C(N)C1] | 624.8 | 738 | 163 |
| CycBz-61 | 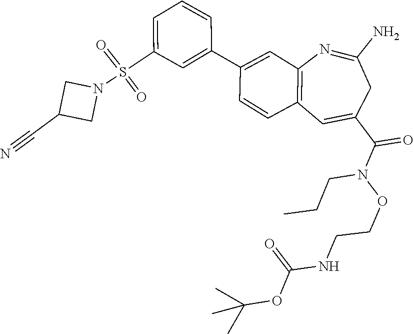 [SMILES: CCCN(OCCNC(=O)OC(C)(C)C)C(=O)C1=Cc2ccc(-c3cccc(S(=O)(=O)N4CC(C#N)C4)c3)cc2N=C(N)C1] | 622.7 | | |
| CycBz-62 | 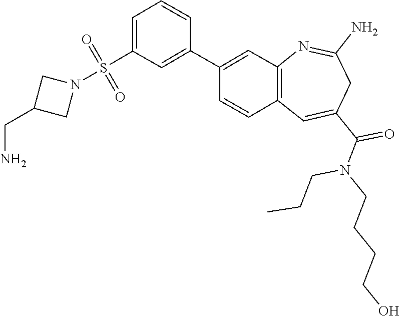 [SMILES: CCCN(CCCCO)C(=O)C1=Cc2ccc(-c3cccc(S(=O)(=O)N4CC(CN)C4)c3)cc2N=C(N)C1] | 539.7 | 9000 | 4661 |
| CycBz-63 | 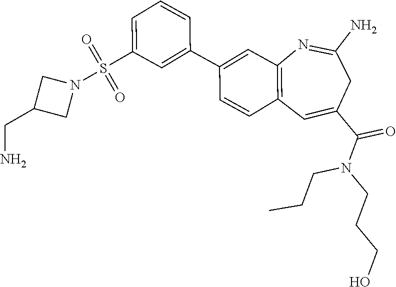 [SMILES: CCCN(CCCO)C(=O)C1=Cc2ccc(-c3cccc(S(=O)(=O)N4CC(CN)C4)c3)cc2N=C(N)C1] | 525.7 | 9000 | 3606 |
| CycBz-64 | 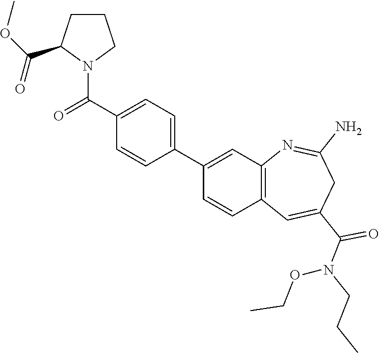 [SMILES: CCCN(OCC)C(=O)C1=Cc2ccc(-c3ccc(C(=O)N4CCC[C@@H]4C(=O)OC)cc3)cc2N=C(N)C1] | 518.6 | 3429 | 97 |
| CycBz-65 | 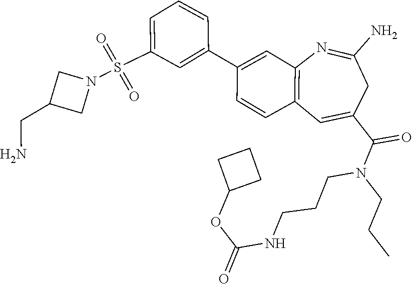 [SMILES: CCCN(CCCNC(=O)OC1CCC1)C(=O)C1=Cc2ccc(-c3cccc(S(=O)(=O)N4CC(CN)C4)c3)cc2N=C(N)C1] | 622.8 | 2311 | 1564 |
| CycBz-66 | 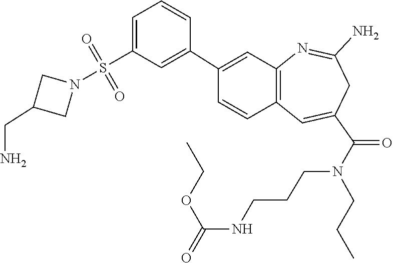 [SMILES: CCCN(CCCNC(=O)OCC)C(=O)C1=Cc2ccc(-c3cccc(S(=O)(=O)N4CC(CN)C4)c3)cc2N=C(N)C1] | 596.7 | | |
| CycBz-67 | 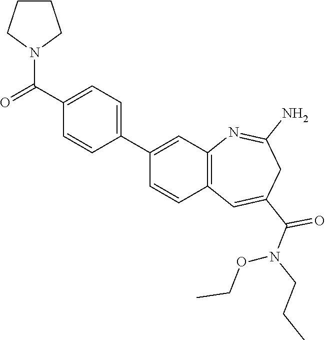 [SMILES: CCCN(OCC)C(=O)C1=Cc2ccc(-c3ccc(C(=O)N4CCCC4)cc3)cc2N=C(N)C1] | 460.6 | 3014 | 256 |
| CycBz-68 | 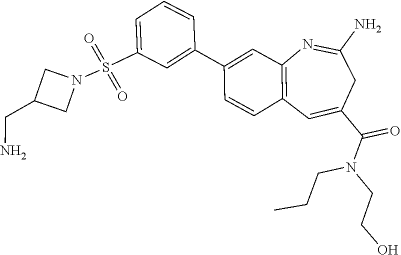 [SMILES: CCCN(CCO)C(=O)C1=Cc2ccc(-c3cccc(S(=O)(=O)N4CC(CN)C4)c3)cc2N=C(N)C1] | 511.6 | 9000 | 4909 |
| CycBz-69 | 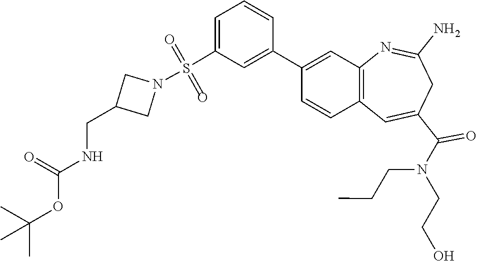 | 611.8 | 3525 | 423 |
| CycBz-70 | 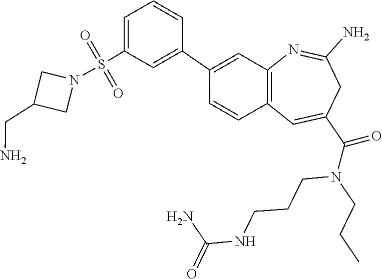 [SMILES: CCCN(CCCNC(N)=O)C(=O)C1=Cc2ccc(-c3cccc(S(=O)(=O)N4CC(CN)C4)c3)cc2N=C(N)C1] | 567.7 | 9000 | 9000 |
| CycBz-71 | 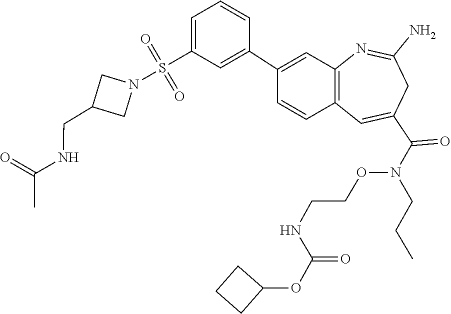 [SMILES: CCCN(OCCNC(=O)OC1CCC1)C(=O)C1=Cc2ccc(-c3cccc(S(=O)(=O)N4CC(CNC(C)=O)C4)c3)cc2N=C(N)C1] | 666.8 | | |
| CycBz-72 | 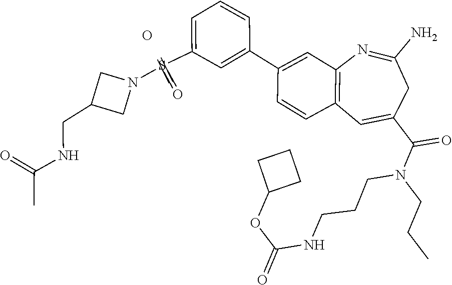 | 664.8 | | |
| CycBz-73 | 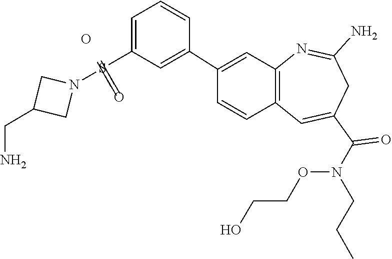 | 527.6 | 9000 | 822 |
| CycBz-74 | 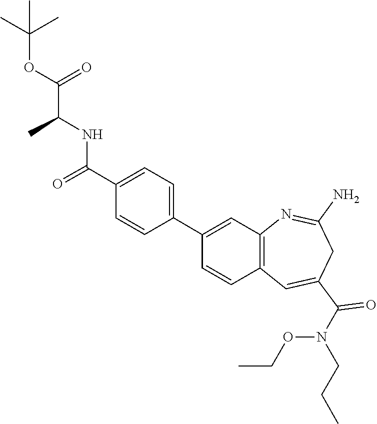 [SMILES: CCCN(OCC)C(=O)C1=Cc2ccc(-c3ccc(C(=O)N[C@@H](C)C(=O)OC(C)(C)C)cc3)cc2N=C(N)C1] | 534.7 | 1917 | 12 |
| CycBz-75 | 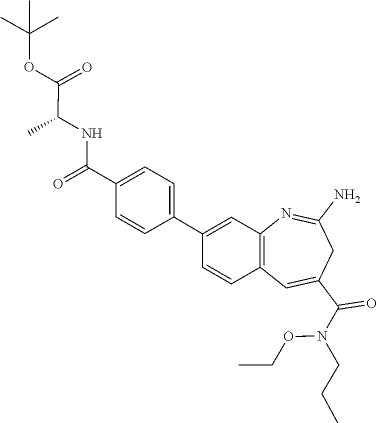 [SMILES: CCCN(OCC)C(=O)C1=Cc2ccc(-c3ccc(C(=O)N[C@H](C)C(=O)OC(C)(C)C)cc3)cc2N=C(N)C1] | 534.7 | 4751 | 86 |

### 8-Cyclyl-2-Aminobenzazepine-Linker Compounds

The immunoconjugates of the invention are prepared by conjugation of an anti-PD-L1 antibody with a 8-cyclyl-2-aminobenzazepine-linker compound, CycBzL. The 8-cyclyl-2-aminobenzazepine-linker compounds comprise a 8-Cyc-2-aminobenzazepine (Cyc) moiety covalently attached to a linker unit. The linker units comprise functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the CycBz-linker compound to form the immunoconjugate. Also, for example, a cysteine thiol of the antibody reacts with a maleimide or bromoacetamide group of the Hx-linker compound to form the immunoconjugate.

Electrophilic reactive functional groups suitable for the CycBz-linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2^{nd} Edition, Academic Press, 2008.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments include a 8-cyclyl-2-aminobenzazepine-linker compound of Formula II:

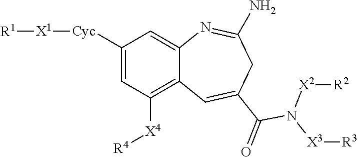

wherein Cyc is selected from the group consisting of phenyldiyl, heterocyclyldiyl and heteroaryldiyl;
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR⁵a)N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₆-C₂₀ aryldiyl)-*;
—(C₁-C₂₀ heteroaryl);
—(C₁-C₂₀ heteroaryldiyl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—(C₁-C₂₀ heteroaryl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₅ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₂
alkyldiyl)-NR⁵—*;
—N(R⁵)₂
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₁₂ alkyl);
—O—(C₁-C₂ alkyl);
—O—(C₁-C₁₂ alkyldiyl)-N(R⁵)₂;
—O—(C₁-C₁₂ alkyldiyl)-N(R⁵)—*;
—O—C(═O)N(R⁵)₂;
—O—C(═O)N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), 0, N(R⁵), S, S(O)₂, and S(O)₂N(R⁵);
R⁵ is independently selected from the group consisting of H, C₆-C₂₀ aryl, C₃-C₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
   Q-C(═O)—PEG-;
   Q-C(═O)—PEG-C(═O)N(R⁶)—(C₁-C₁₂ alkyldiyl)-C(═O)-Gluc-;
   Q-C(═O)—PEG-O—;
   Q-C(═O)—PEG-O—C(═O)—;
   Q-C(═O)—PEG-C(═O)—;
   Q-C(═O)—PEG-C(═O)—PEP-;
   Q-C(═O)—PEG-N(R⁶)—;
   Q-C(═O)—PEG-N(R⁶)—C(═O)—;
   Q-C(═O)—PEG-N(R⁶)-PEG-C(═O)—PEP-;
   Q-C(═O)—PEG-N+(R⁶)₂-PEG-C(═O)—PEP-;
   Q-C(═O)—PEG-C(═O)—PEP-N(R⁶)—(C₁-C₁₂ alkyldiyl)-;
   Q-C(═O)—PEG-C(═O)—PEP-N(R⁶)—(C₁-C₁₂ alkyldiyl)N(R⁶)C(═O)—(C₂-C₅ monoheterocyclyldiyl)-;
   Q-C(═O)—PEG-SS—(C₁-C₁₂ alkyldiyl)-OC(═O)—;
   Q-C(═O)—PEG-SS—(C₁-C₁₂ alkyldiyl)-C(═O)—;
   Q-C(═O)—(C₁-C₁₂ alkyldiyl)-C(═O)—PEP-;
   Q-C(═O)—(C₁-C₁₂ alkyldiyl)-C(═O)—PEP-N(R⁶)—(C₁-C₁₂ alkyldiyl)-;
   Q-C(═O)—(C₁-C₁₂ alkyldiyl)-C(═O)—PEP-N(R⁶)—(C₁-C₁₂ alkyldiyl)-N(R⁵)—C(═O);
   Q-C(═O)—(C₁-C₁₂ alkyldiyl)-C(═O)—PEP-N(R⁶)—(C₁-C₁₂ alkyldiyl)-N(R⁶)C(═O)—(C₂-C₅ monoheterocyclyldiyl)-;
   Q-(CH₂)ₘC(═O)N(R⁶)—PEG-;
   Q-(CH₂)ₘC(═O)N(R⁶)—PEG-C(═O)N(R⁶)—(C₁-C₁₂ alkyldiyl)-C(═O)-Gluc-;
   Q-(CH₂)ₘC(═O)N(R⁶)—PEG-O—;
   Q-(CH₂)ₘC(═O)N(R⁶)—PEG-O—C(═O)—;
   Q-(CH₂)ₘ—C(═O)N(R⁶)—PEG-C(═O)—;
   Q-(CH₂)ₘC(═O)N(R⁶)—PEG-N(R⁵)—;
   Q-(CH₂)ₘC(═O)N(R⁶)—PEG-N(R⁵)—C(═O)—;
   Q-(CH₂)ₘC(═O)N(R⁶)—PEG-C(═O)—PEP-;
   Q-(CH₂)ₘC(═O)N(R⁶)—PEG-SS—(C₁-C₁₂ alkyldiyl)-OC(═O)—;
   Q-(CH₂)ₘ—C(═O)—PEP-N(R⁶)—(C₁-C₁₂ alkyldiyl)-;
   Q-(CH₂)ₘ—C(═O)—PEP-N(R⁶)—(C₁-C₁₂ alkyldiyl)N(R⁶)C(═O)—; and
   Q-(CH₂)ₘ—C(═O)—PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ—; m is an integer from 1 to 5, and n is an integer from 2 to 50;
Gluc has the formula:

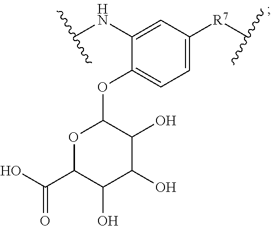

PEP has the formula:

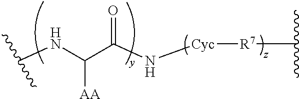

where AA is independently selected from a natural or unnatural amino acid side chain, or one or more of AA, and an adjacent nitrogen atom form a 5-membered ring proline amino acid, and the wavy line indicates a point of attachment;

Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

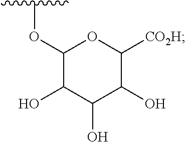 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1;
Q is selected from the group consisting of N-hydroxysuccinimidyl, N-hydroxysulfosuccinimidyl, maleimide, and phenoxy substituted with one or more groups independently selected from F, Cl, NO₂, and SO₃⁻; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)mCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the 8-cyclyl-2-aminobenzazepine-linker compound of Formula II includes wherein Q is selected from:

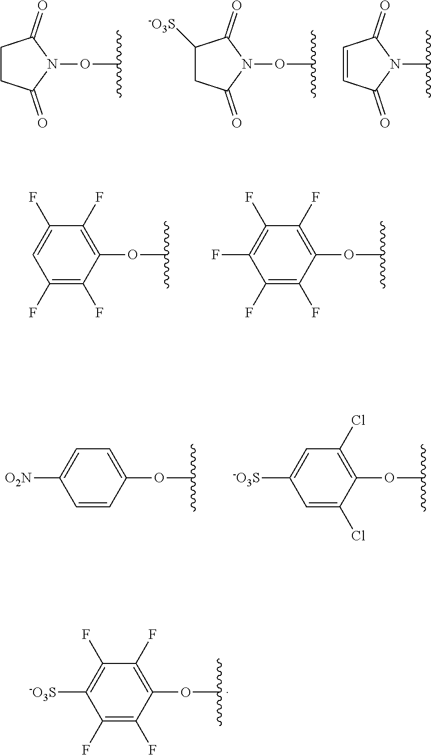

An exemplary embodiment of the 8-cyclyl-2-aminobenzazepine-linker compound of Formula II includes wherein Q is phenoxy substituted with one or more F.

An exemplary embodiment of the 8-cyclyl-2-aminobenzazepine-linker compound of Formula II includes wherein Q is 2,3,5,6-tetrafluorophenoxy.

An exemplary embodiment of the 8-cyclyl-2-aminobenzazepine-linker (CycBzL) compound is selected from Tables 2a and 2b. Each compound was synthesized, purified, and characterized by mass spectrometry and shown to have the mass indicated. Additional experimental procedures are found in the Examples. The 8-Cyc-2-aminobenzazepine-linker compounds of Tables 2a and 2b demonstrate the surprising and unexpected property of TLR8 agonist selectivity which may predict useful therapeutic activity to treat cancer and other disorders. The 8-cyclyl-2-aminobenzazepine-linker intermediate, Formula II compounds of Table 2 are used in conjugation with antibodies by the methods of Example 201 to form the Immunoconjugates of Tables 3a and 3b.

TABLE 2a
8-Cyc-2-aminobenzazepine-linker intermediate, Formula II compounds
(CycBzL)

| BzL | | |
|---|---|---|
| No. | Structure | MW |
| CycBzL-1 | 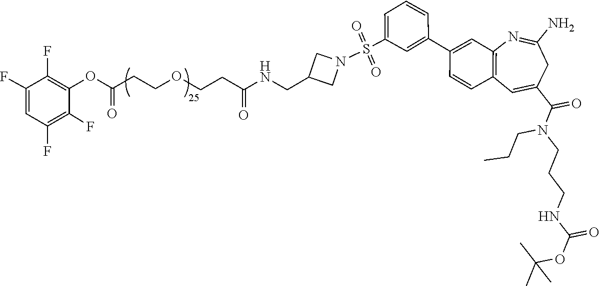 | 1974.2 |
| CycBzL-2 | 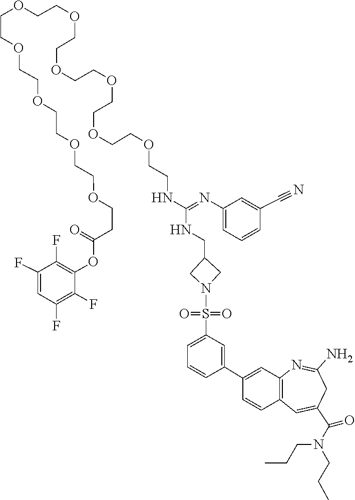 [SMILES: CCCN(CCC)C(=O)C1=Cc2ccc(-c3cccc(S(=O)(=O)N4CC(CN/C(=N\c5cccc(C#N)c5)NCCOCCOCCOCCOCCOCCOCCOCCOCCOCCOCCC(=O)Oc5c(F)c(F)cc(F)c5F)C4)c3)cc2N=C(N)C1] | 1313.5 |
| CycBzL-3 | 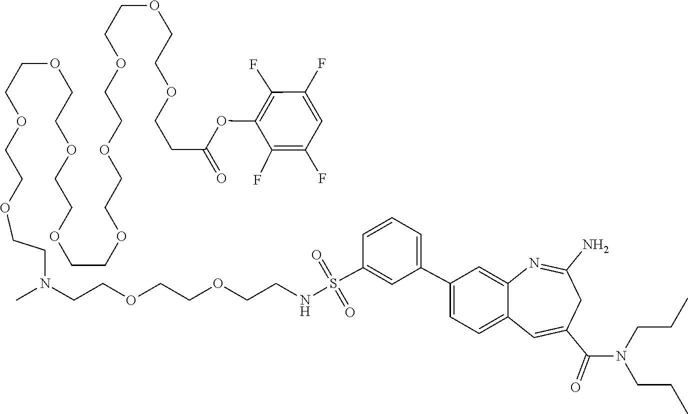 [SMILES: CCCN(CCC)C(=O)C1=Cc2ccc(-c3cccc(S(=O)(=O)NCCOCCOCCN(C)CCOCCOCCOCCOCCOCCOCCOCCOCCOCCOCCC(=O)Oc4c(F)c(F)cc(F)c4F)c3)cc2N=C(N)C1] | 1246.4 |
| CycBzL-4 | 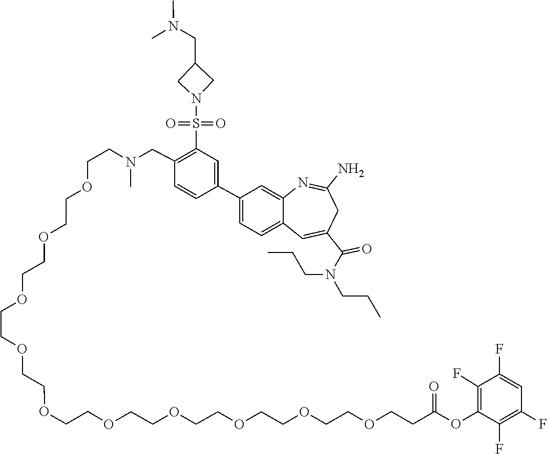 [SMILES: CCCN(CCC)C(=O)C1=Cc2ccc(-c3ccc(CN(C)CCOCCOCCOCCOCCOCCOCCOCCOCCOCCOCCC(=O)Oc4c(F)c(F)cc(F)c4F)c(S(=O)(=O)N4CC(CN(C)C)C4)c3)cc2N=C(N)C1] | 1241.4 |
| CycBzL-5 | 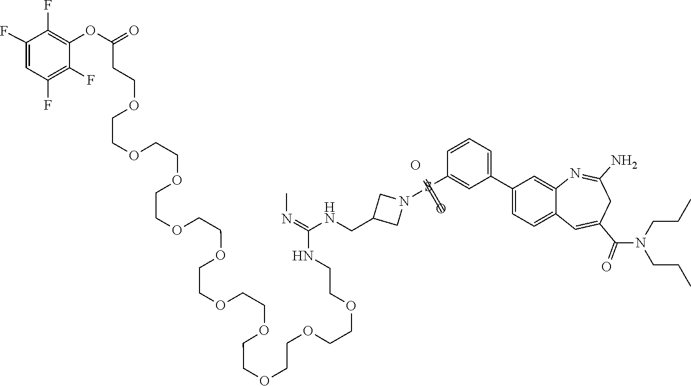 | 1226.4 |
| CycBzL-6 | 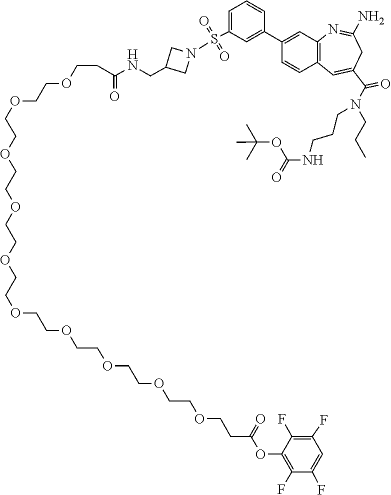 [SMILES: CCCN(CCCNC(=O)OC(C)(C)C)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1313.5 |
| CycBzL-7 | 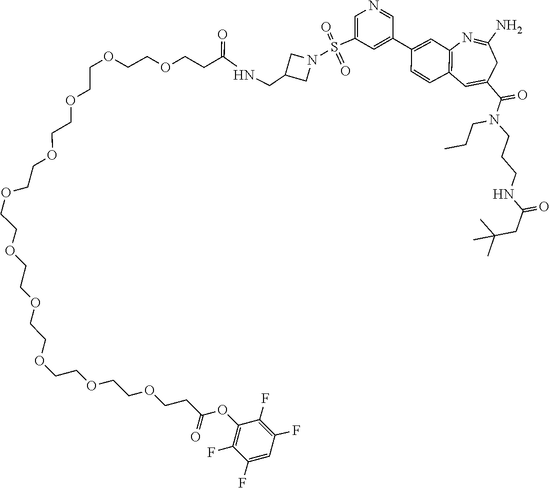 [SMILES: CCCN(CCCNC(=O)CC(C)(C)C)C(=O)C1=Cc2ccc(-c3cncc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1312.5 |
| CycBzL-8 | 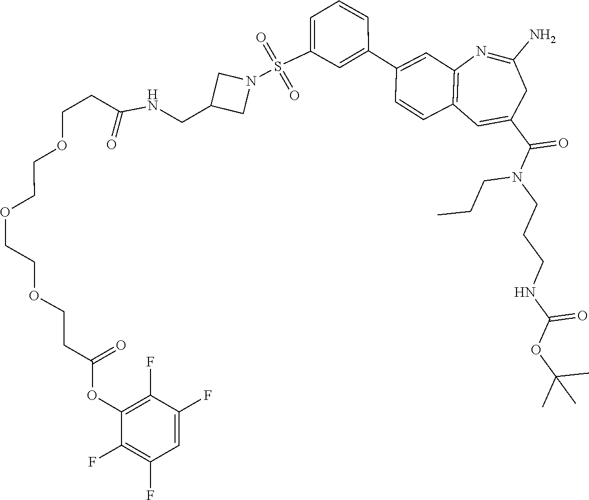 [SMILES: CCCN(CCCNC(=O)OC(C)(C)C)C(=O)C1=Cc2ccc(-c3cccc(S(=O)(=O)N4CC(CNC(=O)CCOCCOCCOCCC(=O)Oc5c(F)c(F)cc(F)c5F)C4)c3)cc2N=C(N)C1] | 1005.1 |
| CycBzL-9 | 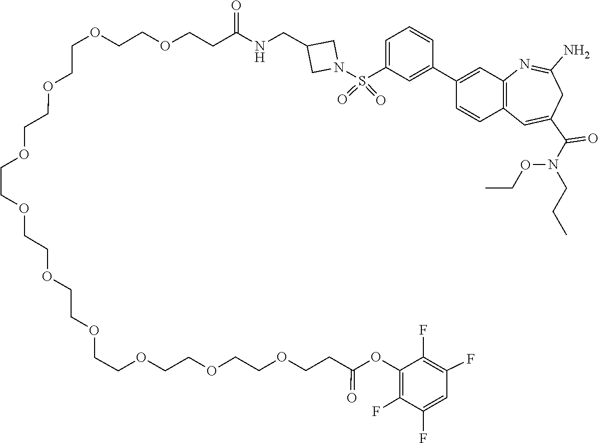 [SMILES: CCCN(OCC)C(=O)C1=Cc2ccc(-c3cccc(S(=O)(=O)N4CC(CNC(=O)CCOCCOCCOCCOCCOCCOCCOCCOCCOCCOCCC(=O)Oc5c(F)c(F)cc(F)c5F)C4)c3)cc2N=C(N)C1] | 1200.3 |
| CycBzL-10 | 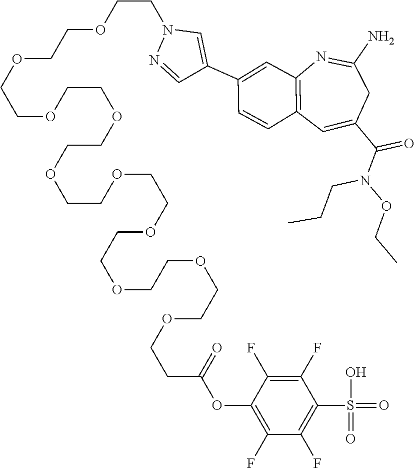 [SMILES: CCCN(OCC)C(=O)C1=Cc2ccc(-c3cnn(CCOCCOCCOCCOCCOCCOCCOCCOCCOCCOCCC(=O)Oc4c(F)c(F)c(S(=O)(=O)O)c(F)c4F)c3)cc2N=C(N)C1] | 1094.1 |
| CycBzL-11 | 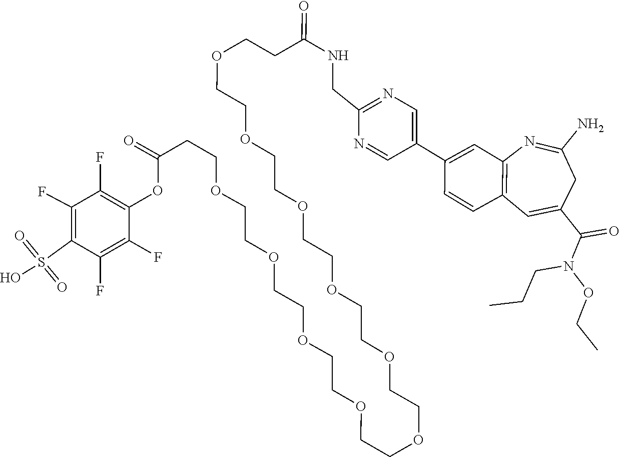 [SMILES: CCCN(OCC)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1163.2 |
| CycBzL-12 | 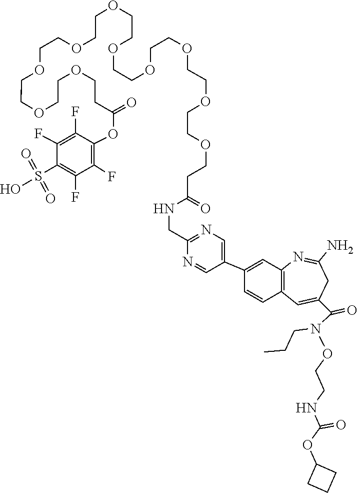 [SMILES: CCCN(OCCNC(=O)OC1CCC1)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1276.3 |
| CycBzL-13 | 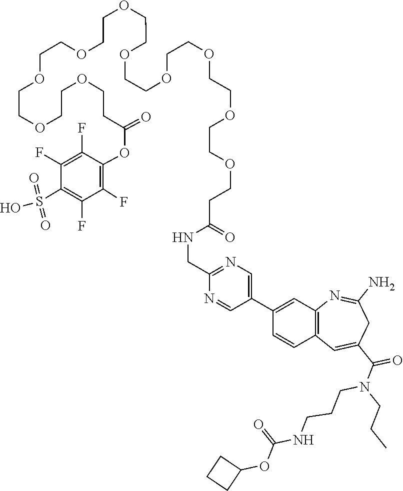 [SMILES: CCCN(CCCNC(=O)OC1CCC1)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1274.3 |
| CycBzL-14 | 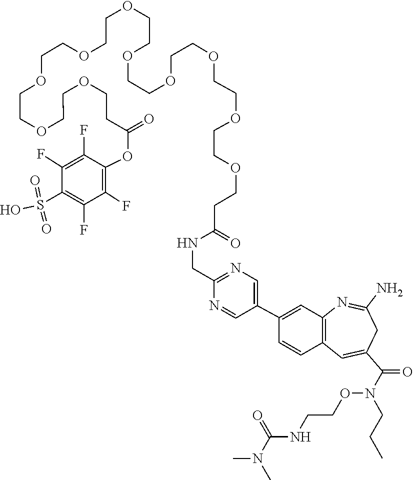 [SMILES: CCCN(OCCNC(=O)N(C)C)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1249.3 |
| CycBzL-15 | 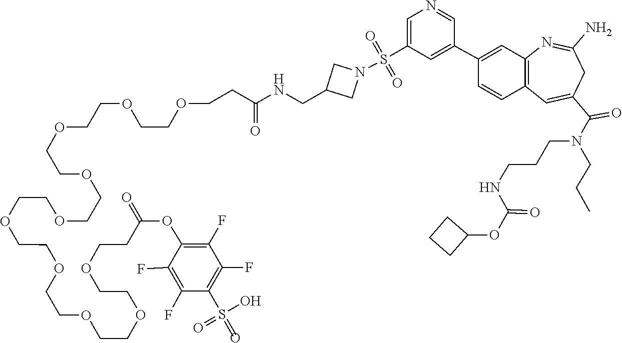 | 1392.5 |

TABLE 2b
8-Cyc-2-aminobenzazepine-linker intermediate, Formula II compounds
(CycBzL)

| BzL | | |
|---|---|---|
| No. | Structure | MW |
| CycBzL-16 | 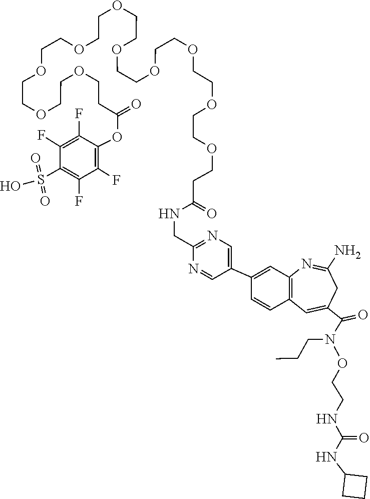 [SMILES: CCCN(OCCNC(=O)NC1CCC1)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1275.3 |
| CycBzL-17 | 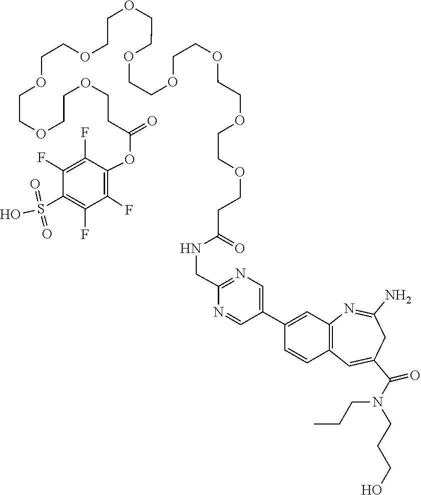 [SMILES: CCCN(CCCO)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1177.2 |
| CycBzL-18 | 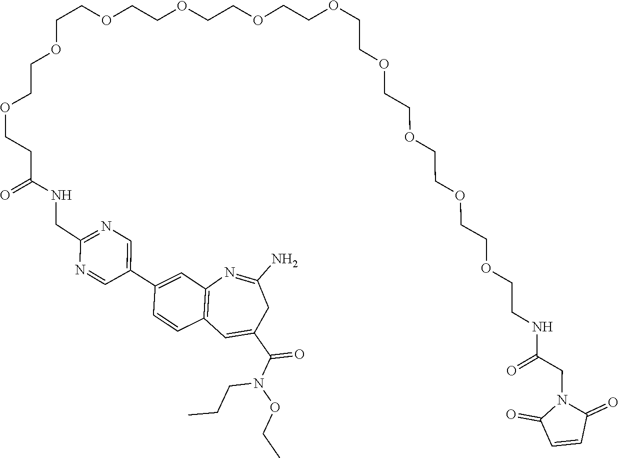 [SMILES: CCCN(OCC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1043.2 |
| CycBzL-19 | 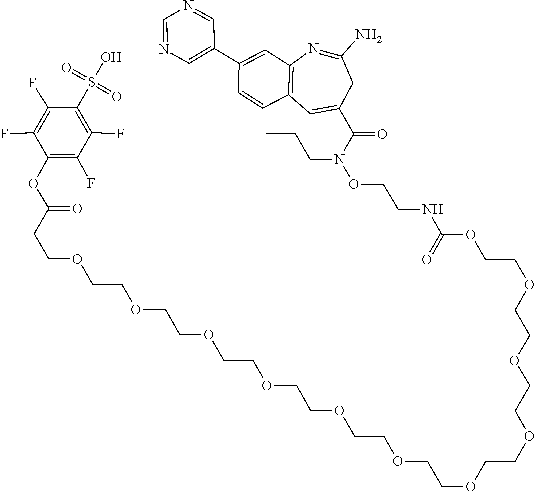 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(-c3cncnc3)cc2N=C(N)C1] | 1165.2 |
| CycBzL-20 | 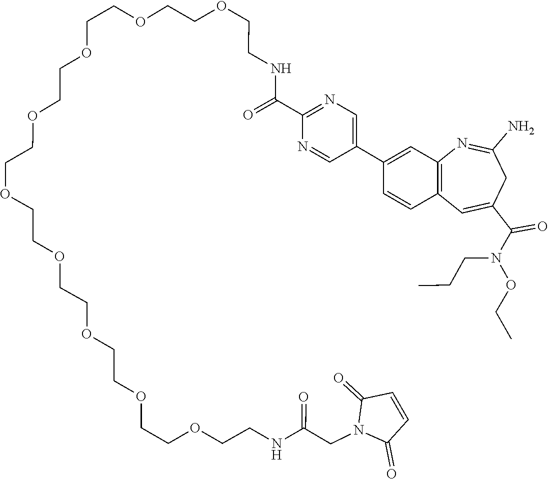 [SMILES: CCCN(OCC)C(=O)C1=Cc2ccc(-c3cnc(C(=O)NCCOCCOCCOCCOCCOCCOCCOCCOCCOCCNC(=O)CN4C(=O)C=CC4=O)nc3)cc2N=C(N)C1] | 985.1 |
| CycBzL-21 | 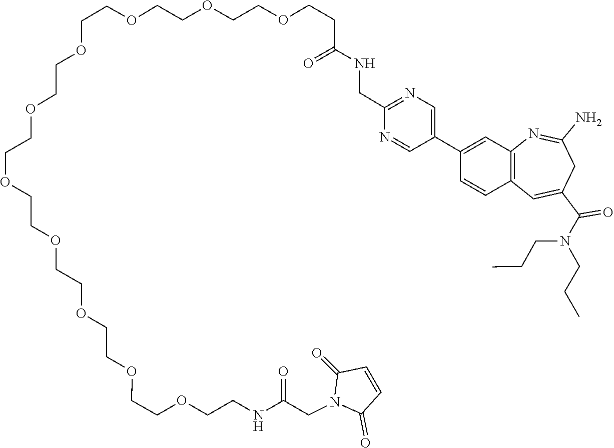 [SMILES: CCCN(CCC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1041.2 |
| CycBzL-22 | 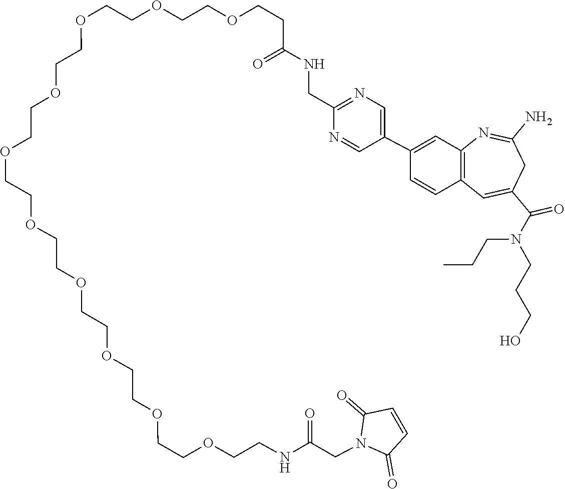 [SMILES: CCCN(CCCO)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1057.2 |
| CycBzL-23 | 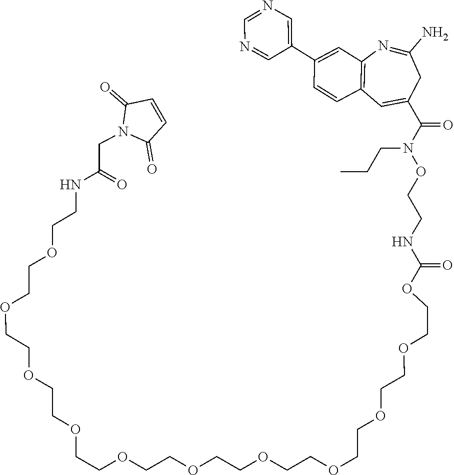 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(-c3cncnc3)cc2N=C(N)C1] | 1045.2 |
| CycBzL-24 | 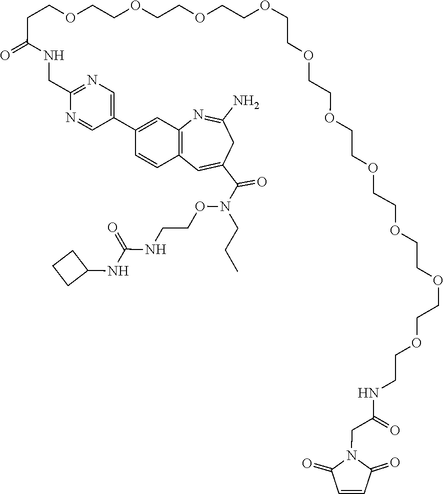 [SMILES: CCCN(OCCNC(=O)NC1CCC1)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1155.3 |

### PD-L1 Immunoconjugates

Immune-stimulating antibody conjugates, i.e. immunoconjugates, direct TLR7/8 agonists into tumors to activate tumor-infiltrating myeloid cells and initiate a broad innate and adaptive anti-tumor immune response (Ackerman, et al., (2021) Nature Cancer 2:18-33.

PD-L1 is an immune checkpoint, transmembrane protein that regulates anti-tumor T cell responses and is expressed on tumor cells as well as tumor-infiltrating immune cells across many tumor types. Immune-stimulating antibody conjugates (ISACs) consist of tumor-targeting antibodies conjugated to immune stimulants and are designed to activate the innate and adaptive immune systems against tumor cells following systemic administration. PD-L1-targeted TLR7/8 immunoconjugates elicit robust myeloid cell activation which leads to improved anti-tumor responses compared to anti-PD-L1 treatment in pre-clinical tumor models.

A panel of anti-PD-L1 antibodies was identified through a phage display screen and subsequently tested for PD-L1 binding affinity and specificity, PD-L1/PD-1 blocking, antibody-dependent cellular phagocytosis (ADCP) by myeloid cells, and anti-tumor efficacy. Anti-PD-L1 antibodies induced robust ADCP by myeloid effector cells and medium to strong PD-L1/PD-1 blockade in vitro. Selected antibodies inhibited the growth of syngeneic MC38-hPD-L1 tumors in vivo, confirming efficient immune-checkpoint blockade.

Certain antibodies were conjugated to 8-Cyc-2-aminobenzazepine (CycBz) TLR7/8 agonists by a linker, and the resulting PD-L1 immunoconjugates were evaluated for in vitro myeloid cell activation and in vivo efficacy (Example 203) against syngeneic and xenograft tumors. Certain PD-L1 immunoconjugates induced robust, target-dependent activation of myeloid cells when co-cultured with PD-L1-expressing tumor cells, as measured by increased secretion of such cytokines as IL-12p70, IFNa, and TNFa. Myeloid activation was observed following co-culture with tumor cells having various levels of endogenous PD-L1 expression that was within the range of PD-L1 expression observed in human tumors. Systemically administered surrogate PD-L1 immunoconjugates were well tolerated in mice and showed improved anti-tumor efficacy over anti-PD-L1 antibodies, with significant tumor growth delay or complete responses frequently observed in syngeneic (e.g. MB49, MC38-hPD-L1) as well as xenograft (e.g. HCC1954-hPD-L1) tumor models. These data demonstrate the potential of PD-L1-targeted immunoconjugates as multifunctional therapeutics that may improve efficacy of PD-L1/PD-1 inhibition by combining three mechanisms of action into a single molecule: TLR-mediated myeloid cell activation, T cell activation through immune-checkpoint inhibition as well as ADCP.

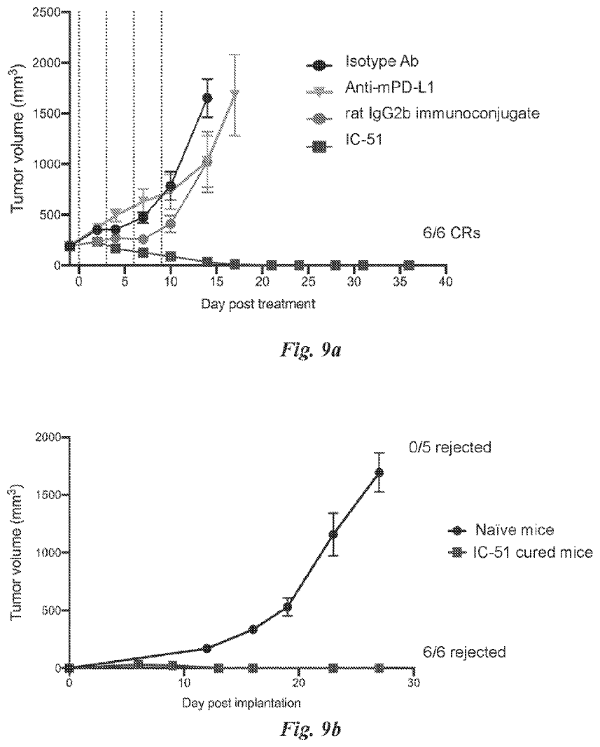

FIG. 9a shows a graph of an in vivo study of tumor volume over time after treatment comparing the efficacy of immunoconjugate PD-L1 IC-51 from Table 3b versus unconjugated anti-PD-L1 antibody in a syngeneic MB49 tumor model. Mice bearing large (˜200 mm³) syngeneic MB49 tumors were treated with immunoconjugate IC-51 from Table 3b, comprising CycBzL-24 (Table 2b), rat IgG2b isotype control immunoconjugate comprising CycBzL-24, unconjugated anti-PD-L1, and unconjugated IgG2b isotype antibody at 10 mg/kg intraperitoneal (IP), Q3Dx4 (n=6 per group). Treatment with anti-mPD-L1 immunoconjugate IC-51 resulted in an improved anti-tumor efficacy versus unconjugated anti-PD-L1 and complete responses (CRs) in all 6 animals.

FIG. 9b shows a graph of an in vivo study of tumor volume over time after implantation of MB49 tumor cells, assessing the immunological memory in a re-challenge study. All 6 mice that experienced complete regression of MB49 tumors following immunoconjugate PD-L1 IC-51 from Table 3b treatment in the study from FIG. 9a were re-challenged with MB49 tumor cells about 1 month after tumor clearance (about 7 weeks post final dose). None of the previously cured mice developed tumors, while all naïve mice (n=5) did suggesting development of an immunological memory following treatment with anti-PD-L1 immunoconjugate IC-51.

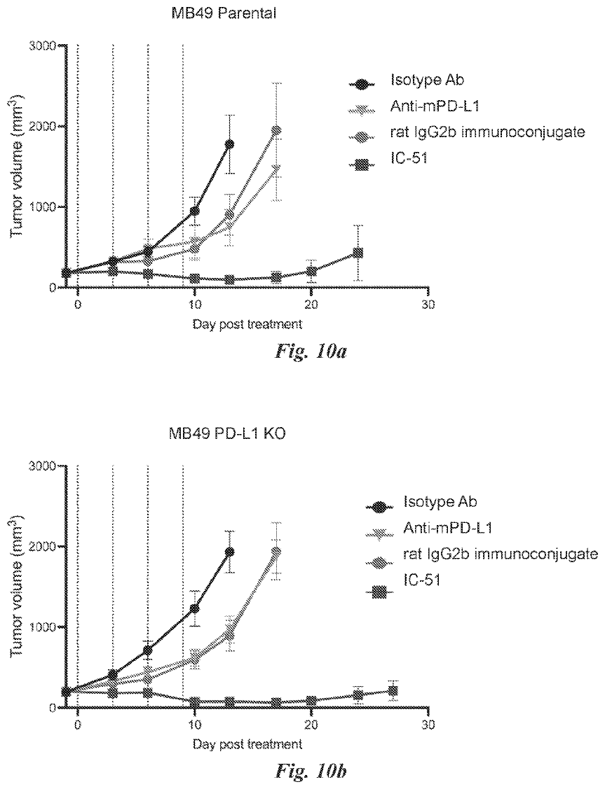

FIG. 10a shows a graph of tumor volume over time after treatment of parental syngeneic MB49 tumors with immunoconjugate IC-51 from Table 3b, comprising CycBzL-24 (Table 2b), rat IgG2b isotype control immunoconjugate comprising CycBzL-24, unconjugated anti-PD-L1, and unconjugated IgG2b isotype antibody at 5 mg/kg IP, Q3Dx4.

FIG. 10b shows a graph of tumor volume over time after treatment of PD-L1 knockout (PD-L1 KO) syngeneic MB49 tumors lacking PD-L1 expression on tumor cells with immunoconjugate IC-51 from Table 3b, comprising CycBzL-24 (Table 2b), rat IgG2b isotype control immunoconjugate comprising CycBzL-24, unconjugated anti-PD-L1, and unconjugated IgG2b isotype antibody at 5 mg/kg IP, Q3Dx4.

Treatment with PD-L1 IC-51 resulted in improved anti-tumor efficacy even in the absence of PD-L1 expression on tumor cells, demonstrating an immunoconjugate of the invention can induce an anti-tumor response through PD-L1 on immune cells.

Exemplary embodiments of immunoconjugates comprise an anti-PD-L1 antibody covalently attached to one or more 8-Cyc-2-aminobenzazepine (CycBz) moieties by a linker, and having Formula I:

Ab-[L-CycBz]ₚ I
or a pharmaceutically acceptable salt thereof,
wherein:
Ab is an antibody construct that has an antigen binding domain that binds PD-L1;
p is an integer from 1 to 8;
CycBz is the 8-Cyc-2-aminobenzazepine moiety having the formula:

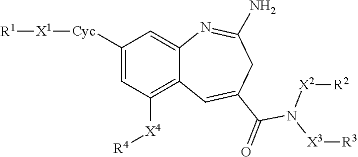

Cyc is selected from the group consisting of phenyldiyl, heterocyclyldiyl and heteroaryldiyl;
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₁-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR 5-C(═NR^{5a})N(R⁵)—*;
—(C₁-C₂₀ heteroaryl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C i-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR⁵a)N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₂ alkyldiyl)-NR⁵—*;
—N(R⁵)₂;
—N(R⁵)—*;
—N(R⁵)C(═O)R;
—N(R⁵)C(═O)*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂,
—NR⁵C(═NR⁵a)N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₁₂ alkyl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—O—C(═O)N(R⁵)₂;
—O—C(═O)N(R⁵)—*
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), 0, N(R⁵), S, S(O)₂, and S(O)₂N(R⁵);
R⁵ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
   —C(═O)—PEG-;
   —C(═O)—PEG-C(═O)N(R⁶)—(C₁-C₁₂ alkyldiyl)-C(═O)-Gluc-;
   —C(═O)—PEG-O—;
   —C(═O)—PEG-O—C(═O)—;
   —C(═O)—PEG-C(═O)—;
   —C(═O)—PEG-C(═O)—PEP-;
   —C(═O)—PEG-N(R⁶)—.
   —C(═O)—PEG-N(R⁶)—C(═O)—;
   —C(═O)—PEG-N(R⁶)—PEG-C(═O)—PEP-;
   —C(═O)—PEG-N(═O)-PEP-(═O)-PEP-;
   —C(═O)—PEG-C(═O)—PEP-N(R⁶)—(C₁-C₁₂ alkyldiyl)-;
   —C(═O)—PEG-C(═O)—PEP-N(R⁶)—(C₁-C₁₂ alkyldiyl)N(R⁶)C(═O)—(C₂-C₅ monoheterocyclyldiyl)-;
   —C(═O)—PEG-SS—(C₁-C₁₂ alkyldiyl)-OC(═O)—;
   —C(═O)—PEG-SS—(C₁-C₁₂ alkyldiyl)-C(═O)—;
   —C(═O)—(C₁-C₁₂ alkyldiyl)-C(═O)—PEP-;
   —C(═O)—(C₁-C₁₂ alkyldiyl)-C(═O)—PEP-N(R⁶)—(C₁-C₁₂ alkyldiyl)-;
   —C(═O)—(C₁-C₁₂ alkyldiyl)-C(═O)—PEP-N(R⁶)—(C₁-C₁₂ alkyldiyl)-N(R⁵)—C(═O);
   —C(═O)—(C₁-C₁₂ alkyldiyl)-C(═O)—PEP-N(R⁶)—(C₁-C₁₂ alkyldiyl)-N(R⁶)C(═O)—(C₂-C₅ monoheterocyclyldiyl)-;
   -succinimidyl-(CH₂)ₘ—C(═O)N(R⁶)—PEG-;
   -succinimidyl-(CH₂)ₘ—C(═O)N(R⁶)—PEG-C(═O)N(R⁶)—(C₁-C₁₂ alkyldiyl)-C(═O)-Gluc-;
   -succinimidyl-(CH₂)ₘ—C(═O)N(R⁶)—PEG-O—;
   -succinimidyl-(CH₂)ₘ—C(═O)N(R⁶)—PEG-O—C(═O)—;
   -succinimidyl-(CH₂)ₘ—C(═O)N(R⁶)—PEG-C(═O)—;
   -succinimidyl-(CH₂)ₘ—C(═O)N(R⁶)—PEG-N(R⁵)—;
   -succinimidyl-(CH₂)ₘ—C(═O)N(R⁶)—PEG-N(R⁵)—C(═O)—;
   -succinimidyl-(CH₂)ₘ—C(═O)N(R⁶)—PEG-C(═O)—PEP-;
   -succinimidyl-(CH₂)ₘ—C(═O)N(R⁶)—PEG-SS—(C₁-C₁₂ alkyldiyl)-OC(═O)—;
   -succinimidyl-(CH₂)ₘ—C(═O)—PEP-N(R⁶)—(C₁-C₁₂ alkyldiyl)-;
   -succinimidyl-(CH₂)ₘ—C(═O)—PEP-N(R⁶)—(C₁-C₁₂ alkyldiyl)N(R⁶)C(═O)—; and
   -succinimidyl-(CH₂)ₘ—C(═O)—PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ(CH₂)ₘ—; m is an integer from 1 to 5, and n is an integer from 2 to 50;
Gluc has the formula:

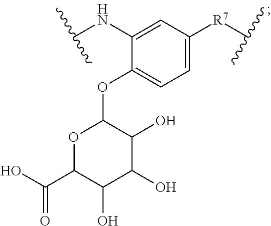

PEP has the formula:

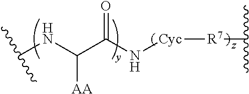

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

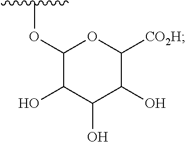 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the immunoconjugate of Formula I includes wherein the antibody is selected atezolizumab, durvalumab, and avelumab and, or a biosimilar or a biobetter thereof.

An exemplary embodiment of the immunoconjugate of Formula I includes wherein X² is a bond, and R² is C₁-C₈ alkyl.

An exemplary embodiment of the immunoconjugate of Formula I includes wherein X¹ is a bond, and R¹ is H.

An exemplary embodiment of the immunoconjugate of Formula I includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

An exemplary embodiment of the immunoconjugate of Formula I includes wherein R² is C₁-C₈ alkyl and R³ is —(C₁-C₈ alkyldiyl)-N(R⁵)CO₂R⁵.

An exemplary embodiment of the immunoconjugate of Formula I includes wherein R² is —CH₂CH₂CH₃ and R³ is selected from —CH₂CH₂CH₂NHCO₂(t-Bu), —OCH₂CH₂NHCO₂(cyclobutyl), and —CH₂CH₂CH₂NHCO₂(cyclobutyl).

An exemplary embodiment of the immunoconjugate of Formula I includes wherein R² and R³ are each independently selected from —CH₂CH₂CH₃, —OCH₂CH₃, —CH₂CH₂CF₃, and —CH₂CH₂CH₂OH.

An exemplary embodiment of the immunoconjugate of Formula I includes wherein R² and R³ are each —CH₂CH₂CH₃.

An exemplary embodiment of the immunoconjugate of Formula I includes wherein R² is —CH₂CH₂CH₃ and R³ is —OCH₂CH₃.

An exemplary embodiment of the immunoconjugate of Formula I includes wherein X³—R³ is selected from the group consisting of

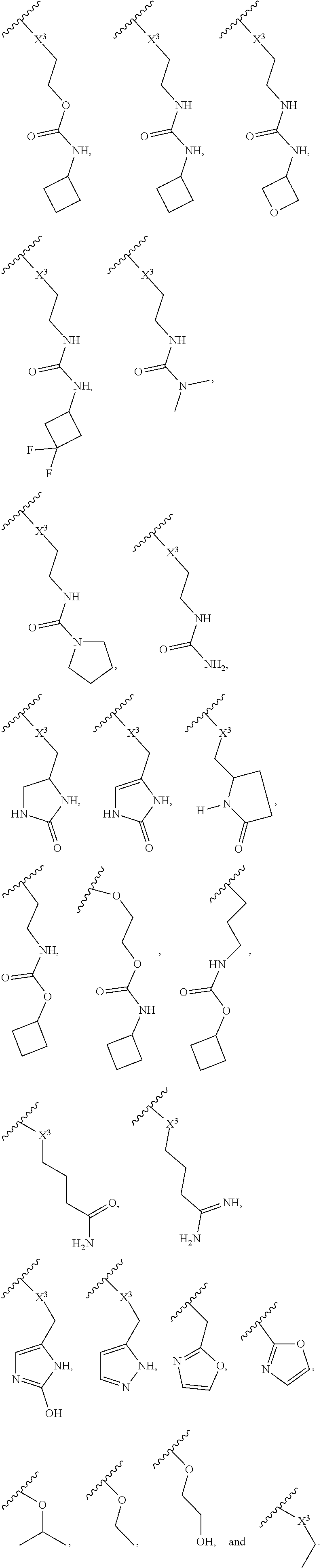

An exemplary embodiment of the immunoconjugate of Formula I includes wherein X⁴ is a bond, and R⁴ is H.

An exemplary embodiment of the immunoconjugate of Formula I includes wherein R¹ is attached to L.

An exemplary embodiment of the immunoconjugate of Formula I includes wherein R² or R³ is attached to L.

An exemplary embodiment of the immunoconjugate of Formula I includes wherein X³—R³-L is selected from the group consisting of:

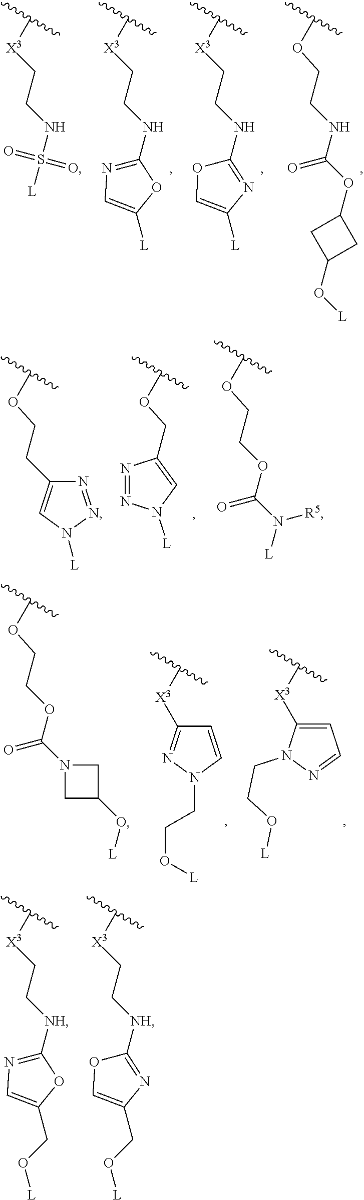

where the wavy line indicates the point of attachment to N.

An exemplary embodiment of the immunoconjugate of Formula I includes wherein R⁴ is C₁-C₁₂ alkyl.

An exemplary embodiment of the immunoconjugate of Formula I includes wherein R⁴ is —(C₁-C₁₂ alkyldiyl)-N(R⁵)—*; where the asterisk * indicates the attachment site of L.

An exemplary embodiment of the immunoconjugate of Formula I includes wherein L is —C(═O)—PEG- or —C(═O)—PEG-C(═O)—.

An exemplary embodiment of the immunoconjugate of Formula I includes wherein L is attached to a cysteine thiol of the antibody.

An exemplary embodiment of the immunoconjugate of Formula I includes wherein, for the PEG, m is 1 or 2, and n is an integer from 2 to 10.

An exemplary embodiment of the immunoconjugate of Formula I includes wherein, for the PEG, n is 10.

An exemplary embodiment of the immunoconjugate of Formula I includes wherein L comprises PEP and PEP is a dipeptide and has the formula:

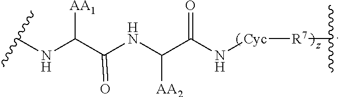

An exemplary embodiment of the immunoconjugate of Formula I includes wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂; or AA₁ and AA₂ form a 5-membered ring proline amino acid.

An exemplary embodiment of the immunoconjugate of Formula I includes wherein AA₁ is —CH(CH₃)₂, and AA₂ is —CH₂CH₂CH₂NHC(O)NH₂.

An exemplary embodiment of the immunoconjugate of Formula I includes wherein AA₁ and AA₂ are independently selected from GlcNAc aspartic acid, —CH₂SO₃H, and —CH₂OPO₃H.

An exemplary embodiment of the immunoconjugate of Formula I includes wherein PEP has the formula:

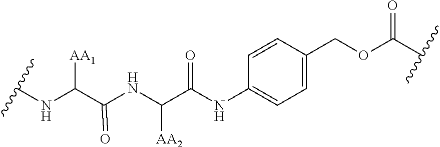

wherein AA₁ and AA₂ are independently selected from a side chain of a naturally-occurring amino acid.

An exemplary embodiment of the immunoconjugate of Formula I includes wherein L comprises PEP and PEP is a tripeptide and has the formula:

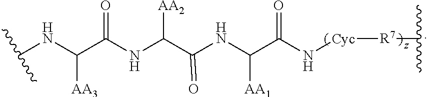

An exemplary embodiment of the immunoconjugate of Formula I includes wherein L comprises PEP and PEP is a tetrapeptide and has the formula:

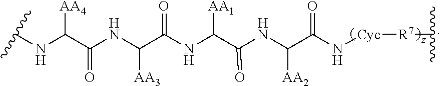

An exemplary embodiment of the immunoconjugate of Formula I includes wherein:
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

An exemplary embodiment of the immunoconjugate of Formula I includes wherein L comprises PEP and PEP is selected from the group consisting of Ala-Pro-Val, Asn-Pro-Val, Ala-Ala-Val, Ala-Ala-Pro-Ala (SEQ ID NO: 473), Ala-Ala-Pro-Val (SEQ ID NO: 474), and Ala-Ala-Pro-Nva (SEQ ID NO: 475).

An exemplary embodiment of the immunoconjugate of Formula I includes wherein L comprises PEP and PEP is selected from the structures:

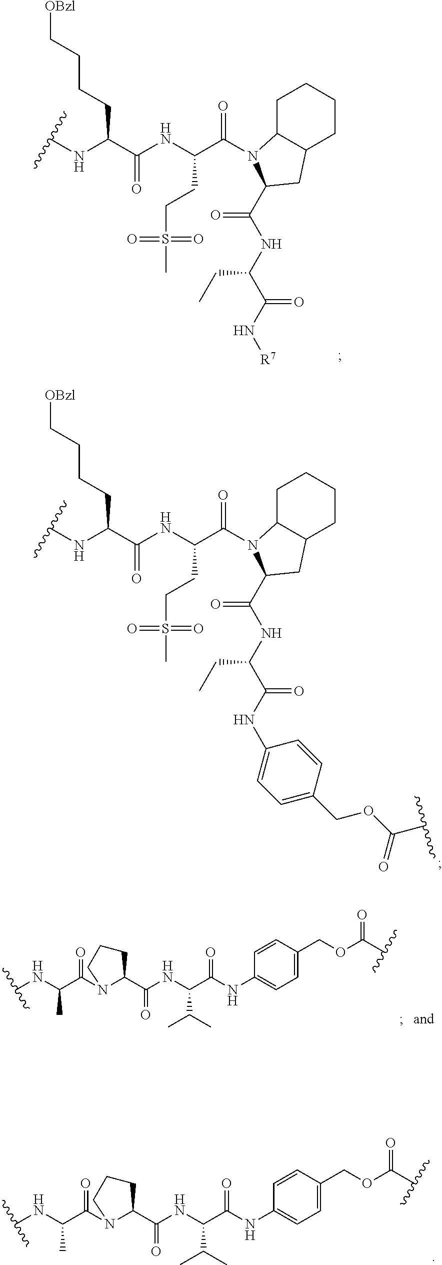

An exemplary embodiment of the immunoconjugate of Formula I includes wherein L is selected from the structures:

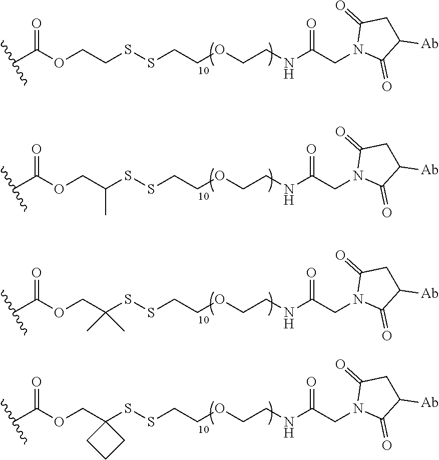

where the wavy line indicates the attachment to R⁵.

An exemplary embodiment of the immunoconjugate of Formula I having Formula Ia:

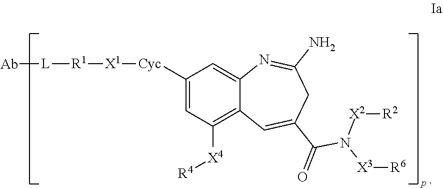

An exemplary embodiment of the immunoconjugate of Formula Ia includes wherein X⁴ is a bond and R⁴ is H.

An exemplary embodiment of the immunoconjugate of Formula Ia includes wherein Cyc is selected from the group consisting of pyridyldiyl, pyrimidyldiyl, pyrazolyldiyl, piperazinyldiyl, piperidinyldiyl, and pyrazinyldiyl.

An exemplary embodiment of the immunoconjugate of Formula Ia includes wherein X² and X³ are each a bond, and R² and R³ are independently selected from C₁-C₅ alkyl, —O—(C₁-C₁₂ alkyl), —(C₁-C₁₂ alkyldiyl)-OR⁵, —(C₁-C₈ alkyldiyl)-N(R⁵)CO₂R, —(C₁-C₁₂ alkyl)-OC(O)N(R⁵)₂, —O—(C₁-C₁₂ alkyl)-N(R⁵)CO₂R⁵, and —O—(C₁-C₂ alkyl)-OC(O)N(R⁵)₂.

An exemplary embodiment of the immunoconjugate of Formula Ia selected from Formulae Ib-Ii:

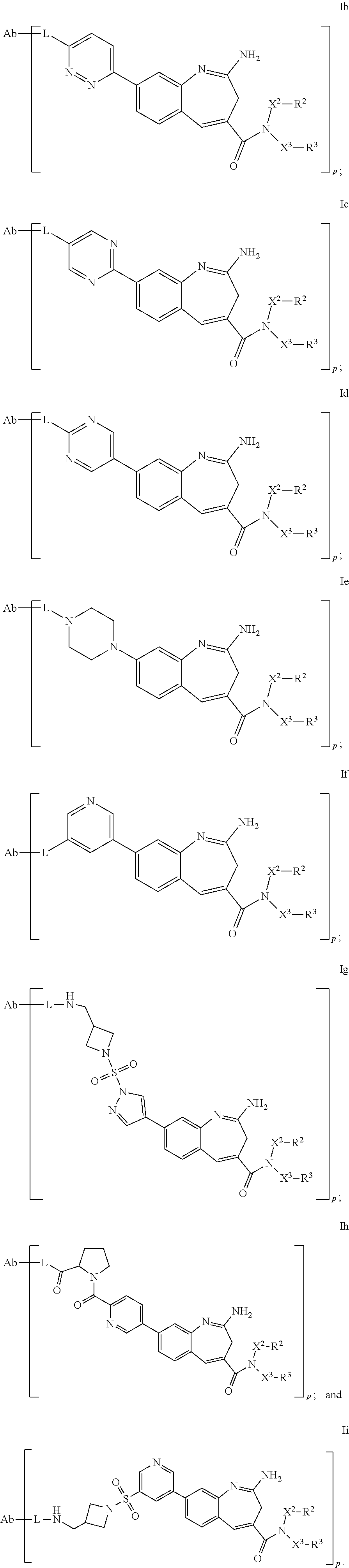

An exemplary embodiment of the immunoconjugate of Formula Ia selected from Formulae Ij-In:

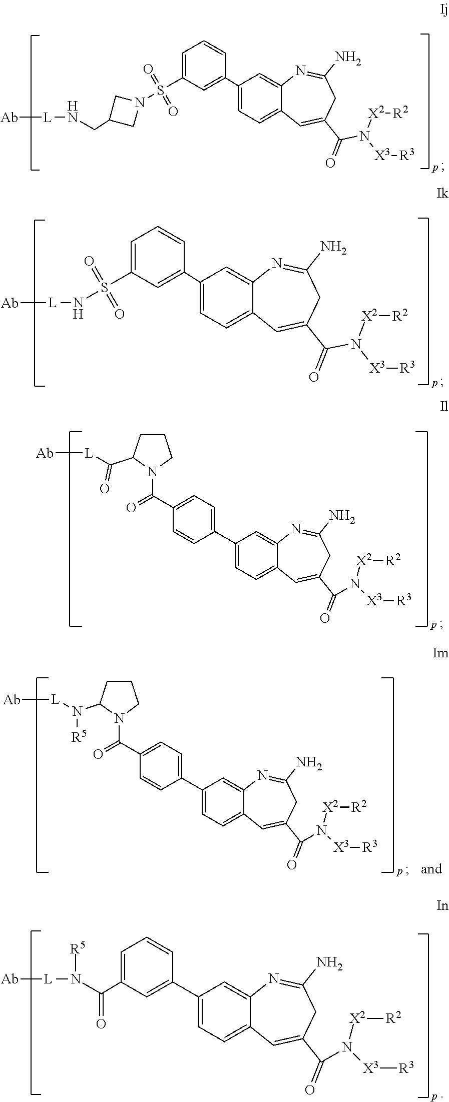

An exemplary embodiment of the immunoconjugate of Formula Ia includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

An exemplary embodiment of the immunoconjugate of Formula Ia includes wherein X² and X³ are each a bond, R² is C₁-C₅ alkyl, and R³ is selected from —O—(C₁-C₁₂ alkyl) and —O—(C₁-C₁₂ alkyl)-N(R⁵)CO₂R⁵.

The invention includes all reasonable combinations, and permutations of the features, of the Formula I embodiments.

In certain embodiments, the immunoconjugate compounds of the invention include those with immunostimulatory activity. The antibody-drug conjugates of the invention selectively deliver an effective dose of a 8-cyclyl-2-aminobenzazepine drug to tumor tissue, whereby greater selectivity (i.e., a lower efficacious dose) may be achieved while increasing the therapeutic index (“therapeutic window”) relative to unconjugated 8-cyclyl-2-aminobenzazepine.

Drug loading is represented by p, the number of Cyc moieties per antibody in an immunoconjugate of Formula I. Drug (CycBz) loading may range from 1 to about 8 drug moieties (D) per antibody. Immunoconjugates of Formula I include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary immunoconjugates of Formula I include, but are not limited to, antibodies that have 1, 2, 3, or 4 engineered cysteine amino acids (Lyon, R. et al. (2012)Methods in Enzym. 502:123-138). In some embodiments, one or more free cysteine residues are already present in an antibody forming intrachain disulfide bonds, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with an CycBz-linker compound of Formula II. In certain embodiments, higher drug loading, e.g.p>5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the CycBz-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of immunoconjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous immunoconjugate with a single loading value may be isolated from the conjugation mixture by electrophoresis or chromatography.

An exemplary embodiment of the immunoconjugate of Formula I is selected from the Tables 3a and 3b Immunoconjugates. Assessment of Immunoconjugate Activity In Vitro was conducted according to the methods of Example 203.

TABLE 3a
Anti-PD-L1, CycBz Immunoconjugates (IC)

| | | | | cDC co-culture |
|---|---|---|---|---|
| Immuno- | | | | assay and IL- |
| conjugate | CycBz-linker | | | 12p70 |
| No. | Table 2a | Antibody | DAR | EC50 [nM] |
| IC-1 | CycBzL-2 | avelumab | 2.7 | |
| IC-2 | CycBzL-4 | avelumab | 2.5 | |
| IC-3 | CycBzL-3 | avelumab | 2.4 | |
| IC-4 | CycBzL-3 | PDL1.24-G1f | 2.4 | |
| IC-5 | CycBzL-2 | PDL1.24-G1f | 1.6 | |
| IC-6 | CycBzL-1 | anti-mPD-L1 | 2.3 | |
| IC-7 | CycBzL-5 | PDL1.24-G1f | 2.3 | |
| IC-8 | CycBzL-5 | PDL1.85-G1f | 2.2 | |
| IC-9 | CycBzL-1 | PDL1.24-G1f | 2.7 | |
| IC-10 | CycBzL-1 | PDL1.85-G1f | 2.8 | |
| IC-11 | CycBzL-6 | PDL1.85-G1f | 2.7 | |
| IC-12 | CycBzL-9 | PDL1.85-G1f | 2.6 | |
| IC-13 | CycBzL-7 | PDL1.85-G1f | 2.7 | |
| IC-14 | CycBzL-6 | PDL1.24-G1f | 3.1 | |
| IC-15 | CycBzL-7 | PDL1.24-G1f | 2.6 | |
| IC-16 | CycBzL-9 | PDL1.24-G1f | 3.1 | |
| IC-17 | CycBzL-6 | avelumab | 2.1 | |
| IC-18 | CycBzL-8 | avelumab | 2.6 | |
| IC-19 | CycBzL-13 | avelumab | 1.9 | 1.9 |
| IC-20 | CycBzL-11 | avelumab | 2.4 | 1.2 |
| IC-21 | CycBzL-14 | avelumab | 2.1 | 1.4 |
| IC-22 | CycBzL-15 | avelumab | 2.7 | 1.0 |
| IC-23 | CycBzL-12 | avelumab | 2.0 | 0.5 |
| IC-24 | CycBzL-11 | PDL1.85-G1f | 2.4 | 0.7 |
| IC-25 | CycBzL-13 | PDL1.85-G1f | 2.5 | |
| IC-26 | CycBzL-14 | PDL1.85-G1f | 2.3 | |
| IC-27 | CycBzL-15 | PDL1.85-G1f | 2.2 | |
| IC-28 | CycBzL-12 | PDL1.85-G1f | 2.4, 3.0 | |

TABLE 3b
Anti-PD-L1, CycBz Immunoconjugates (IC)

| | | | | cDC co-culture |
|---|---|---|---|---|
| | | | | assay and IL- |
| Immunoconjugate | CycBz-linker | | | 12p70 |
| No. | Table 2a, 2b | Antibody | DAR | EC50 [nM] |
| IC-29 | CycBzL-11 | anti-MPD-L1 | 2.4 | |
| IC-30 | CycBzL-11 | PDL1.107-G1f | 2.5 | |
| IC-31 | CycBzL-11 | PDL1.53-IgG1f | 2.6 | 0.4 |
| IC-32 | CycBzL-17 | avelumab | 2.0 | |
| IC-33 | CycBzL-17 | PDL1.107-G1f | 2.3 | |
| IC-34 | CycBzL-17 | PDL1.53-IgG1f | 1.9 | |
| IC-35 | CycBzL-17 | PDL1.96-G1f | 3.4 | |
| IC-36 | CycBzL-17 | PDL1.110-G1f | 2.2 | |
| IC-37 | CycBzL-17 | PDL1.116-G1f | 2.6 | |
| IC-38 | CycBzL-19 | avelumab | 2.5 | 0.5 |
| IC-39 | CycBzL-19 | PDL1.107-G1f | 2.3 | 0.6 |
| IC-40 | CycBzL-19 | PDL1.53-IgG1f | 2.3 | 0.4 |
| IC-41 | CycBzL-19 | PDL1.96-G1f | 2.0 | 0.6 |
| IC-42 | CycBzL-19 | PDL1.110-G1f | 3.2 | 0.9 |
| IC-43 | CycBzL-19 | PDL1.116-G1f | 2.0 | |
| IC-44 | CycBzL-11 | PDL1.96-G1f | 2.3 | 0.8 |
| IC-45 | CycBzL-11 | PDL1.110-G1f | 2.4 | 0.9 |
| IC-46 | CycBzL-11 | PDL1.116-G1f | 2.2 | 5.0 |
| IC-47 | CycBzL-18 | PDL1.96-G1f | 3.2 | 0.8 |
| IC-48 | CycBzL-18 | PDL1.53-IgG1f | 3.1 | 0.9 |
| IC-49 | CycBzL-18 | avelumab | 3.2 | 1.1 |
| IC-50 | CycBzL-16 | anti-MPD-L1 | 2.3 | |
| IC-51 | CycBzL-24 | anti-MPD-L1 | 3.1 | |
| IC-52 | CycBzL-18 | PDL1.110-G1f | 3.7 | 0.4 |
| IC-53 | CycBzL-23 | PDL1.110-G1f | 3.7 | 0.6 |
| IC-54 | CycBzL-20 | PDL1.110-G1f | 3.9 | 0.6 |
| IC-55 | CycBzL-23 | avelumab | 4.2 | 0.6 |
| IC-56 | CycBzL-21 | avelumab | 4.0 | 0.7 |
| IC-57 | CycBzL-20 | avelumab | 4.0 | 0.8 |
| IC-58 | CycBzL-22 | avelumab | 3.7 | 0.7 |
| IC-59 | CycBzL-21 | PDL1.110-G1f | 3.6 | 0.6 |
| IC-60 | CycBzL-22 | PDL1.110-G1f | 3.1 | 0.6 |
| IC-61 | CycBzL-24 | avelumab | 3.4 | 0.2 |
| IC-62 | CycBzL-24 | PDL1.110-G1f | 3.9 | 0.1 |

### Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of adjuvants linked to the same positions on the antibody construct and/or immunoconjugates that have the same number of CycBz adjuvants linked to different positions on the antibody construct, that have different numbers of adjuvants linked to the same positions on the antibody construct, or that have different numbers of adjuvants linked to different positions on the antibody construct.

In an exemplary embodiment, a composition comprising the immunoconjugate compounds comprises a mixture of the immunoconjugate compounds, wherein the average drug (Hx) loading per antibody in the mixture of immunoconjugate compounds is about 2 to about 5.

A composition of immunoconjugates of the invention can have an average adjuvant to antibody construct ratio (DAR) of about 0.4 to about 10. A skilled artisan will recognize that the number of 8-cyclyl-2-aminobenzazepine adjuvants conjugated to the antibody construct may vary from immunoconjugate to immunoconjugate in a composition comprising multiple immunoconjugates of the invention and thus the adjuvant to antibody construct (e.g., antibody) ratio can be measured as an average which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.10% (w/w) to about 10% (w/w).

Method of Treating Cancer with Immunoconjugates

The invention provides a method for treating cancer. The method includes administering a therapeutically effective amount of an immunoconjugate as described herein (e.g., as a composition as described herein) to a subject in need thereof, e.g., a subject that has cancer and is in need of treatment for the cancer. The method includes administering a therapeutically effective amount of an immunoconjugate (IC) selected from Table 3.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., atezolizumab, durvalumab, and avelumab or, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); infantile fibrosarcoma, gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; Ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); Kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The immunoconjugate is administered to a subject in need thereof in any therapeutically effective amount using any suitable dosing regimen, such as the dosing regimens utilized for atezolizumab, durvalumab, and avelumab, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding PD-L1, or tumors over-expressing PD-L1 (e.g. avelumab thereof).

In some embodiments, the cancer is susceptible to a pro-inflammatory response induced by TLR7 and/or TLR8.

In some embodiments, a therapeutically effective amount of an immunoconjugate is administered to a patient in need to treat cervical cancer, endometrial cancer, ovarian cancer, prostate cancer, pancreatic cancer, esophageal cancer, bladder cancer, urinary tract cancer, urothelial carcinoma, lung cancer, non-small cell lung cancer, Merkel cell carcinoma, colon cancer, colorectal cancer, gastric cancer, or breast cancer. The Merkel cell carcinoma cancer may be metastatic Merkel cell carcinoma. The breast cancer may be triple-negative breast cancer. The esophageal cancer may be gastroesophageal junction adenocarcinoma.

### EXAMPLES

### Example L-9 Synthesis of 2,3,5,6-tetrafluorophenyl 1-(1-((3-(2-amino-4-(ethoxy(propyl)carbamoyl)-3H-benzo[b]azepin-8-yl)phenyl)sulfonyl)azetidin-3-yl)-3-oxo-6,9,12,15,18,21,24,27,30,33-decaoxa-2-azahexatriacontan-36-oate, CycBzL-9

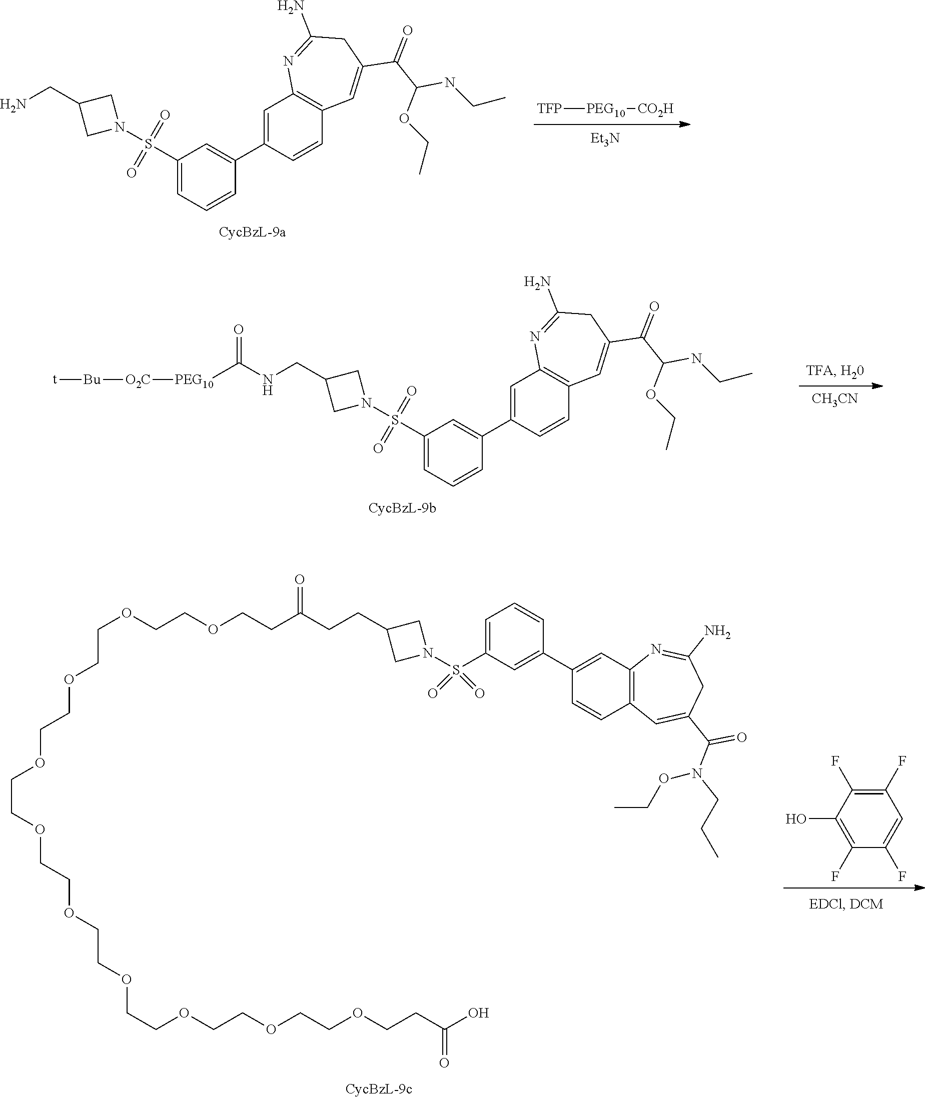

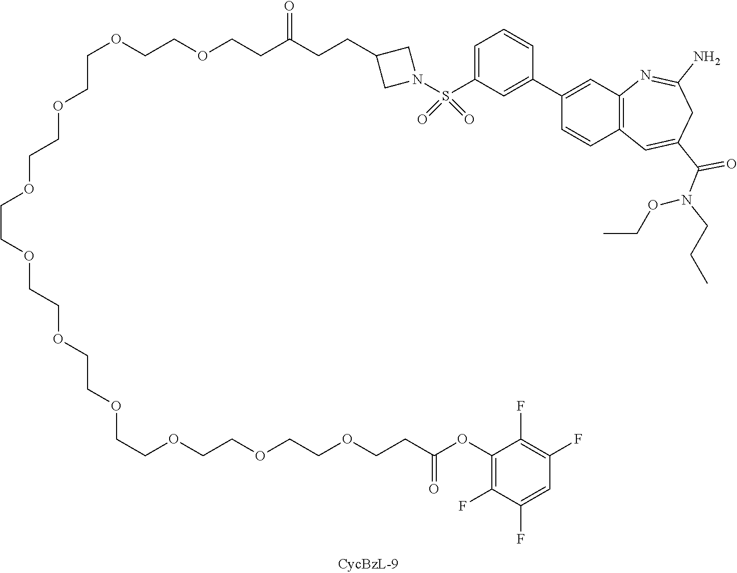 [SMILES: CCCN(OCC)C(=O)C1=Cc2ccc(-c3cccc(S(=O)(=O)N4CC(CCC(=O)CCOCCOCCOCCOCCOCCOCCOCCOCCOCCOCCC(=O)Oc5c(F)c(F)cc(F)c5F)C4)c3)cc2N=C(N)C1]

### Preparation of tert-butyl 3-[2-[2-[2-[2-[2-[2-[2-[2-[2-[3-[[1-[3-[2-amino-4-[ethoxy (propyl) carbamoyl]-3H-1-benzazepin-8-yl]phenyl]sulfonylazetidin-3-yl]methylamino]-3-oxo-propoxy]ethoxy]ethoxy]ethoxy]ethoxy]ethoxy]ethoxy]ethoxy]ethoxy]ethoxy]propanoate, CycBzL-9b

To a solution of 2-amino-8-[3-[3-(aminomethyl)azetidin-1-yl]sulfonylphenyl]-N-ethoxy-N-propyl-3H-1-benzazepine-4-carboxamide, CycBzL-9a (270 mg, 431 umol, 1 eq, TFA) in DMF (2 mL) was added Et₃N (131 mg, 1.29 mmol, 180 uL, 3 eq) and (2,3,5,6-tetrafluorophenyl) 3-[2-[2-[2-[2-[2-[2-[2-[2-[2-(3-tert-butoxy-3-oxo-propoxy)ethoxy]ethoxy]ethoxy]ethoxy]ethoxy]ethoxy]ethoxy]ethoxy]ethoxy]propanoate, TFP-PEG10-CO₂H (329 mg, 431 umol, 1 eq), and then stirred at 0° C. for 1 hr. The mixture was filtered, and purified by prep-HPLC (column: Phenomenex Luna 80*30 mm*3 um; mobile phase: [water(0.1% TFA)-ACN]; B %: 35%-57%, 8 min) to give CycBzL-9b (270 mg, 243 umol, 56.45% yield) as colorless oil.

### Preparation of 3-[2-[2-[2-[2-[2-[2-[2-[2-[2-[3-[[1-[3-[2-amino-4-[ethoxy(propyl) carbamoyl]-3H-1-benzazepin-8-yl]phenyl]sulfonylazetidin-3-yl]methylamino]-3-oxo-propoxy]ethoxy]ethoxy]ethoxy]ethoxy]ethoxy]ethoxy]ethoxy]ethoxy]ethoxy]propanoic acid, CycBzL-9c

To a solution of CycBzL-9b (270 mg, 243 umol, 1 eq) in CH₃CN (2 mL) and H₂O (2 mL) was added TFA (222 mg, 1.95 mmol, 144 uL, 8 eq), and then stirred at 80° C. for 1 hr. The mixture was concentrated and the residue was diluted with water (10 mL) and then the pH of the water phase was adjusted around ˜5 by progressively adding aqueous solution of NaHCO₃ and extracted with DCM:i-PrOH=3:1 (10 mL×3), the organic phase was dried over Na₂SO₄, filtered and concentrated. The residue was purified by prep-HPLC (column: Phenomenex Luna C_{18 75*30} mm*3 um; mobile phase: [water(0.2% FA)-ACN]; B %: 20%-50%, 8 min) to give CycBzL-9c (50 mg, 47.52 umol, 19.51% yield) as colorless oil. ¹H NMR (400 MHz, MeOD) δ8.16-8.09 (m, 2H), 7.94-7.79 (m, 2H), 7.75 (s, 1H), 7.73-7.62 (m, 2H), 7.41 (s, 1H), 3.97 (q, J=7.0 Hz, 2H), 3.86 (t, J=8.2 Hz, 2H), 3.79-3.69 (m, 4H), 3.66-3.49 (m, 40H), 3.32 (s, 2H), 3.18 (d, J=6.4 Hz, 2H), 2.71-2.61 (m, 1H), 2.48 (t, J=6.5 Hz, 2H), 2.30 (t, J=6.0 Hz, 2H), 1.78 (sxt, J=7.2 Hz, 2H), 1.21 (t, J=7.2 Hz, 3H), 1.01 (t, J=7.2 Hz, 3H).

### Preparation of CycBzL-9

To a solution of CycBzL-9c (50 mg, 72 umol, 1 eq, TFA) in DCM (2 mL) and DMA (0.1 mL) was added 2,3,5,6-tetrafluorophenol (95 mg, 503 umol, 8 eq) and 1-ethyl-3-(3-dimnethylaminopropyl)carbodiimide hydrochloride, EDCI, CAS Reg. No. 1892-57-5 (140 mg, 700 umol, 10 eq) and then the mixture was stirred at 25° C. for 0.5 h. The reaction mixture was diluted with water and purified by HPLC to give CycBzL-9 (0.046 g, 0.038 mmol, 53%). LC/MS [M+H] 1200.50 (calculated); LC/MS [M+H] 1200.80 (observed).

### Example L-10 Synthesis of 4-[3-[2-[2-[2-[2-[2-[2-[2-[2-[2-[2-[4-[2-amino-4-[ethoxy(propyl)carbamoyl]-3H-1-benzazepin-8-yl]pyrazol-1-yl]ethoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, CycBzL-10

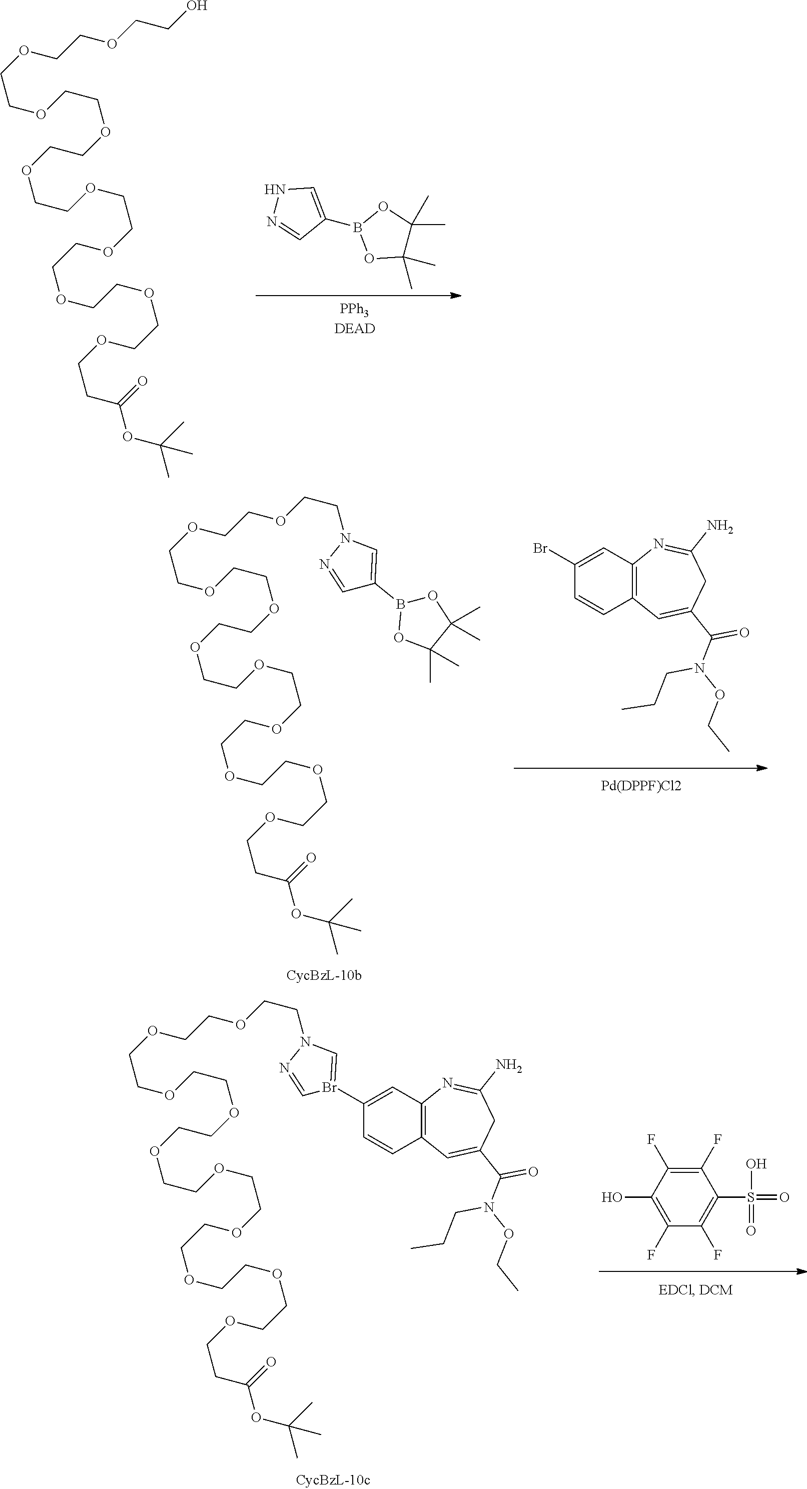

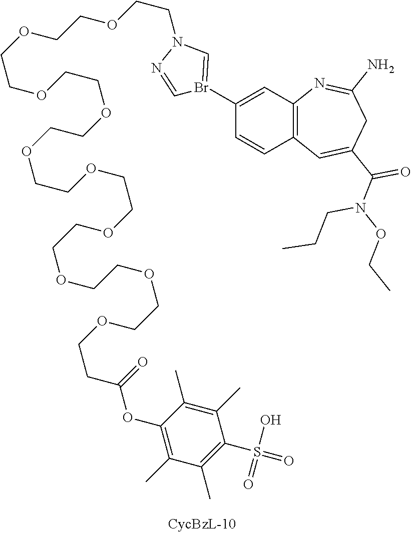

### Preparation of tert-butyl 3-[2-[2-[2-[2-[2-[2-[2-[2-[2-[2-[4-(4,4,5,5-tetramethyl-1,3,2-dioxaborolan-2-yl)pyrazol-1-yl]ethoxy]ethoxy]ethoxy]ethoxy]ethoxy]ethoxy]ethoxy]ethoxy]ethoxy]ethoxy]propanoate, CycBzL-10a

To a solution of 4-(4,4,5,5-tetramethyl-1,3,2-dioxaborolan-2-yl)-1H-pyrazole (1 g, 5.15 mmol, 1 eq) in THF (15 mL) was added PPh₃ (1.35 g, 5.15 mmol, 1 eq) and DEAD (0.89 g, 5.15 mmol, 0.94 mL, 1 eq) at 0° C. and stirred at 25° C. for 0.5 hr, then tert-butyl 3-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]propanoate (3.02 g, 5.15 mmol, 1 eq) was added and then stirred at 25° C. for 16 hr. The reaction mixture was diluted with water 20 mL and extracted with EtOAc (50 mL*3). The combined organic layers were washed with brine (20 mL*3), dried over Na₂SO₄, filtered and concentrated under reduced pressure to give a residue. The residue was purified by column chromatography (SiO₂, Petroleum ether/Ethyl acetate=50/1 to Ethyl acetate:MeOH=10:1) to afford CycBzL-10a (3.5 g, 4.59 mmol, 89.04% yield) as yellow oil.

### Preparation of tert-butyl 3-[2-[2-[2-[2-[2-[2-[2-[2-[2-[2-[4-[2-amino-4-[ethoxy (propyl)carbamoyl]-3H-1-benzazepin-8-yl]pyrazol-1-yl]ethoxy]ethoxy]ethoxy]ethoxy]ethoxy]ethoxy]ethoxy]ethoxy]ethoxy]ethoxy]propanoate, CycBzL-10b

A mixture of CycBzL-10a (625 mg, 819 umol, 2.5 eq), 2-amino-8-bromo-N-ethoxy-N-propyl-3H-1-benzazepine-4-carboxamide (120 mg, 328 umol, 1 eq), a solution of Na₂CO₃ (69.5 mg, 655 umol, 2 eq) in Water (0.3 mL) and [1,1′-bis(diphenylphosphino)ferrocene]palladium(II) dichloride, Pd(dppf)Cl₂ (23.9 mg, 32.8 umol, 0.1 eq) in DMF (3 mL) was de-gassed and then heated to 120° C. for 5 hr under N₂. The mixture was filtered and concentrated under reduced pressure, and the residue was purified by prep-HPLC (TFA condition; column: Phenomenex luna C18 250*50 mm*10 um; mobile phase: [water(0.1% TFA)-ACN]; B %: 35%-65%, 10 min) to afford CycBzL-10b (300 mg, 290 umol, 88.4% yield, TFA) as a yellow solid.

### Preparation of 3-[2-[2-[2-[2-[2-[2-[2-[2-[2-[2-[4-[2-amino-4-[ethoxy(propyl) carbamoyl]-3H-1-benzazepin-8-yl]pyrazol-1-yl]ethoxy]ethoxy]ethoxy]ethoxy]ethoxy]ethoxy]ethoxy]ethoxy]ethoxy]ethoxy]propanoic acid, CycBzL-10c

To a solution of CycBzL-10b (300 mg, 325 umol, 1 eq) in water (3 mL) and MeCN (0.5 mL) was added HCl (12 M, 407 uL, 15 eq), and then stirred at 80° C. for 0.5 hr. The mixture was concentrated under reduced pressure to afford CycBzL-10c (200 mg, 222 umol, 68.1% yield, HCl) as a colorless oil.

### Preparation of CycBzL-10

To a solution of CycBzL-10c (80.0 mg, 88.7 umol, 1 eq, HCl) and sodium; 2,3,5,6-tetrafluoro-4-hydroxy-benzenesulfonate (119 mg, 443 umol, 5 eq) in DCM (1 mL) and DMA (1 mL) was added EDCI (84.9 mg, 443 umol, 5 eq), and then stirred at 25° C. for 0.5 hr. The mixture was filtered and concentrated under reduced pressure, the residue was purified by prep-HPLC (TFA condition; column: Phenomenex Synergi C18 150*25*10 um; mobile phase: [water(0.1% TFA)-ACN]; B %: 25%-50%, 8 min) to afford CycBzL-10 (30 mg, 24.8 umol, 28.01% yield, TFA) as a yellow oil. ¹H NMR (400 MHz, MeOD) δ 8.20 (s, 1H), 7.93 (s, 1H), 7.65-7.61 (m, 1H), 7.59 (s, 1H), 7.55-7.52 (m, 1H), 7.40 (s, 1H), 4.36 (t, J=4.8 Hz, 2H), 3.96 (q, J=7.2 Hz, 2H), 3.89-3.82 (m, 4H), 3.74 (t, J=7.2 Hz, 2H), 3.63-3.52 (m, 36H), 3.42 (s, 2H), 2.95 (t, J=5.6 Hz, 2H), 1.76 (sxt, J=7.2 Hz, 2H), 1.20 (t, J=7.2 Hz, 3H), 0.99 (t, J=7.6 Hz, 3H). LC/MS [M+H] 1094.4 (calculated); LC/MS [M+H] 1094.3 (observed).

### Example L-11 Synthesis of 4-[3-[2-[2-[2-[2-[2-[2-[2-[2-[2-[3-[[5-[2-amino-4-[ethoxy(propyl) carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, CycBzL-11

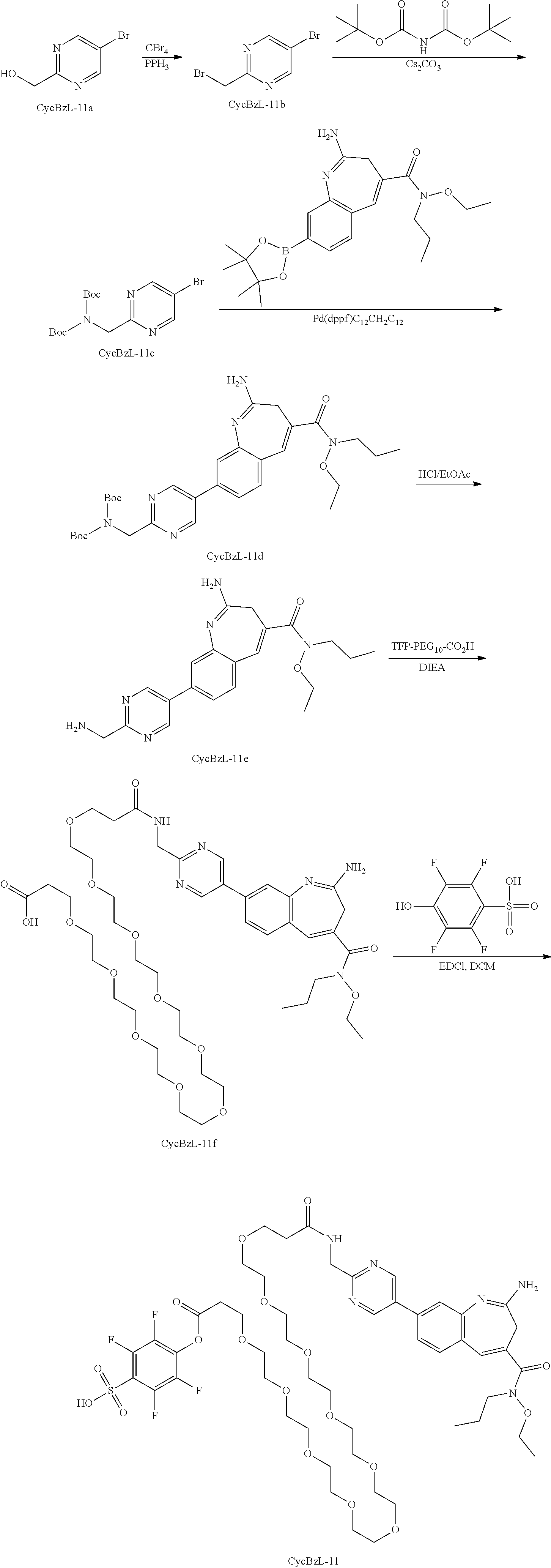

### Preparation of 5-bromo-2-(bromomethyl)pyrimidine, CycBzL-11b

To a solution of (5-bromopyrimidin-2-yl)methanol, CycBzL-11a (300 mg, 1.59 mmol, 1.0 eq) in THE (10 mL) was added PPh₃ (499 mg, 1.90 mmol, 1.2 eq) and CBr₄ (631 mg, 1.90 mmol, 1.2 eq) in one portion at 0° C. under N₂. The mixture was stirred at 20° C. for 10 hours. Water (10 mL) was added and the aqueous phase was extracted with ethyl acetate (10 mL*3), the combined organic phase was washed with brine (10 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 8/1) to afford CycBzL-11b (290 mg, 1.15 mmol, 72.4% yield) as white solid. ¹H NMR (400 MHz, CDCl₃) δ 8.81 (s, 2H), 4.59 (s, 2H).

### Preparation of tert-butyl N-[(5-bromopyrimidin-2-yl) methyl]-N-tert-butoxycarbonyl-carbamate, CycBzL-11c

To a mixture of CycBzL-11b (290 mg, 1.15 mmol, 1.0 eq) and tert-butyl N-tert-butoxycarbonylcarbamate (250 mg, 1.15 mmol, 1.0 eq) in DMF (3 mL) was added Cs₂CO₃ (562 mg, 1.73 mmol, 1.5 eq) in portions at 20° C. under N₂, the mixture was stirred at 20° C. for 2.5 hours. Water (5 mL) was added and the aqueous phase was extracted with ethyl acetate (5 mL*3), the combined organic phase was washed with brine (5 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 5/1) to afford CycBzL-11c (350 mg, 901 umol, 78.3% yield) as white solid. H NMR (400 MHz, CDCl₃) 58.74 (s, 2H), 5.01 (s, 2H), 1.48 (s, 18H).

### Preparation of tert-butyl N-[[5-[2-amino-4-[ethoxy(propyl)carbamoyl]-3H-1-benzaze-pin-8-yl]pyrimidin-2-yl]methyl]-N-tert-butoxycarbonyl-carbamate, CycBzL-11d

To a mixture of CycBzL-11c (184 mg, 473 umol, 1.0 eq) and 2-amino-N-ethoxy-N-propyl-8-(4,4,5,5-tetramethyl-1,3,2-dioxaborolan-2-yl)-3H-1-benzazepine-4-carboxamide (195 mg, 474 umol, 1.0 eq) in dioxane (10 mL) and H₂O (2 mL) was added Pd(dppf)Cl₂·CH₂Cl₂ (19.3 mg, 23.7 umol, 0.05 eq) and K₂CO₃ (163 mg, 1.18 mmol, 2.5 eq) in one portion under N₂, the mixture was de-gassed and heated to 90° C. for 2 hours under N₂. Dioxane (10 mL) was removed in vacuum and water (20 mL) was added and the aqueous phase was extracted with ethyl acetate (10 mL*3), the combined organic phase was washed with brine (10 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 0/1 to Ethyl acetate/Methanol=10/1) to afford CycBzL-1 id (280 mg, 470.83 umol, 99.35% yield) as gray solid. ¹H NMR (400 MHz, MeOD) δ9.08 (s, 2H), 7.61 (s, 1H), 7.59 (d, J=2.8 Hz, 2H), 7.38 (s, 1H), 5.08 (s, 2H), 3.98 (q, J=7.2 Hz, 2H), 3.76 (t, J=7.2 Hz, 2H), 1.83-1.75 (m, 2H), 1.47 (s, 18H), 1.20 (t, J=7.2 Hz, 3H), 1.02 (t, J=7.2 Hz, 3H).

### Preparation of 2-amino-8-[2-(aminomethyl)pyrimidin-5-yl]-N-ethoxy-N-propyl-3H-1-benzazepine-4-carboxamide, CycBzL-lie

To a solution of CycBzL-11d (20.0 mg, 33.6 umol, 1.0 eq) in EtOAc (5 mL) was added HCl/EtOAc (4 M, 8.41 uL, 1.0 eq) in one portion at 20° C. under N₂, the mixture was stirred at 20° C. for 1 hour. The reaction mixture was concentrated in vacuum. The residue was purified by prep-HPLC (column: Phenomenex Synergi C18 150*25*10 um; mobile phase: [water(0.1% TFA)-ACN]; B %: 1%-30%, 8 min) to afford CycBzL-1 1e (6.2 mg, 9.84 umol, 29.2% yield, 98.8% purity, 2TFA) as white solid. ¹H NMR (400 MHz, MeOD) δ9.22 (s, 2H), 7.82 (d, J=2.0 Hz, 1H), 7.79-7.75 (m, 2H), 7.47 (s, 1H), 4.49 (s, 2H), 4.00 (q, J=7.2 Hz, 2H), 3.78 (t, J=7.2 Hz, 2H), 3.46 (s, 2H), 1.85-1.77 (m, 2H), 1.22 (t, J=7.2 Hz, 3H), 1.03 (t, J=7.2 Hz, 3H). LC/MS [M+H] 395.2 (calculated); LC/MS [M+H] 395.1 (observed).

### Preparation of 3-[2-[2-[2-[2-[2-[2-[2-[2-[2-[3-[[5-[2-amino-4-[ethoxy(propyl)carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, CycBzL-11f

To a mixture of CycBzL-11e (70 mg, 149 umol, 1.0 eq, 2HCl) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethox y]propanoic acid (127 mg, 179 umol, 1.2 eq) in DMF (0.5 mL) was added DIEA (77.4 mg, 599 umol, 104 uL, 4.0 eq) in one portion at 25° C. under N₂, the mixture was stirred at 25° C. for 0.5 hour. The reaction mixture was filtered and filtrate was purified by prep-HPLC (column: Phenomenex luna C18 80*40 mm*3 um; mobile phase: [water(0.04% HCl)-ACN]; B %: 12%-39%, 5.5 min) to afford CycBzL-11f (50.0 mg, 53.4 umol, 35.7% yield) as yellow oil. ¹H NMR (400 MHz, MeOD) δ9.14 (s, 2H), 7.86-7.81 (m, 1H), 7.78-7.74 (m, 2H), 7.48 (s, 1H), 4.72 (s, 2H), 4.00 (q, J=7.2 Hz, 2H), 3.85-3.71 (m, 8H), 3.69-3.58 (m, 38H), 3.47 (s, 2H), 2.62 (t, J=6.0 Hz, 2H), 2.55 (t, J=6.4 Hz, 2H), 1.85-1.76 (m, 2H), 1.23 (t, J=7.2 Hz, 3H), 1.03 (t, J=7.2 Hz, 3H).

### Preparation of CycBzL-11

To a mixture of CycBzL-11f (60 mg, 61.7 umol, 1.0 eq, HCl) and (2,3,5,6-tetrafluoro-4-hydroxy-phenyl)sulfonyloxysodium (99.3 mg, 370 umol, 6.0 eq) in DCM (2 mL) and DMA (0.5 mL) was added EDCI (71.0 mg, 370 umol, 6.0 eq) in one portion at 25° C. under N₂, the mixture was stirred at 25° C. for 1 hours. The reaction mixture was filtered and the filtrate was purified by prep-HPLC (column: Phenomenex Synergi C18 150*25*10 um; mobile phase: [water(0.1% TFA)-ACN]; B %: 20%-45%, 8 min) to afford CycBzL-11 (38.0 mg, 30.5 umol, 49.3% yield, 93.3% purity) as yellow oil. ¹H NMR (400 MHz, MeOD) δ9.11 (s, 2H), 7.83-7.79 (m, 1H), 7.77 (s, 1H), 7.76-7.71 (m, 1H), 7.47 (s, 111), 4.71 (s, 2H), 4.00 (q, J=7.2 Hz, 2H), 3.88 (t, J=5.6 Hz, 2H), 3.85-3.75 (m, 5H), 3.70-3.57 (m, 38H), 3.47 (s, 2H), 2.99 (t, J=6.0 Hz, 2H), 2.62 (t, J=4 Hz, 2H), 1.85-1.75 (m, 2H), 1.23 (t, J=7.2 Hz, 3H), 1.02 (t, J=7.2 Hz, 3H). LC/MS [M+H] 1163.3 (calculated); LC/MS [M+H] 1163.3 (observed).

### Example L-12 Synthesis of 4-[3-[2-[2-[2-[2-[2-[2-[2-[2-[2-[3-[[5-[2-amino-4-[2-(cyclobutoxy-carbonylamino)ethoxy-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, CycBzL-12

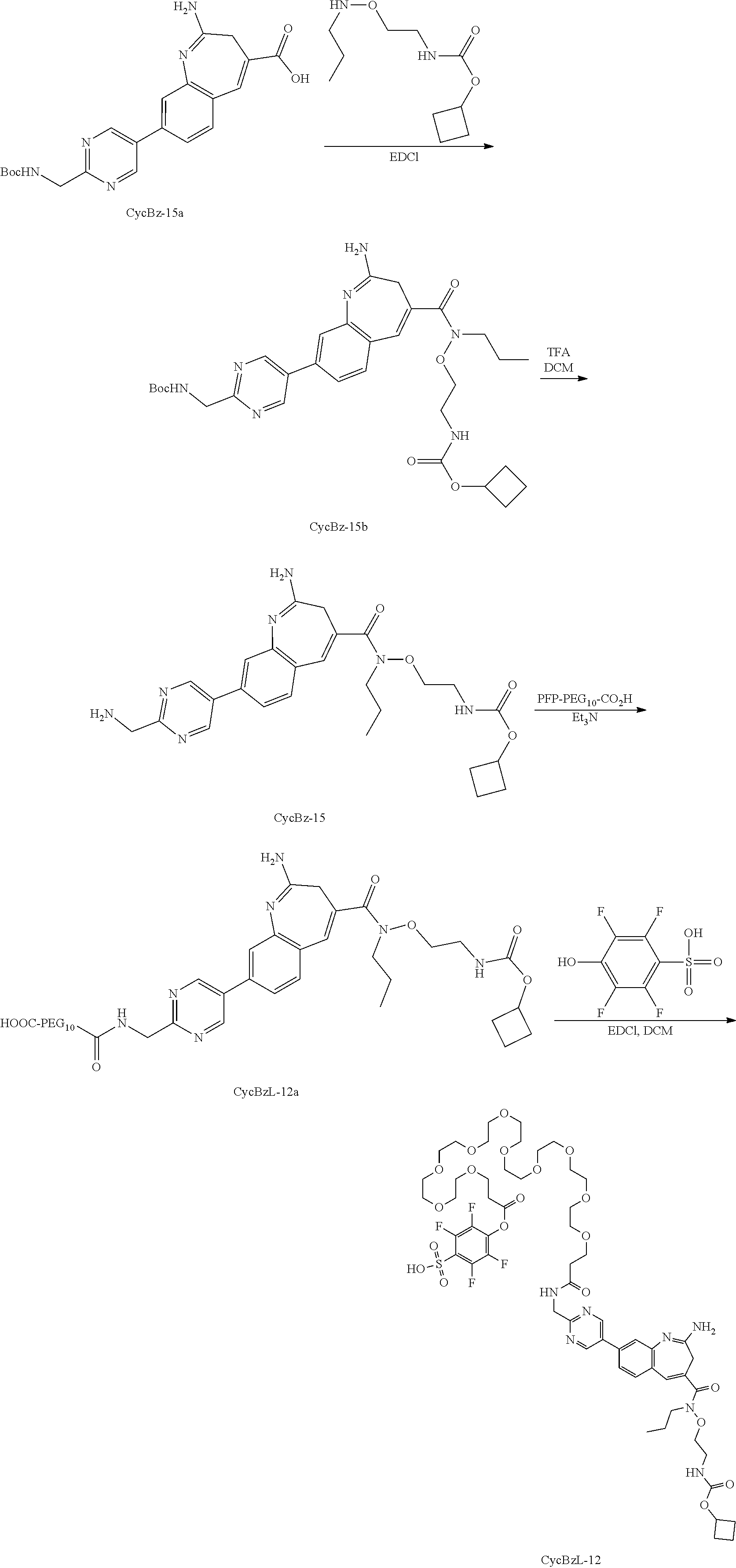

### Preparation of cyclobutyl N-[2-[[2-amino-8-[2-[(tert-butoxycarbonylamino)methyl]pyrimidin-5-yl]-3H-1-benzazepine-4-carbonyl]-propyl-amino]oxyethyl]carbamate, CycBz-15b

To a mixture of 2-amino-8-[2-[(tert-butoxycarbonylamino)methyl]pyrimidin-5-yl]-3H-1-benzazepine-4-carboxylic acid, CycBz-15a (250 mg, 611 umol, 1.0 eq) and cyclobutyl N-[2-(propylamino-oxy)ethyl]carbamate (201 mg, 794 umol, 1.3 eq, HCl) in DCM (4 mL) and DMA (2 mL) was added EDCI (468 mg, 2.44 mmol, 4.0 eq) in one portion at 25° C. under N₂, and it was stirred at 25° C. for 2 hours. DCM (4 mL) was removed in vacuum, water (10 mL) was added and the aqueous phase was extracted with ethyl acetate (10 mL*3), the combined organic phase was washed with brine (5 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 0/1 to Ethyl acetate/Methanol=10/1) to afford CycBz-15b (190 mg, 313 umol, 51.2% yield) as brown oil. 1H NMR (400 MHz, MeOD) δ9.08 (s, 2H), 7.63 (d, J=8.0 Hz, 1H), 7.58-7.52 (m, 2H), 7.37 (s, 1H), 4.74-4.67 (m, 2H), 4.54 (s, 2H), 3.96 (t, J=4.8 Hz, 2H), 3.76 (t, J=7.2 Hz, 2H), 3.33 (s, 2H), 2.20 (dd, J=2.8, 5.2 Hz, 2H), 1.94-1.86 (m, 2H), 1.82-1.75 (m, 2H), 1.50 (s, 9H), 1.38 (d, J=1.6 Hz, 2H), 1.01 (t, J=7.2 Hz, 3H).

### Preparation of cyclobutyl N-[2-[[2-amino-8-[2-(aminomethyl)pyrimidin-5-yl]-3H-1-benzazepine-4-carbonyl]-propyl-amino]oxyethyl]carbamate, CycBz-15

To a solution of CycBz-15b (190 mg, 313 umol, 1.0 eq) in DCM (5 mL) was added CF₃COOH (535 mg, 4.69 mmol, 347 uL, 15 eq) in one portion at 25° C. under N₂, and then stirred at 25° C. for 1.5 hours. DCM (5 mL) was removed in vacuum and the residue was diluted with water (10 mL), the aqueous phase was extracted with MTBE (5 mL*4) to remove excess TFA, then the aqueous phase was freeze-dried to afford CycBz-15 (130 mg, 169 umol, 54.1% yield, 95.7% purity, 2TFA) as brown solid. ¹H NMR (400 MHz, MeOD) δ=9.21 (s, 2H), 7.85-7.76 (m, 3H), 7.49 (s, 1H), 4.66 (t, J=7.2 Hz, 1H), 4.48 (s, 2H), 3.96 (t, J=5.2 Hz, 2H), 3.76 (t, J=7.2 Hz, 2H), 3.43 (s, 2H), 3.31 (s, 2H), 2.20-2.10 (m, 2H), 1.91-1.83 (m, 2H), 1.81-1.74 (m, 2H), 1.70-1.60 (m, 1H), 1.57-1.47 (m, 1H), 1.00 (t, J=7.2 Hz, 3H). LC/MS [M+H] 508.3 (calculated); LC/MS [M+H] 508.1 (observed).

### Preparation of 3-[2-[2-[2-[2-[2-[2-[2-[2-[2-[3-[[5-[2-amino-4-[2-(cyclobutoxycarbonyl amino)ethoxy-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, CycBzL-12a

To a mixture of CycBz-15 (105 mg, 181 umol, 1.0 eq, 2HCl) and Et₃N (73.2 mg, 723 umol, 100 uL, 4.0 eq) in DMF (1.5 mL) was added 3-[2-[2-[2-[2-[2-[2-[2-[2-[2-[3-oxo-3-(2,3,4,5,6-pentafluorophenoxy)propoxy]ethoxy]ethoxy]ethoxy]ethoxy]ethoxy]ethoxy]ethoxy]ethoxy]ethox y]propanoic acid, PFP-PEG10-CO₂H (131 mg, 181 umol, 1.0 eq) at 0° C. under N₂, and it was stirred at 0° C. for 0.5 hour and then was heated 25° C. for another 0.5 hour. The reaction mixture was concentrated, the residue was diluted with water (5 mL) and the aqueous phase was extracted with ethyl acetate (3 mL*2)-discarded, then the aqueous phase was further extracted with DCM/iPrOH=3/1 (5 mL*3), the combined organic phase was dried with anhydrous Na₂SO₄, filtered and concentrated in vacuum to afford CycBzL-12a (100 mg, 95.4 umol, 52.7% yield) as yellow oil.

### Preparation of CycBzL-12

To a mixture of CycBzL-12a (100 mg, 95.4 umol, 1.0 eq) and (2,3,5,6-tetrafluoro-4-hydroxy-phenyl)sulfonyloxy sodium (128 mg, 477 umol, 5.0 eq) in DCM (1 mL) and DMA (0.5 mL) was added EDCI (91.4 mg, 477 umol, 5.0 eq) in one portion at 25° C. under N₂, and then stirred at 25° C. for 1 hour. The reaction mixture was filtered and the filtrate was purified by prep-HPLC (column: Phenomenex Synergi C18 150*25*10 um; mobile phase: [water(0.1% TFA)-ACN]; B %: 15%-35%, 8 min) to afford CycBzL-12 (35.1 mg, 25.6 umol, 26.9% yield, 93.3% purity) as light yellow oil. ¹H NMR (400 MHz, MeOD) δ9.12 (s, 2H), 7.84-7.77 (m, 3H), 7.52 (s, 1H), 4.75-4.67 (m, 3H), 3.99 (t, J=5.2 Hz, 2H), 3.88 (t, J=6.0 Hz, 2H), 3.82 (t, J=6.0 Hz, 2H), 3.78 (t, J=7.2 Hz, 2H), 3.70-3.57 (m, 38H), 3.45 (s, 2H), 3.01-2.97 (m, 2H), 2.62 (t, J=6.0 Hz, 2H), 2.24-2.14 (m, 2H), 1.96-1.86 (m, 2H), 1.84-1.75 (m, 2H), 1.73-1.61 (m, 1H), 1.59-1.49 (m, 1H), 1.01 (t, J=7.2 Hz, 3H). LC/MS [M+H] 1276.5 (calculated); LC/MS [M+H] 1276.6 (observed).

### Example L-13 Synthesis of 4-[3-[2-[2-[2-[2-[2-[2-[2-[2-[2-[3-[[5-[2-amino-4-[3-(cyclobutoxycar bonylamino)propyl-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy] propanoyloxy]-2,3,5,6-tetrafluoro-benzenesulfonic acid, CycBzL-13

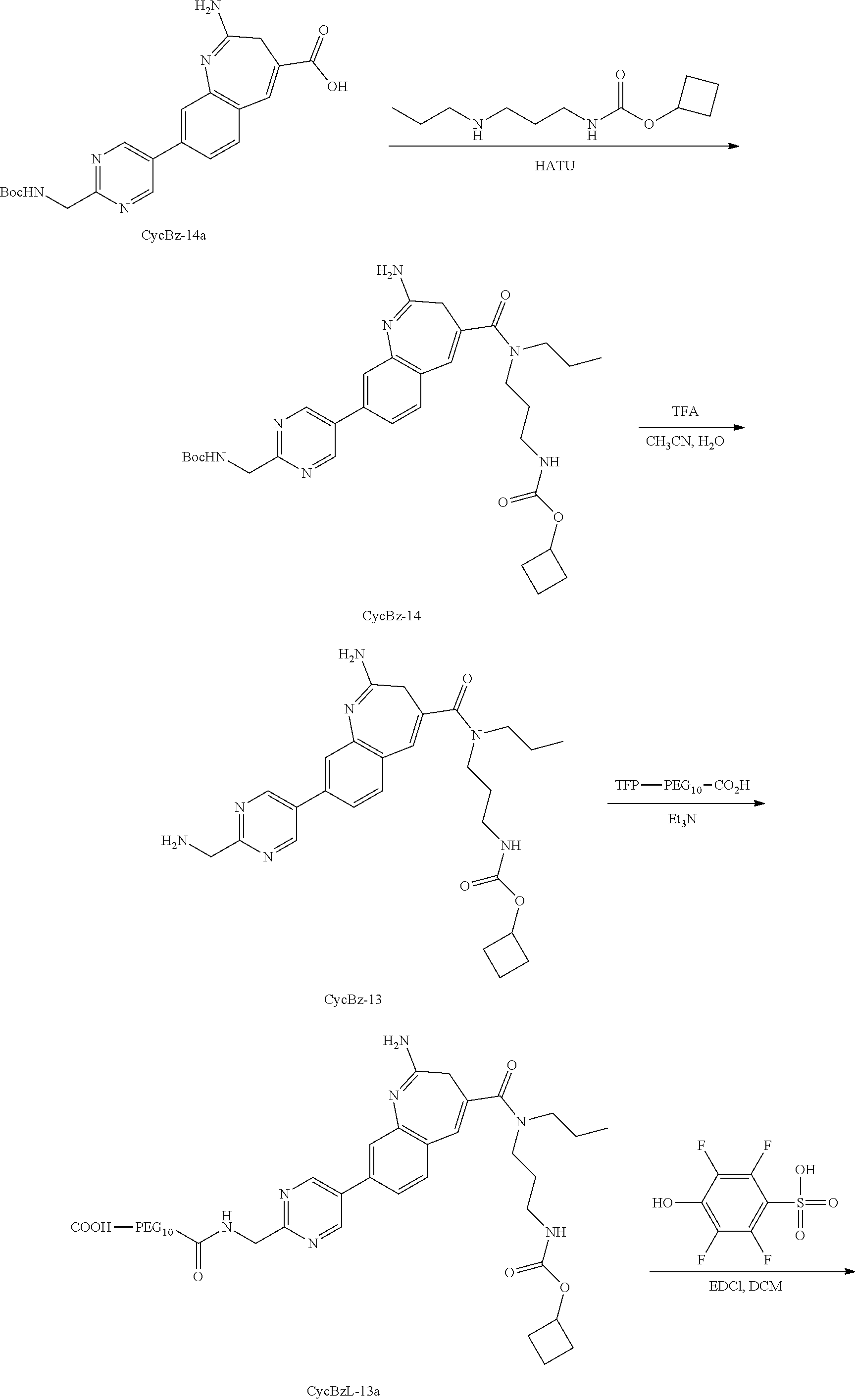

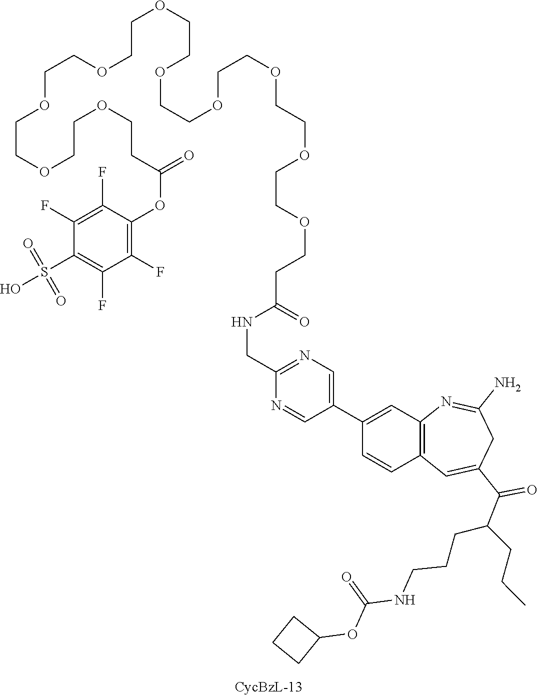 [SMILES: CCCC(CCCNC(=O)OC1CCC1)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1]

### Preparation of cyclobutyl N-[3-[[2-amino-8-[2-[(tert-butoxycarbonylamino)methyl]pyrimidin-5-yl]-3H-1-benzazepine-4-carbonyl]-propyl-amino]propyl], CycBz-14

To a mixture of 2-amino-8-[2-[(tert-butoxycarbonylamino)methyl]pyrimidin-5-yl]-3H-1-benzazepine-4-carboxylic acid, CycBz-14a (0.25 g, 611 umol, 1.0 eq) in DMF (4 mL) was added Et₃N (185 mg, 1.83 mmol, 255 uL, 3.0 eq), cyclobutyl N-[3-(propylamino)propyl]carbamate (170 mg, 678 umol, 1.11 eq, HCl) and Hexafluorophosphate Azabenzotriazole Tetramethyl Uronium, HATU (232 mg, 611 umol, 1.0 eq) in one portion at 0° C., and it was stirred at 0° C. for 0.5 h. Then the mixture was diluted with water and extracted with EtOAc (20 mL×3). The organic layer was washed with brine, dried over Na₂SO₄, filtered and concentrated. The residue was purified by silica gel chromatography (column height: 250 mm, diameter: 100 mm, 100-200 mesh silica gel, Petroleum ether/Ethyl acetate=1/0, 3/1) to afford CycBz-14 (0.28 g, 462 umol, 75.71% yield) as yellow solid. ¹H NMR (MeOD, 400 MHz) δ9.04 (s, 2H), 7.52 (d, J=8.4 Hz, 1H), 7.48 (d, J=1.6 Hz, 1H), 7.45-7.40 (m, 1H), 6.93 (s, 1H), 4.84-4.84 (m, 1H), 4.64 (s, 4H), 3.54-3.47 (m, 2H), 3.46-3.39 (m, 2H), 3.30 (m, 2H), 3.22-3.07 (m, 2H), 2.32-2.28 (m, 2H), 2.10-2.00 (m, 2H), 1.88-1.79 (m, 3H), 1.75-1.60 (m, 3H), 1.48 (s, 9H), 0.90 (s, 3H). LC/MS [M+H] 606.3 (calculated); LC/MS [M+H] 606.2 (observed).

### Preparation of cyclobutyl N-[3-[[2-amino-8-[2-(aminomethyl)pyrimidin-5-yl]-3H-1-benzazepine-4-carbonyl]-propyl-amino]propyl]carbamate, CycBz-13

To a mixture of CycBz-14 (0.26 g, 429 umol, 1.0 eq) in CH₃CN (3 mL) and H₂O (1 mL) was added TFA (489 mg, 4.29 mmol, 318 uL, 10 0.0 eq) in one portion at 25° C. and then stirred at 80° C. for 0.5 h. Then the mixture was concentrated and the residue was diluted with water (10 mL) and the mixture was extracted with MTBE(10 mL×2) to remove excess TFA. The water layer was freeze-dried to give CycBz-13 (0.2 g, 323 umol, 75.20% yield, TFA) as a yellow solid. ¹H NMR (MeOD, 400 MHz) δ 9.21 (s, 2H), 7.84-7.71 (m, 3H), 7.12 (s, 1H), 4.85-4.85 (m, 1H), 4.47 (s, 2H), 3.54 (t, J=7.2 Hz, 2H), 3.48 (s, 2H), 3.37 (s, 2H), 3.15 (d, J=15.6 Hz, 2H), 2.30-2.25 (m, 2H), 2.08-2.00 (m, 2H), 1.89-1.66 (m, 6H), 1.01-0.88 (m, 3H). LC/MS [M+H]506.3 (calculated); LC/MS [M+H] 506.2 (observed).

### Preparation of 3-[2-[2-[2-[2-[2-[2-[2-[2-[2-[3-[[5-[2-amino-4-[3-(cyclobutoxycarbonylamino)propyl-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, CycBzL-13a

To a mixture of CycBz-13 (0.1 g, 161 umol, 1.0 eq, TFA) in THF (3 mL) was added Et₃N (48.9 mg, 484 umol, 67.4 uL, 3.0 eq) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid, TFP-PEG10-CO₂H (114 mg, 161 umol, 1.0 eq) in one portion at 0° C. and then stirred at 0° C. for 0.5 h. The pH of the mixture was adjusted 5-6 with TFA at 0° C. Then the mixture was diluted with water (5 mL) and washed with MTBE (10 mL×3). Then the water layer was further extracted with DCM:i-PrOH=3:1(20 mL×3). The organic layer was dried over Na₂SO₄, filtered and concentrated to give CycBz-13a (0.15 g, 129 umol, 80.11% yield, TFA) as yellow oil.

### Preparation of CycBzL-13

To a mixture CycBz-13a (0.15 g, 129 umol, 1.0 eq, TFA) in DCM (3 mL) and DMA (0.5 mL) was added sodium; 2,3,5,6-tetrafluoro-4-hydroxy-benzenesulfonate (139 mg, 517 umol, 4.0 eq) and EDCI (149 mg, 776 umol, 6.0 eq) in one portion at 25° C. and then stirred at 25° C. for 0.5 h. The mixture was concentrated and filtered. Then the residue was purified by prep-HPLC(column: Phenomenex Synergi C18 150*25*10 um; mobile phase: [water(0.1% TFA)-ACN]; B %: 15%-40%, 8 min) to give CycBzL-13 (75.3 mg, 59.1 umol, 45.71% yield) as yellow oil. ¹H NMR (MeOD, 400 MHz) δ 9.09 (s, 2H), 7.82-7.67 (m, 3H), 7.11 (s, 1H), 4.86-4.82 (m, 1H), 4.69 (s, 2H), 3.86 (t, J=6.0 Hz, 2H), 3.80 (t, J=6.0 Hz, 2H), 3.66-3.48 (m, 40H), 3.38 (s, 2H), 3.22-3.06 (m, 2H), 2.97 (t, J=6.0 Hz, 2H), 2.64-2.58 (m, 2H), 2.32-2.25 (m, 2H), 2.09-1.95 (m, 2H), 1.91-1.80 (m, 3H), 1.75-1.61 (m, 3H), 0.93 (s, 3H). LC/MS [M+H] 1274.5 (calculated); LC/MS [M+H] 1274.3 (observed).

### Example L-14 Synthesis of 4-[3-[2-[2-[2-[2-[2-[2-[2-[2-[2-[3-[[5-[2-amino-4-[2-(dimethylcarbamoylamino)ethoxy-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, CycBzL-14

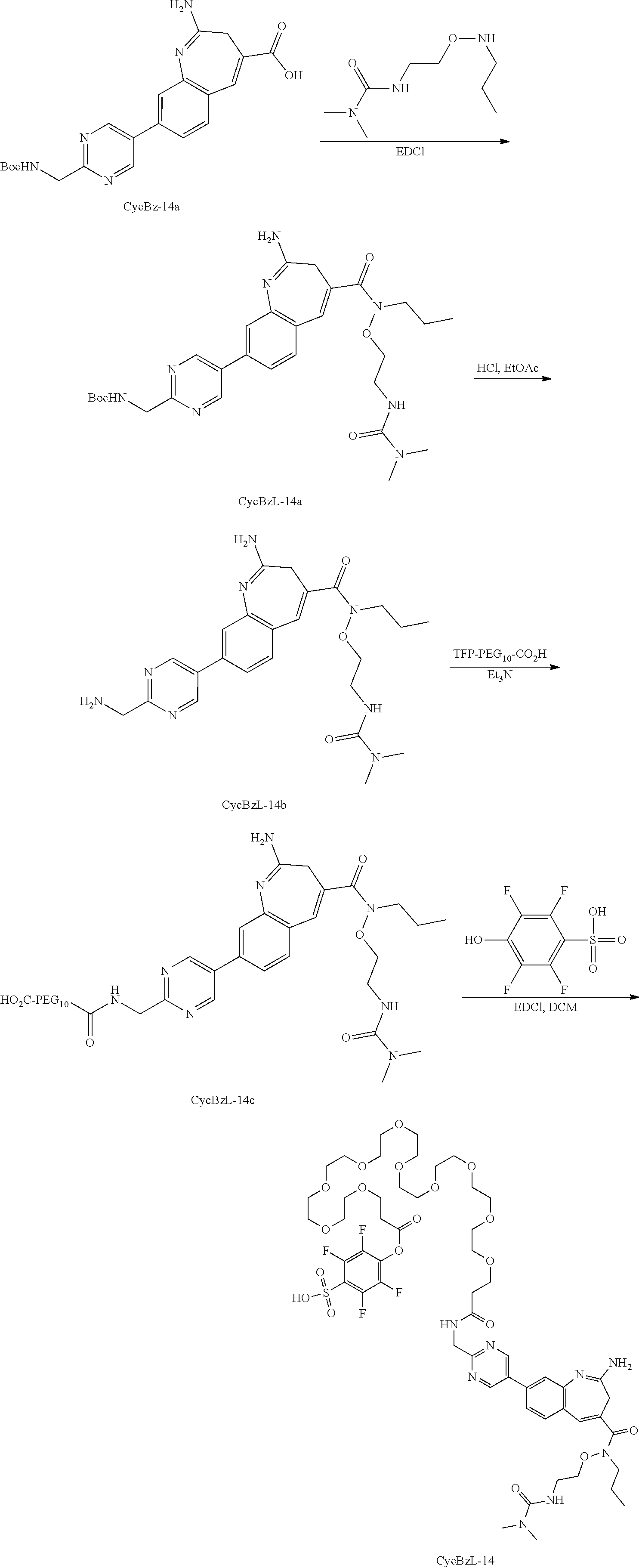

### Preparation of tert-butyl ((5-(2-amino-4-((2-(3,3-dimethylureido)ethoxy)(propyl) carbamoyl)-3H-benzo[b]azepin-8-yl)pyrimidin-2-yl)methyl)carbamate, CycBzL-14a

To a mixture of 2-amino-8-[2-[(tert-butoxycarbonylamino)methyl]pyrimidin-5-yl]-3H-1-benzazepine-4-carboxylic acid, CycBz-14a (250 mg, 611 umol, 1 eq) and 1,1-dimethyl-3-[2-(propylaminooxy)ethyl]urea (165 mg, 733 umol, 1.2 eq, HCl) in DCM (3 mL) and DMA (1 mL) was added EDCI (468 mg, 2.44 mmol, 4 eq), and it was stirred at 25° C. for 1 hr. The mixture was concentrated in vacuum to remove DCM, the residue was diluted with water (10 mL), the pH of mixture was adjusted to ˜8 with aq Na₂CO₃. The aqueous phase was extracted with ethyl acetate (10 mL*4). The combined organic phase was washed with brine (20 mL*1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 0/1, Ethyl acetate/Methanol=1/0, 3/1) to afford CycBzL-14a (260 mg, 447.75 umol, 73.33% yield) as yellow solid.

### Preparation of 2-amino-8-[2-(aminomethyl)pyrimidin-5-yl]-N-[2-(dimethylcarbamoylamino)ethoxy]-N-propyl-3H-1-benzazepine-4-carboxamide, CycBzL-14b

To a solution of CycBzL-14a (130 mg, 224 umol, 1 eq) in EtOAc (3.00 mL) was added HCl/EtOAc (4 M, 3.00 mL, 53.60 eq), and then stirred at 25° C. for 1 h. The mixture was concentrated to give CycBzL-14b (115 mg, 207.77 umol, 92.81% yield, 2HCl) as light red solid. ¹H NMR (MeOD, 400 MHz) δ 9.22 (s, 2H), 7.86-7.80 (m, 2H), 7.80-7.74 (m, 1H), 7.50 (s, 1H), 4.48 (s, 2H), 3.97 (t, J-5.2 Hz, 2H), 3.76 (t, J-7.2 Hz, 2H), 3.45 (s, 2H), 3.38-3.34 (m, 2H), 2.74 (s, 6H), 1.83-1.73 (m, 2H), 1.00 (t, J=7.6 Hz, 3H). LC/MS [M+H] 481.3 (calculated); LC/MS [M+H] 481.1 (observed).

### Preparation of 3-[2-[2-[2-[2-[2-[2-[2-[2-[2-[3-[[5-[2-amino-4-[2-(dimethylcarbamoyl amino)ethoxy-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, CycBzL-14c

To a solution of CycBzL-14b (65.0 mg, 117 umol, 1 eq, 2HCl) in DMF (1.00 mL) was added Et₃N (48.0 mg, 470 umol, 4 eq) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethox y]propanoic acid (83.0 mg, 117 umol, 1 eq), and then stirred at 0° C. for 1 h. The mixture was diluted with water (10 mL) and the pH of the mixture was adjusted to about 6 by progressively adding TFA and extracted with MTBE (10 mL)-discarded, the aqueous was further extracted with DCM:i-PrOH=3:1 (20 mL×3). The organic layer was dried over Na₂SO₄, filtered and concentrated to give CycBzL-14c (95 mg, 93.03 umol, 79.22% yield) as light yellow oil.

### Preparation of CycBzL-14

To a solution of CycBzL-14c (90.0 mg, 88.1 umol, 1 eq) and (2,3,5,6-tetrafluoro-4-hydroxy-phenyl)sulfonyloxysodium (95.0 mg, 353 umol, 4 eq) in DCM (2.00 mL) and DMA (0.10 mL) was added EDCI (68.0 mg, 353 umol, 4 eq), and it was stirred at 25° C. for 1 h. The mixture was concentrated and filtered. The residue was purified by prep-HPLC (column: Phenomenex Synergi C18 150*25*10 um; mobile phase: [water(0.1% TFA)-ACN]; B %: 5%-35%, 8 min) to give CycBzL-14 (51 mg, 37.41 umol, 42.45% yield, TFA) as light yellow oil. ¹H NMR (MeOD, 400 MHz) δ 9.10 (s, 2H), 7.83-7.70 (m, 3H), 7.48 (s, 1H), 4.69 (s, 2H), 3.97 (t, J=5.2 Hz, 2H), 3.86 (t, J=5.6 Hz, 2H), 3.80 (t, J=6.0 Hz, 2H), 3.78-3.74 (m, 2H), 3.65-3.55 (m, 36H), 3.45 (s, 2H), 3.37-3.34 (m, 2H), 2.97 (t, J=5.6 Hz, 2H), 2.74 (s, 6H), 2.60 (t, J=6.0 Hz, 2H), 1.83-1.72 (m, 1H), 1.00 (t, J=7.2 Hz, 3H). LC/MS [M+H] 1249.5 (calculated); LC/MS [M+H] 1249.6 (observed).

### Example L-15 Synthesis of 4-[3-[2-[2-[2-[2-[2-[2-[2-[2-[2-[3-[[1-[[5-[2-amino-4-[3-(cyclobutoxycarbonylamino)propyl-propyl-carbamoyl]-3H-1-benzazepin-8-yl]-3-pyridyl]sulfonyl]azetidin-3-yl]methylamino]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, CycBzL-15

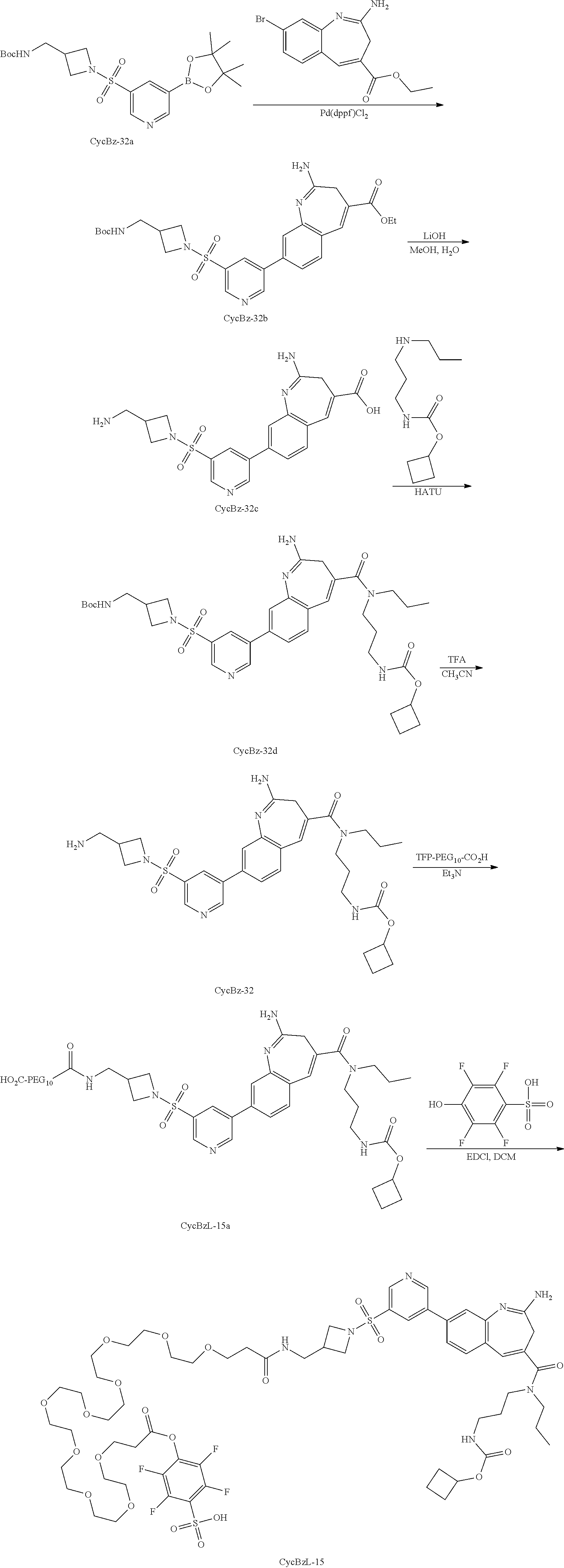

### Preparation of ethyl 2-amino-8-(5-((3-(((tert-butoxycarbonyl)amino)methyl) azetidin-1-yl)sulfonyl)pyridin-3-yl)-3H-benzo[b]azepine-4-carboxylate, CycBz-32b

To a solution of tert-butyl N-[[1-[[5-(4,4,5,5-tetramethyl-1,3,2-dioxaborolan-2-yl)-3-pyridyl] sulfonyl]azetidin-3-yl]methyl]carbamate, CycBz-32a (5 g, 11.0 mmol, 1 eq) and ethyl 2-amino-8-bromo-3H-1-benzazepine-4-carboxylate (3.41 g, 11.0 mmol, 1 eq) in dioxane (50 mL) and H₂O (5 mL) was added K₂CO₃ (3.05 g, 22.1 mmol, 2 eq) and Pd(dppf)Cl₂ (403 mg, 551 umol, 0.05 eq) at 25° C. under N₂, and then stirred at 90° C. for 2 hr. The mixture was filtered and concentrated to give a residue. The residue was diluted with water (100 mL) and extracted with EtOAc (50 mL×3). The organic layer was washed with brine (50 mL), dried over Na₂SO₄, filtered and concentrated to give CycBz-32b which was triturated with CH₃CN at 25° C. for 15 min to give CycBz-32b (5.5 g, 9.90 mmol, 89.75% yield) as gray solid. ¹H NMR (DMSO-d₆, 400 MHz) δ9.29 (s, 1H), 8.94 (s, 1H), 8.32 (s, 1H), 7.80 (s, 1H), 7.60 (d, J=8.0 Hz, 1H), 7.50-7.41 (m, 2H), 7.04-6.85 (m, 3H), 4.25 (q, J=7.2 Hz, 2H), 3.82 (t, J=8.0 Hz, 2H), 3.58-3.52 (m, 2H), 2.99-2.85 (m, 4H), 2.56-2.51 (m, 1H), 1.35-1.30 (m, 12H).

### Preparation of 2-amino-8-(5-((3-(((tert-butoxycarbonyl)amino)methyl)azetidin-1-yl)sulfonyl)pyridin-3-yl)-3H-benzo[b]azepine-4-carboxylic acid, CycBz-32c

To a solution of CycBz-32b (3.2 g, 5.76 mmol, 1 eq) in MeOH (40 mL) and H₂O (5 mL) was added LiOH·H₂O (725 mg, 17.3 mmol, 3 eq), and then stirred at 60° C. for 4 hr. The reaction mixture was concentrated under reduced pressure to remove EtOH. The pH of the mixture was adjusted to about 5 with HCl (12 M) at 0° C. and then filtered, the filter cake was dried under reduced pressure to give the crude product which was triturated with CH₃CN at 25° C. for 20 min to give CycBz-32c (2.7 g, 5.12 mmol, 88.86% yield) as a gray solid. ¹H NMR (DMSO-d₆, 400 MHz) δ9.34 (s, 1H), 9.02 (s, 1H), 8.42 (s, 1H), 7.98-7.92 (m, 2H), 7.89-7.83 (m, 2H), 3.83 (t, J=8.0 Hz, 2H), 3.59-3.49 (m, 4H), 2.90 (d, J=6.0 Hz, 2H), 2.56-2.54 (m, 1H), 1.30 (s, 9H).

### Preparation of cyclobutyl N-[3-[[2-amino-8-[5-[3-[(tert-butoxycarbonylamino) methyl]azetidin-1-yl]sulfonyl-3-pyridyl]-3H-1-benzazepine-4-carbonyl]-propyl-amino]propyl]carbamate, CycBz-32d

To a solution of CycBz-32c (400 mg, 758 umol, 1 eq) in DMF (10.0 mL) was added HATU (317 mg, 834 umol, 1.1 eq), DIEA (490 mg, 3.79 mmol, 660 uL, 5 eq) and cyclobutyl N-[3-(propylamino)propyl]carbamate (380 mg, 1.52 mmol, 2 eq, HCl), and it was stirred at 25° C. for 1 h. The mixture was diluted with water (50 mL) and extracted with EtOAc (30 mL×3). The organic layer was washed with brine (20 mL×3), dried over Na₂SO₄, filtered and concentrate. The residue was purified by flash silica gel chromatography (ISCO®; 1 g SepaFlash® Silica Flash Column, Eluent of 0-30% Ethyl acetate/MeOH @ 35 mL/min) to give CycBz-32d (340 mg, 469.69 umol, 61.95% yield) as light yellow solid. ¹H NMR (MeOD, 400 MHz) δ 9.18 (d, J=2.0 Hz, 1H), 8.95 (d, J=2.0 Hz, 1H), 8.42 (t, J=2.0 Hz, 1H), 7.58-7.50 (m, 2H), 7.49-7.43 (m, 1H), 6.93 (s, 1H), 4.85-4.76 (m, 1H), 3.90 (t, J=8.4 Hz, 2H), 3.64-3.56 (m, 2H), 3.54-3.48 (m, 2H), 3.47-3.39 (m, 2H), 3.32 (br s, 2H), 3.22-3.02 (m, 4H), 2.70-2.57 (m, 1H), 2.35-2.01 (m, 4H), 1.90-1.80 (m, 2H), 1.77-1.47 (m, 4H), 1.37 (s, 9H), 1.05-0.76 (m, 3H).

### Preparation of cyclobutyl N-[3-[[2-amino-8-[5-[3-(aminomethyl)azetidin-1-yl]sulfonyl-3-pyridyl]-3H-1-benzazepine-4-carbonyl]-propyl-amino]propyl]carbamate, CycBz-32

To a solution of CycBz-32d (340 mg, 470 umol, 1 eq) in CH₃CN (2.00 mL) and H₂O (1.00 mL) was added TFA (428 mg, 3.76 mmol, 278 uL, 8 eq), and then stirred at 80° C. for 1 h. The mixture was concentrated and filtered. The residue was purified by prep-HPLC (column: Phenomenex luna C18 100*40 mm*5 um; mobile phase: [water(0.1% TFA)-ACN]; B %: 5%-35%, 8 min) to give CycBz-32 (400 mg, 470 umol, 99.98% yield, 2TFA) as light yellow solid. ¹H NMR (MeOD, 400 MHz) δ 9.24 (d, J=1.6 Hz, 1H), 9.04 (d, J=1.6 Hz, 1H), 8.49 (s, 1H), 7.88-7.71 (m, 3H), 7.13 (br s, 1H), 4.85-4.80 (m, 1H), 4.03 (t, J=8.4 Hz, 2H), 3.73 (dd, J=5.6, 8.4 Hz, 2H), 3.59-3.43 (m, 4H), 3.38 (br s, 2H), 3.12 (br d, J=7.6 Hz, 4H), 2.83-2.73 (m, 1H), 2.37-2.12 (m, 2H), 2.00-2.10 (m, 4H), 1.78-1.43 (m, 4H), 1.05-0.83 (m, 3H). LC/MS [M+H] 624.3 (calculated); LC/MS [M+H] 624.2 (observed).

### Preparation of 3-[2-[2-[2-[2-[2-[2-[2-[2-[2-[3-[[1-[[5-[2-amino-4-[3- (cyclobutoxycarbonylamino)propyl-propyl-carbamoyl]-3H-1-benzazepin-8-yl]-3-pyridyl]sulfonyl]azetidin-3-yl]methylamino]-3-oxo-propoxy]ethoxy]ethoxy]ethoxy]ethoxy]ethoxy]ethoxy]ethoxy]ethoxy]ethoxy]propanoic acid, CycBzL-15a

To a solution of CycBz-32 (200 mg, 235 umol, 1 eq, 2TFA) in THF (2.00 mL) was added Et₃N (71.0 mg, 704 umol, 98.0 uL, 3 eq) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (166 mg, 235 umol, 1 eq), and then stirred at 0° C. for 1 h. The mixture was concentrated and diluted with water (10 mL) and the pH of the mixture was adjusted ˜6 by progressively adding TFA and extracted with MTBE (10 mL)-discarded, the aqueous phase was further extracted with DCM:i-PrOH=3:1 (20 mL×3). The organic layer was dried over Na₂SO₄, filtered and concentrated to give CycBzL-15a (210 mg, 180.36 umol, 76.81% yield) as light yellow oil.

### Preparation of CycBzL-15

To a solution of CycBzL-15a (210 mg, 180 umol, 1 eq) and 2,3,5,6-tetrafluoro-4-hydroxy-benzenesulfonic acid (178 mg, 721 umol, 4 eq) in DCM (4.00 mL) and DMA (0.20 mL) was added EDCI (138 mg, 721 umol, 4 eq), and then stirred at 25° C. for 1 h. The mixture was concentrated and filtered. The residue was purified by prep-HPLC (column: Phenomenex Luna 80*30 mm*3 um; mobile phase: [water(0.2% FA)-ACN]; B %: 15%-40%, 8 min) to give CycBzL-15 (98 mg, 68.13 umol, 37.77% yield, FA) as white solid. ¹H NMR (MeOD, 400 MHz) δ 9.23 (d, J=2.0 Hz, 1H), 9.02 (d, J=2.0 Hz, 1H), 8.48 (t, J=2.0 Hz, 1H), 7.91-7.67 (m, 3H), 7.13 (s, 1H), 4.85-4.80 (m, 1H), 3.93 (t, J=8.4 Hz, 2H), 3.86 (t, J=5.6 Hz, 2H), 3.66-3.55 (m, 40H), 3.54-3.48 (m, 4H), 3.40 (br s, 2H), 3.25-3.08 (m, 4H), 2.97 (t, J=5.6 Hz, 2H), 2.79-2.68 (m, 1H), 2.29 (br t, J=6.0 Hz, 3H), 1.93-1.80 (m, 3H), 1.77-1.52 (m, 4H), 1.01-0.88 (m, 3H). LC/MS [M+H] 1392.5 (calculated); LC/MS [M+H] 1392.3 (observed).

### Example L-16 Synthesis of 4-[3-[2-[2-[2-[2-[2-[2-[2-[2-[2-[3-[[5-[2-amino-4-[2-(cyclobutylcarbamoylamino)ethoxy-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, CycBzL-16

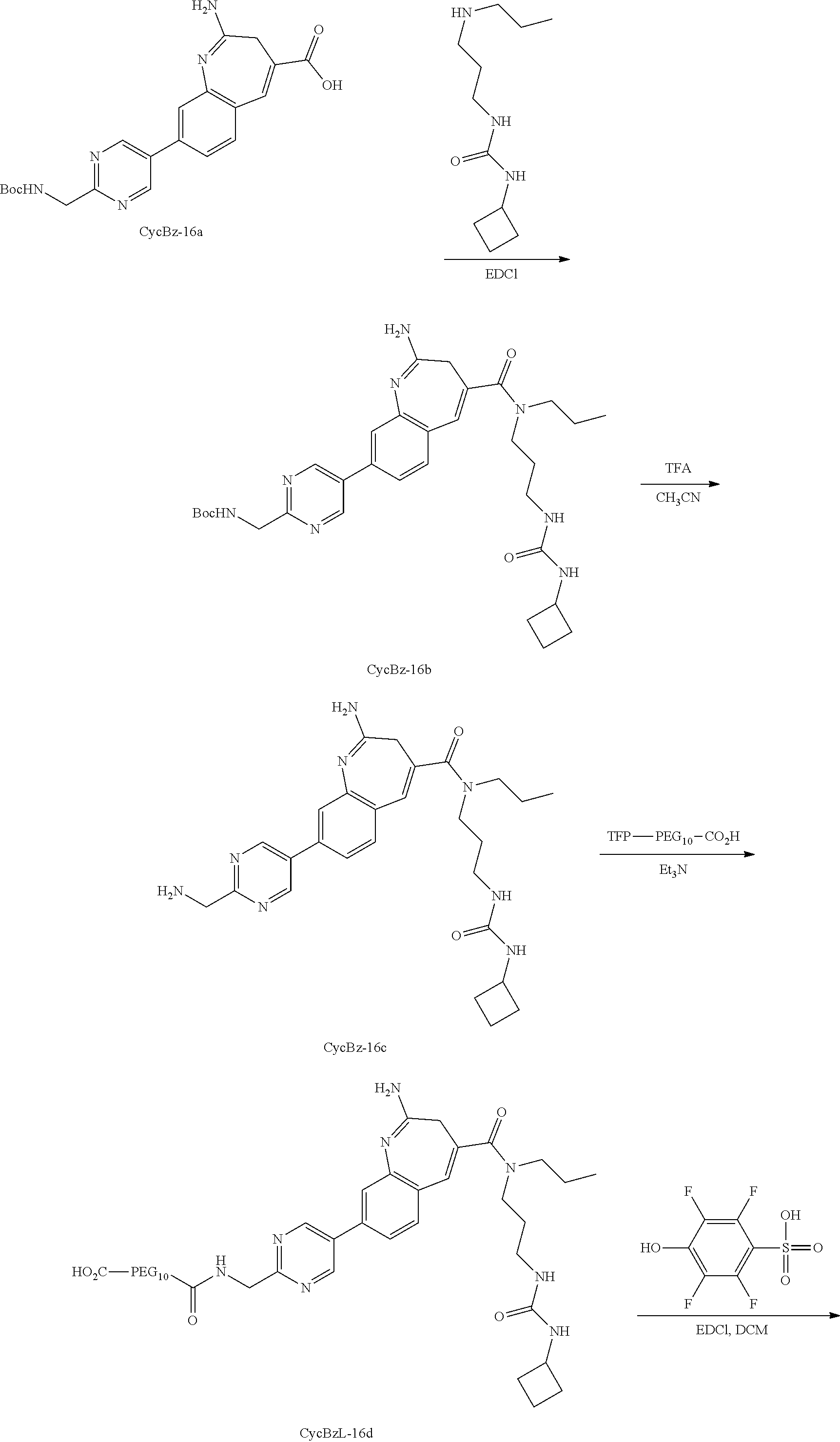

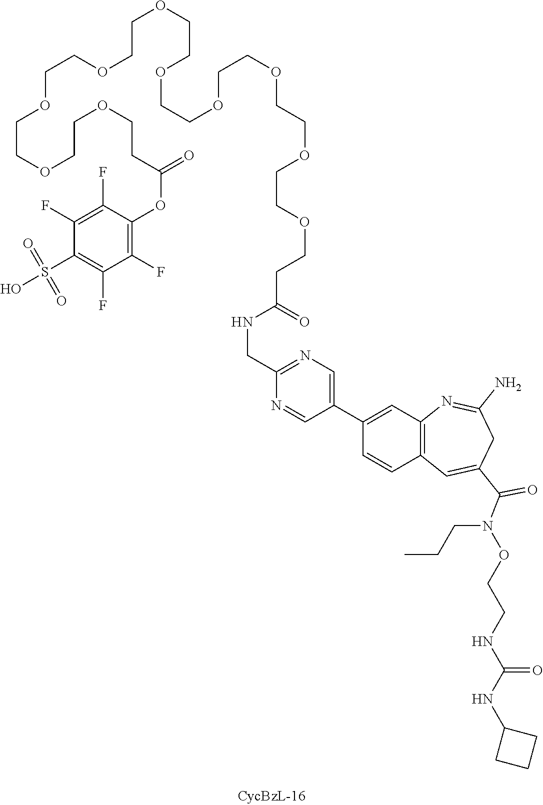 [SMILES: CCCN(OCCNC(=O)NC1CCC1)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1]

### Preparation of tert-butyl ((5-(2-amino-4-((2-(3-cyclobutylureido)ethoxy)(propyl) carbamoyl)-3H-benzo[b]azepin-8-yl)pyrimidin-2-yl)methyl)carbamate, CycBzL-16b

To a solution of 2-amino-8-[2-[(tert-butoxycarbonylamino)methyl] pyrimidin-5-yl]-3H-1-benzazepine-4-carboxylic acid, CycBzL-16a (250 mg, 611 umol, 1 eq) 1-cyclobutyl-3-[2-(propylaminooxy)ethyl]urea (231 mg, 916 umol, 1.5 eq, HCl) in DCM (2 mL) and DMA (2 mL) was added EDCI (351 mg, 1.83 mmol, 3 eq), and it was stirred at 25° C. for 0.5 hr. The reaction mixture was concentrated under reduced pressure to remove DCM. The residue was diluted with water (10 mL) and extracted with EtOAc (20 mL*3). The combined organic layers were washed with brine (20 mL*2), dried over Na₂SO₄, filtered and concentrated under reduced pressure to give a residue. The residue was purified by column chromatography (SiO₂, Petroleum ether/Ethyl acetate=50/1 to Ethyl acetate:MeOH=5:1) to afford CycBzL-16b (230 mg, 380 umol, 62.1% yield) as a brown solid.

### Preparation of 2-amino-8-[2-(aminomethyl)pyrimidin-5-yl]-N-[2-(cyclobutylcarbamoylamino)ethoxy]-N-propyl-3H-1-benzazepine-4-carboxamide, CycBzL-16c

To a solution of CycBzL-16b (230 mg, 0.38 mmol, 1 eq) in Water (2 mL) and MeCN (2 mL) was added TFA (432 mg, 3.79 mmol, 0.28 mL, 10 eq), and then stirred at 80° C. for 0.5 hr. The mixture was concentrated under reduced pressure, the residue was diluted with water (2 mL) and extracted with MTBE (3 mL*3)-discarded, the aqueous phase was concentrated under reduced pressure to afford CycBzL-16c (230 mg, 371 umol, 97.8% yield, TFA) as a brown solid. ¹H NMR (400 MHz, MeOD) δ 9.21 (s, 2H), 7.84-7.73 (m, 3H), 7.47 (s, 1H), 4.48 (s, 2H), 4.01-3.89 (m, 3H), 3.75 (t, J=7.2 Hz, 2H), 3.44 (s, 2H), 3.33 (br s, 2H), 2.19-2.10 (m, 2H), 1.81-1.68 (m, 4H), 1.64-1.55 (m, 2H), 1.00 (t, J=7.2 Hz, 3H). LC/MS [M+H] 507.3 (calculated); LC/MS [M+H] 507.2 (observed).

### Preparation of 3-[2-[2-[2-[2-[2-[2-[2-[2-[2-[3-[[5-[2-amino-4-[2-(cyclobutyl carbamoylamino)ethoxy-propyl-carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, CycBzL-16d

To a solution of CycBzL-16c (100 mg, 136 umol, 1 eq, 2TFA) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid, TFP-PEG₁₀-CO₂H (96.2 mg, 0.14 mmol, 1 eq) in THF (1 mL) was added Et₃N (41.3 mg, 0.41 mmol, 56.8 uL, 3 eq), and then stirred at 25° C. for 0.5 hr. The pH of the mixture was adjusted to about 6 with TFA at 0° C., extracted with EtOAc (5 mL three times)-discarded, and the aqueous was further extracted with DCM/i-PrOH (10 mL*3, 3/1). The organic layers were dried over Na₂SO₄ filtered and concentrated under reduced pressure. The crude product CycBzL-16d (120 mg, 115 umol, 84.2% yield) was obtained as yellow oil and used in the next step without further purification.

### Preparation of CycBzL-16

To a solution of CycBzL-16d (70 mg, 66.9 umol, 1 eq) and sodium; 2,3,5,6-tetrafluoro-4-hydroxy-benzenesulfonate (71.7 mg, 267 umol, 4 eq) in DMA (0.5 mL) and DCM (1.5 mL) was added EDCI (51.3 mg, 267 umol, 4 eq), and it was stirred at 25° C. for 0.5 hr. The mixture was filtered and concentrated under reduced pressure. The residue was purified by prep-HPLC (TFA condition; column: Phenomenex Synergi C18 150*25*10 um; mobile phase: [water(0.1% TFA)-ACN]; B %: 15%-35%, 8 min). Then the residue was purified by prep-HPLC (TFA condition; column: Phenomenex Synergi C18 150*25*10 um; mobile phase: [water (0.1% TFA)-ACN]; B %: 15%-35%, 8 min) to afford HxBzL-13 (20 mg, 13.3 umol, 19.9% yield, 2TFA) as a colorless oil. ¹H NMR (400 MHz, MeOD) δ 9.09 (s, 2H), 7.80-7.71 (m, 3H), 7.47 (s, 1H), 4.69 (s, 2H), 3.95 (br t, J=5.2 Hz, 2H), 3.86 (t, J=6.0 Hz, 2H), 3.80 (t, J=6.0 Hz, 2H), 3.75 (br t, J=7.2 Hz, 2H), 3.68-3.57 (m, 38H), 3.45 (s, 2H), 2.97 (t, J=6.0 Hz, 2H), 2.60 (t, J=6.0 Hz, 2H), 2.15 (br d, J=7.2 Hz, 2H), 1.83-1.68 (m, 4H), 1.64-1.52 (m, 2H), 0.99 (t, J=7.2 Hz, 3H). LC/MS [M+H] 1275.5 (calculated); LC/MS [M+H] 1275.2 (observed).

### Example L-18 Synthesis of 2-amino-8-(2-(38-(2,5-dioxo-2,5-dihydro-1H-pyrrol-1-yl)-3,37-dioxo-6,9,12,15,18,21,24,27,30,33-decaoxa-2,36-diazaoctatriacontyl)pyrimidin-5-yl)-N-ethoxy-N-propyl-3H-benzo[b]azepine-4-carboxamide, CycBzL-18

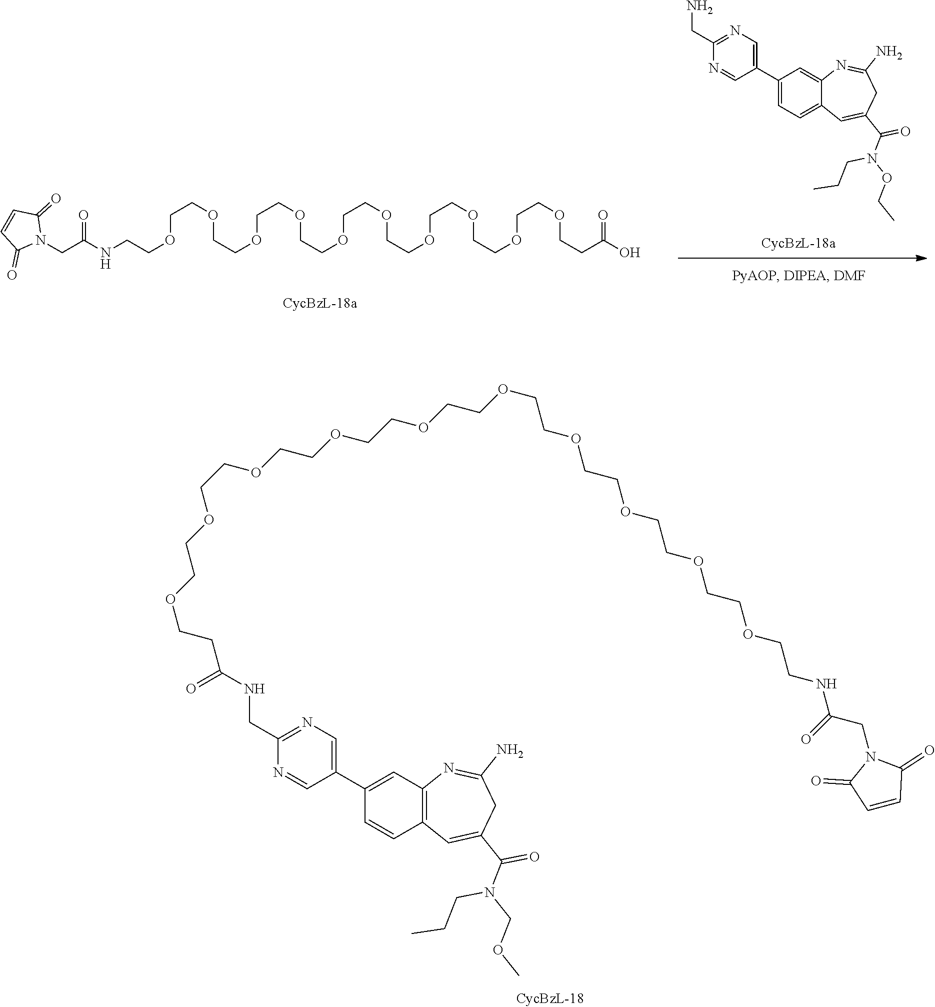 [SMILES: O=C(O)CCOCCOCCOCCOCCOCCOCCOCCOCCOCCOCCNC(=O)CN1C(=O)C=CC1=O ; CCCN(OCC)C(=O)C1=Cc2ccc(-c3cnc(CN)nc3)cc2N=C(N)C1 ; CCCN(COC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1]

2-Amino-8-(2-(amiinomethyl)pyrimidin-5-yl)-N-ethoxy-N-propyl-3H-benzo[b]azepine-4-carboxamide, CycBzL-18a (0.0283 g, 0.072 mmol, 1 eq.) and 1-(2,5-dioxo-2,5-dihydro-1H-pyrrol-1-yl)-2-oxo-6,9,12,15,18,21,24,27,30,33-decaoxa-3-azahexatriacontan-36-oic acid, CycBzL-18b (0.0478 g, 0.072 mmol, 1 eq.) were dissolved in dimethylformamide, DMF. Diisopropylethylamine, DIPEA (0.075 mol, 0.43 mmol, 6 eq.) was added, followed by ((7-Azabenzotriazol-1-yloxy)tripyrrolidinophosphonium hexafluorophosphate), PyAOP, CAS Reg. No. 1563 11-83-0 (0.091 g, 0.18 mmol, 2.4 eq.). The reaction was stirred at room temperature, then concentrated and purified by RP-HPLC to give CycBzL-18 (0.0346 g, 0.033 mmol, 46%). LC/MS [M+H] 1043.53 (calculated); LC/MS [M+H] 1043.84 (observed).

### Example L-20 Synthesis of 2-amino-8-(2-((1-(2,5-dioxo-2,5-dihydro-1H-pyrrol-1-yl)-2-oxo-6,9,12,15,18,21,24,27,30-nonaoxa-3-azadotriacontan-32-yl)carbamoyl)pyrimidin-5-yl)-N-ethoxy-N-propyl-3H-benzo[b]azepine-4-carboxamide, CycBzL-20

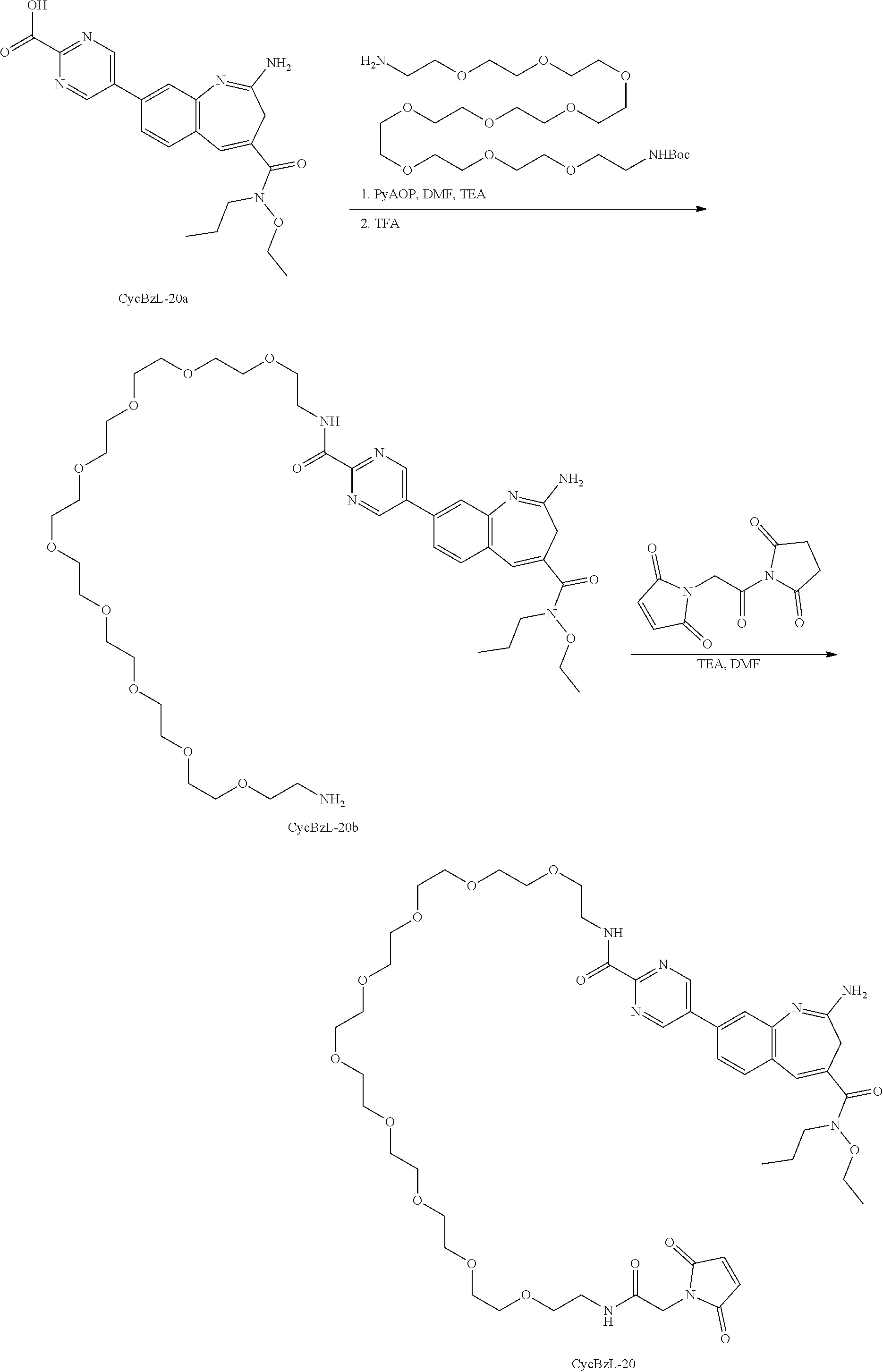 [SMILES: CCCN(OCC)C(=O)C1=Cc2ccc(-c3cnc(C(=O)O)nc3)cc2N=C(N)C1 ; CC(C)(C)OC(=O)NCCOCCOCCOCCOCCOCCOCCOCCOCCOCCN ; CCCN(OCC)C(=O)C1=Cc2ccc(-c3cnc(C(=O)NCCOCCOCCOCCOCCOCCOCCOCCOCCOCCN)nc3)cc2N=C(N)C1 ; O=C1C=CC(=O)N1CC(=O)N1C(=O)CCC1=O ; CCCN(OCC)C(=O)C1=Cc2ccc(-c3cnc(C(=O)NCCOCCOCCOCCOCCOCCOCCOCCOCCOCCNC(=O)CN4C(=O)C=CC4=O)nc3)cc2N=C(N)C1]

### Preparation of 2-amino-8-(2-((29-amino-3,6,9,12,15,18,21,24,27-nonaoxanonacosyl)carbamoyl)pyrimidin-5-yl)-N-ethoxy-N-propyl-3H-benzo[b]azepine-4-carboxamide, CycBzL-20b

To a solution of 5-(2-amino-4-(ethoxy(propyl)carbamoyl)-3H-benzo[b]azepin-8-yl)pyrimidine-2-carboxylic acid, CycBzL-20a (0.0106 g, 0.026 mmol, 1 eq) and tert-butyl (29-amino-3,6,9,12,15,18,21,24,27-nonaoxanonacosyl)carbamate (0.014 g, 0.026 mmol, 1 eq) in DMF (0.5 ml) was added triethylamine, TEA (36 μl, 0.26 mmol, 10 eq), followed by (7-azabenzotriazol-1-yloxy)tripyrrolidinophosphonium hexafluorophosphate, PyAOP, CAS Reg. No. 156311-83-0 (0.013 g, 0.026 mmol, 1 eq). The reaction was stirred at room temperature and monitored by LC/MS. The reaction was concentrated and purified by prep-HPLC to give tert-butyl (1-(5-(2-amino-4-(ethoxy(propyl)carbamoyl)-3H-benzo[b]azepin-8-yl)pyrimidin-2-yl)-1-oxo-5,8,11,14,17,20,23,26,29-nonaoxa-2-azahentriacontan-31-yl)carbamate, which was then dissolved in TFA and concentrated to give CycBzL-20b (17.1 mg, 0.020 mmol, 77%). LC/MS [M+H] 848.5 (calculated); LC/MS [M+H] 848.8 (observed).

### Preparation of CycBzL-20

To a solution of CycBzL-20b (17.1 mg, 0.020 mmol, 1.33 eq) in DMF (0.5 ml) was added TEA (28 μl, 0.20 mmol, 13.3 eq) followed by N-(α-maleimidoacetoxy) succinimide ester (3.8 mg, 0.015 mmol, 1 eq). The reaction was stirred at room temperature and monitored by LC/MS, then concentrated and purified by prep-HPLC to give CycBzL-20 (8.5 mg, 0.0086 mmol, 57%). LC/MS [M+H] 985.5 (calculated); LC/MS [M+H] 985.6 (observed).

### Example L-23 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl (2-((2-amino-N-propyl-8-(pyrimidin-5-yl)-3H-benzo[b]azepine-4-carboxamido)oxy)ethyl)carbamate, CycBzL-23

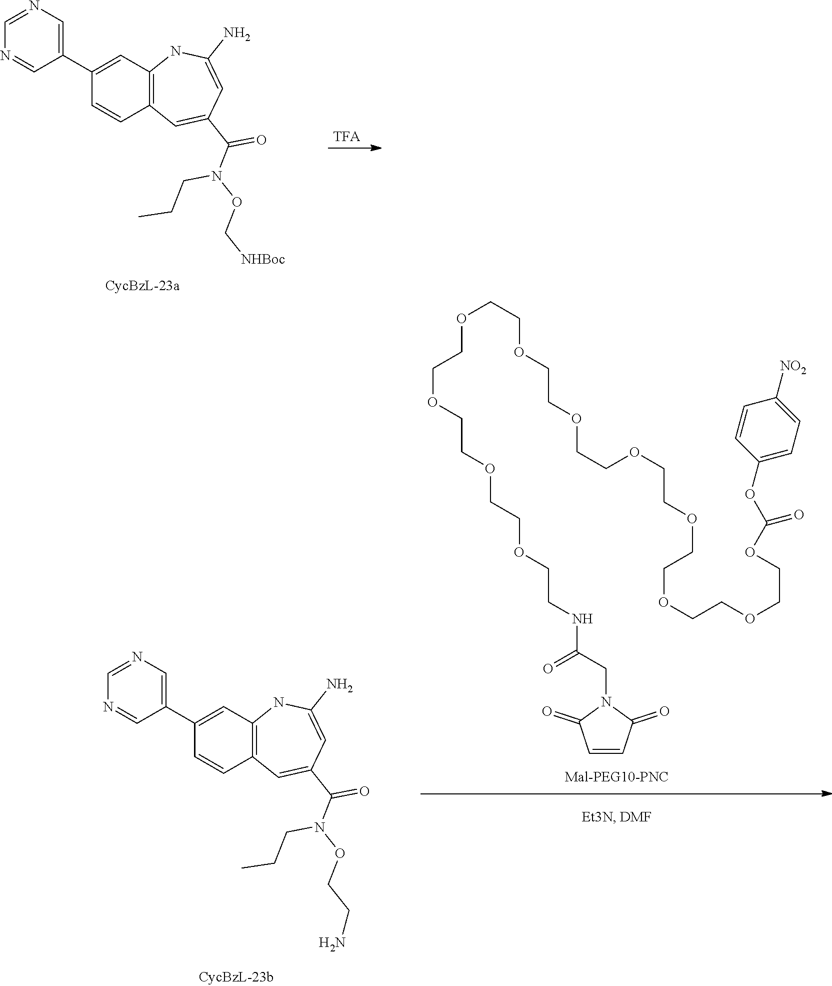 [SMILES: CCCN(OCNC(=O)OC(C)(C)C)C(=O)C1=Cc2ccc(-c3cncnc3)cc2NC(N)=C1 ; CCCN(OCCN)C(=O)C1=Cc2ccc(-c3cncnc3)cc2NC(N)=C1 ; O=C(CN1C(=O)C=CC1=O)NCCOCCOCCOCCOCCOCCOCCOCCOCCOCCOCCOC(=O)Oc1ccc([N+](=O)[O-])cc1]

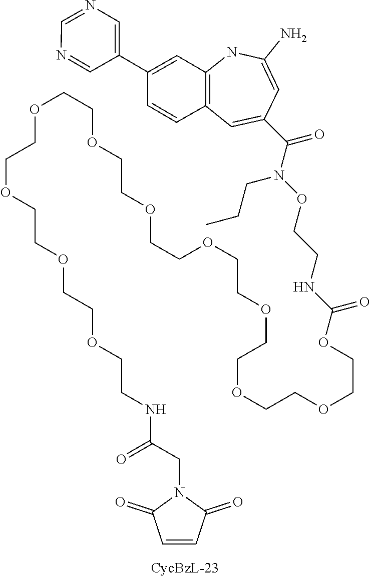 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(-c3cncnc3)cc2NC(N)=C1]

### Preparation of 2-amino-N-(2-aminoethoxy)-N-propyl-8-(pyrimidin-5-yl)-3H-benzo[b]azepine-4-carboxamide, CycBzL-23b

tert-Butyl (2-((2-amino-N-propyl-8-(pyrimidin-5-yl)-3H-benzo[b]azepine-4-carboxamido)oxy)ethyl)carbamate, CycBzL-23a (5.1 mg, 0.011 mmol, 1 eq) was suspended in 100 μl TFA. After 30 minutes, the solution was concentrated to give CycBzL-23b as a TFA salt (7.4 mg, 0.012 mmol, 100%). LC/MS [M+H] 381.2 (calculated); LC/MS [M+H] 381.3 (observed).

### Preparation of CycBzL-23

To a solution of CycBzL-23b (7.4 mg, 0.012 mmol, 1 eq) in DMF (0.5 ml) was added triethylamine (13.5 μl, 0.097 mmol, 8 eq) and 1-(2,5-dioxo-2,5-dihydro-1H-pyrrol-1-yl)-2-oxo-6,9,12,15,18,21,24,27,30,33-decaoxa-3-azapentatriacontan-35-yl (4-nitrophenyl) carbonate, Mal-PEG10-PNC (15.6 mg, 0.019 mmol, 1.6 eq). The solution was stirred at room temperature and monitored by LC/MS. Upon consumption of amine starting material, the reaction was concentrated and purified by prep-HPLC to obtain CycBzL-23. LC/MS [M+H] 1045.5 (calculated); LC/MS [M+H] 1045.8 (observed).

### Example 201 Preparation of Immunoconjugates (IC)

To prepare a lysine-conjugated Immunoconjugate, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO) or Zeba™ Spin Desalting Columns (Thermo Fisher Scientific). The eluates are then each adjusted to a concentration of about 1-10 mg/ml using the buffer and then sterile filtered. The antibody is pre-warmed to 20-30° C. and rapidly mixed with 2-20 (e.g., 7-10) molar equivalents of a tetrafluorophenyl (TFP) or sulfonic tetrafluorophenyl (sulfoTFP) ester, 8-cyclyl-2-aminobenzazepine-linker (CycBzL) compound of Formula II dissolved in dimethylsulfoxide (DMSO) or dimethylacetamide (DMA) to a concentration of 5 to 20 mM. The reaction is allowed to proceed for about 16 hours at 30° C. and the immunoconjugate (IC) is separated from reactants by running over two successive G-25 desalting columns or Zeba™ Spin Desalting Columns equilibrated in phosphate buffered saline (PBS) at pH 7.2 to provide the Immunoconjugate (IC) of Tables 3a and 3b. Adjuvant-antibody ratio (DAR) is determined by liquid chromatography mass spectrometry analysis using a C₄ reverse phase column on an ACQUITY™ UPLC H-class (Waters Corporation, Milford, MA) connected to a XEVO™ G2-XS TOF mass spectrometer (Waters Corporation).

To prepare a cysteine-conjugated Immunoconjugate, an antibody is buffer exchanged into a conjugation buffer containing PBS, pH 7.2 with 2 mM EDTA using Zeba™ Spin Desalting Columns (Thermo Fisher Scientific). The interchain disulfides are reduced using 2-4 molar excess of Tris (2-carboxyethyl) phosphine (TCEP) or dithiothreitol (DTT) at 37° C. for 30 min—2 hours. Excess TCEP or DTT was removed using a Zeba™ Spin Desalting column pre-equilibrated with the conjugation buffer. The concentration of the buffer-exchanged antibody was adjusted to approximately 5 to 20 mg/ml using the conjugation buffer and sterile-filtered. The maleimide-CycBzL compound is either dissolved in dimethylsulfoxide (DMSO) or dimethylacetamide (DMA) to a concentration of 5 to 20 mM. For conjugation, the antibody is mixed with 10 to 20 molar equivalents of maleimide-HxBzL. In some instances, additional DMA or DMSO up to 20% (v/v), was added to improve the solubility of the maleimide-CycBzL in the conjugation buffer. The reaction is allowed to proceed for approximately 30 min to 4 hours at 20° C. The resulting conjugate is purified away from the unreacted maleimide-CycBzL using two successive Zeba™ Spin Desalting Columns. The columns are pre-equilibrated with phosphate-buffered saline (PBS), pH 7.2. Adjuvant to antibody ratio (DAR) is estimated by liquid chromatography mass spectrometry analysis using a C₄ reverse phase column on an ACQUITY™ UPLC H-class (Waters Corporation, Milford, MA) connected to a XEVO™ G2-XS TOF mass spectrometer (Waters Corporation).

For conjugation, the antibody may be dissolved in an aqueous buffer system known in the art that will not adversely impact the stability or antigen-binding specificity of the antibody. Phosphate buffered saline may be used. The CycBzL compound is dissolved in a solvent system comprising at least one polar aprotic solvent as described elsewhere herein. In some such aspects, CycBzL is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the 8-Het-2-aminobenzazepine-linker intermediate is dissolved in DMSO (dimethylsulfoxide), DMA (dimethylacetamide), acetonitrile, or another suitable dipolar aprotic solvent.

Alternatively in the conjugation reaction, an equivalent excess of CycBzL solution may be diluted and combined with antibody solution. The CycBzL solution may suitably be diluted with at least one polar aprotic solvent and at least one polar protic solvent, examples of which include water, methanol, ethanol, n-propanol, and acetic acid. The molar equivalents of 8-Het-2-aminobenzazepine-linker intermediate to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If antibody thiol groups are reacting with a thiol-reactive group such as maleimide of the 8-Het-2-aminobenzazepine-linker intermediate, unreacted antibody thiol groups may be reacted with a capping reagent. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the immunoconjugate, such in 20 mM sodium succinate, pH 5. The diluted solution is applied to a cation exchange column followed by washing with, e.g., at least column volumes of 20 mM sodium succinate, pH 5. The conjugate may be suitably eluted with a buffer such as PBS.

### Example 202 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory molecules. Activity was measured using a plate reader at 620-655 nm wavelength.

### Example 203 Assessment of Immunoconjugate Activity In Vitro

This example shows that Immunoconjugates of the invention are effective at eliciting immune activation, and therefore are useful for the treatment of cancer.
a) Isolation of Human Antigen Presenting Cells: Human myeloid antigen presenting cells (APCs) were negatively selected from human peripheral blood obtained from healthy blood donors (Stanford Blood Center, Palo Alto, California) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.
b) Myeloid APC Activation Assay: 2×10⁵ APCs are incubated in 96-well plates (Corning, Corning, NY) containing iscove's modified dulbecco's medium, IMDM (Lonza) supplemented with 10% FBS, 100 U/mL penicillin, 100 μg/mL (micrograms per milliliter) streptomycin, 2 mM L-glutamine, sodium pyruvate, non-essential amino acids, and where indicated, various concentrations of unconjugated (naked) antibodies and immunoconjugates of the invention (as prepared according to the Example above). Cell-free supernatants are analyzed after 18 hours via ELISA to measure TNF (a secretion as a readout of a proinflammatory response.
c) PBMC Activation Assay: Human peripheral blood mononuclear cells were isolated from human peripheral blood obtained from healthy blood donors (Stanford Blood Center, Palo Alto, California) by density gradient centrifugation. PBMCs were incubated in 96-well plates (Corning, Corning, NY) in a co-culture with CEA-expressing tumor cells (e.g. MKN-45, HPAF-II) at a 10:1 effector to target cell ratio. Cells were stimulated with various concentrations of unconjugated (naked) antibodies and immunoconjugates of the invention (as prepared according to the Example above). Cell-free supernatants were analyzed by cytokine bead array using a LegendPlex™ kit according to manufacturer's guidelines (BioLegend®, San Diego, CA).
d) Isolation of Human Conventional Dendritic Cells: Human conventional dendritic cells (cDCs) were negatively selected from human peripheral blood obtained from healthy blood donors (Stanford Blood Center, Palo Alto, California) by density gradient centrifugation. Briefly, cells are first enriched by using a ROSETTESEP™ Human CD3 Depletion Cocktail (Stem Cell Technologies, Vancouver, Canada) to remove T cells from the cell preparation. cDCs are then further enriched via negative selection using an EASYSEP™ Human Myeloid DC Enrichment Kit (Stem Cell Technologies).
e) cDC Activation Assay: 8×10⁴ APCs were co-cultured with tumor cells expressing the ISAC target antigen at a 10:1 effector (cDC) to target (tumor cell) ratio. Cells were incubated in 96-well plates (Corning, Corning, NY) containing RPMI-1640 medium supplemented with 10% FBS, and where indicated, various concentrations of the indicated immunoconjugate of the invention (as prepared according to the example above). Following overnight incubation of about 18 hours, cell-free supernatants were collected and analyzed for cytokine secretion (including TNFα) using a BioLegend LEGENDPLEX cytokine bead array.

Activation of myeloid cell types can be measured using various screen assays in addition to the assay described in which different myeloid populations are utilized. These may include the following: monocytes isolated from healthy donor blood, M-CSF differentiated Macrophages, GM-CSF differentiated Macrophages, GM-CSF+IL-4 monocyte-derived Dendritic Cells, conventional Dendritic Cells (cDCs) isolated from healthy donor blood, and myeloid cells polarized to an immunosuppressive state (also referred to as myeloid derived suppressor cells or MDSCs). Examples of MDSC polarized cells include monocytes differentiated toward immunosuppressive state such as M2a MΦ(IL4/IL13), M2c MΦ(IL10/TGFb), GM-CSF/IL6 MDSCs and tumor-educated monocytes (TEM). TEM differentiation can be performed using tumor-conditioned media (e.g. 786.0, MDA-MB-231, HCC1954). Primary tumor-associated myeloid cells may also include primary cells present in dissociated tumor cell suspensions (Discovery Life Sciences).

Assessment of activation of the described populations of myeloid cells may be performed as a mono-culture or as a co-culture with cells expressing the antigen of interest which the immunoconjugate may bind to via the CDR region of the antibody. Following incubation for 18-48 hours, activation may be assessed by upregulation of cell surface co-stimulatory molecules using flow cytometry or by measurement of secreted proinflammatory cytokines. For cytokine measurement, cell-free supernatant is harvested and analyzed by cytokine bead array (e.g. LegendPlex from Biolegend) using flow cytometry.

All references, including publications, patent applications, and patents, cited herein are hereby incorporated by reference to the same extent as if each reference were individually and specifically indicated to be incorporated by reference and were set forth in its entirety herein.

1. An immunoconjugate comprising an antibody covalently attached to one or more 8-Cyc-2-aminobenzazepine moieties by a linker, and having Formula I:
Ab-[L-CycBz]ₚ I
or a pharmaceutically acceptable salt thereof,
wherein:
Ab is an antibody construct that has an antigen binding domain that binds PD-L1;
p is an integer from 1 to 8;
CycBz is the 8-Cyc-2-aminobenzazepine moiety having the formula: 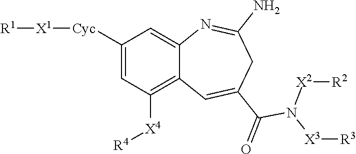
Cyc is selected from the group consisting of phenyldiyl, heterocyclyldiyl and heteroaryldiyl;
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₆-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—(C₁-C₂₀ heteroaryl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵—(C₁-C₁₂ alkyldiyl-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₁₂ alkyl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—O—C(═O)N(R⁵)₂;
—O—C(═O)N(R⁵¹—*:
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), 0, N(R⁵), S, S(O)₂, and S(O)₂N(R⁵);
R⁵ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
—C(═O)—PEG-;
—C(═O)—PEG-C(═O)N(R⁶)—(C₁-C₁₂ alkyldiyl)-C(═O)-Gluc-;
—C(═O)—PEG-O—;
—C(═O)—PEG-O—C(═O)—;
—C(═O)—PEG-C(═O)—;
—C(═O)—PEG-C(═O)—PEP-;
—C(═O)—PEG-N(R⁶)—;
—C(═O)—PEG-N(R⁶)—C(═O)—;
—C(═O)—PEG-N(R⁶)—PEG-C(═O)—PEP-;
—C(═O)—PEG-N⁺(R⁶)₂-PEG-C(═O)—PEP-;
—C(═O)—PEG-C(═O)—PEP-N(R⁶)—(C₁-C₁₂ alkyldiyl)-;
—C(═O)—PEG-C(═O)—PEP-N(R⁶)—(C₁-C₁₂ alkyldiyl)N(R⁶)C(═O)—(C₂-C₅ monoheterocyclyldiyl)-;
—C(═O)—PEG-SS—(C₁-C₁₂ alkyldiyl)-OC(═O)—;
—C(═O)—PEG-SS—(C₁-C₁₂ alkyldiyl)-C(═O)—;
—C(═O)—(C₁-C₁₂ alkyldiyl)-C(═O)—PEP-;
—C(═O)—(C₁-C₁₂ alkyldiyl)-C(═O)—PEP-N(R⁶)—(C₁-C₁₂ alkyldiyl)-;
—C(═O)—(C₁-C₁₂ alkyldiyl)-C(═O)—PEP-N(R⁶)—(C₁-C₁₂ alkyldiyl)-N(R⁵)—C(═O);
—C(═O)—(C₁-C₁₂ alkyldiyl)-C(═O)—PEP-N(R⁶)—(C₁-C₁₂ alkyldiyl)-N(R⁶)C(═O)—(C₂-C₅ monoheterocyclyldiyl)-;
-succinimidyl-(CH₂)ₘ—C(═O)N(R⁶)—PEG-;
-succinimidyl-(CH₂)ₘ—C(═O)N(R⁶)—PEG-C(═O)N(R⁶)—(C₁-C₁₂ alkyldiyl)-C(═O)-Gluc-;
-succinimidyl-(CH₂)ₘ—C(═O)N(R⁶)—PEG-O—;
-succinimidyl-(CH₂)ₘ—C(═O)N(R⁶)—PEG-O—C(═O)—;
-succinimidyl-(CH₂)ₘ—C(═O)N(R⁶)—PEG-C(═O)—;
-succinimidyl-(CH₂)ₘ—C(═O)N(R⁶)—PEG-N(R⁵)—;
-succinimidyl-(CH₂)ₘ—C(═O)N(R⁶)—PEG-N(R⁵)—C(═O)—;
-succinimidyl-(CH₂)ₘ—C(═O)N(R⁶)—PEG-C(═O)—PEP-;
-succinimidyl-(CH₂)ₘ—C(═O)N(R⁶)—PEG-SS—(C₁-C₁₂ alkyldiyl)-OC(═O)—;
-succinimidyl-(CH₂)ₘ—C(═O)—PEP-N(R⁶)—(C₁-C₁₂ alkyldiyl)-;
-succinimidyl-(CH₂)ₘ—C(═O)—PEP-N(R⁶)—(C₁-C₁₂ alkyldiyl)N(R⁶)C(═O)—; and
-succinimidyl-(CH₂)ₘ—C(═O)—PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ—; m is an integer from 1 to 5, and n is an integer from 2 to 50;
Glue has the formula: 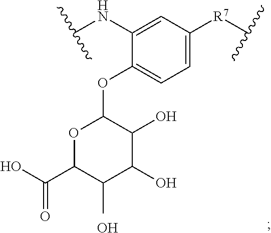
PEP has the formula: 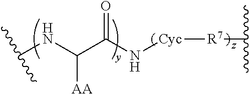
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 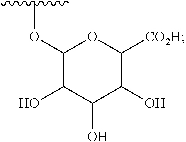 [SMILES: *OC1OC(C(=O)O)C(O)C(O)C1O]
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

2. The immunoconjugate of claim 1 wherein the antibody is selected from the group consisting of atezolizumab, durvalumab, and avelumab.

3. The immunoconjugate of claim 1 wherein the antibody construct is a Type A PD-L1 antibody and comprises an immunoglobulin heavy chain variable region polypeptide and an immunoglobulin light chain variable region polypeptide, wherein:
the immunoglobulin heavy chain variable region polypeptide comprises a complementarity determining region 1 (HCDR1) comprising any one of Type A SEQ ID NOs: 1-23, a complementarity determining region 2 (HCDR2) comprising any one of SEQ ID NOs: 24-57, and a complementarity determining region 3 (HCDR3) comprising any one of SEQ ID NOs: 58-95; or
the immunoglobulin light chain variable region polypeptide comprises a complementarity determining region 1 (LCDR1) comprising any one of SEQ ID NOs: 96-128, a complementarity determining region 2 (LCDR2) comprising any one of SEQ ID NOs: 129-151, and a complementarity determining region 3 (LCDR3) comprising any one of SEQ ID NOs: 152-155.

4. The immunoconjugate of claim 1 wherein the antibody construct is a Type A PD-L1 antibody and comprises an immunoglobulin heavy chain variable region of any one of SEQ ID NOs: 223-264 or at least the CDRs thereof, and an immunoglobulin light chain variable region of any one of SEQ ID NOs: 265-306 or at least the CDRs thereof.

5. The immunoconjugate of claim 1 wherein the antibody construct is a Type A PD-L1 antibody and comprises an immunoglobulin heavy chain variable region polypeptide with an amino acid sequence that is at least 90% identical to any one of SEQ ID NOs: 223-264, and an immunoglobulin light chain variable region polypeptide with an amino acid sequence that is at least 90% identical to any one of SEQ ID NOs: 265-306.

6. The immunoconjugate of claim 1 wherein the antibody construct is a Type A PD-L1 antibody and comprises the heavy and light chain immunoglobulin polypeptides, or at least the CDRs thereof.

7. The immunoconjugate of claim 1 wherein the antibody construct is a Type B PD-L1 antibody and comprises an immunoglobulin heavy chain variable region polypeptide and an immunoglobulin light chain variable region polypeptide, wherein:
the immunoglobulin heavy chain variable region polypeptide comprises a complementarity determining region 1 (HCDR1) comprising any one of SEQ ID NOs: 1-14, a complementarity determining region 2 (HCDR2) comprising any one of SEQ ID NOs: 15-31, and a complementarity determining region 3 (HCDR3) comprising any one of SEQ ID NOs: 32-52; or
the immunoglobulin light chain variable region polypeptide comprises a complementarity determining region 1 (LCDR1) comprising any one of SEQ ID NOs: 53-67, a complementarity determining region 2 (LCDR2) comprising any one of SEQ ID NOs: 68-79, and a complementarity determining region 3 (LCDR3) comprising any one of SEQ ID NOs: 80-91.

8. The immunoconjugate of claim 1 wherein the antibody construct is a Type B PD-L1 antibody and comprises an immunoglobulin heavy chain variable region of any one of SEQ ID NOs: 123-143 or at least the CDRs thereof; and an immunoglobulin light chain variable region of any one of SEQ ID NOs: 144-164 or at least the CDRs thereof.

9. The immunoconjugate of claim 1 wherein the antibody construct is a Type B PD-L1 antibody and comprises an immunoglobulin heavy chain variable region polypeptide with an amino acid sequence that is at least 90% identical to any one of SEQ ID NOs: 123-143, and an immunoglobulin light chain variable region polypeptide with an amino acid sequence that is at least 90% identical to any one of SEQ ID NOs: 144-164.

10. The immunoconjugate of claim 1 wherein the antibody construct is a Type B PD-L1 antibody and comprises the heavy and light chain immunoglobulin polypeptides, or at least the CDRs thereof.

11. The immunoconjugate of claim 1 wherein X¹ is a bond, and R¹ is H.

12. The immunoconjugate of claim 1 wherein X² is a bond, and R² is C₁-C₈ alkyl.

13. The immunoconjugate of claim 1 wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

14. The immunoconjugate of claim 13 wherein R² is C₁-C₈ alkyl and R³ is —(C₁-C₈ alkyldiyl)-N(R⁵)CO₂R⁵.

15. The immunoconjugate of claim 14 wherein R² is —CH₂CH₂CH₃ and R³ is selected from —CH₂CH₂CH₂NHCO₂(t-Bu), —OCH₂CH₂NHCO₂(cyclobutyl), and —CH₂CH₂CH₂NHCO₂(cyclobutyl).

16. The immunoconjugate of claim 13 wherein R² and R³ are each independently selected from —CH₂CH₂CH₃, —OCH₂CH₃, —OCH₂CF₃, —CH₂CH₂CF₃, —OCH₂CH₂OH, and —CH₂CH₂CH₂OH.

17. The immunoconjugate of claim 16 wherein R² and R³ are each —CH₂CH₂CH₃.

18. The immunoconjugate of claim 16 wherein R² is —CH₂CH₂CH₃ and R³ is —OCH₂CH₃.

19. The immunoconjugate of claim 1 wherein X³—R³ is selected from the group consisting of:

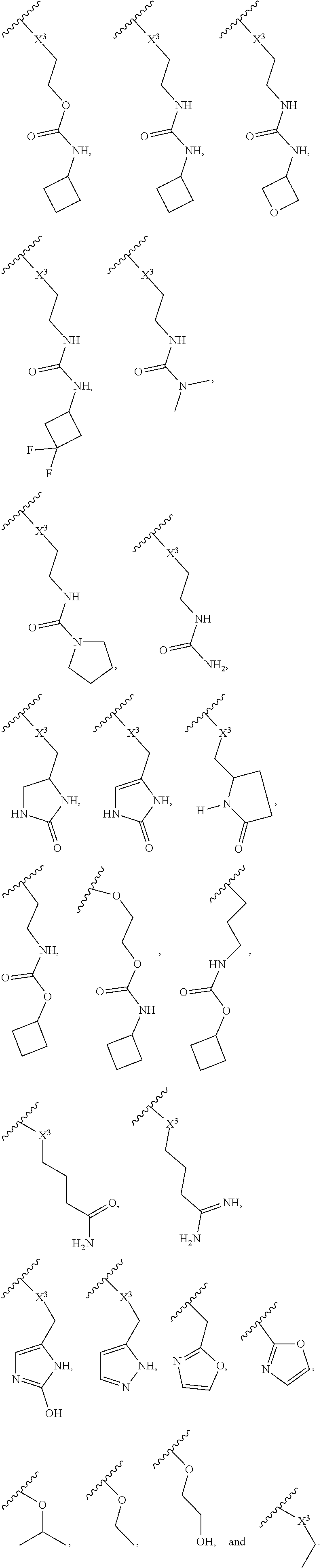

20. The immunoconjugate of claim 1 wherein X⁴ is a bond, and R⁴ is H.

21. The immunoconjugate of claim 1 wherein R¹ is attached to L.

22. The immunoconjugate of claim 1 wherein R² or R³ is attached to L.

23. The immunoconjugate of claim 22 wherein X³—R³-L is selected from the group consisting of: 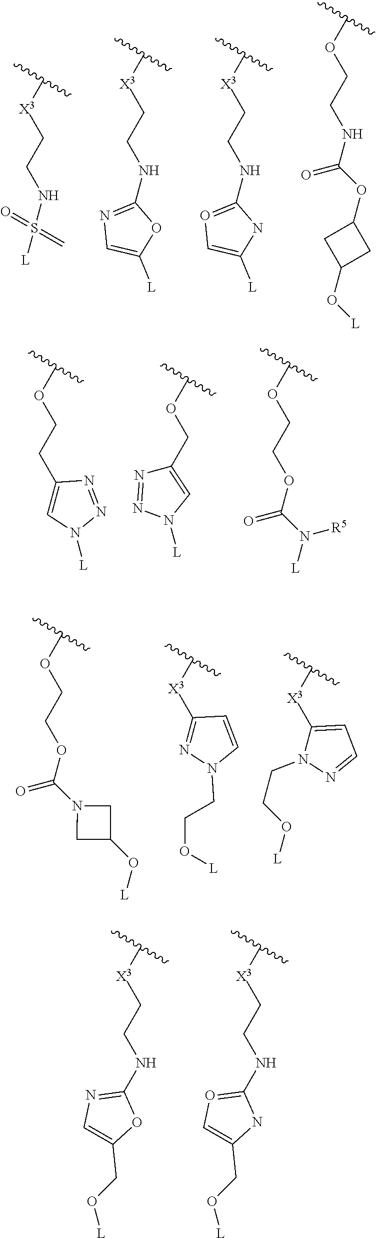
where the wavy line indicates the point of attachment to N.

24. (canceled)

25. (canceled)

26. The immunoconjugate of claim 1 wherein L is —C(═O)—PEG- or —C(═O)—PEG-C(═O)—.

27. The immunoconjugate of claim 1 wherein L is attached to a cysteine thiol of the antibody.

28. The immunoconjugate of claim 1 wherein for the PEG, m is 1 or 2, and n is an integer from 2 to 10.

29. The immunoconjugate of claim 28 wherein n is 10.

30-39. (canceled)

40. The immunoconjugate of claim 1 wherein L is selected from the structures: 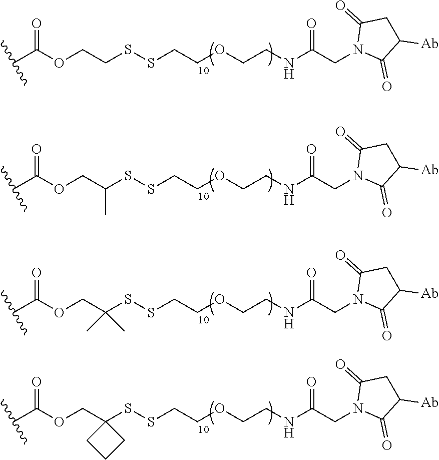
where the wavy line indicates the attachment to R⁵.

41. The immunoconjugate of claim 1 having Formula Ia:

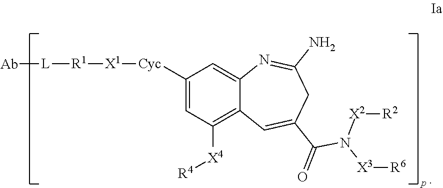

42. The immunoconjugate of claim 41 wherein X⁴ is a bond and R⁴ is H.

43. The immunoconjugate of claim 41 wherein Cyc is selected from the group consisting of pyridyldiyl, pyrimidyldiyl, pyrazolyldiyl, piperazinyldiyl, piperidinyldiyl, and pyrazinyldiyl.

44. The immunoconjugate of claim 41 wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

45. The immunoconjugate of claim 41 wherein X² is O.

46. The immunoconjugate of claim 41 selected from Formulae Ib-Ii:

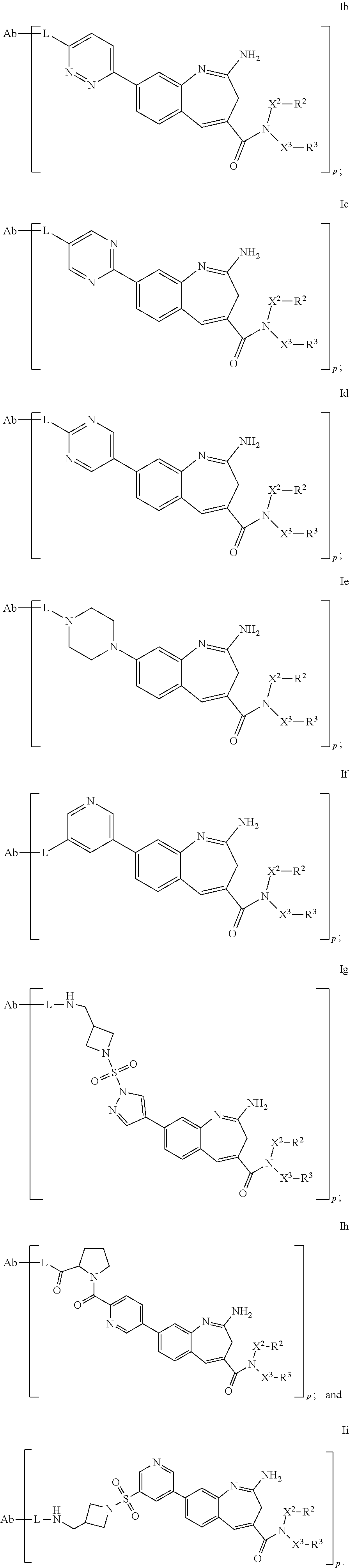

47. The immunoconjugate of claim 41 selected from Formulae Ij-In:

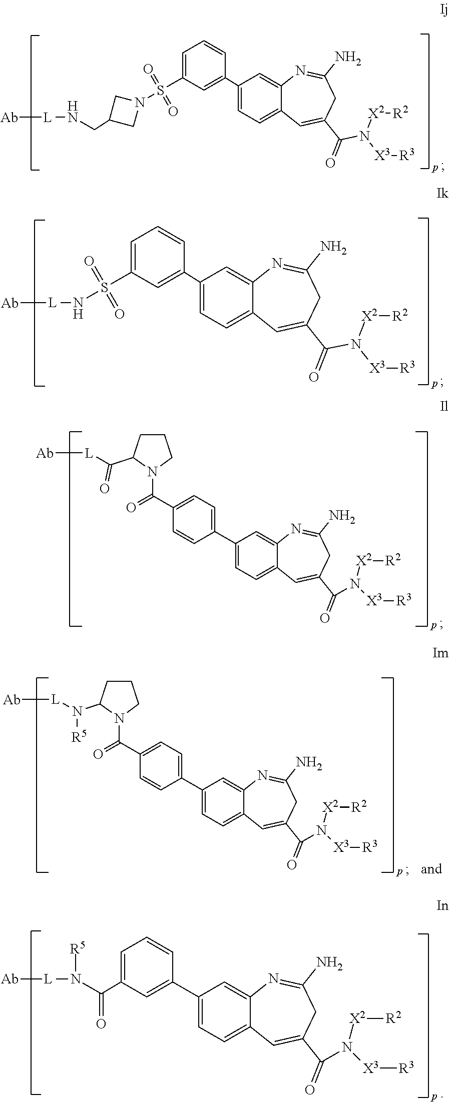

48. The immunoconjugate of claim 41 wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

49. The immunoconjugate of claim 41 wherein X² and X³ are each a bond, R² is C₁-C₈ alkyl, and R³ is selected from —O—(C₁-C₁₂ alkyl) and —O—(C₁-C₁₂ alkyl)-N(R⁵)CO₂R⁵.

50. A 8-Cyc-2-aminobenzazepine-linker compound selected from the group consisting of:

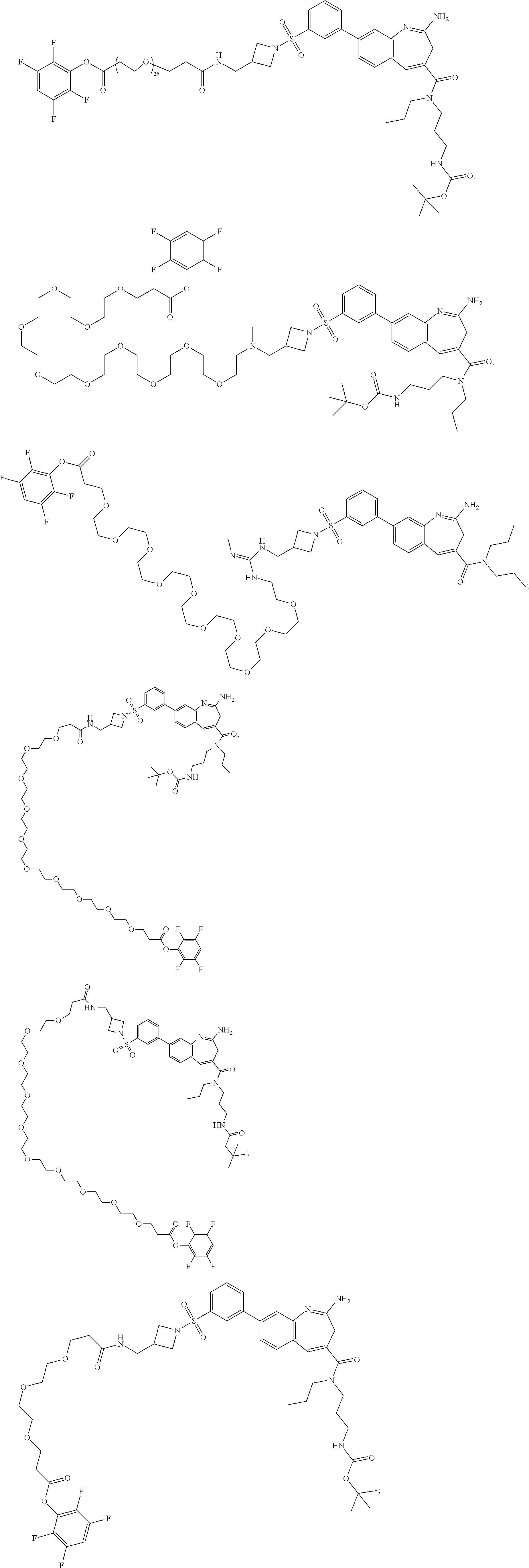

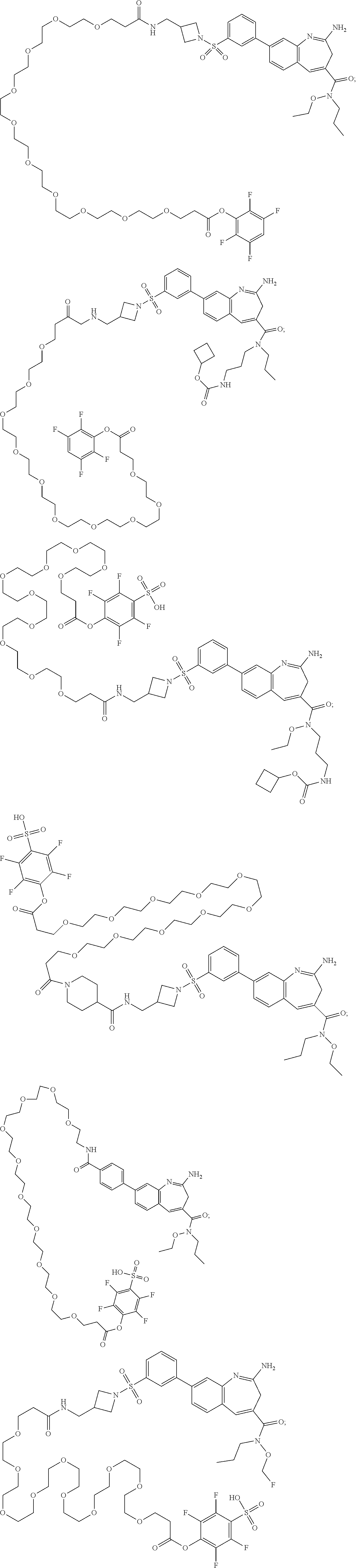

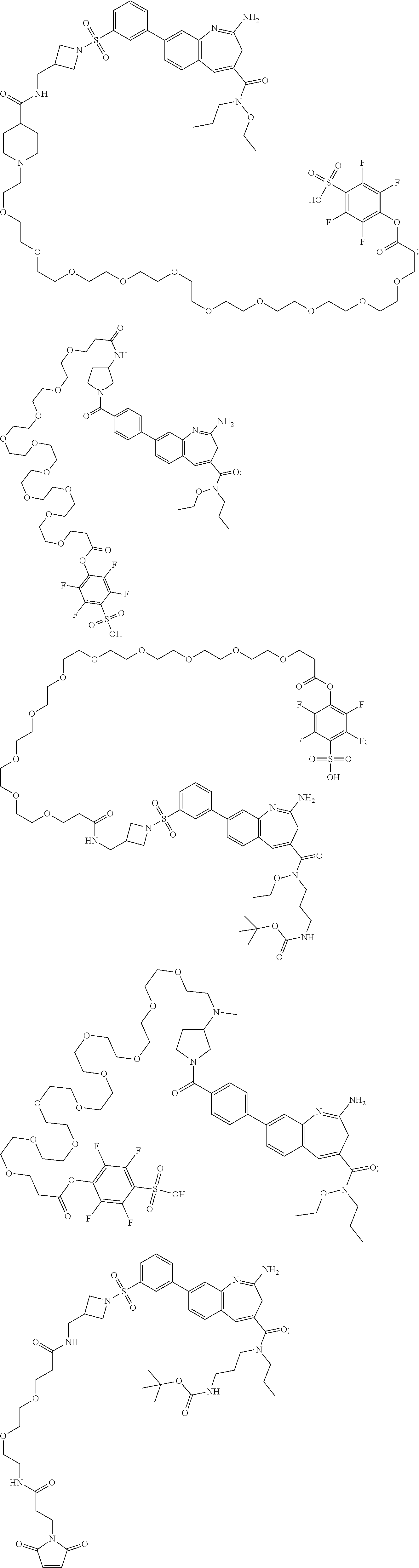

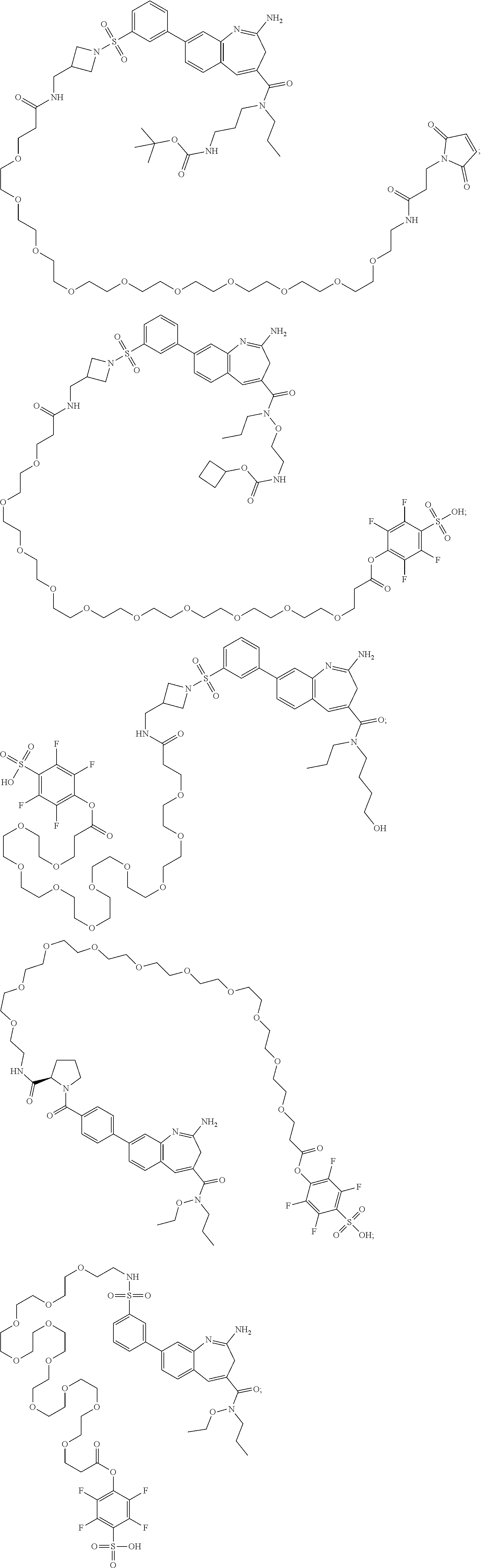

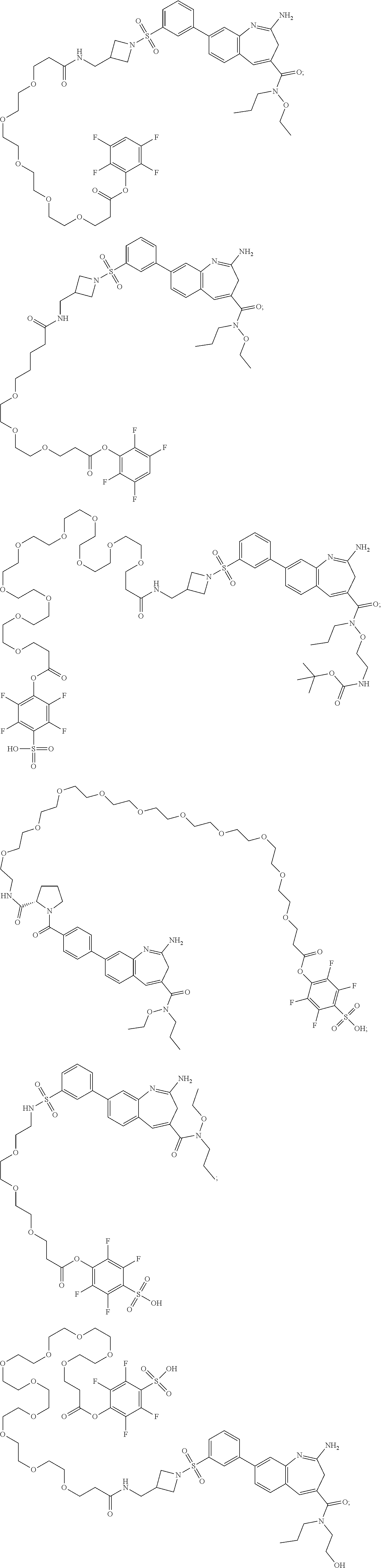

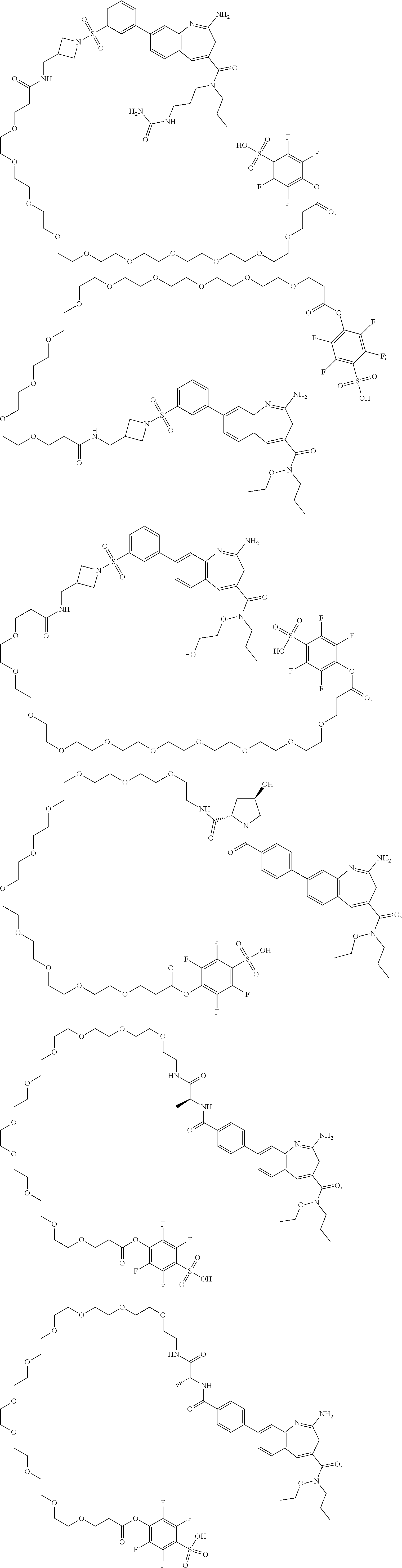

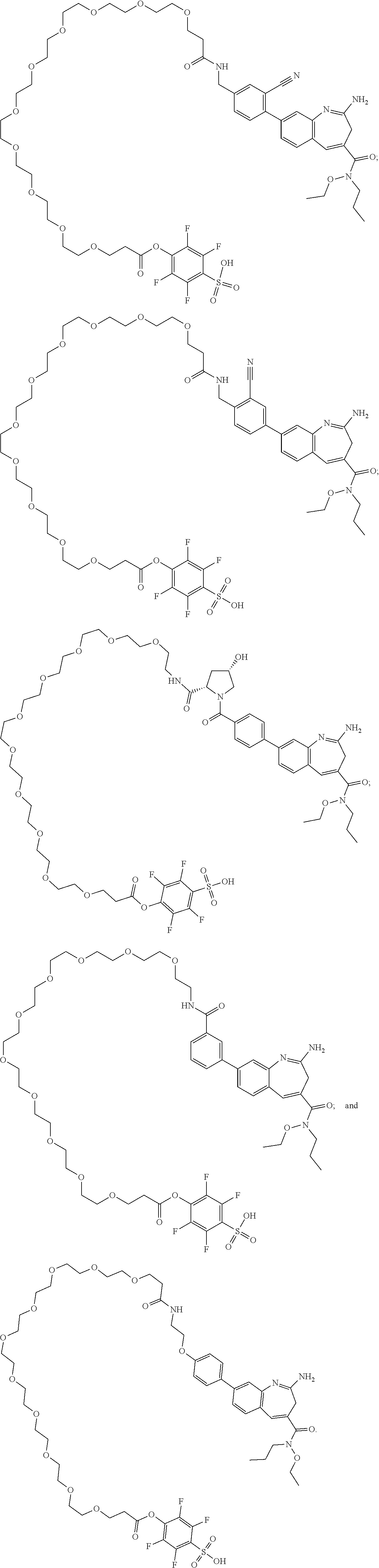

51. An immunoconjugate prepared by conjugation of an anti PD-L1 antibody with a 8-Cyc-2-aminobenzazepine-linker compound selected from claim 50.

52. A pharmaceutical composition comprising a therapeutically effective amount of an immunoconjugate according to claim 1, and one or more pharmaceutically acceptable diluent, vehicle, carrier or excipient.

53. A method for treating cancer comprising administering a therapeutically effective amount of an immunoconjugate according to claim 1, to a patient in need thereof, wherein the cancer is selected from bladder cancer, urinary tract cancer, urothelial carcinoma, lung cancer, non-small cell lung cancer, Merkel cell carcinoma, colon cancer, colorectal cancer, gastric cancer, and breast cancer.

54. The method of claim 53, wherein the cancer is susceptible to a pro-inflammatory response induced by TLR7 and/or TLR8 agonism.

55. The method of claim 53, wherein the cancer is a PD-L1-expressing cancer.

56. The method of claim 53, wherein the cancer is selected from the group consisting of triple-negative breast cancer, metastatic Merkel cell carcinoma, and gastroesophageal junction adenocarcinoma.

57. (canceled)

58. (canceled)

59. The method of claim 53, wherein the immunoconjugate is administered to the patient intravenously, intratumorally, or subcutaneously.

60. The method of claim 53, wherein the immunoconjugate is administered to the patient at a dose of about 0.01 to 20 mg per kg of body weight.

61. (canceled)

62. A method of preparing an immunoconjugate of Formula I of claim 1 wherein the 8-Cyc-2-amino-thienoazepine-linker compound of claim 50 is conjugated with the anti PD-L1 antibody.
